(19) United States
(12) Patent Application Publication
(10) Pub. No.: US 2021/0087337 A1
(43) Pub. Date: Mar. 25, 2021

(54) COMPOUNDS, COMPOSITIONS AND METHODS RELATED TO ANTIMICROBIAL APPLICATIONS

(71) Applicant: VYOME THERAPEUTICS LIMITED, New Delhi (IN)

(72) Inventors: Sumana GHOSH, Delhi (IN); Mau SINHA, Uttar Pradesh Noida (IN); Anamika BHATTACHARYYA, Patparganj Delhi (IN); Suresh SADHASIVAM, Tamil Nadu Salem (IN); Shamik GHOSH, Uttar Pradesh Ghaziabad (IN); Ritwik SAMANTA, West Bengal Bankura (IN); Nupur TANDON, Uttar Pradesh Noida (IN)

(73) Assignee: VYOME THERAPEUTICS LIMITED, New Delhi (IN)

(21) Appl. No.: 16/329,704

(22) PCT Filed: Aug. 31, 2017
(86) PCT No.: PCT/IB2017/055245

§ 371 Date: Jun. 4, 2019

(30) Foreign Application Priority Data
Aug. 31, 2016 (IN) 201611029743

(51) Int. Cl. C08G 73/02; A61K 31/131; A61K 9/16

(52) U.S. Cl. CPC C08G 73/02; A61K 9/1635; A61K 9/1617; A61K 31/131

(57) ABSTRACT

The present disclosure is in the field of polymers and pharmaceuticals/antimicrobials. The disclosure provides compounds based on SNAP (synthetic novel antimicrobial polymer) technology, compositions and methods of managing microbial infections including surgical site infections (SSIs). The present compounds are used as a management/therapeutic strategy to target microbial infections and have advantages including excellent antimicrobial potency, biofilm disruption ability, broad spectrum activity against various organisms covering both gram negative and gram positive bacteria as well as fungal pathogens, and low toxicity profile to ensure a healthy therapeutic window for use in humans.

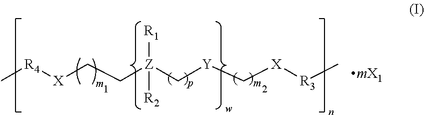

### TECHNICAL FIELD

The present disclosure is in the field of polymers and pharmaceuticals/antimicrobials. The present disclosure provides compounds based on SNAP (synthetic novel antimicrobial polymer) technology and methods of managing microbial infections including but not limiting to surgical site infections. The present compounds based on SNAP technology are used as a prophylaxis/prevention or as therapeutic strategy to target microbial infections.

### BACKGROUND OF THE DISCLOSURE

Wound is any physical disruption of the integument or mucous membrane causing tissue damage and trauma. Wounds can contract infections and can be categorized as “community-acquired” or “health-care associated” based on the source of the wound and/or infection. Community-acquired wound infections are often preceded by injuries resulting from occupational exposure (cuts and injuries at construction sites, burns, military activity related wounds) or recreational activities (spa, water parks, community swimming pools).

Health-care associated infections particularly, surgical site infections (SSIs) are infections that occur in the wound created by an invasive surgical procedure resulting in prolonged wound healing, abscess formation, and in severe cases, sepsis. SSIs are among the most prevalent type of acute or chronic wound infections that account for around 20% of all health-care associated infections (de Lissovoy et al., Am J Infect Control 2009; 37:387-97). These infections may be superficial or deep incisional infections, or infections involving organs or body spaces. SSIs generally occur within 30 days of a surgical procedure but can occur within a year (in case of medical device related surgeries).

SSIs can result from different surgical procedures including dermatologic, ophthalmic, otitic, subcutaneous tissue and breast; orthopedic; cardiovascular; neurological; colorectal; gastrointestinal and obstetric, gynecologic and dental. Medical devices used for surgeries are also highly susceptible to bacterial and fungal infections. The most common devices or implants used are orthopedic prostheses, fracture fixation devices, coronary stents, central venous and urinary catheters, heart valves, vascular grafts, central nervous system implants, cochlear and dental implants. Cutaneous wound infections beyond surgical procedures can also arise from cellulitis, insect bites, cuts and insect bites, burn wounds, diabetic foot ulcers, gangrenes, and infected wounds in military populations.

For most SSIs, the source of pathogens is the endogenous flora of the patient's skin, mucous membranes, or viscera. A variety of pathogens (both Gram positive and Gram negative bacteria) are involved in SSIs, the most common ones being Staphylococcus aureus (˜30%), Staphylococcus epidermidis (coagulase-negative Staphylococcus (˜14%), Propionibacterium acnes, Enterococcus (˜11%), Pseudomonas aeruginosa (˜5%), E. coli (˜9%), Enterobacter spp (˜4%), Acinetobacter baumannii, and Bacillus fragilis. SSIs are also associated with fungal infections including yeast species especially Candida spp (˜2%). Although majority of microbes residing in wounds are aerobic, anaerobes like Bacteroids spp., Fusobacterium sp., and Clostridium sp., are also detected in deeper tissues (Percival et al., Wound Rep Reg 2012; 20:647-57). In some cases, SSIs have also been caused by unusual pathogens, such as Rhizopus oryzae, Rhodococcus bronchialis, Nocardia farcinica, Legionella pneumophila Legionella dumoffii, and Pseudomonas multivorans. These rare outbreaks have been traced to contaminated adhesive dressings and bandages, tap water, or contaminated disinfectant solutions or even surgical personnel.

SSIs represent a significant clinical burden, in that patients are typically readmitted, often into intensive care units, and are at higher risk of further complications leading to significant patient morbidity, increased duration of hospitalization and considerable increase in treatment costs. SSIs are the third most frequently reported nosocomial infections, accounting for 14-16% of such infections among hospitalized patients and 38% among surgical patients (Mangram et al., 1999 Infect Control Hosp Epidemiol 20:247 278).

Early recognition along with prompt, appropriate and effective intervention is an absolute necessity for successful clinical outcomes of SSIs. The three practices generally recommended for managing SSI are use of an antimicrobial agent that covers pathogens specific to the planned operative procedure (pre-operative strategies), administration of an antibiotic to establish bactericidal tissue levels prior to skin incision (pre-operative strategies), and continued administration of an antimicrobial for a specified time after the procedure is completed (post-operative strategies). SSI management depends on the site of infection, severity of the infection and wound etiology.

For wound management, judicious prophylactic use of different topical antiseptics like ChloralPrep® [2% chlorhexidine digluconate (CHG)], DuraPrep™ [Iodine Povacrylex, (Iodine based co-polymer), 0.7% available iodine], Prontosan gel (0.1% PHMB), Silvadazin (1% silver sulphadiazine cream), triclosan etc. are used in both pre- and post-operative stages. Antiseptics are used mainly in the treatment of infected surgical and/or non-surgical open acute and chronic wounds. In case of severe infections or systemic infections, antiseptics are generally prescribed in conjunction with antibiotics. The use of prophylactic antibiotics in surgery is common in management of SSI. Currently, in orthopedic infections and implant associated bone infections, local antibiotic therapy using antibiotic loaded bone cements containing vancomycin, gentamicin, tobramycin etc. are widely used.

Despite advances in interventions towards reducing the rate of SSIs (17% decrease for 2014 as per the CDC Progress Report, 2016) by improving sterilization methods, surgical techniques and prescribing antimicrobial prophylaxis during many pre-operative and post-operative skin preparations, SSIs remain the second most chronic health care associated infections. There are several challenges that result in treatment failure of SSI. In case of wound management, antiseptics widely used suffer from drawbacks in terms of safety profiles, skin discoloration effect, reduced efficacy against resistant strains, inactivation in the presence of serum, leaching from impregnated dressings and limited activity against biofilm. Use of conventional antibiotics also result in treatment failure due to emergence of multi-drug resistant organisms like methicillin resistant S. aureus (MRSA), vancomycin resistant S. aureus (VRSA), vancomycin resistant Enterococci (VRE), drug-resistant S. epidermidis, Propionibacterium sp. and multidrug-resistant Gram negative pathogens like carbapenem-resistant Klebsiella, multi-drug resistant Acinetobacter and Pseudomonas, drug-resistant Clostridium sp., extended spectrum β-lactamase-producing Enterobacter, and drug-resistant fungal species like Candida sp. resulting in treatment failure by the current antibiotic regimen. Further, wounds are often infected with multiple drug resistant organisms and such diversity in wound flora presents a big therapeutic challenge and calls for development of alternate agents with broad spectrum antibacterial activity to achieve clinical efficacy.

Another major cause of treatment failure is the prevalence of ‘biofilm’ associated infections at surgical sites especially in the presence of foreign materials (e.g. implants or sutures). Biofilms are also found associated with infections at non-surgical deep wounds as those contracted by military personnel. Biofilms act as major treatment barrier including poor drug penetration and distribution through the bone. Presence of such biofilms delay the healing process and causes antimicrobial resistance due to unavailability of optimal concentrations of the active agent at infection site. Therefore, the bacteria or the fungi present deep within the structure remains unchallenged. Current reports suggest use of physically active anti-adhesive surfaces, coatings with various bactericidal materials and molecules, with quorum sensing quenchers. However, no efficient treatment has so far been identified or for effective biofilm eradication.

Additionally, sustained presence of infection at a wound site continually stimulates the host immune responses leading to the activation of inflammatory pathways. Hence eradication of the infection is important for effective healing processes to be recruited at the site of the wound, whether surgical or non-surgical. However, often the active agents that effectively clear infections also impair healing responses due to toxic side effects on host cells.

This backdrop necessitates developing a cost-effective technology for making safe, effective, stable, broad spectrum active agents with antimicrobial and potent biofilm inhibitory actions to prevent microbial infections or associated diseases/complications arising from the same. It is a pre-requisite that the antimicrobial agents should not impede the biological process of growth and tissue regeneration that will eventually result in wound healing. This in turn will reduce the incidence of infections, length of hospital stay, improve the quality of medical care and eventually lower the economic burden.

However, it is not a very easy goal to develop polymers having desired efficacy and at the same time lacking toxicity to human cells. Minimizing hemolytic properties of polymers becomes imperative because of their potential use in biomedical devices such as catheters, sutures, indwelling structures, prosthetics, etc. Any release of the polymer into systemic circulation can directly expose the red blood cells (RBCs) to the polymer. Therefore, there is a lot of interest and challenge in development of polymers through an in depth understanding of structure-activity relationships of the polymers and phospholipid residues of cell membranes.

The present disclosure tries to address the aforementioned challenges of the prior art by providing compounds/molecules and their applications in managing microbial infections including but not limiting to surgical site infections.

### STATEMENT OF THE DISCLOSURE

The present disclosure relates to a compound of Formula I:

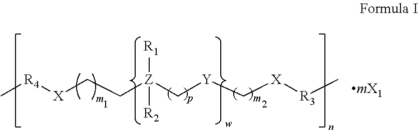

wherein, n=1 to 1000;
p=1 to 10;
w=1 to 10;
m₁=0 to 10;
m₂=0 to 10;
X is oxygen or —NH—;
m=1-1000;
X₁ is bromide, chloride, iodide, sulfate, bisulfate, phosphate, nitrate, trifluoroacetate, acetate, propionate, glycolate, succinate, valerate, oleate, palmitate, stearate, laurate, benzoate, lactate, phosphate, tosylate, citrate, maleate, fumarate, succinate, tartrate, ascorbate, napthylate, hydroxymaleate, mesylate, glucoheptonate, lactobionate, laurylsulphonate, phenylacetate, glutamate, benzoate, salicylate, sulfanilate, 2-acetoxybenzoate, fumarate, toluenesulfonate, methanesulfonate, ethane disulfonate, oxalate, isothionate, quaternary ammonium salt, or any other pharmaceutically acceptable salt, or any combinations thereof;
Z is carbon or nitrogen;
Y is —CH₂— or —NH— or functionalized amine;
R₁ and R₂ is independently

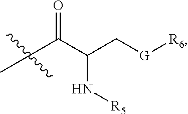

hydrogen, alkyl, straight alkyl chain, branched alkyl chain, —(CH₂)ₙ wherein n=1 to 30, alkenyl, alkynyl, aryl, heteroaryl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 and —(CH₂)ₚ—CH═CH₂ with p=1-10, polymer or polymer derivatives are —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), PEG, polylactic acid (PLA), PEG-PLA co-polymer, alginic acid, chitosan, PLGA, ethylene vinyl acetic acid, acrylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, carboxylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, polyester, polyamide, polycarbamate, polycarbonate, alkyl linked hybrid scaffold, cross-link polymeric scaffold, polybiguanidine, polyurethane, mixed polybiguanidine-polyurethane, polyurea, polyester, polyamide, polycarbonate, polycarbamate, polymethacrylate, polyvinyl, or any combinations of R₁ and R₂ thereof; and wherein each of the R₁ and R₂ is optionally substituted with primary, secondary, tertiary, quaternary amino group, hydroxyl group, thiol group, acrylic group, halogen selected from fluorine, chlorine, bromine or iodine, —COR₈ wherein R₈ is alkyl, alkenyl, monoenes, polyenes, terminally substituted alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, or C-terminal amino acids with D or L configuration, oligo-peptide, or any combinations thereof, wherein R₁ is optionally present;
and wherein in

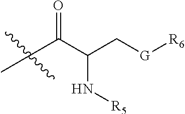

‘G’ is oxygen (—O—) or sulphur (—S—), R₅ and R₆ is independently selected from hydrogen, alkyl, straight alkyl chain, branched alkyl chain, —(CH₂)ₙ wherein n=1 to 30, alkenyl, alkynyl, aryl, heteroaryl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 or —(CH₂)ₚ—CH═CH₂ with p=1-10, and wherein each of R₅ and R₆ is optionally substituted with primary, secondary, tertiary or quaternary amino group, hydroxyl group, thiol group, carboxylic group, acrylic group or halogen selected from fluorine, chlorine, bromine or iodine, or any combinations thereof;
and wherein in —COR₇ and —COR₈, R₇ and R₈ is independently selected from alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl or heteroaryl, and wherein each of the R₇ and R₈ is optionally substituted with primary, secondary, tertiary or quaternary amino group, hydroxyl group, thiol group, carboxylic group, acrylic group, halogen selected from fluorine, chlorine, bromine or iodine; C-terminal amino acids with D or L configuration, or oligo-peptide, or any combinations thereof;
and
R₃ and R₄ is independently hydrogen, alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl or heteroaryl group, wherein each of the R₃ and R₄ is optionally substituted with primary, secondary, tertiary or quaternary amino group, hydroxyl group, thiol group, carboxylic group, acrylic group, halogen selected from fluorine, chlorine, bromine or iodine, —COR₈ with R₈ selected from alkyl, alkenyl, monoenes, polyenes, terminally substituted alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, oligopeptide, polymer or polymer derivatives selected from —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PLGA, ethylene vinyl acetic acid, polymethacrylate, polyvinyl, acrylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, carboxylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA, ethylene vinyl acetic acid, polyester, polyamide, polycarbamate, polycarbonate, alkyl linked hybrid scaffold, cross-link polymeric scaffold, polybiguanidine, polyurethane, mixed polybiguanidine-polyurethane, polyurea, polyester, polyamide, polycarbonate or polycarbamate or any combinations thereof; —C(NH)—NH—C(NH)—, —C(NH)—NH—C(NH)—NH—(CH₂)ₙ—NH—C(NH)—NH—C(NH)— with n=1-20, —C(NH)—NH—C(NH)—NH-M-NH—C(NH)—NH—C(NH)— wherein M is

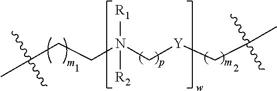

wherein R₁, R₂, Y, p, w, m₁ and m₂ is as defined above and R₁ is optional, —C(NH)—NH—C(NH)—NH—CH₂—(CH₂)ₘ—O(CO)NH—(CH₂)ₙ—NH(CO)—OCH₂—(CH₂)ₙ—NH—C(NH)—NH—C(NH)— with m=1-20, n=1-20, —CO—NH—(CH₂)ₙ—NH(CO)— with n=1-20, —CO—(CH₂)ₙ—(CO)— with n=1-20, —CO—NH—(CH₂)ₙ—NH(CO)— with n=1-20, [—CO—NH—(CH₂)ₙ—NH(CO)—(OCH₂—CH₂)ₘ—O(CO)—NH—] with m=1-20, n=1-20, —CO—(CH₂)ₙ—(CO)— with n=1-20, —COO—, —OCO—(CH₂CH₂—O)ₙ—CO— with n=1-20, —C(NH)—NH—C(NH)—NH—(CH₂)ₘ—O(CO)NH—(CH₂)ₙ—NH(CO)—O—(CH₂)ₙ—NH—C(NH)—NH—C(NH)— with m=1-20, n=1-20;

a compound of Formula II:

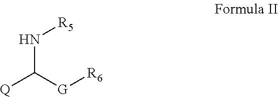

wherein ‘G’ is oxygen (—O—), sulphur (—S—), carbon (—C—), aryl, heteroaryl groups;
Q is carboxylic acid, vinylacrylate, methylacrylate, halogen selected from fluoride, bromide, chloride or iodide, alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 and —(CH₂)p-CH═CH₂ with p=1-10, and substituted with carboxylic group, acrylic group, halogen selected from fluorine, chlorine, bromine or iodine, wherein ‘G’ is oxygen (—O—), sulphur (—S—), wherein R₅ and R₆ is independently hydrogen, alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 and —(CH₂)p-CH═CH₂ with p=1-10, and wherein each of R₅ and R₆ is optionally substituted with primary, secondary, tertiary or quaternary amino group, hydroxyl group, thiol group, carboxylic group, acrylic group, halogen selected from fluorine, chlorine, bromine or iodine, —COR₇, —COR₈ wherein R7 and R8 is alkyl, straight alkyl chain, branched alkyl chain, —(CH₂)n wherein n=1 to 30, alkenyl, alkynyl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10, —(CH₂)p-CH═CH₂ with p=1-10, aryl, heteroaryl and wherein each of the R₇ and R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide;

a composition comprising a compound as defined above along with pharmaceutically acceptable excipient; a method of treating a microbial infection or disease comprising administering a compound or a composition as defined above, to a subject in need thereof; use of a compound or a composition as defined above in the manufacture of a medicament; and use of a compound or a composition as defined above for treating a microbial infection selected from bacterial infection, fungal infection, biofilm associated infection, or any combination thereof.

### BRIEF DESCRIPTION OF THE FIGURES

FIG. 1 depicts an overview of SNAP technology involving compounds of the present disclosure along with various modes of application of said compounds.

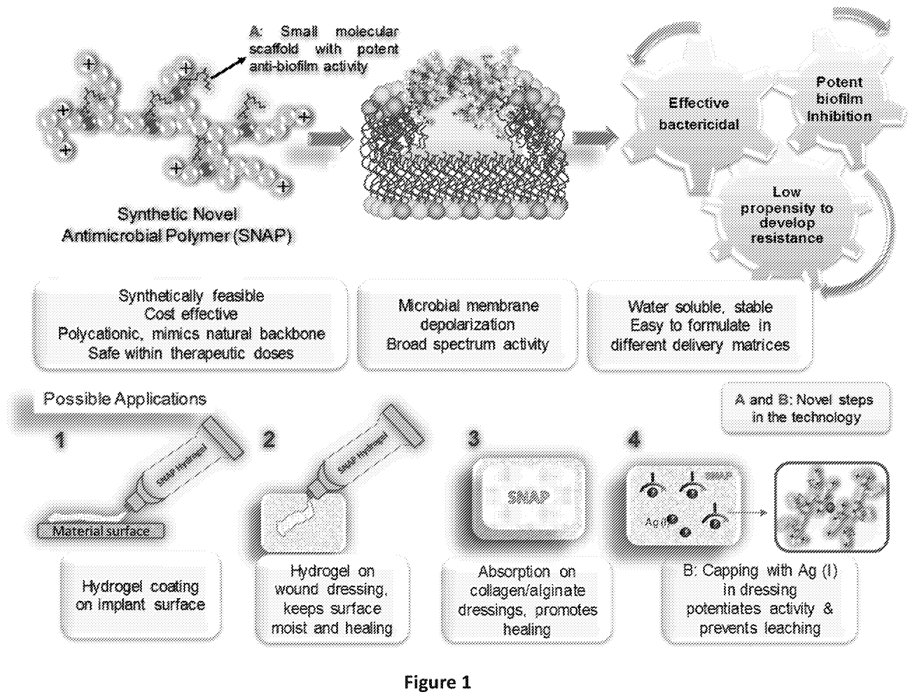

FIG. 2 provides bright-field micrographs of NIH-3T3 cells in a scratch assay to study the effect of the compounds as a representative wound healing study.

### DESCRIPTION OF THE DISCLOSURE

The present disclosure relates to a compound of Formula I:

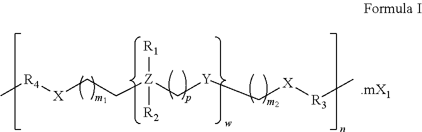

wherein, n=1 to 1000;
p=1 to10;
w=1 to 10;
m₁=0to10;
m₂=0 to 10;
X is oxygen or —NH—;
m=1-1000;
X₁ is bromide, chloride, iodide, sulfate, bisulfate, phosphate, nitrate, trifluoroacetate, acetate, propionate, glycolate, succinate, valerate, oleate, palmitate, stearate, laurate, benzoate, lactate, phosphate, tosylate, citrate, maleate, fumarate, succinate, tartrate, ascorbate, napthylate, hydroxymaleate, mesylate, glucoheptonate, lactobionate, laurylsulphonate, phenylacetate, glutamate, benzoate, salicylate, sulfanilate, 2-acetoxybenzoate, fumarate, toluenesulfonate, methanesulfonate, ethane disulfonate, oxalate, isothionate, quaternary ammonium salt, or any other pharmaceutically acceptable salt, or any combinations thereof;
Z is carbon or nitrogen;
Y is —CH₂— or —NH— or functionalized amine;
R₁ and R₂ is independently

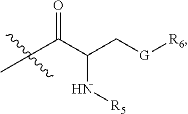

hydrogen, alkyl, straight alkyl chain, branched alkyl chain, —(CH₂)ₙ wherein n=1 to 30, alkenyl, alkynyl, aryl, heteroaryl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 and —(CH₂)ₚ—CH═CH₂ with p=1-10, polymer or polymer derivatives are —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), PEG, polylactic acid (PLA), PEG-PLA co-polymer, alginic acid, chitosan, PLGA, ethylene vinyl acetic acid, acrylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, carboxylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, polyester, polyamide, polycarbamate, polycarbonate, alkyl linked hybrid scaffold, cross-link polymeric scaffold, polybiguanidine, polyurethane, mixed polybiguanidine-polyurethane, polyurea, polyester, polyamide, polycarbonate, polycarbamate, polymethacrylate, polyvinyl, or any combinations of R₁ and R₂ thereof; and wherein each of the R₁ and R₂ is optionally substituted with primary, secondary, tertiary, quaternary amino group, hydroxyl group, thiol group, acrylic group, halogen selected from fluorine, chlorine, bromine or iodine, —COR₈ wherein R₈ is alkyl, alkenyl, monoenes, polyenes, terminally substituted alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, or C-terminal amino acids with D or L configuration, oligo-peptide, or any combinations thereof, wherein R₁ is optionally present;
and wherein in

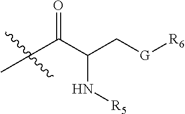

‘G’ is oxygen (—O—) or sulphur (—S—), R₅ and R₆ is independently selected from hydrogen, alkyl, straight alkyl chain, branched alkyl chain, —(CH₂)ₙ wherein n=1 to 30, alkenyl, alkynyl, aryl, heteroaryl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 or —(CH₂)ₚ—CH═CH₂ with p=1-10, and wherein each of R₅ and R₆ is optionally substituted with primary, secondary, tertiary or quaternary amino group, hydroxyl group, thiol group, carboxylic group, acrylic group or halogen selected from fluorine, chlorine, bromine or iodine, or any combinations thereof;
and wherein in —COR₇ and —COR₈, R₇ and R₈ is independently selected from alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl or heteroaryl, and wherein each of the R₇ and R₈ is optionally substituted with primary, secondary, tertiary or quaternary amino group, hydroxyl group, thiol group, carboxylic group, acrylic group, halogen selected from fluorine, chlorine, bromine or iodine; C-terminal amino acids with D or L configuration, or oligo-peptide, or any combinations thereof;
and
R₃ and R₄ is independently hydrogen, alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl or heteroaryl group, wherein each of the R₃ and R₄ is optionally substituted with primary, secondary, tertiary or quaternary amino group, hydroxyl group, thiol group, carboxylic group, acrylic group, halogen selected from fluorine, chlorine, bromine or iodine, —COR₈ with R₈ selected from alkyl, alkenyl, monoenes, polyenes, terminally substituted alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, oligopeptide, polymer or polymer derivatives selected from —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PLGA, ethylene vinyl acetic acid, polymethacrylate, polyvinyl, acrylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, carboxylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA, ethylene vinyl acetic acid, polyester, polyamide, polycarbamate, polycarbonate, alkyl linked hybrid scaffold, cross-link polymeric scaffold, polybiguanidine, polyurethane, mixed polybiguanidine-polyurethane, polyurea, polyester, polyamide, polycarbonate or polycarbamate or any combinations thereof; —C(NH)—NH—C(NH)—, —C(NH)—NH—C(NH)—NH—(CH₂)ₙ—NH—C(NH)—NH—C(NH)— with n=1-20, —C(NH)—NH—C(NH)—NH-M-NH—C(NH)—NH—C(NH)— wherein M is

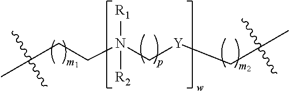

wherein R₁, R₂, Y, p, w, m₁ and m₂ is as defined above and R₁ is optional, —C(NH)—NH—C(NH)—NH—CH₂—(CH₂)ₘ—O(CO)NH—(CH₂)ₙ—NH(CO)—OCH₂—(CH₂)ₙ—NH—C(NH)—NH—C(NH)— with m=1-20, n=1-20, —CO—NH—(CH₂)ₙ—NH(CO)— with n=1-20, —CO—(CH₂)ₙ—(CO)— with n=1-20, —CO—NH—(CH₂)ₙ—NH(CO)— with n=1-20, [—CO—NH—(CH₂)ₙ—NH(CO)—(OCH₂—CH₂)ₘ—O(CO)—NH—] with m=1-20, n=1-20, —CO—(CH₂)ₙ—(CO)— with n=1-20, —COO—, —OCO—(CH₂CH₂—O)ₙ—CO— with n=1-20, —C(NH)—NH—C(NH)—NH—(CH₂)ₘ—O(CO)NH—(CH₂)ₙ—NH(CO)—O—(CH₂)ₙ—NH—C(NH)—NH—C(NH)— with m=1-20, n=1-20.

In an embodiment of the present disclosure, the compound of Formula I is selected from

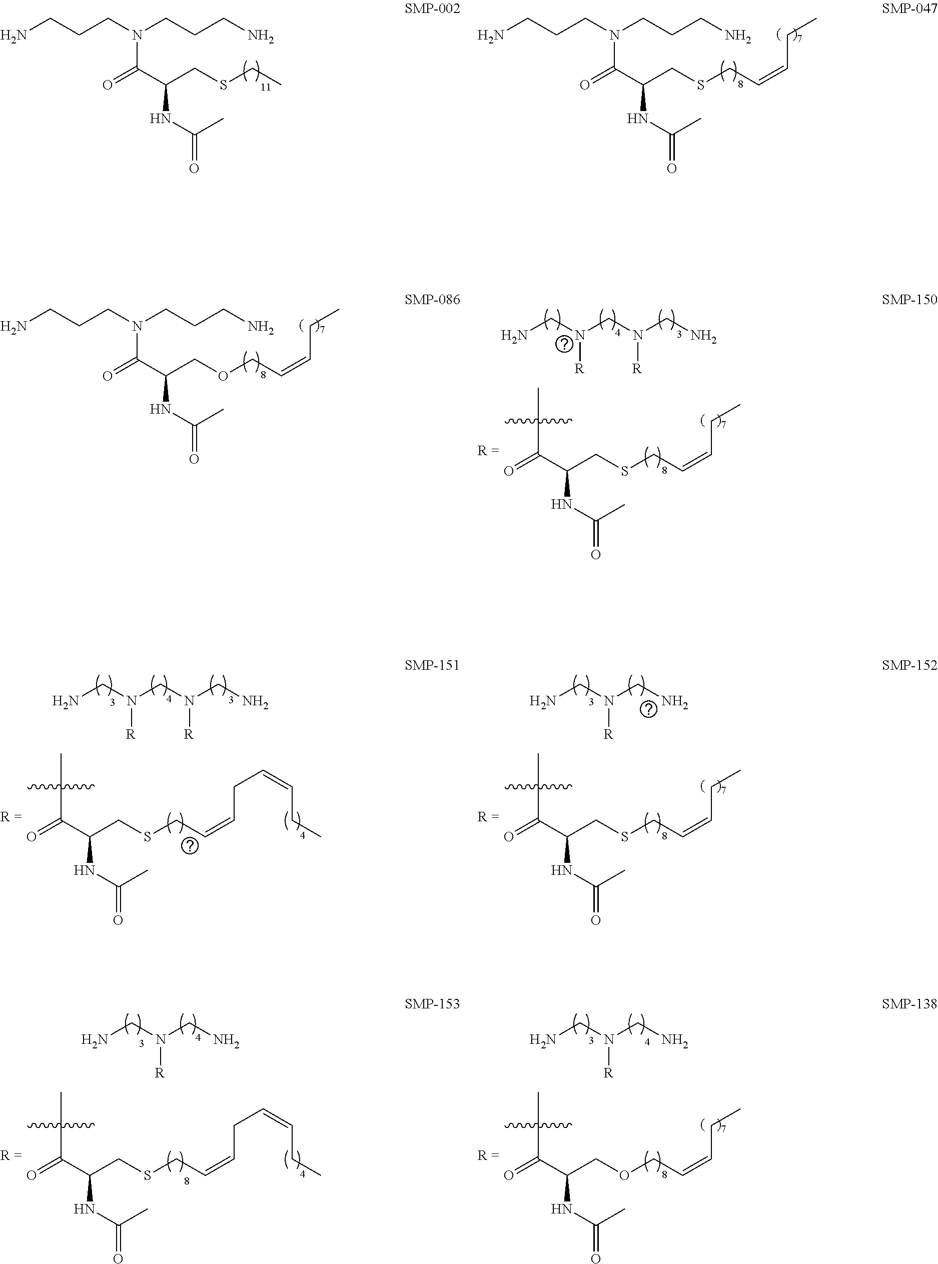

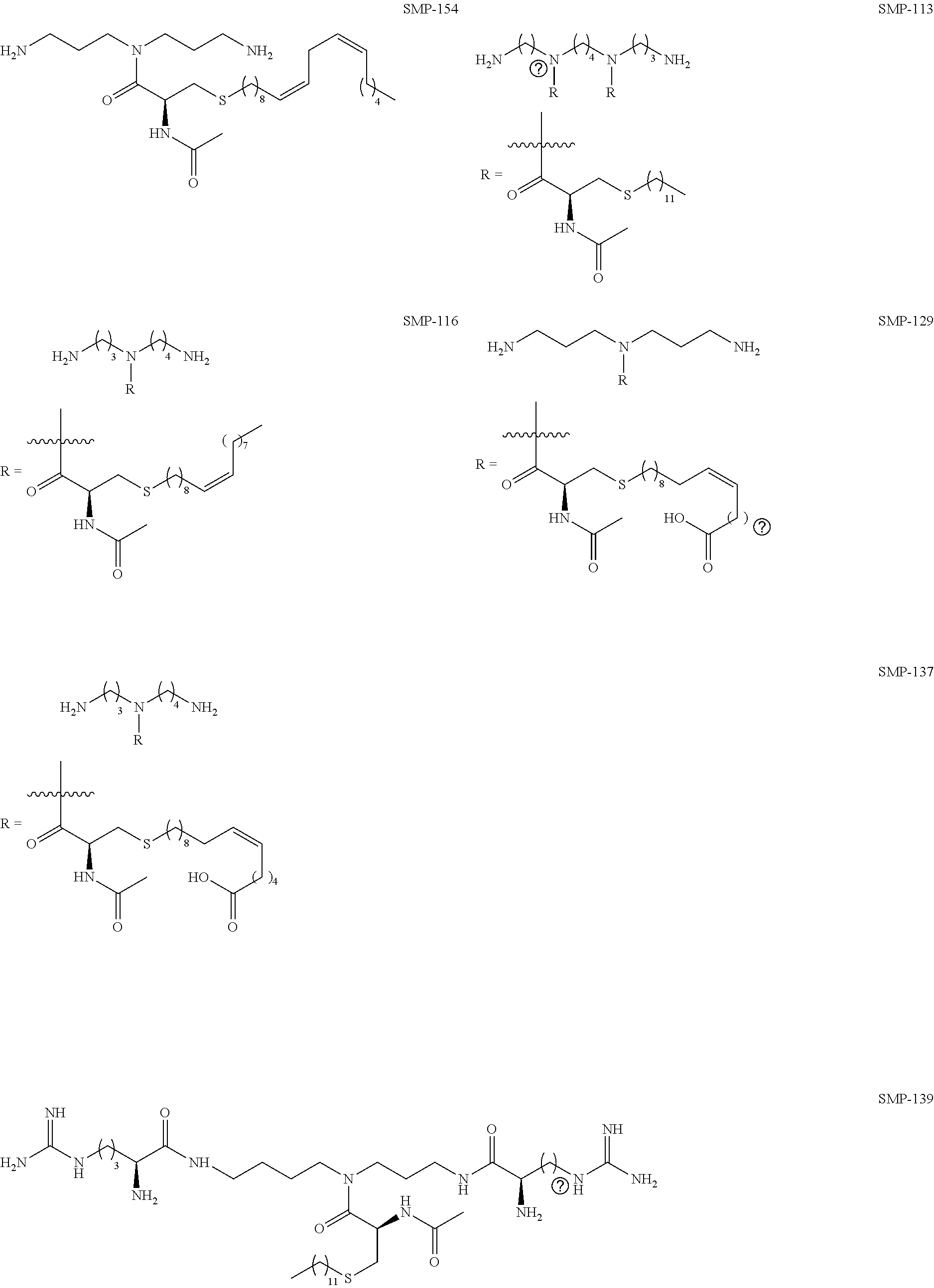

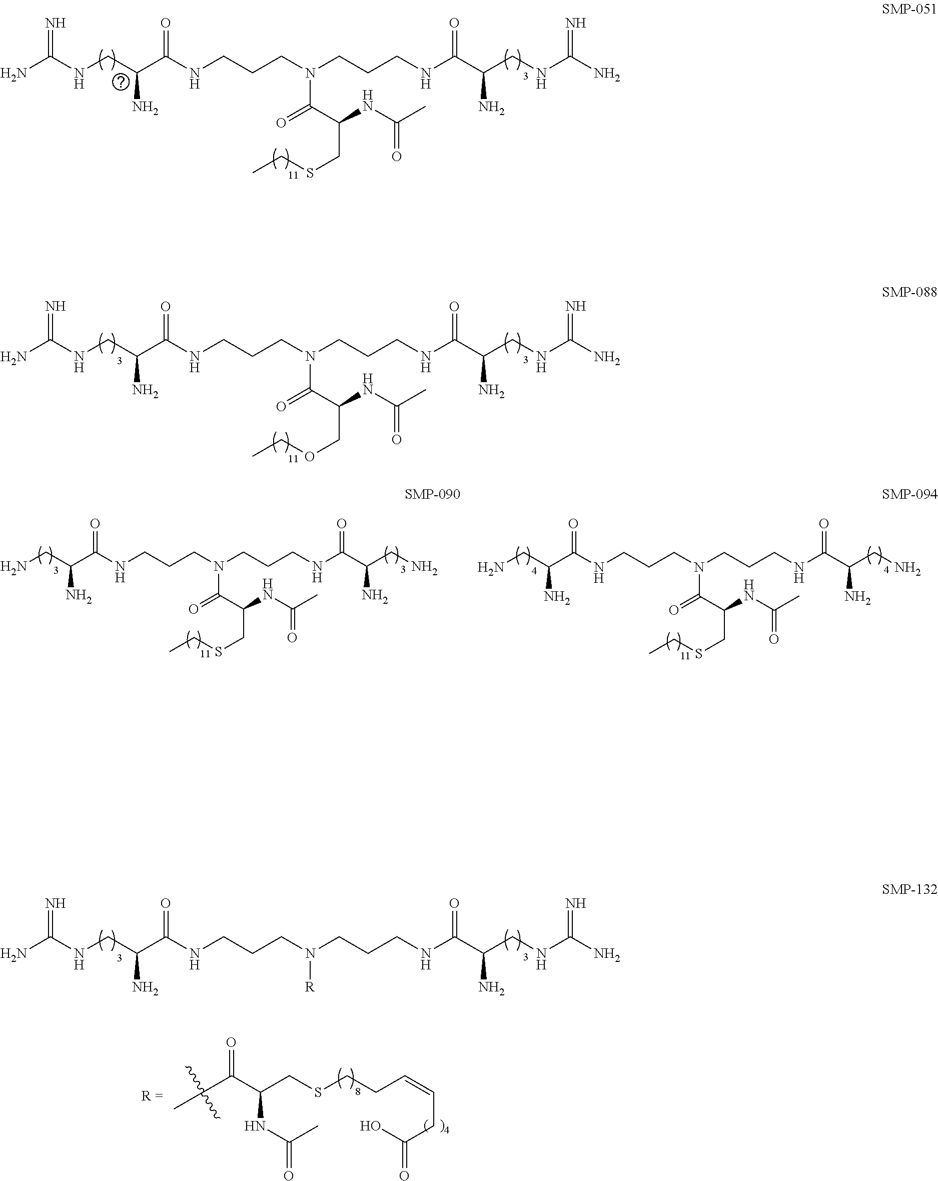

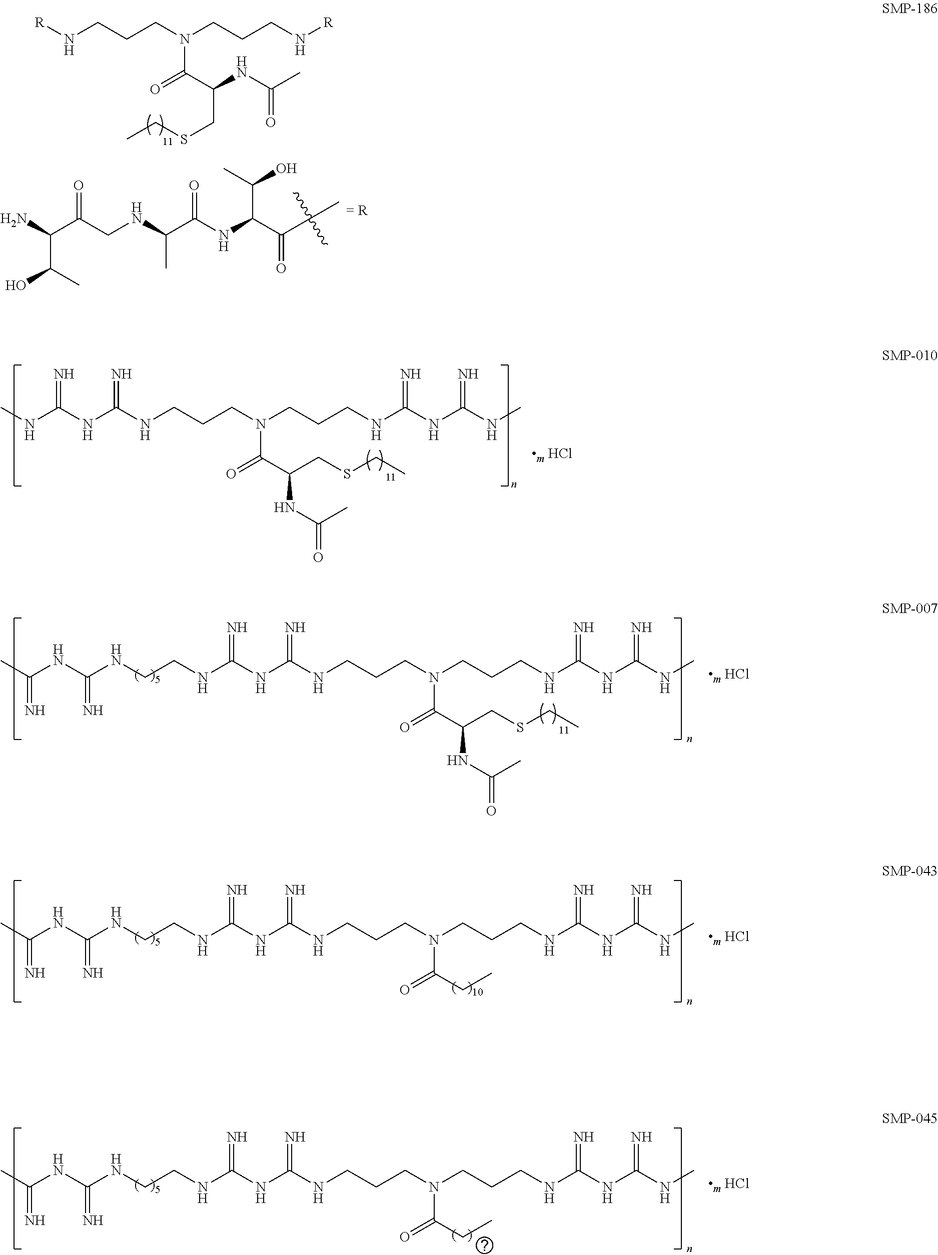

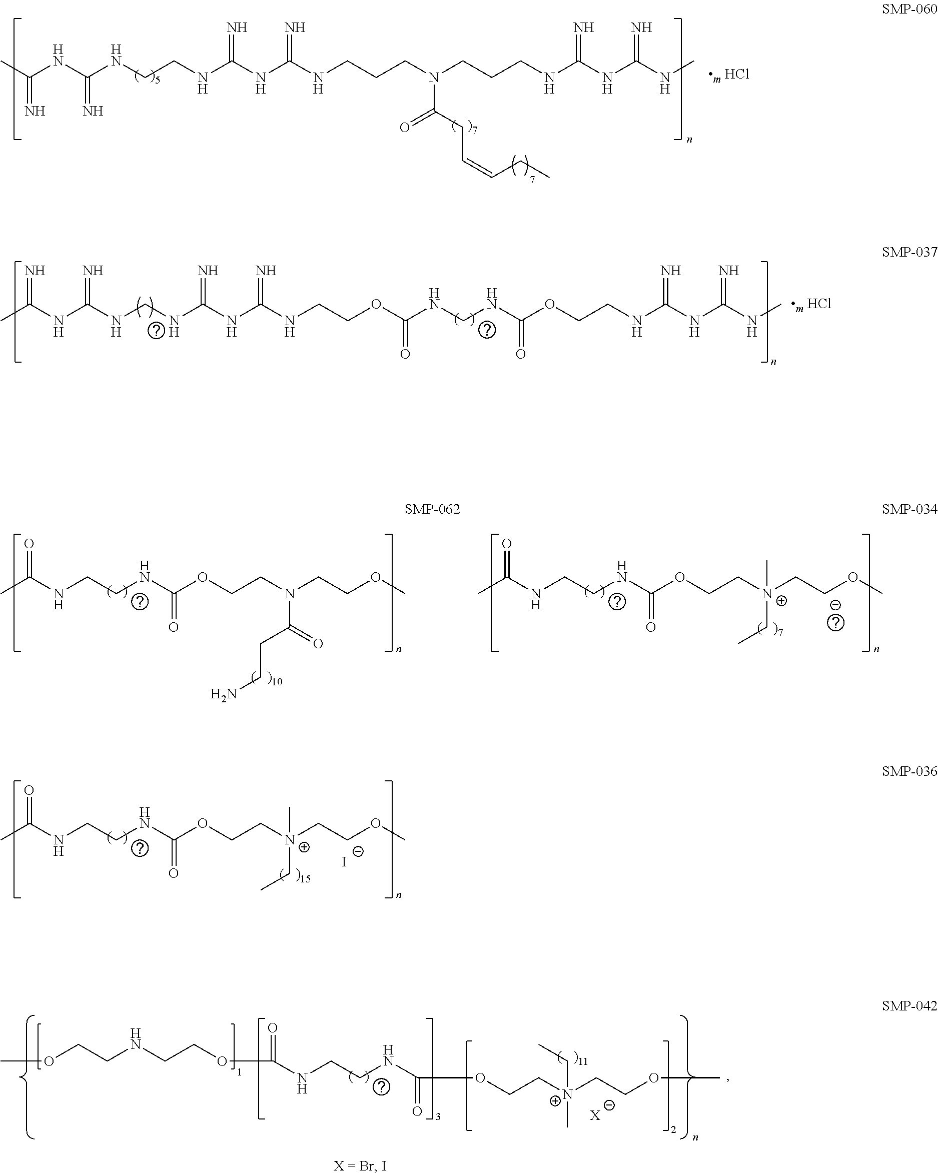

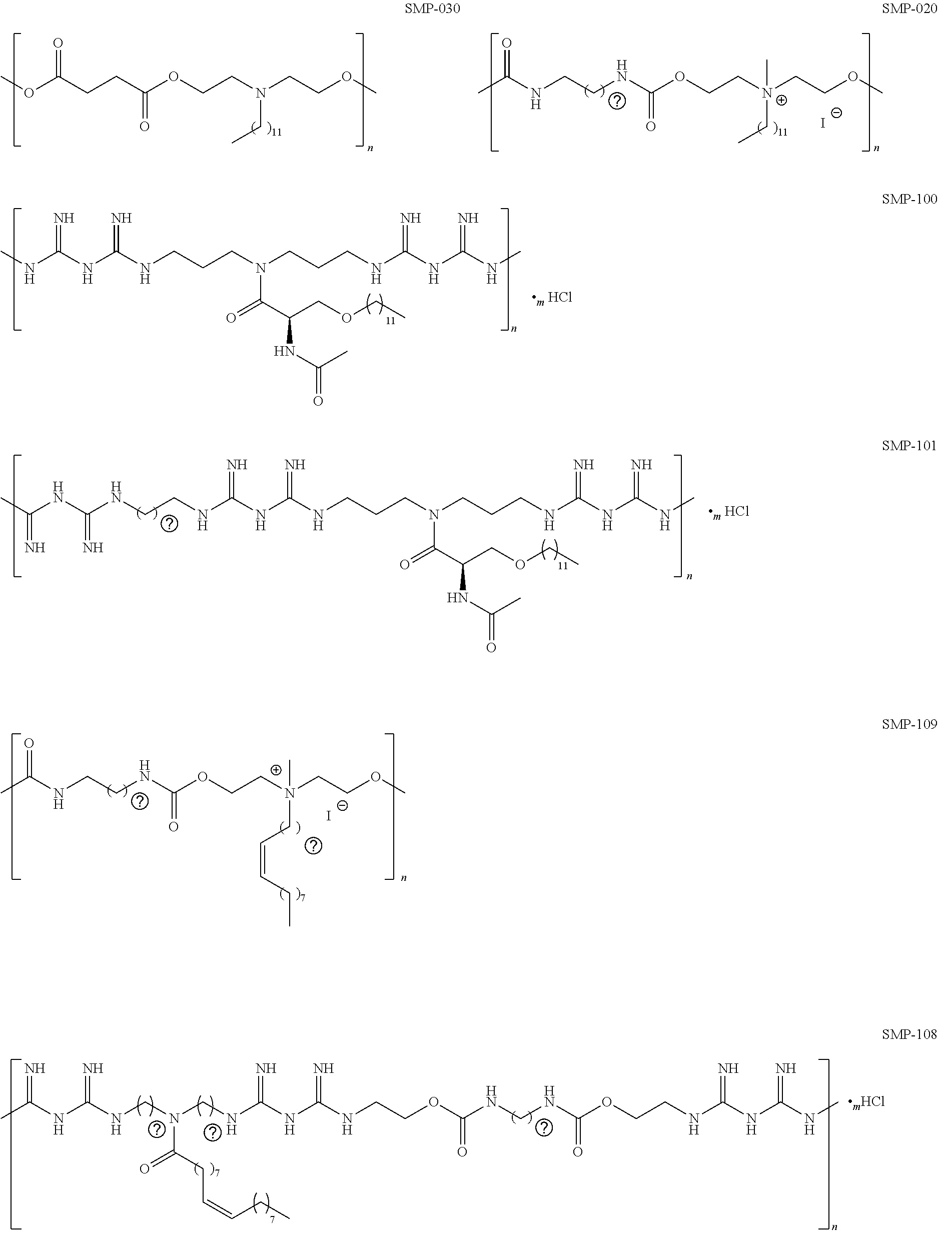

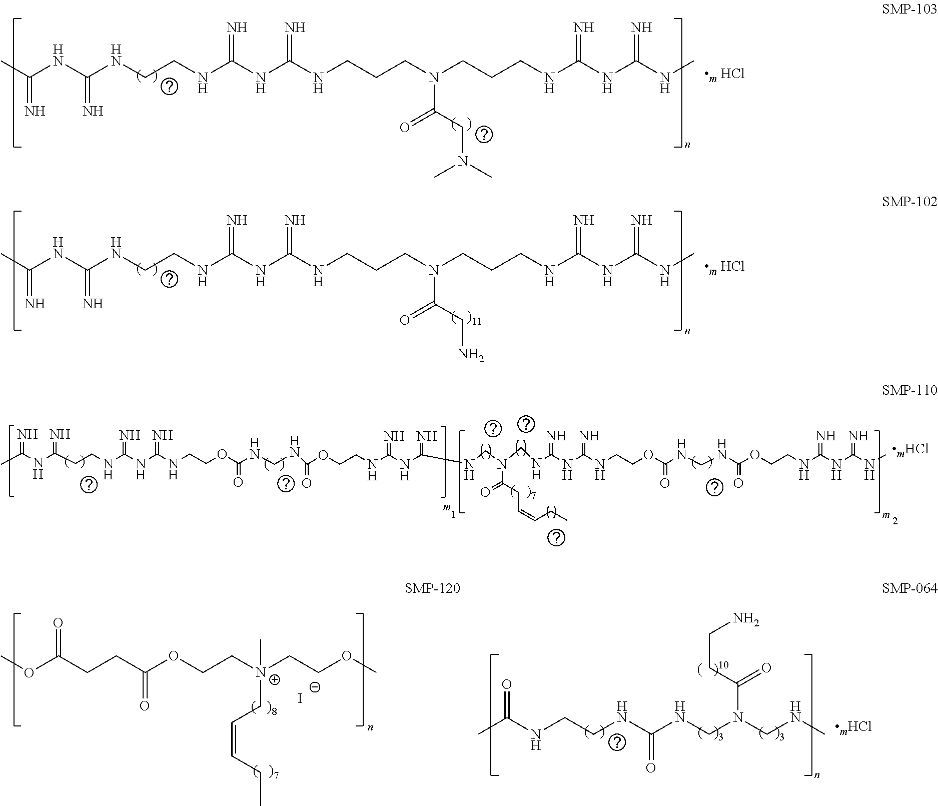

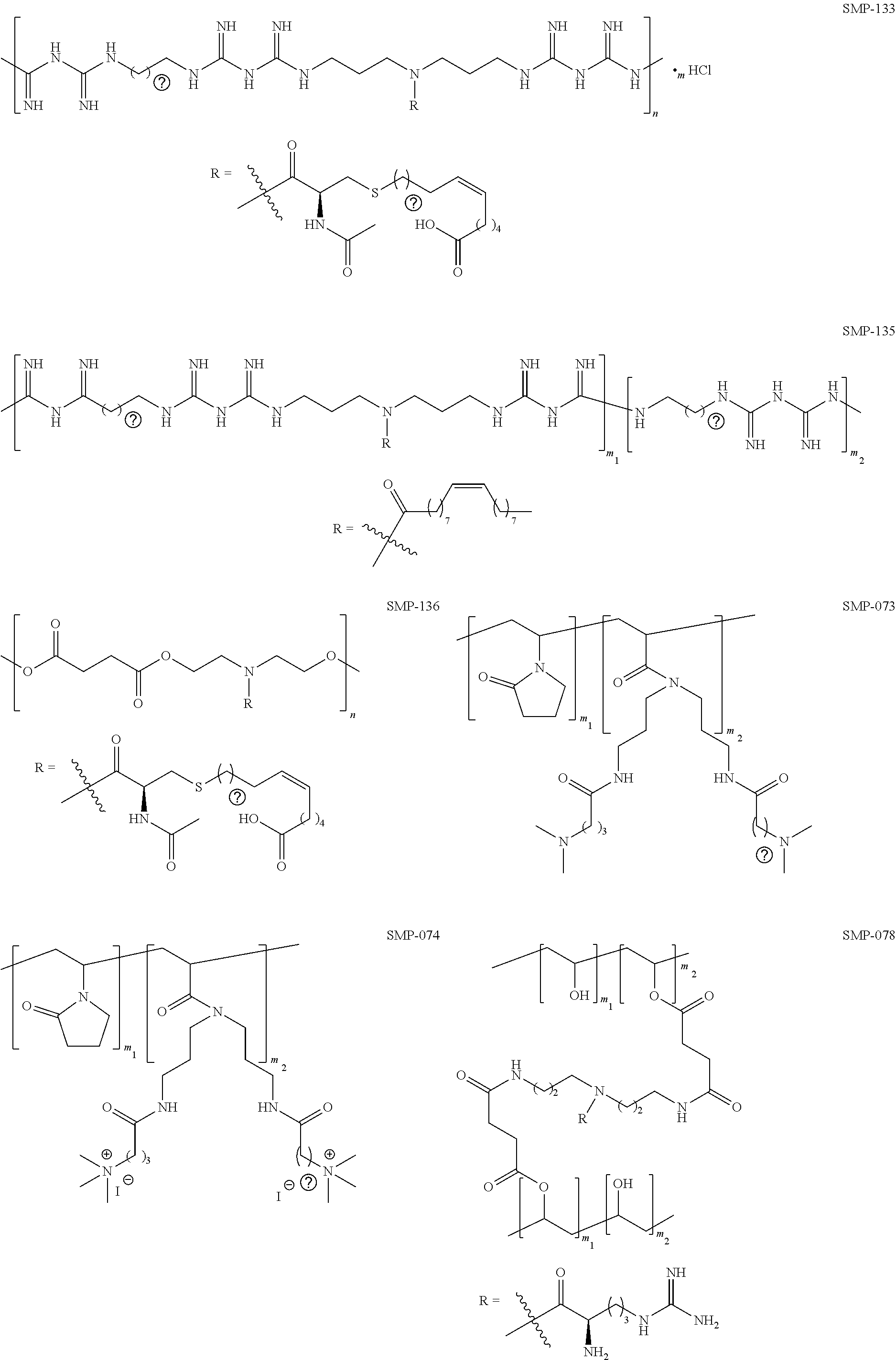

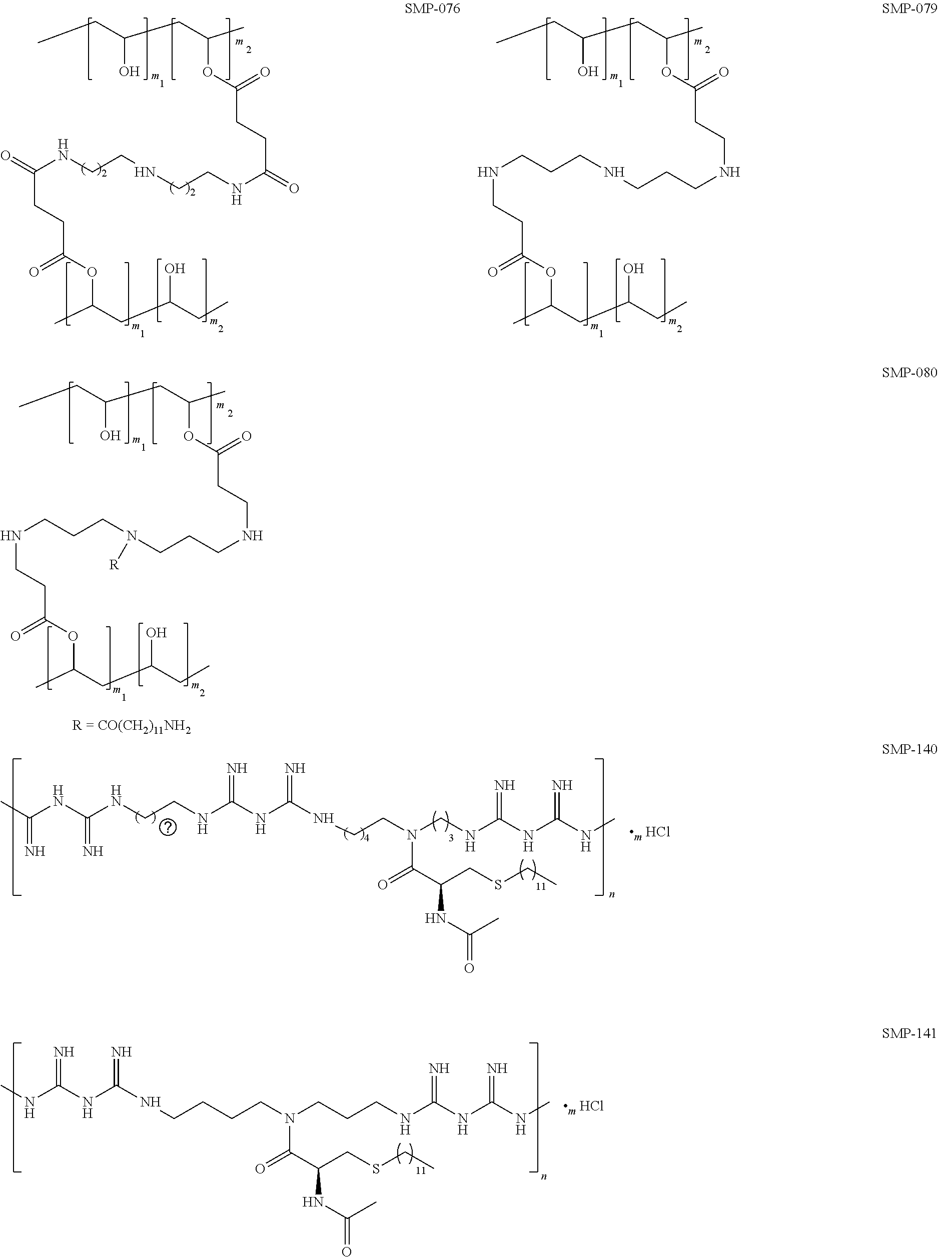

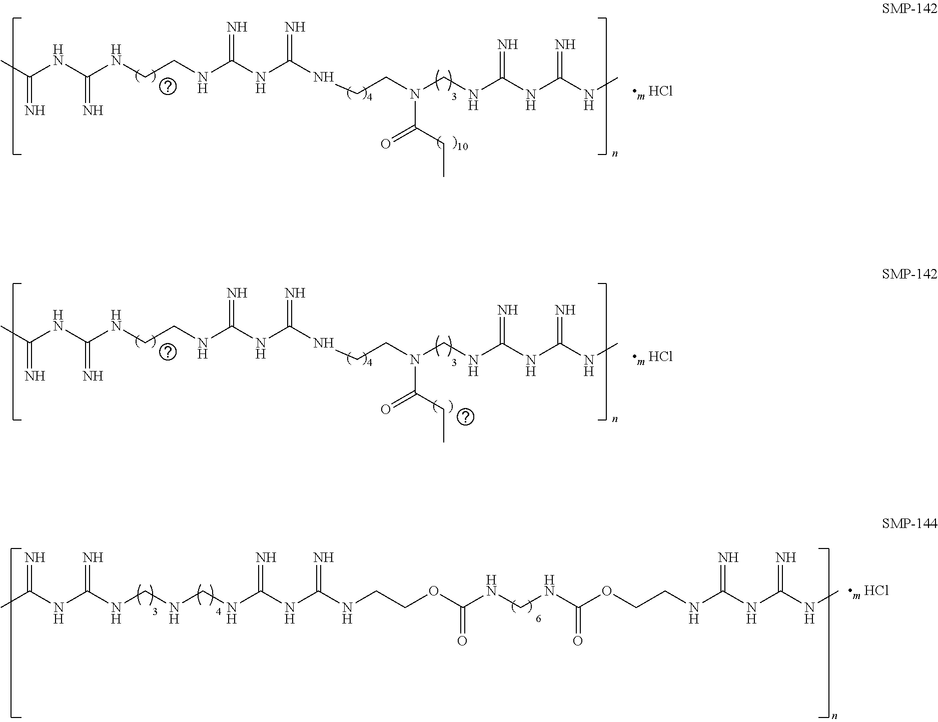

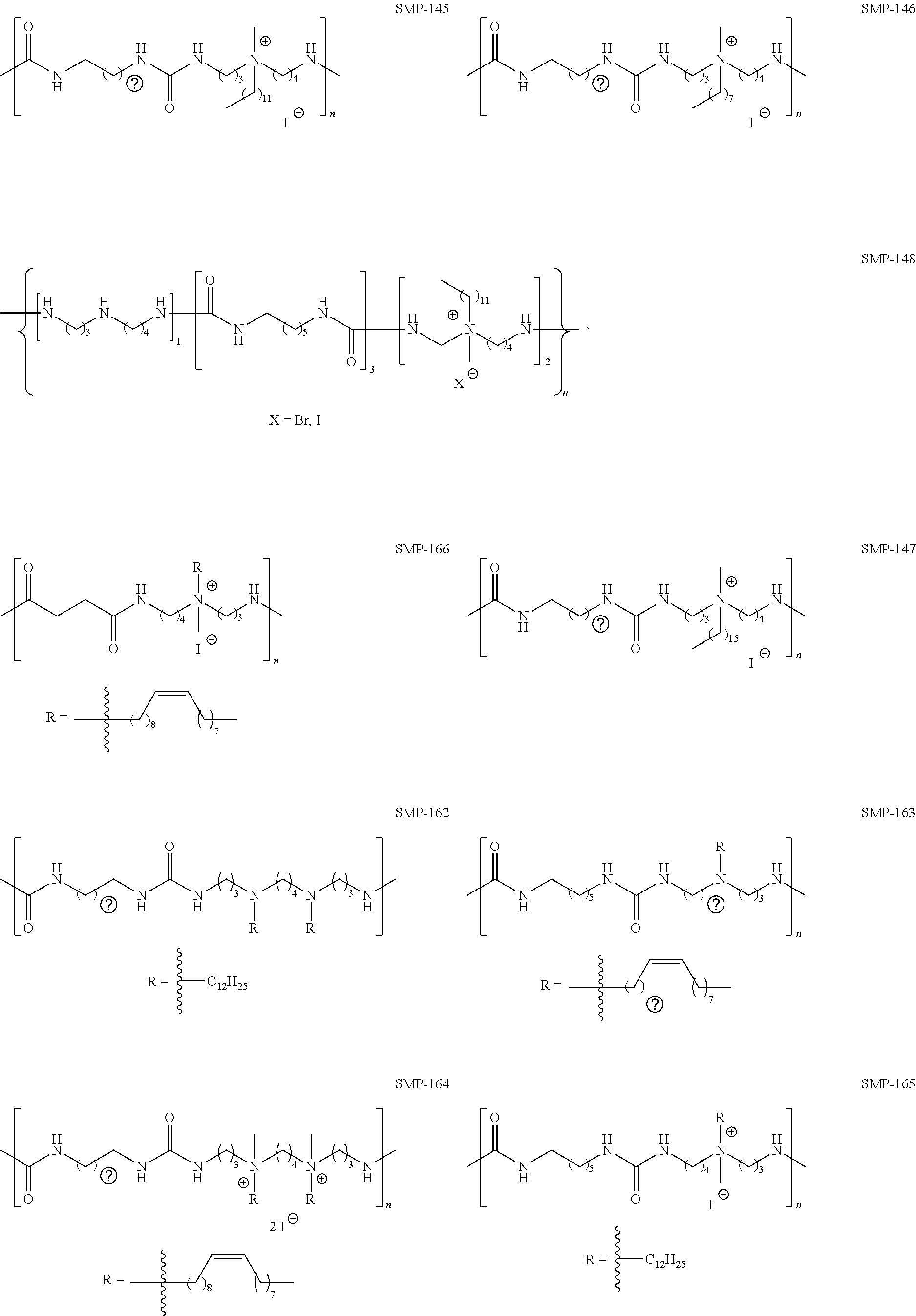

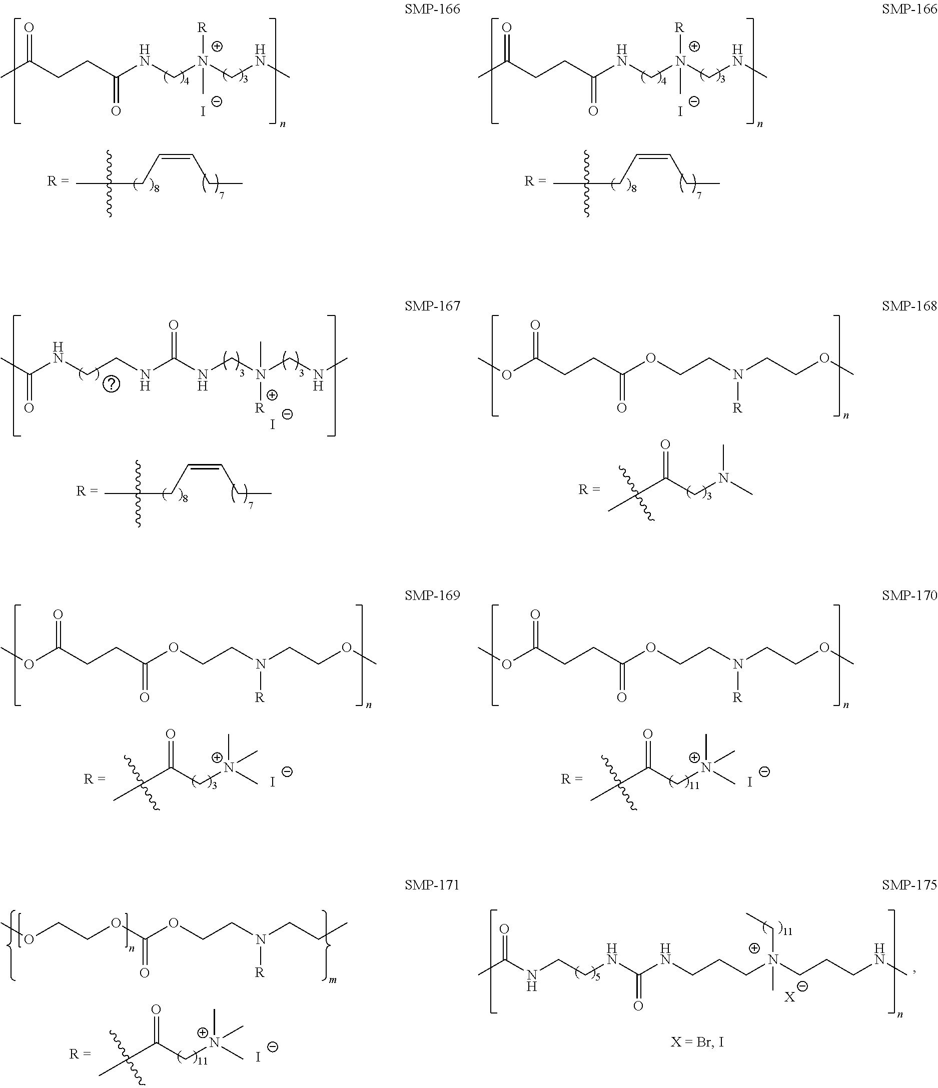

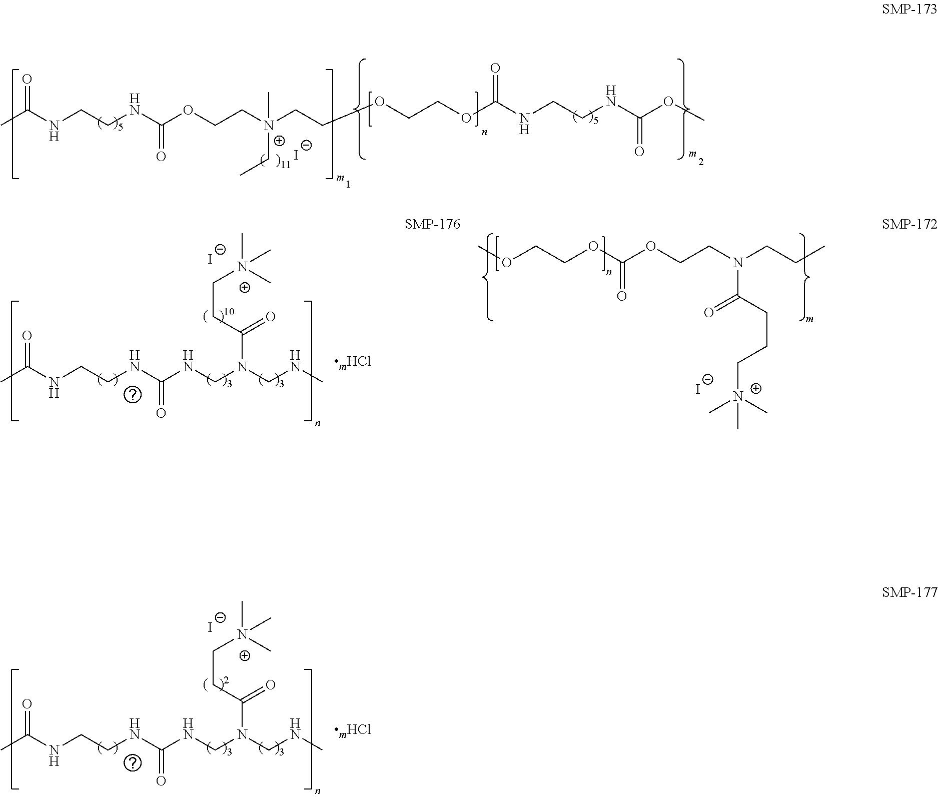

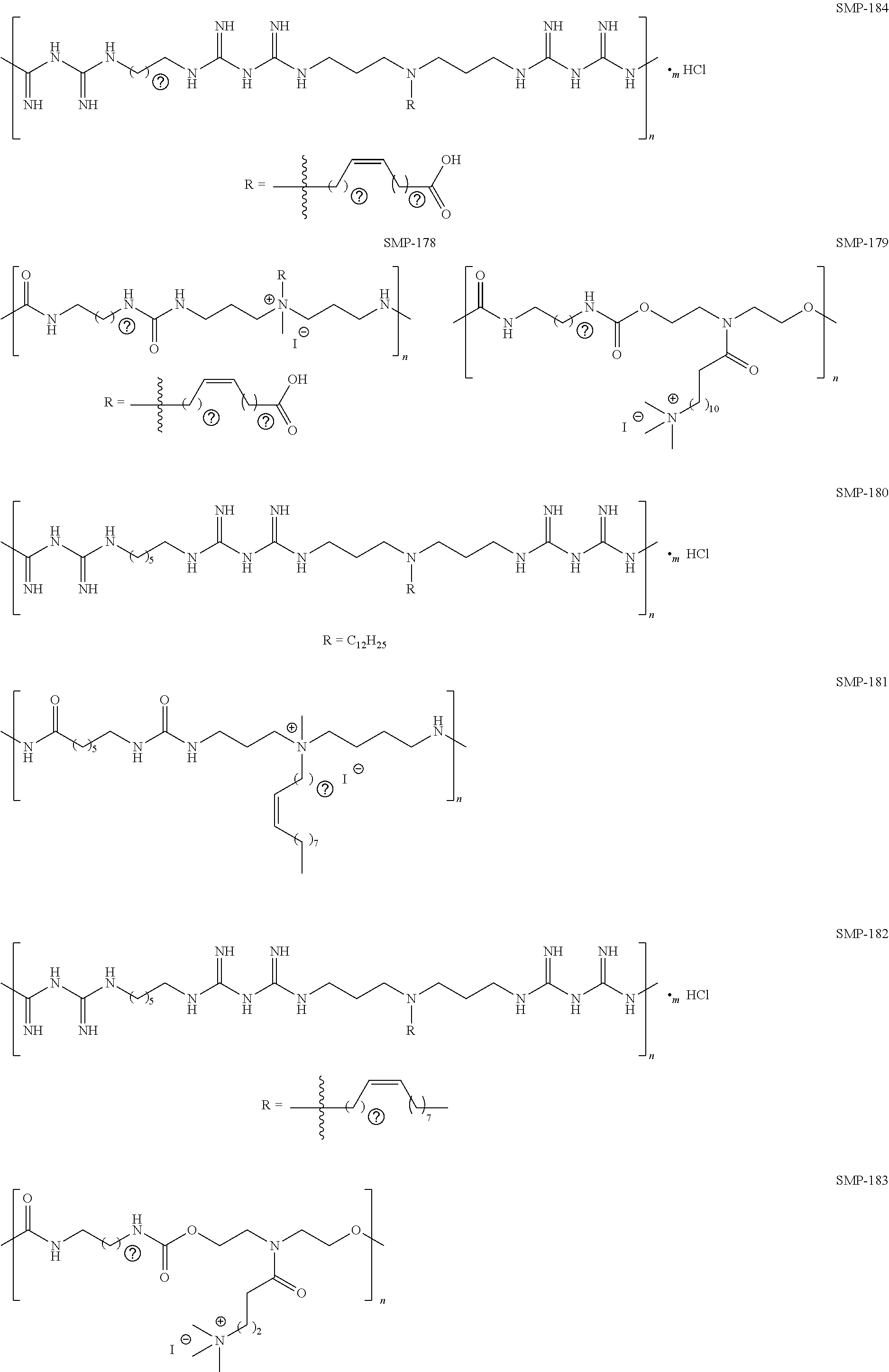

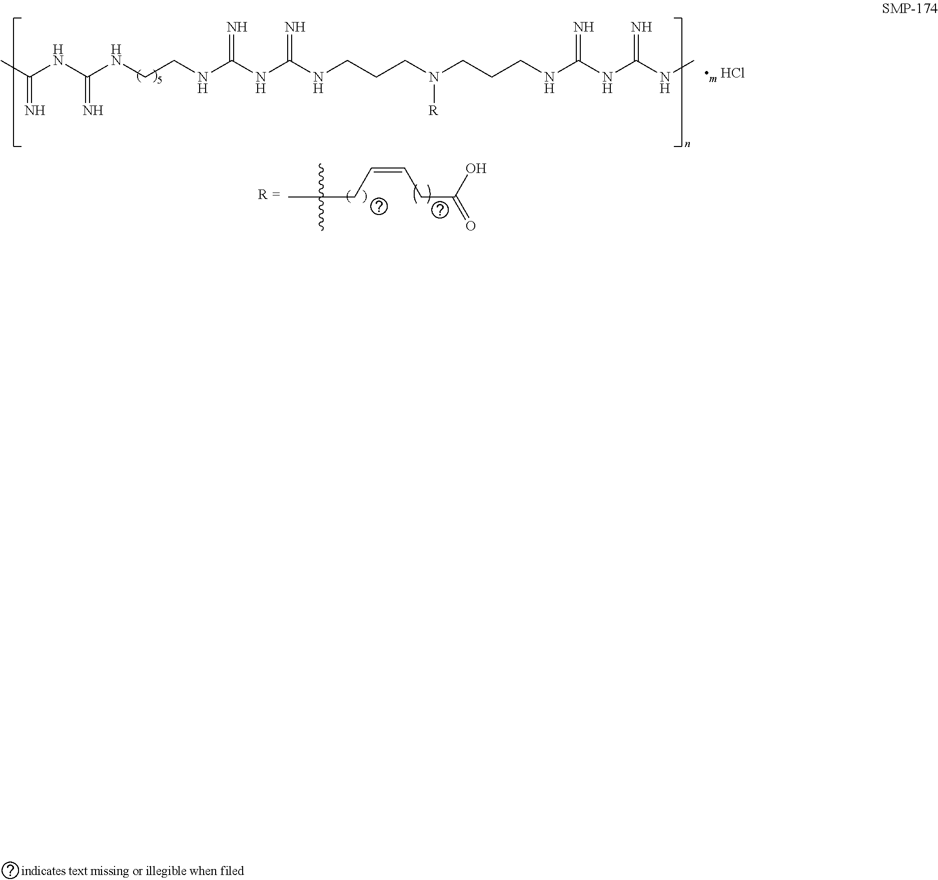

In another embodiment of the present disclosure, the compound is a compound of Formula Ia

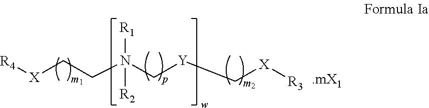

wherein,
p=1 to 10;
w=1 to10;
m₁=0to10;
m₂=0 to 10;
R₁ is absent;
R₂ is independently hydrogen,

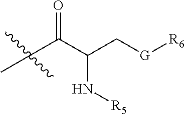

wherein ‘G’ is oxygen (—O—) or sulphur (—S—), wherein R₅ and R₆ is independently hydrogen, alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 or —(CH₂)p-CH═CH₂ with p=1-10, and wherein each of R₅ and R₆ is optionally substituted with primary, secondary, tertiary or quaternary amino group, hydroxyl group, thiol group, carboxylic group, acrylic group, halogen selected from fluorine, chlorine, bromine or iodine, —COR₇ wherein R7 is alkyl, straight alkyl chain, branched alkyl chain, —(CH₂)n wherein n=1 to 30, alkenyl, alkynyl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10, —(CH₂)p-CH═CH₂ with p=1-10, aryl, heteroaryl and wherein each of the R7 is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide;
R₃ and R₄ is independently hydrogen, alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl, or heteroaryl group, and wherein each of the R₃ and R₄ is optionally substituted with primary, secondary, tertiary, quaternary amino group, hydroxyl group, thiol group, carboxylic group, acrylic group, halogen selected from fluorine, chlorine, bromine or iodine, —COR₈ wherein R₈ is alkyl, alkenyl, monoenes, polyenes, terminally substituted alkyl, straight alkyl chain, branched alkyl chain, —(CH₂)ₙ wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligopeptide;
Y is —CH₂—, —NH— and a functionalized amine, wherein the functionalized amine is
N(R₂), wherein R₂ is

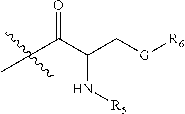

with ‘G’ being oxygen (—O—) or sulphur (—S—);
X=—NH— or —O—,
m=1-1000; and
X₁, R₅ and R₆ is as defined in Formula I above.

In another embodiment of the present disclosure, the compound Formula I consists of:
n=2 to 1000;
Z═NH or C;
Y═NH or CH₂;
X═NH or 0;
p=1 to 10;
w=1 to 10;
m₁=0 to 10;
m₂=0 to 10;
X₁ as defined in Formula I above;
R₃ and R₄ is independently hydrogen, alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl or heteroaryl group and wherein each of the R₃ and R₄ is optionally substituted with primary, secondary, tertiary or quaternary amino group, hydroxyl group, thiol group, carboxylic group, acrylic group, halogen selected from fluorine, chlorine, bromine or iodine, —COR₈ wherein R₈ is alkyl, alkenyl, monoenes, polyenes, terminally substituted alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide;
R₁ is absent;
R₂ is a polymer or polymer derivative is —CH₂—(CH₂)ₙ—COO-PVA, —CO—CH₂—(CH₂)ₙ-PVA wherein n=1-20; polyvinylpyrolidone (PVP), polyglycolic acid (PGA), polyacrylic acid (PAA), polymethacrylate, polyvinyl, alginic acid, chitosan, PEG, polylactic acid (PLA), PEG-PLA co-polymer, PLGA, ethylene vinyl acetic acid, acrylic derivatives of —CH₂—(CH₂)ₙ—COO-PVA, —CO—CH₂—(CH₂)ₙ-PVA wherein n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA, ethylene vinyl acetic acid, carboxylic derivatives of —CH₂—(CH₂)ₙ—COO-PVA, —CO—CH₂—(CH₂)ₙ-PVA wherein n=1-20, polyvinylpyrolidone (PVP), polyglycolic acid (PGA), polyacrylic acid (PAA), alginic acid, chitosan, PLGA, PEG, polylactic acid (PLA), PEG-PLA co-polymer, ethylene vinyl acetic acid, polyester, polyamide, polycarbamate, polycarbonate, alkyl linked hybrid, cross-link polymeric scaffolds, polybiguanidine, polyurethane, mixed polybiguanidine-polyurethane, polyurea, polyester, polyamide, polycarbonate or polycarbamate, or any combinations thereof.

In yet another embodiment of the present disclosure, the above compound is a compound wherein n=2 to 500, R₁, R₂, R₃, R₄, X, Y, Z, m₁, p, m₂, m, X₁ are as defined in Formula I.

In still another embodiment of the present disclosure, the above compound is a compound wherein n=2 to 200, R₁, R₂, R₃, R₄, X, Y, Z, m₁, p, m₂, m, X₁ are as defined in Formula I.

In still another embodiment of the present disclosure, the compound is a compound consisting of conjugate polymers of compound of Formula I, wherein compound of Formula I with ‘n’=2 to 1000 conjugates with polymer or polymer derivative selected from polyvinylpyrolidone (PVP), polyglycolic acid (PGA), polyacrylic acid (PAA), polymethacrylate, polyvinyl, alginic acid, chitosan, PLGA, ethylene vinyl acetic acid, polyester, polyamide, polycarbamate, polycarbonate, PEG, PLA, PLA-PEG co-polymer, PHMB polyguanidine, polybiguanidine, polyurethane, polybiguanidine-polyurethane, polyurea, polyester, polyamide or polycarbonate, or any combinations thereof.

In still another embodiment of the present disclosure, the compound is a compound of Formula Ib

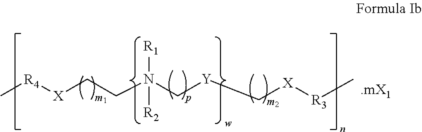

wherein, n=2 to 1000;
p=1 to 10;
w=1 to 10;
m₁=0 to 10;
m₂=0 to10;
R₃ and R₄ is independently —C(NH)—NH—C(NH)—; —C(NH)—NH—C(NH)—NH—(CH₂)ₙ—NH—C(NH)—NH—C(NH)— wherein n=1-20; —C(NH)—NH—C(NH)—NH-M-NH—C(NH)—NH—C(NH)— wherein M is

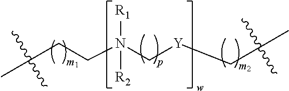

wherein R₁, R₂, Y, p, w, m₁ and m₂ as defined above, wherein R₁ is optional; —C(NH)—NH—C(NH)—NH—CH₂—(CH₂)ₘ—O(CO)NH—(CH₂)ₙ—NH(CO)—OCH₂—(CH₂)ₙ—NH—C(NH)—NH—C(NH)—, wherein m=1-20, n=1-20; —CO—NH—(CH₂)ₙ—NH(CO)— wherein n=1-20; —CO—(CH₂)ₙ—(CO)—, wherein n=1-20; —CO—NH—(CH₂)ₙ—NH(CO)— wherein n=1-20;
[—CO—NH—(CH₂)ₙ—NH(CO)—(OCH₂—CH₂)ₘ—O(CO)—NH—] wherein m=1-20, n=1-20; —CO—(CH₂)ₙ—(CO)— wherein n=1-20; —COO—; —OCO—(CH₂CH₂—O)ₙ—CO—, wherein n=1-20;
X is oxygen or —NH—;
m=1-1000;
X₁ is as defined in Formula I;
Y is —NH—;
wherein R₁ and R₂ is independently alkyl, alkenyl, alkynyl, straight alkyl chain, branched alkyl chain, —(CH₂)ₙ wherein n=1 to 30, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 and —(CH₂)ₚ—CH═CH₂, with p=1-10, and wherein each of the R₁ and R₂ is optionally substituted with primary, secondary, tertiary or quaternary amino group, hydroxyl group, thiol group, acrylic group, halogen selected from fluorine, chlorine, bromine or iodine, —COR₈ wherein R₈ is alkyl, alkenyl (mono or polyenes) or terminally substituted alkyl, straight or branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligopeptide,

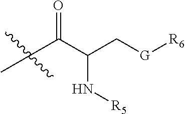

wherein ‘G’ is oxygen (—O—) or sulphur (—S—), wherein R₅ and R₆ is independently hydrogen or alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 or —(CH₂)p-CH═CH₂ with p=1-10, and wherein each of R₅ and R₆ is optionally substituted with primary, secondary, tertiary or quaternary amino group, hydroxyl group, thiol group, carboxylic group, acrylic group, halogen selected from fluorine, chlorine, bromine or iodine, —COR₇, —COR₈ wherein R7 and R8 is alkyl, straight alkyl chain, branched alkyl chain, —(CH2)n with n=1 to 30, alkenyl, alkynyl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10, —(CH₂)p-CH═CH₂ with p=1-10, aryl, heteroaryl and wherein each of the R7 and R8 is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide.

In still another embodiment of the present disclosure, wherein the C-terminal amino acids with D or L configuration describe above is lysine, arginine, ornithine, proline, histidine, serine, threonine, tyrosine, tryptophan, phenyl alanine, cysteine, cystine, isoleucine, leucine, glycine, asparagine, glutamine, aspartic acid or glutamic acid, or any combinations thereof.

In still another embodiment of the present disclosure, the C-terminal oligopeptide as described above consists of 2-30 amino acids with D or L configuration.

In still another embodiment of the present disclosure, the C-terminal oligopeptide described above is TAT (Threonine-Alanine-Threonine), Cholesterol-conjugated G3R6TAT (dodecapeptide), MP196 (hexapeptide, RWRWRW—NH₂), PAF-26 (hexapeptide, RKKWFW), Mastoparan (Polybia-MP1, tetradecapeptide, IDWKKLLDAAKQIL), D-IK8 (octapeptide, IRIKIRIK), L5K5W (undecapeptide, KKLLKWLKKLL-NH₂), Gramicidin-D (pentadecapeptide, VGALAVVVWLWLWLW), WR12 (dodecapeptide, RWWRWWRRWWRR), Protegrins (PG-1, octadecapeptide or NH₂—RGGRLCYCRRRFCVCVGR—CONH₂)), or any combinations thereof.

In still another embodiment of the present disclosure, the Formula Ib described above is a compound wherein X═NH; m₁, R₁, R₂, p, Y, m₂, m, X₁ are as defined in Formula I, and R₃ and R₄ are combinations selected from:
R₃═R₄═—C(NH)—NH—C(NH)—;
R₃═—C(NH)—NH—C(NH)— and R₄═—C(NH)—NH—C(NH)—NH—(CH₂)ₙ—NH—C(NH)—NH—C(NH)—, wherein n=1-20;
R₃═R₄═—C(NH)—NH—C(NH)—NH—(CH₂)ₙ—NH—C(NH)—NH—C(NH)—, wherein n=1-20;
R₃═—C(NH)—NH—C(NH)— and R₄ is —C(NH)—NH—C(NH)—NH-M-NH—C(NH)—NH—C(NH)—, wherein M is

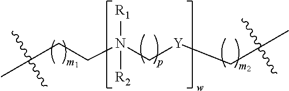

wherein R₁, R₂, Y, p, w, m₁ and m₂ are same as defined in Formula Ia above;
R₃═—C(NH)—NH—C(NH)— and R₄═—C(NH)—NH—C(NH)—NH—CH₂—(CH₂)ₘ—O(CO)NH—(CH₂)ₙ—NH(CO)—OCH₂—(CH₂)ₙ—NH—C(NH)—NH—C(NH)—, wherein m=1-20, n=1-20;
R₃═R₄═—CO—NH—(CH₂)ₙ—NH(CO)— wherein n=1-20; or
R₃═R₄═—CO—(CH₂)ₙ—(CO)—, wherein n=1-20.

In still another embodiment of the present disclosure, the Formula Ia is a compound, wherein X═O; m₁, R₁, R₂, p, Y, m₂, m, X₁ are as defined in Formula I or Formula Ia; and R₃ and R₄ are combinations selected from:
R₃═R₄═—CO—NH—(CH₂)ₙ—NH(CO)— wherein n=1-20;
R₃═—CO—NH—(CH₂)ₙ—NH(CO)— wherein n=1-20 and R₄═[—CO—NH—(CH₂)ₙ—NH(CO)—(OCH₂—CH₂)ₘ—O(CO)—NH—] wherein n=1-20, m=1-20;
R₃═R₄═—CO—(CH₂)ₙ—(CO)—, wherein n=1-20;
R₃═R₄═—COO—; or
R₃═—COO— and R₄═—OCO—(CH₂CH₂—O)ₙ—CO—, wherein n=1-20.

In still another embodiment of the present disclosure, wherein the compound is a compound of Formula Ic

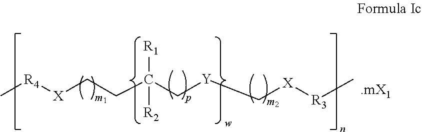

wherein n=2-1000,
Y═C,
X═NH or O,
p=1to10,
w=1to 10,
m₁=0 to 10,
m₂=0 to 10, and
R₁, R₂, R₃, R₄ are as defined in Formula I.

In still another embodiment of the present disclosure, the compound of Formula I is selected from

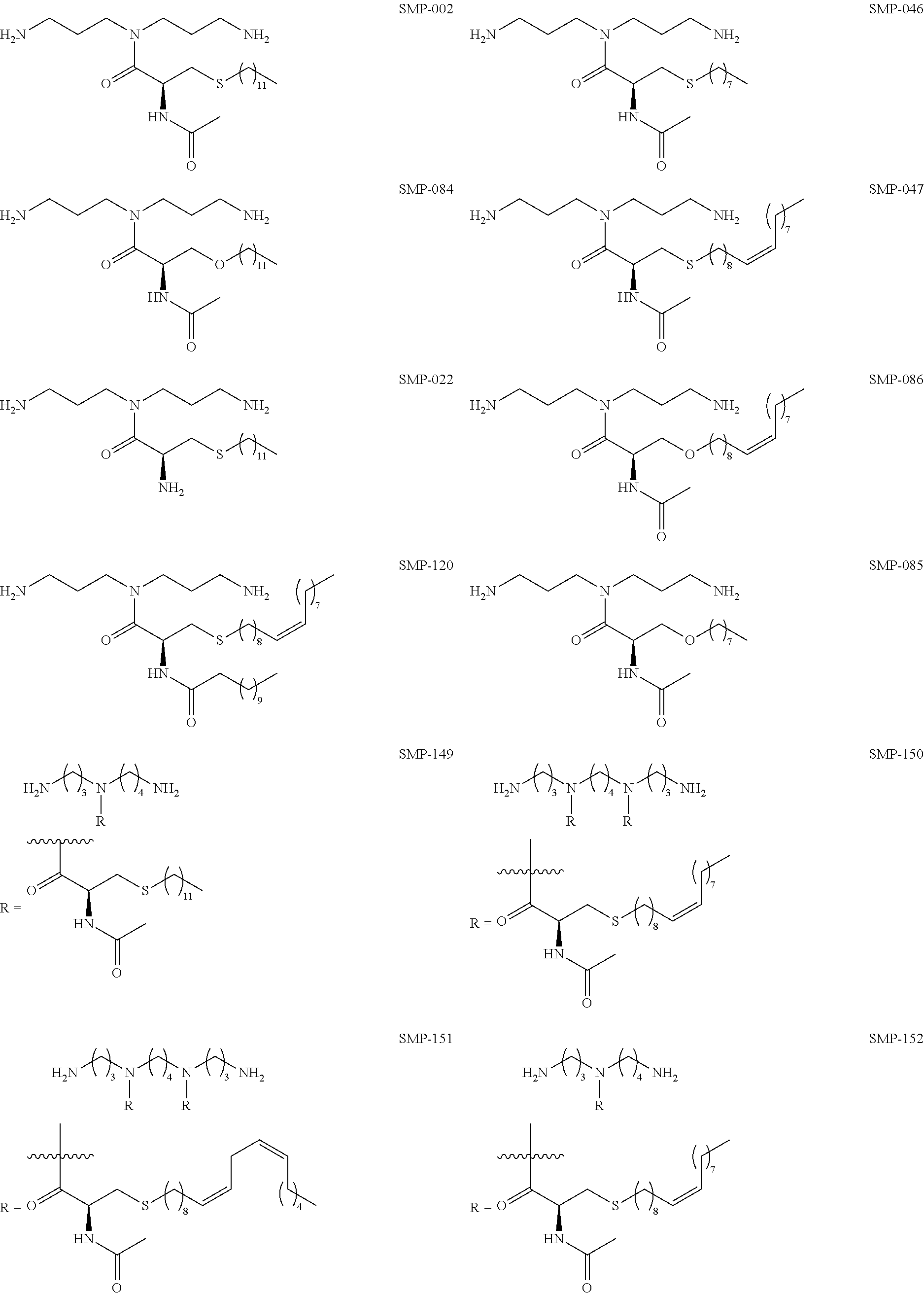

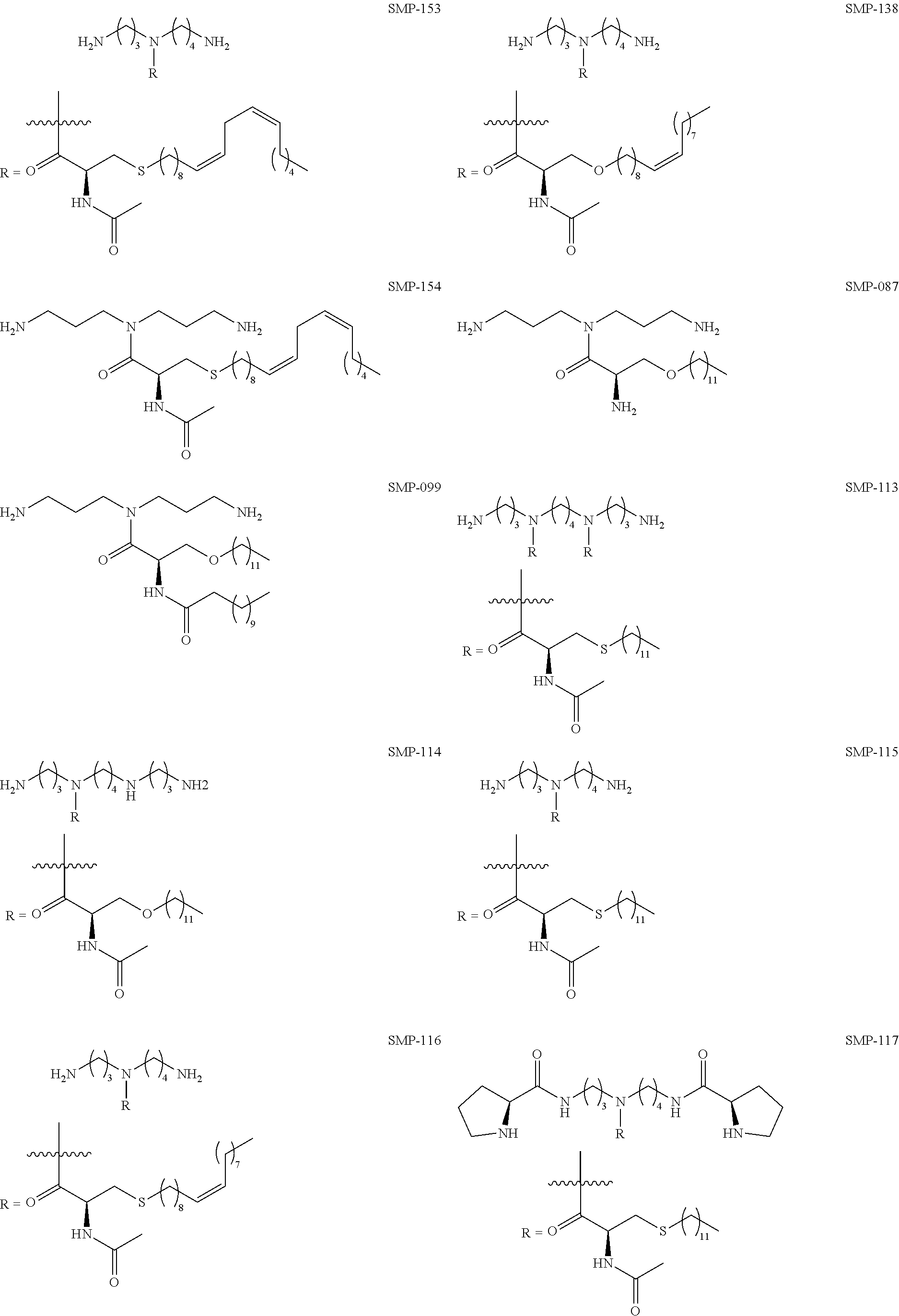

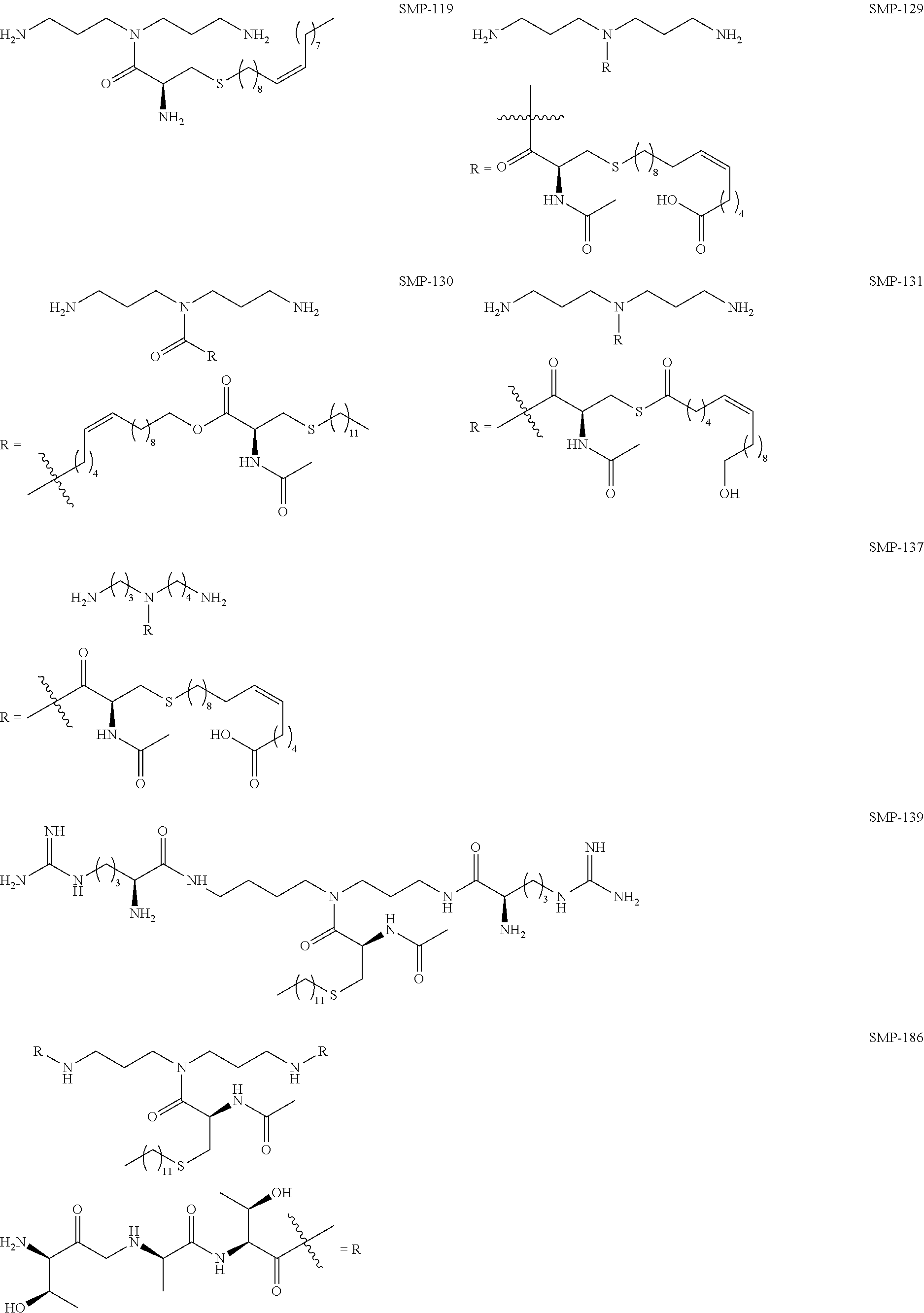

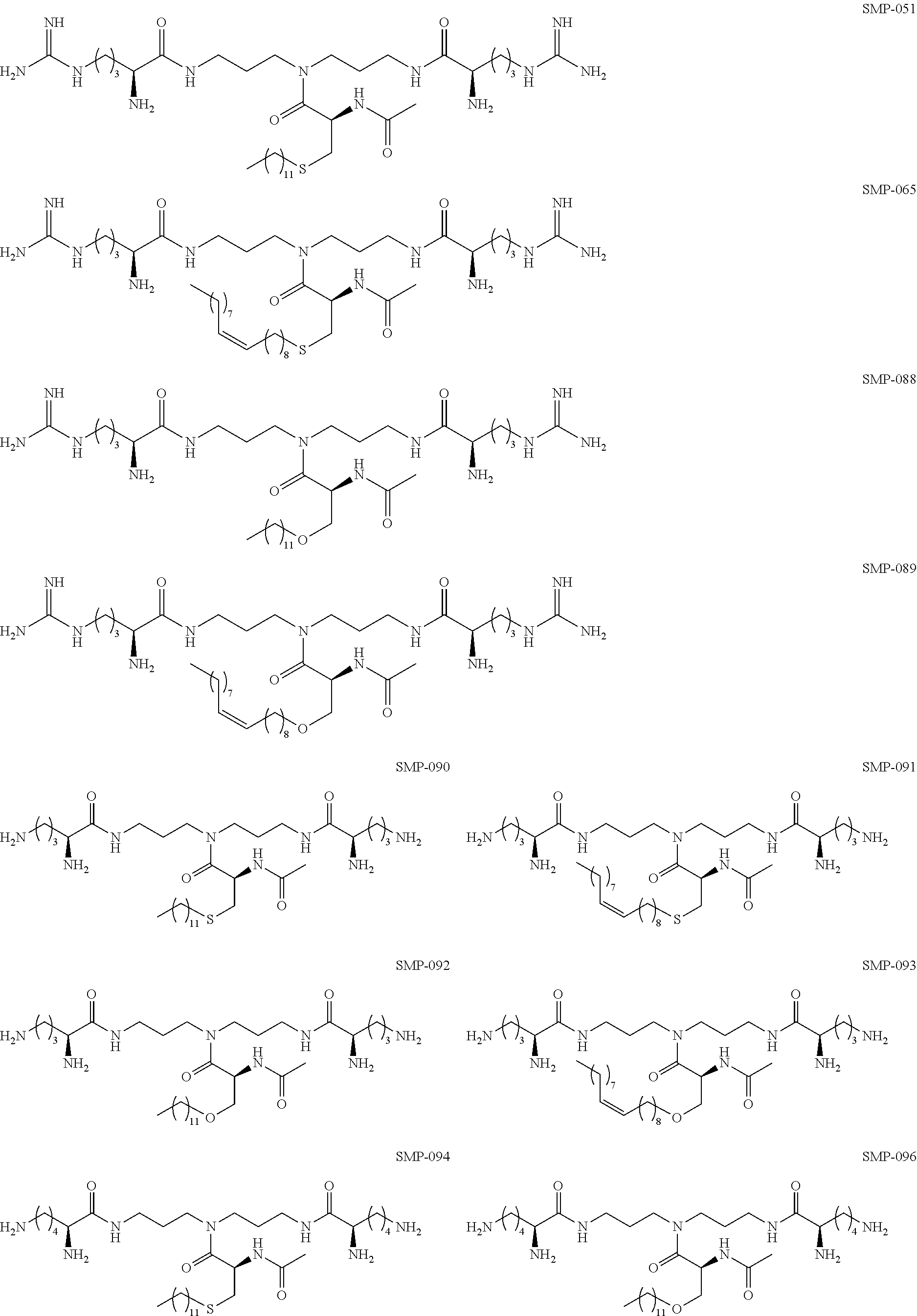

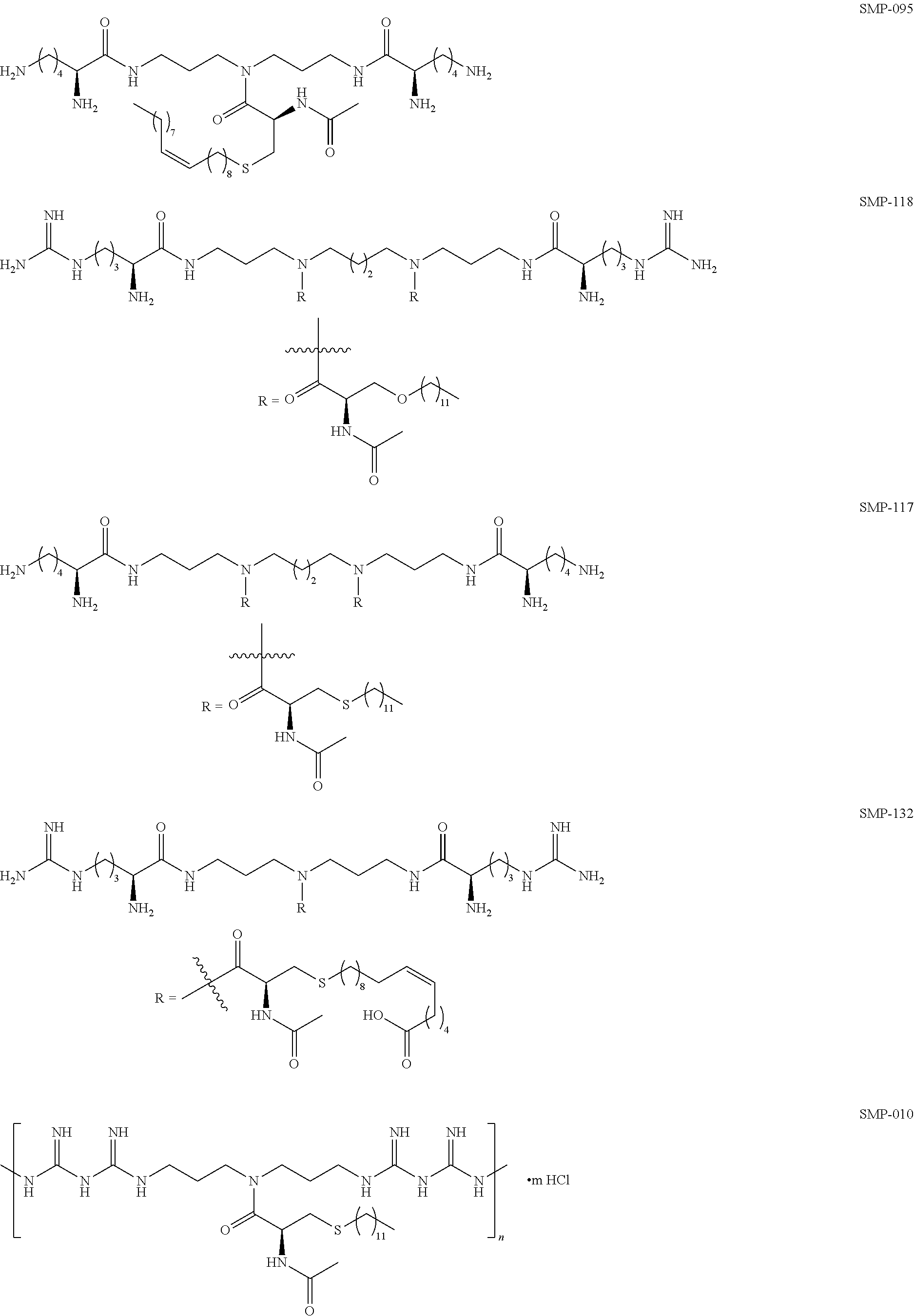

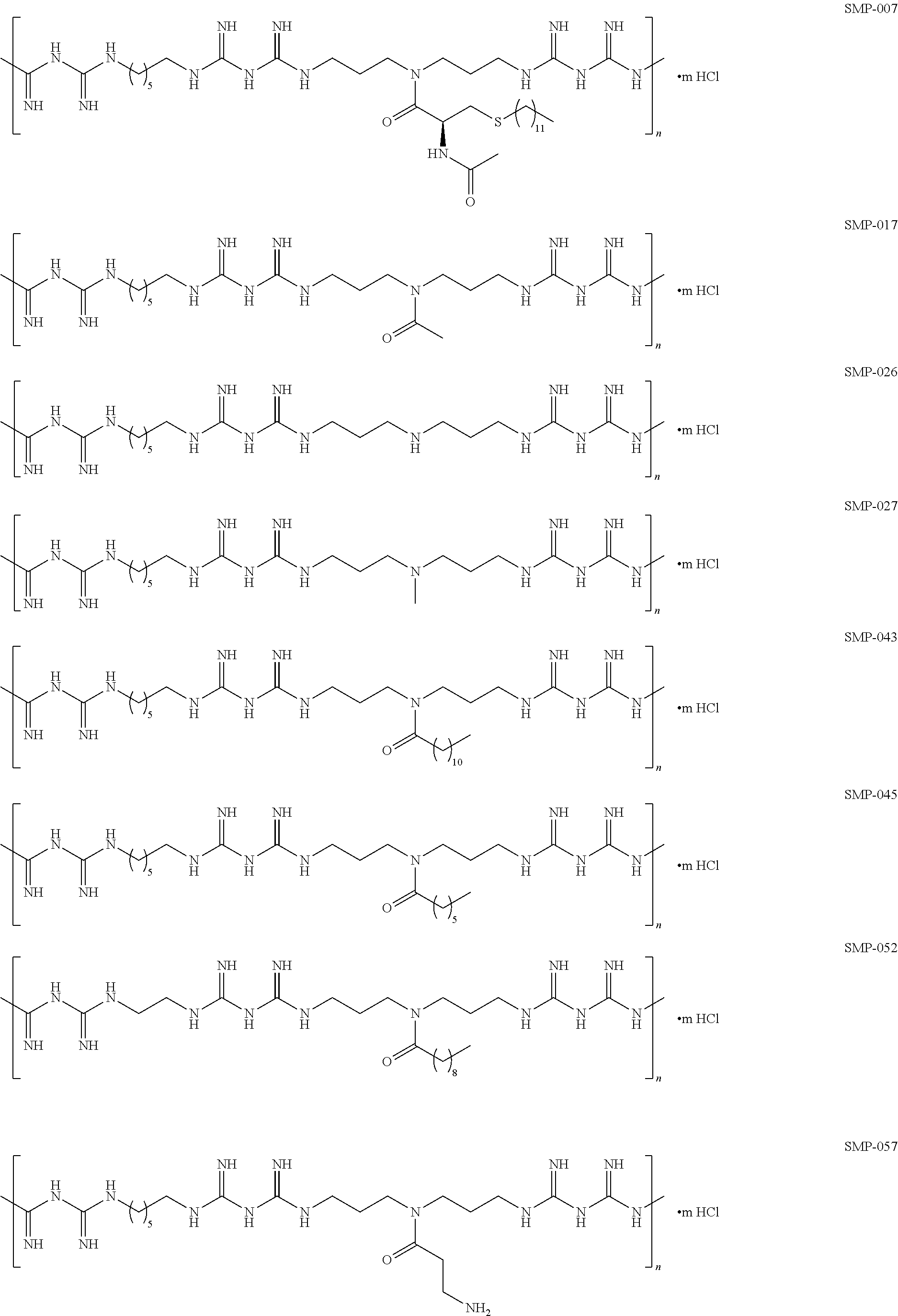

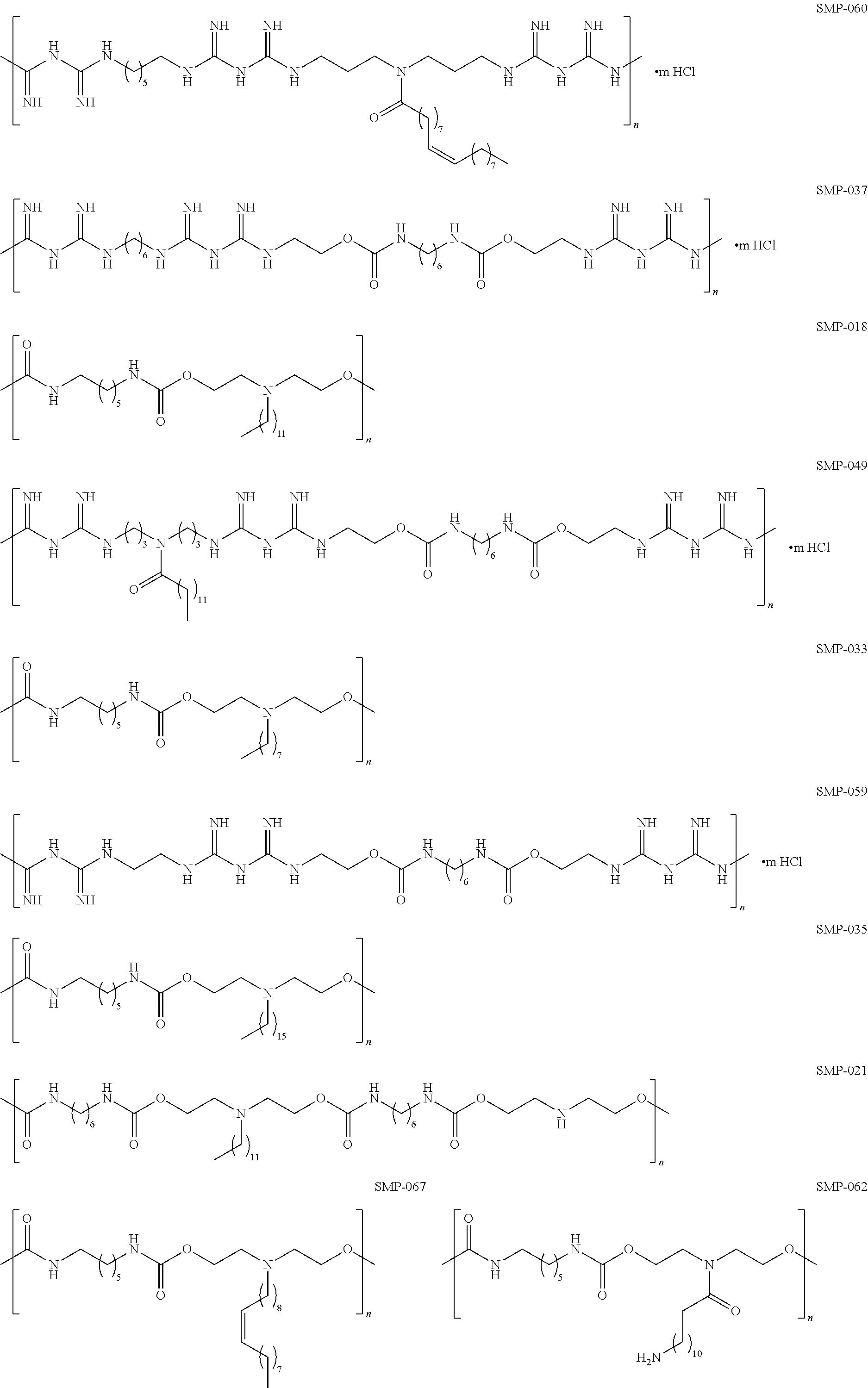

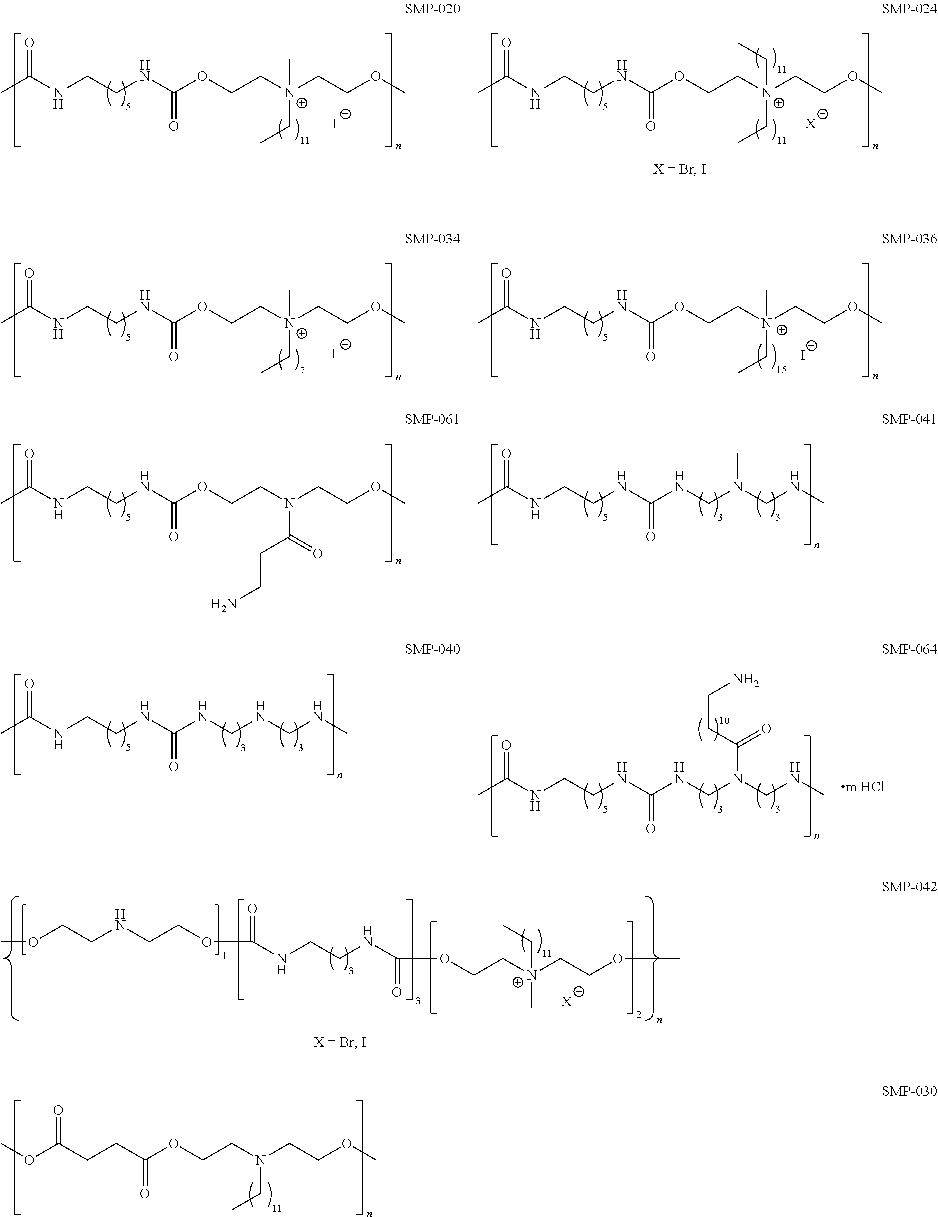

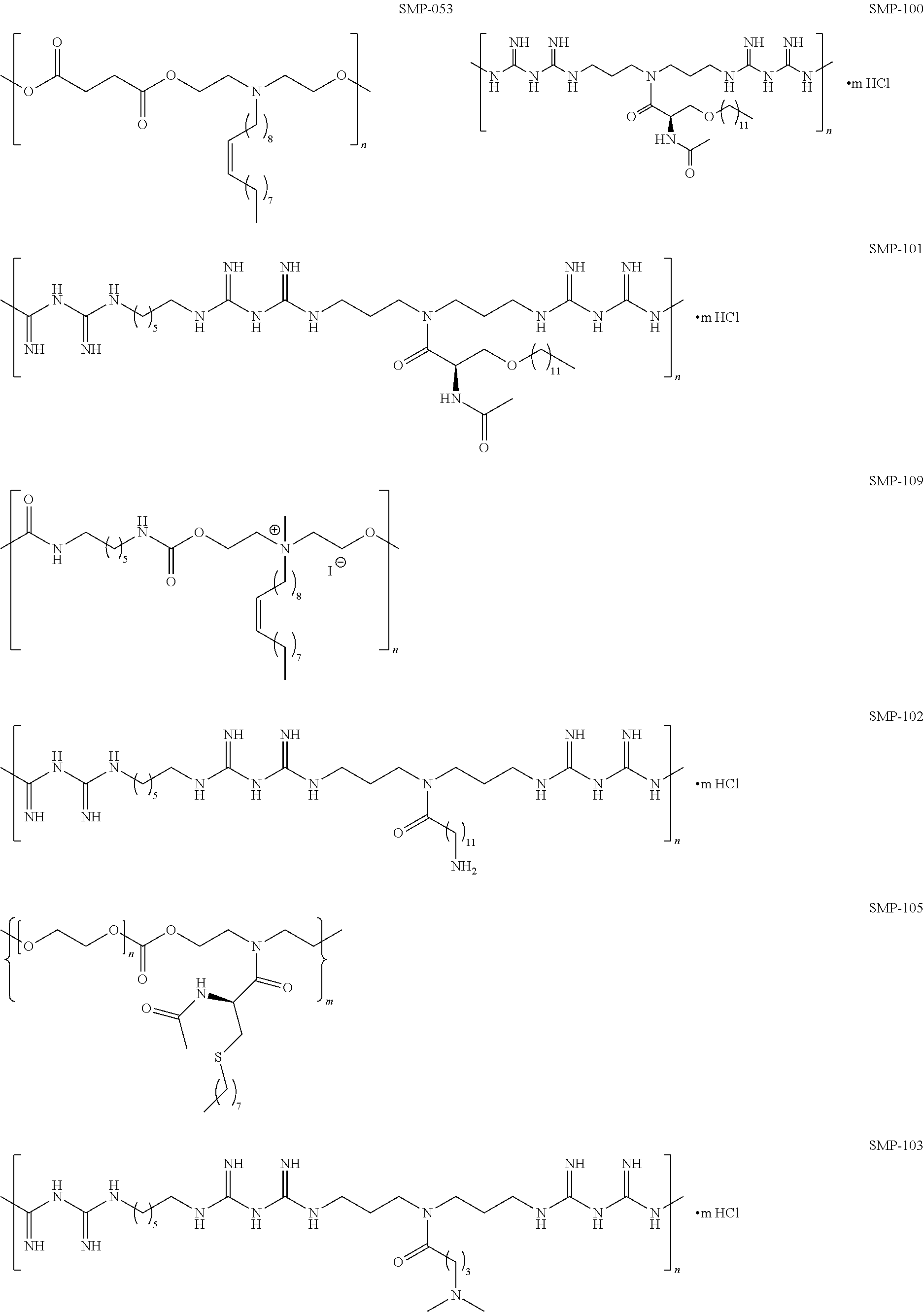

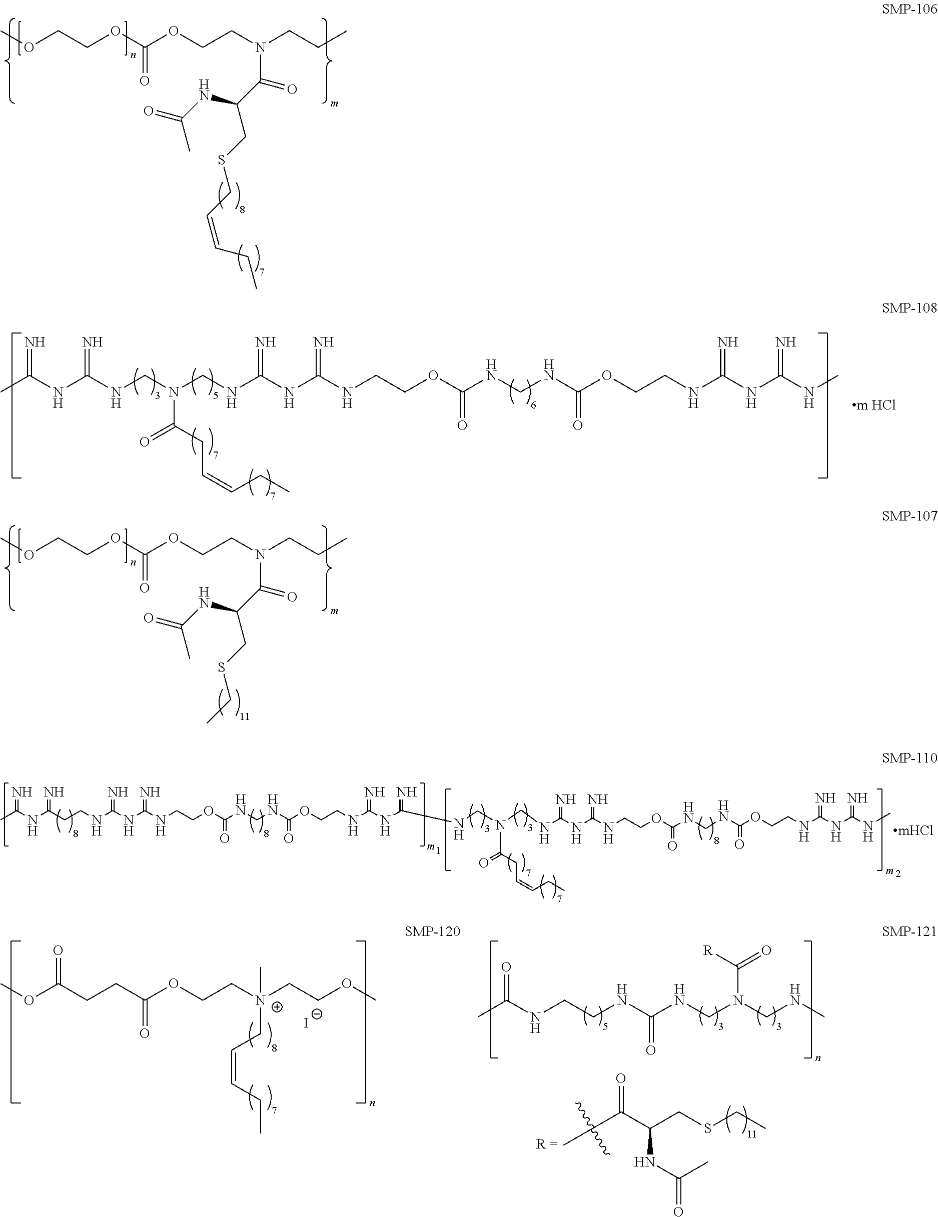

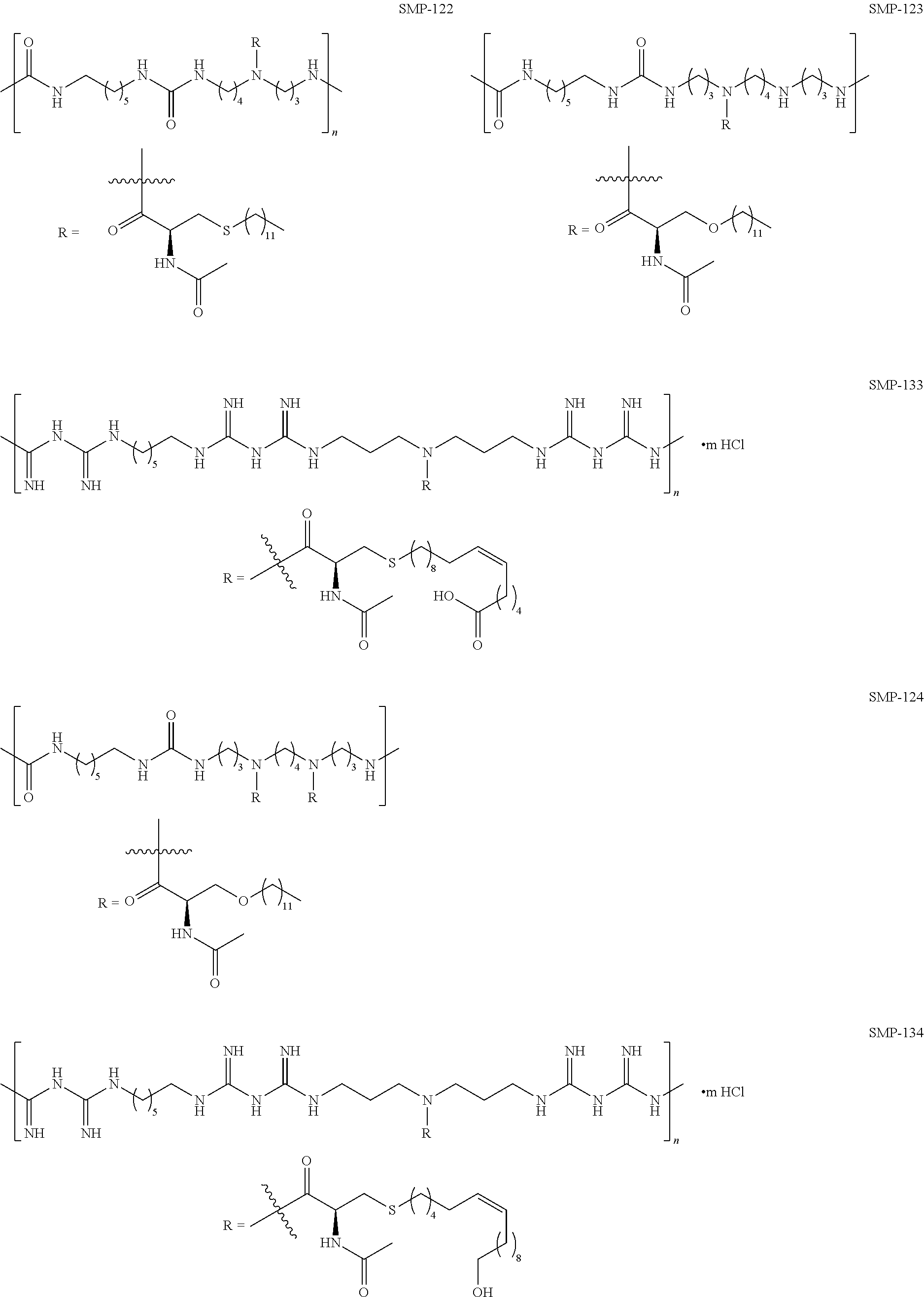

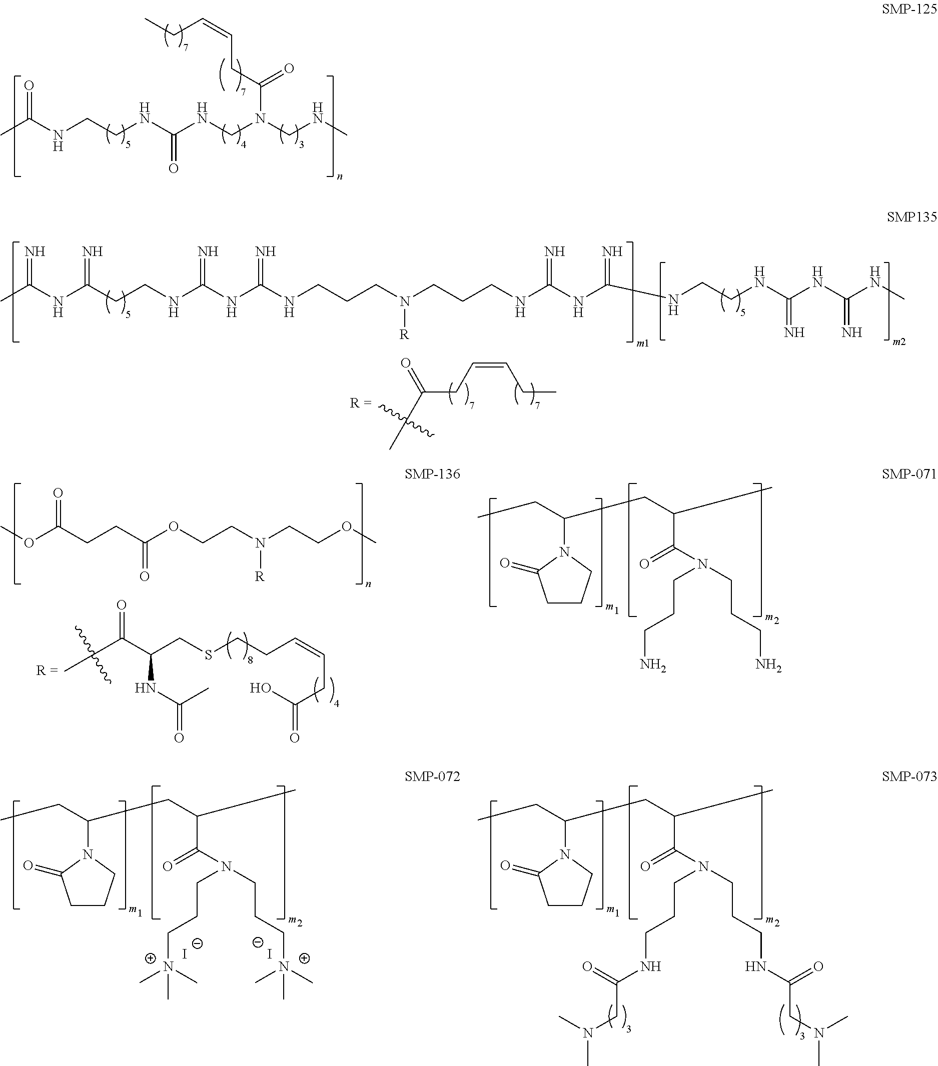

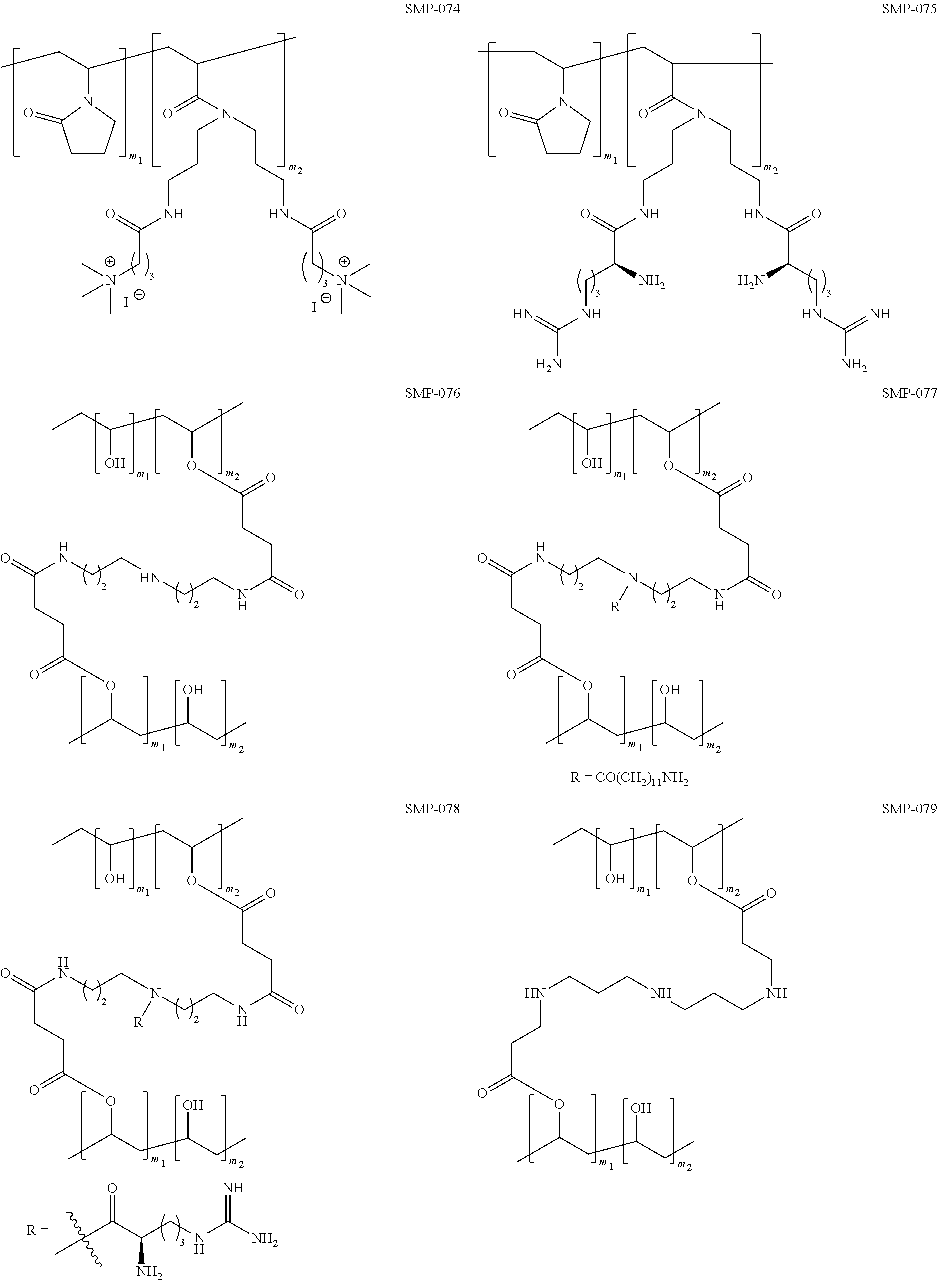

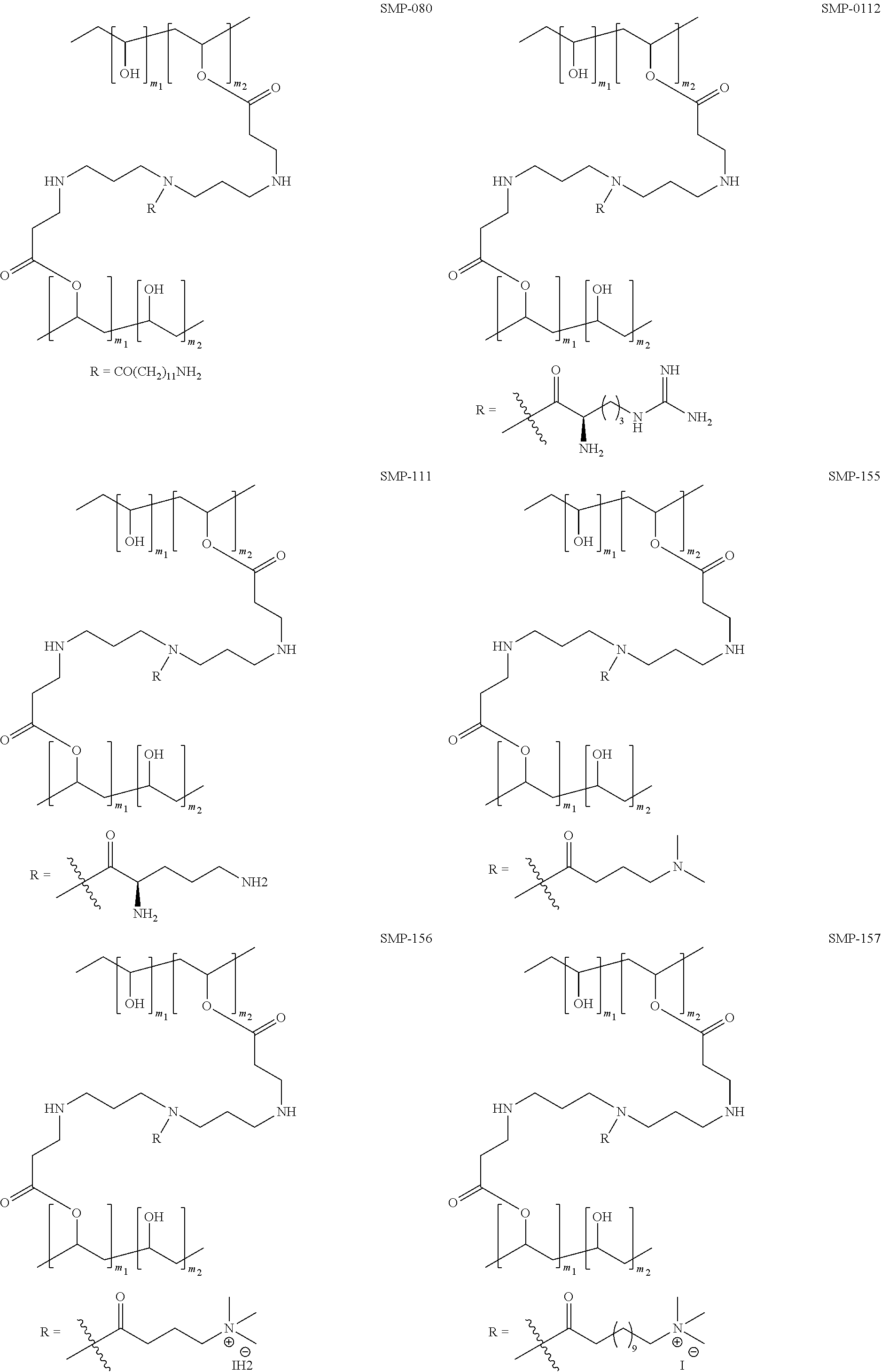

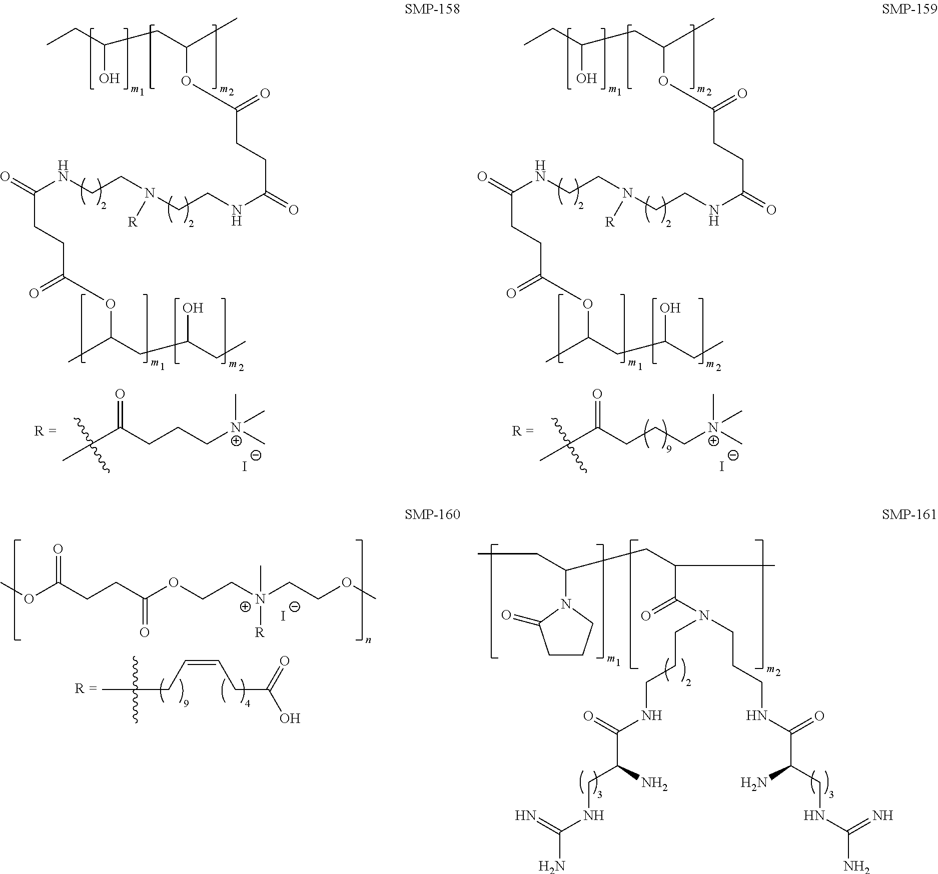

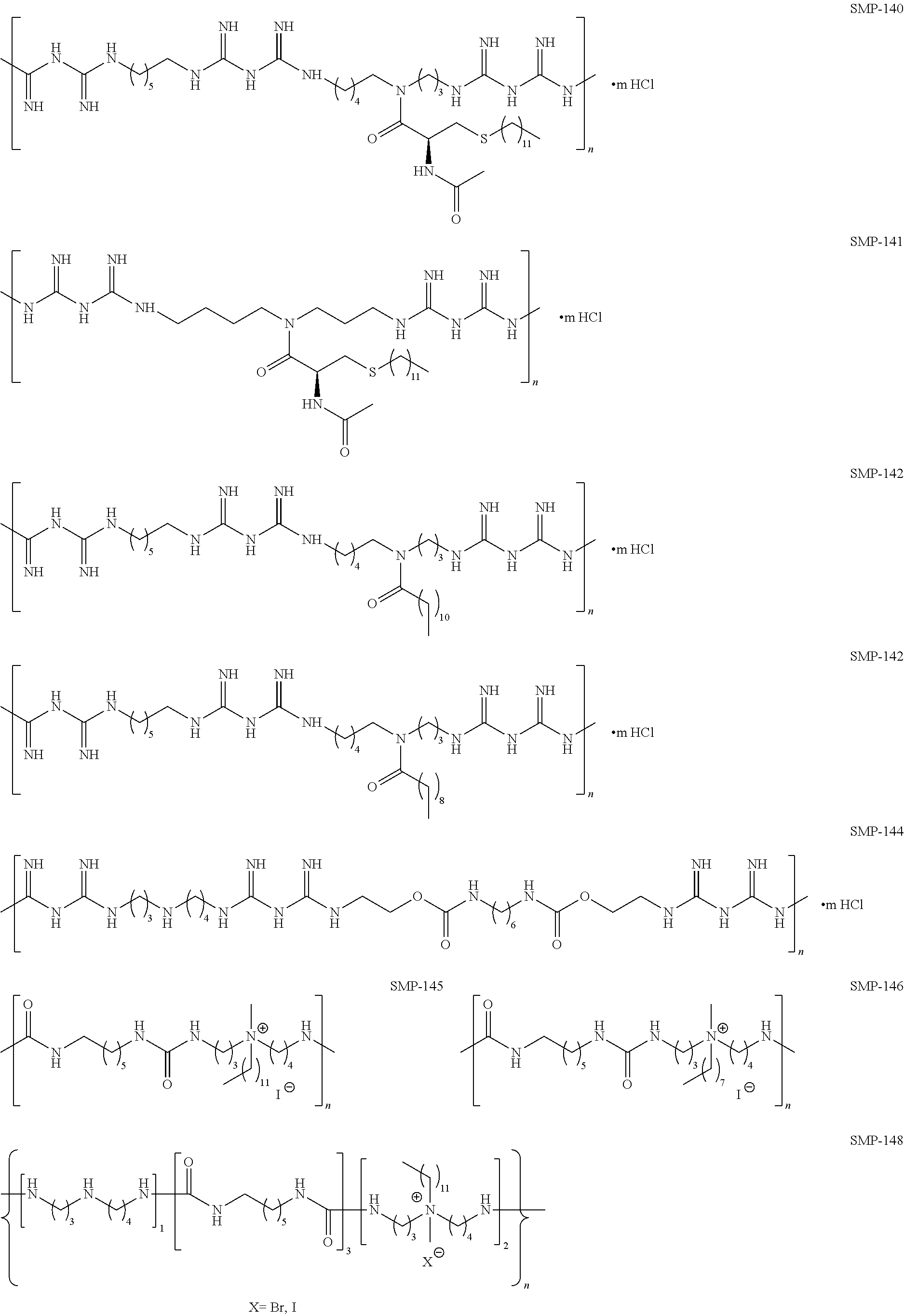

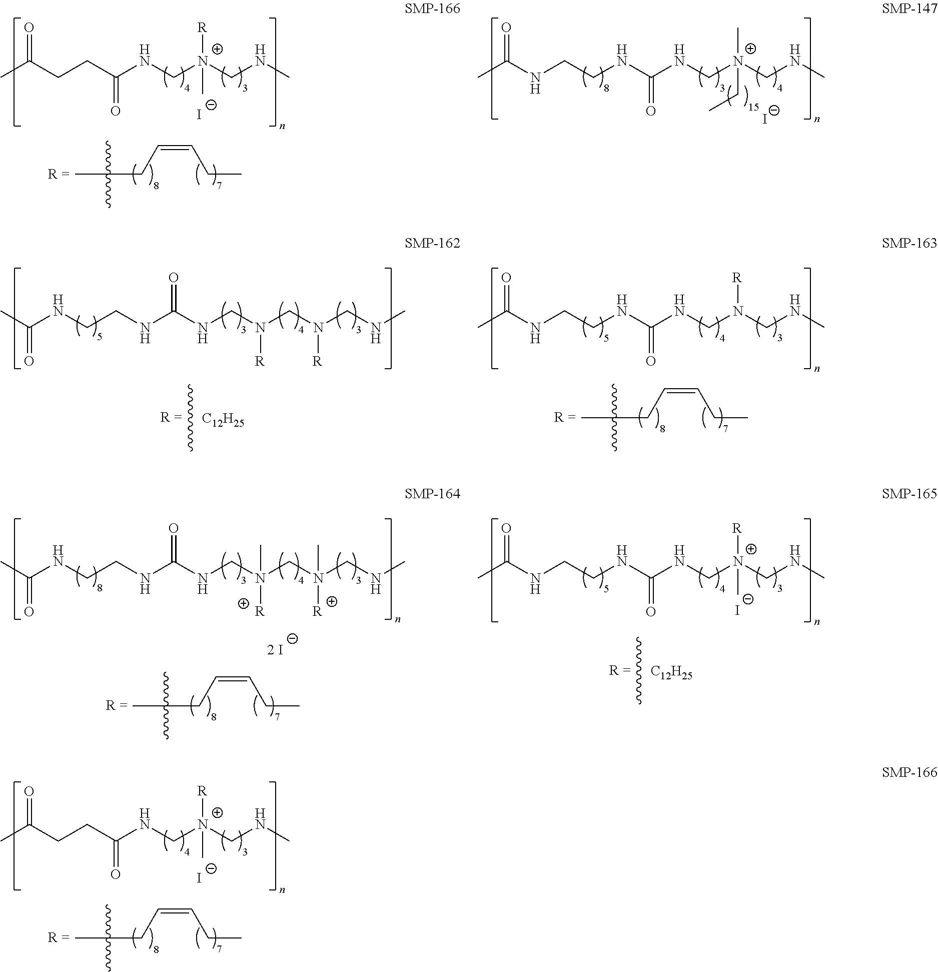

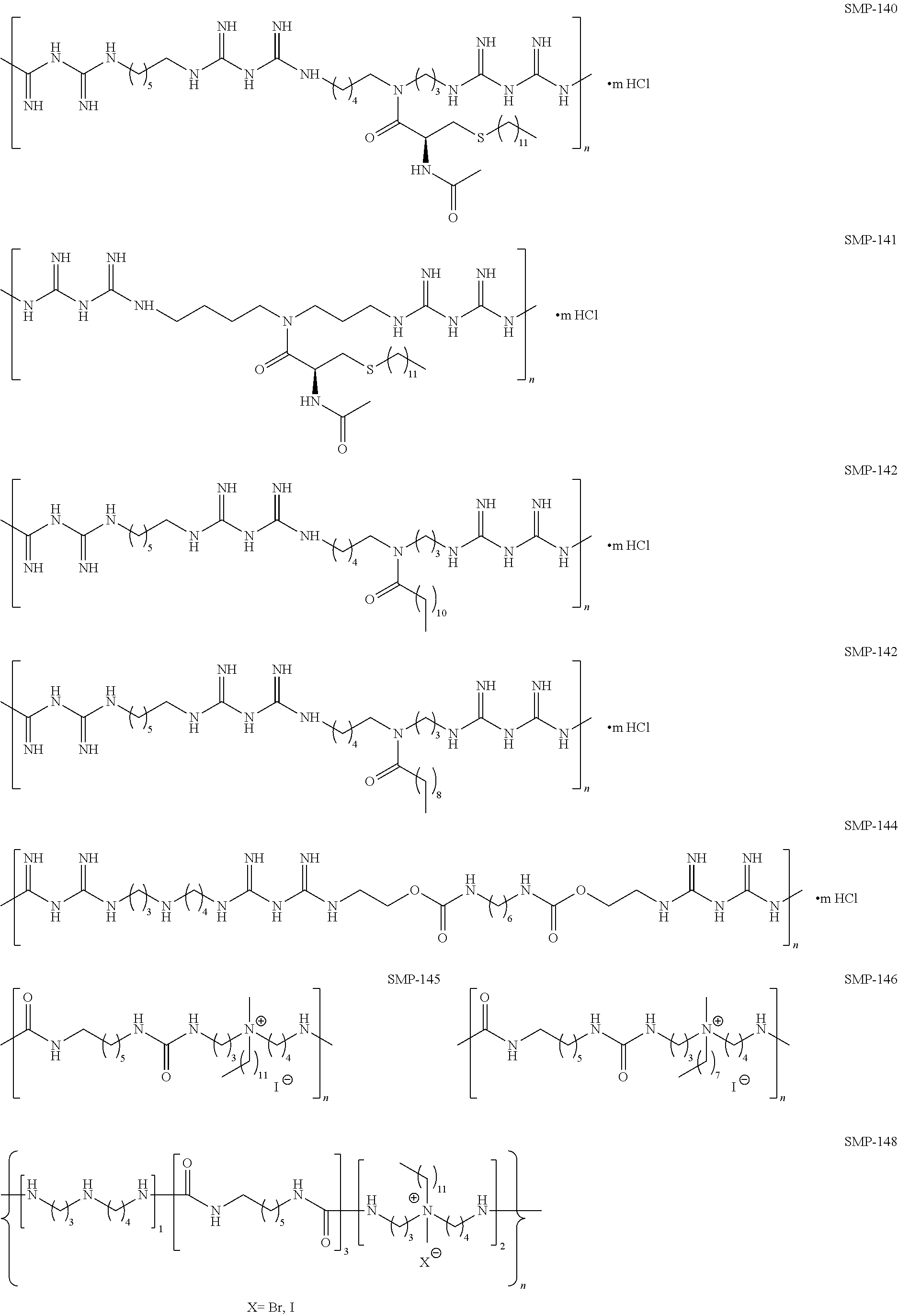

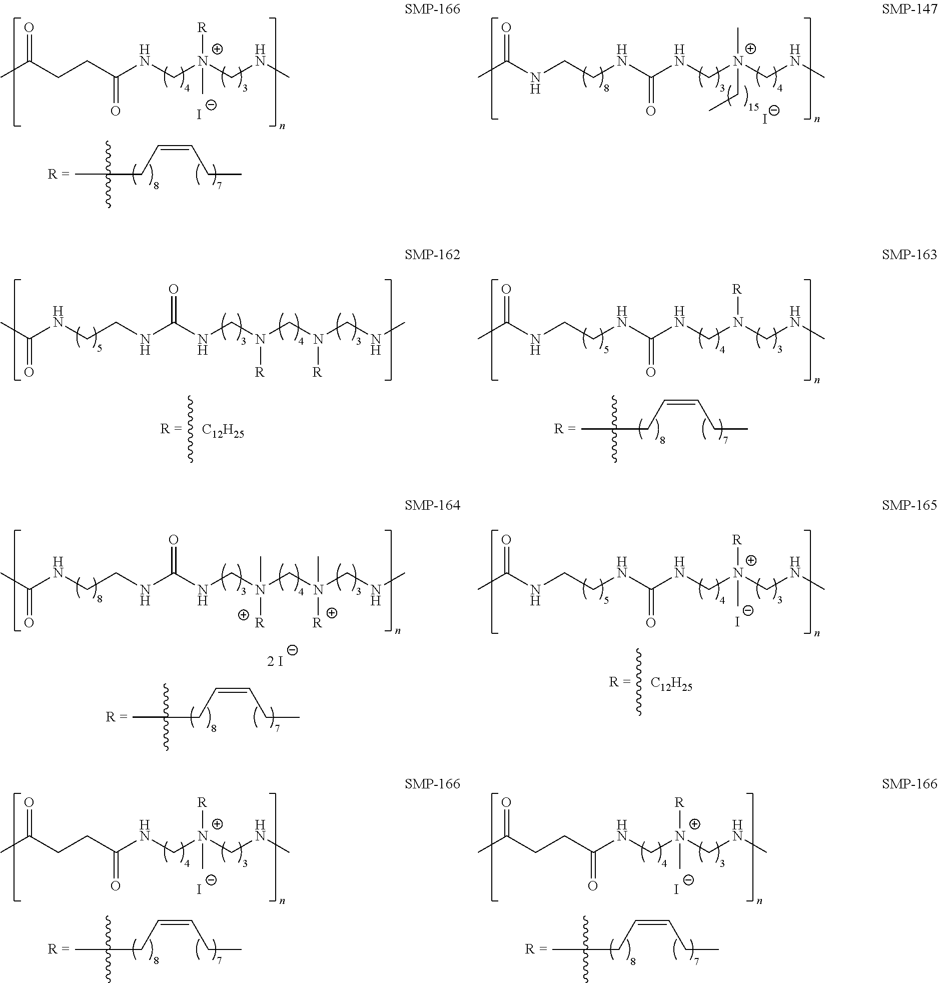

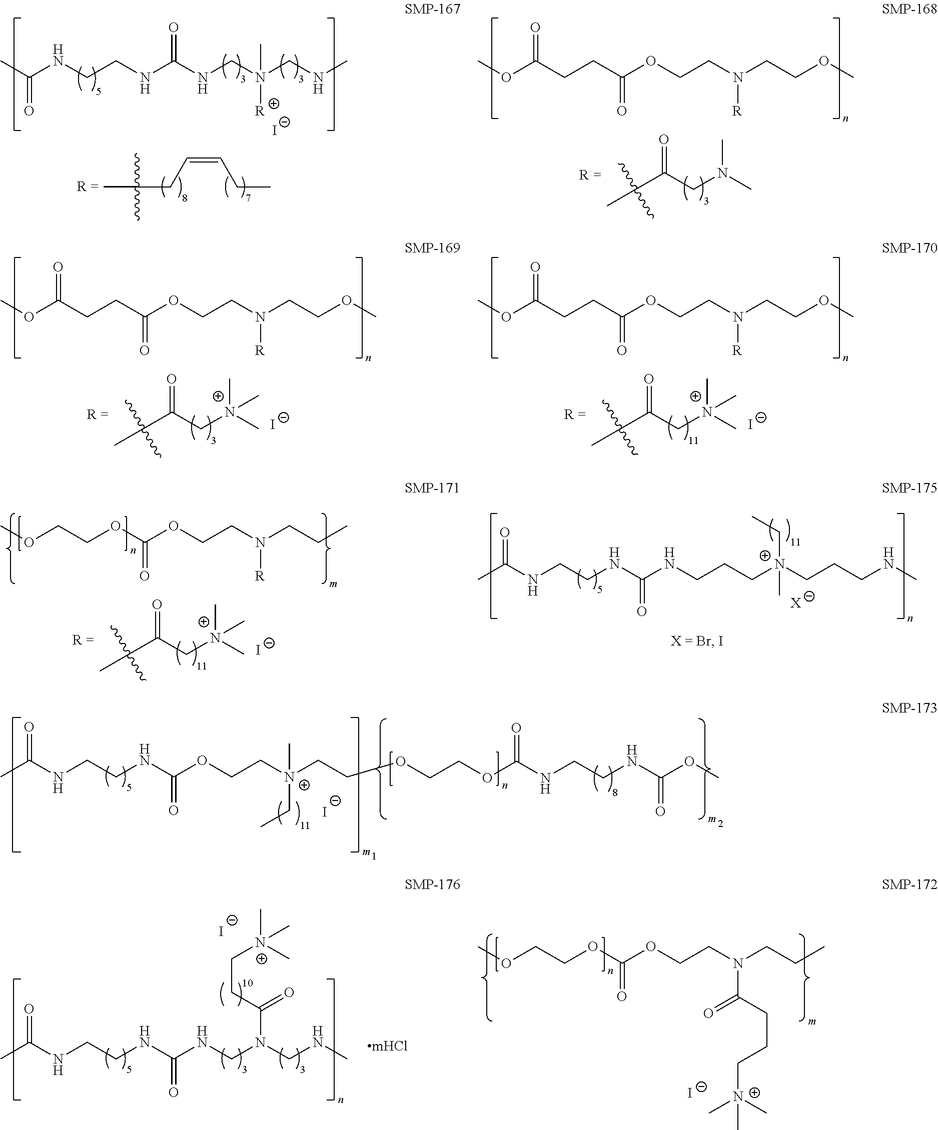

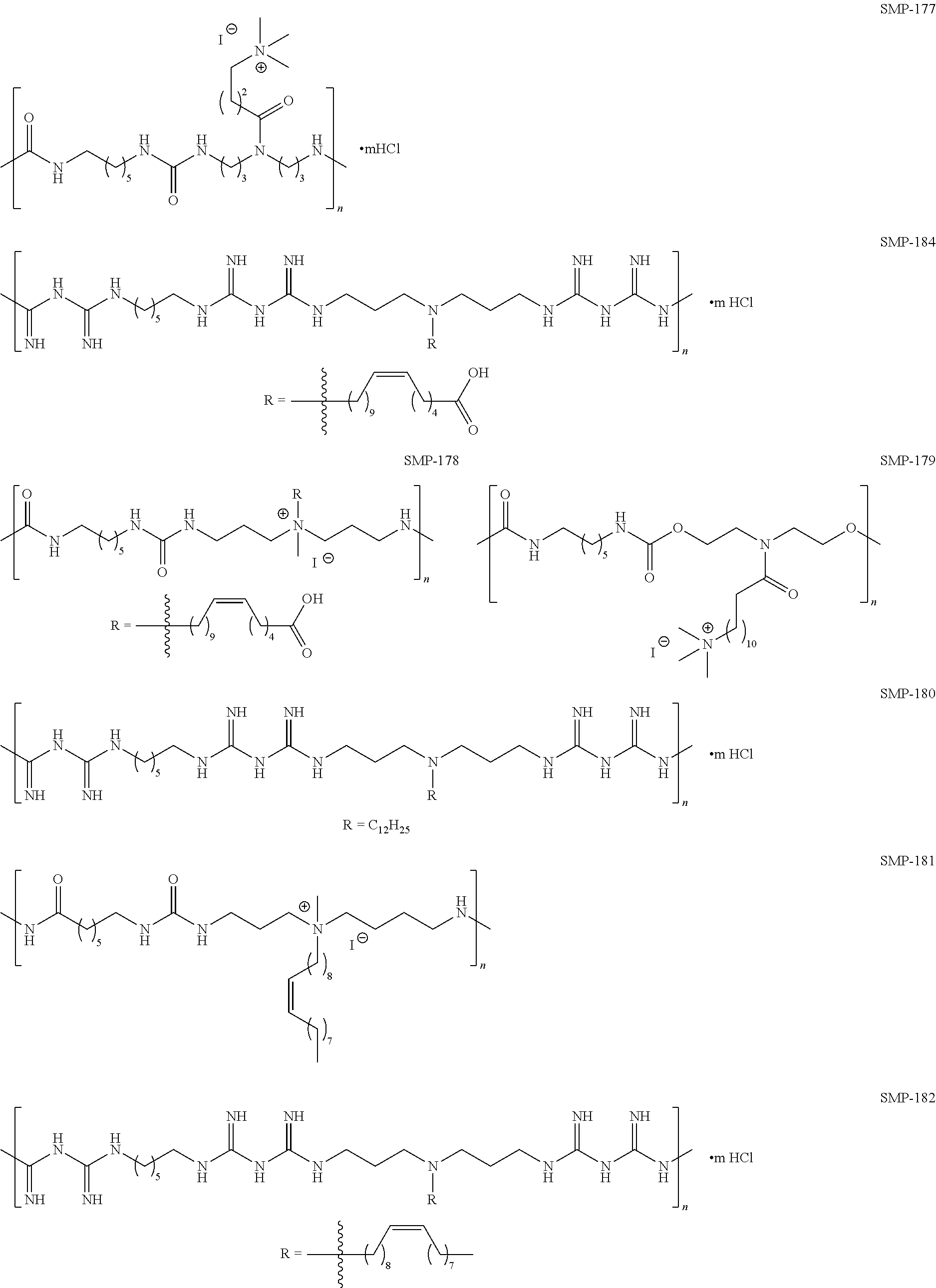

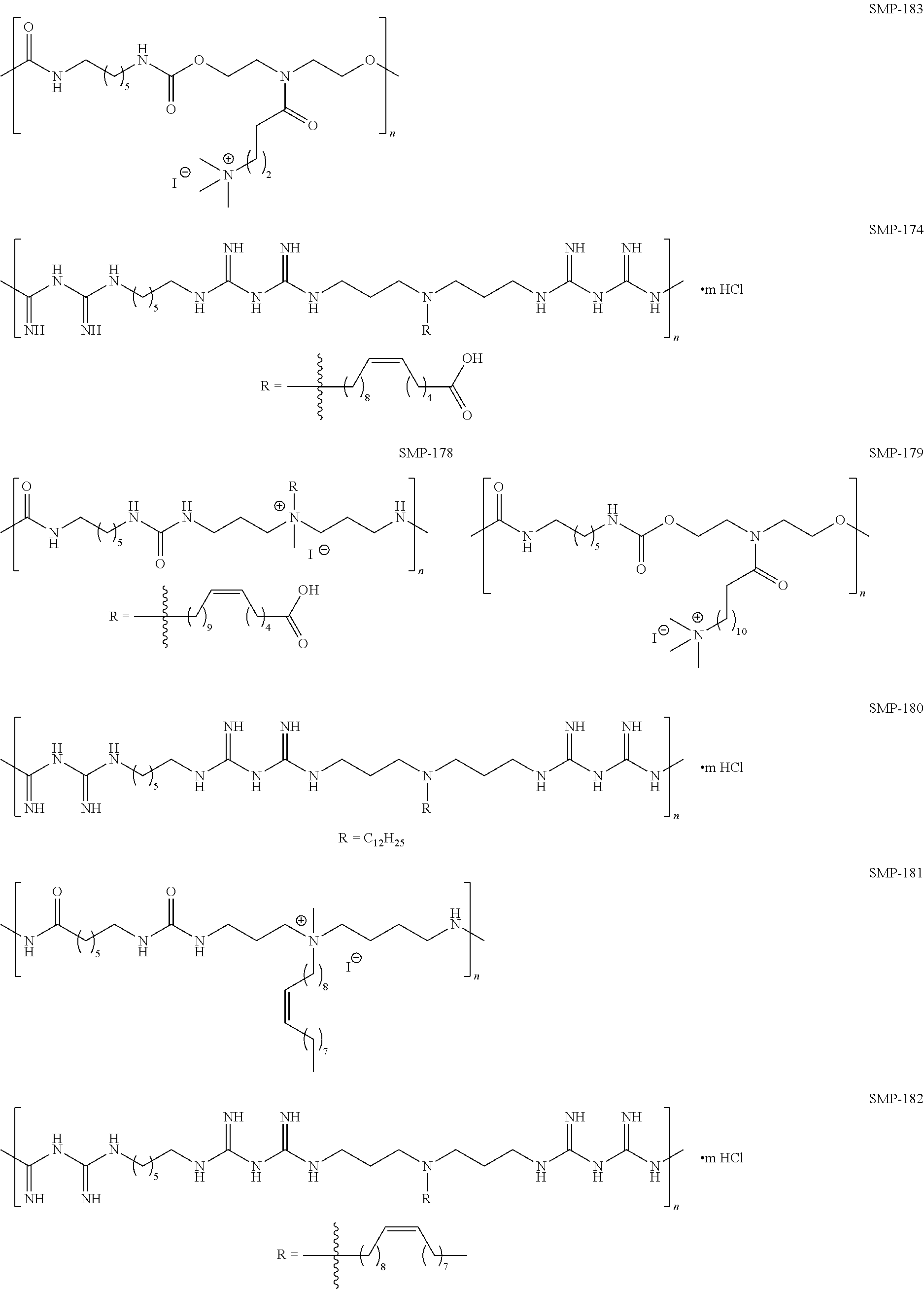

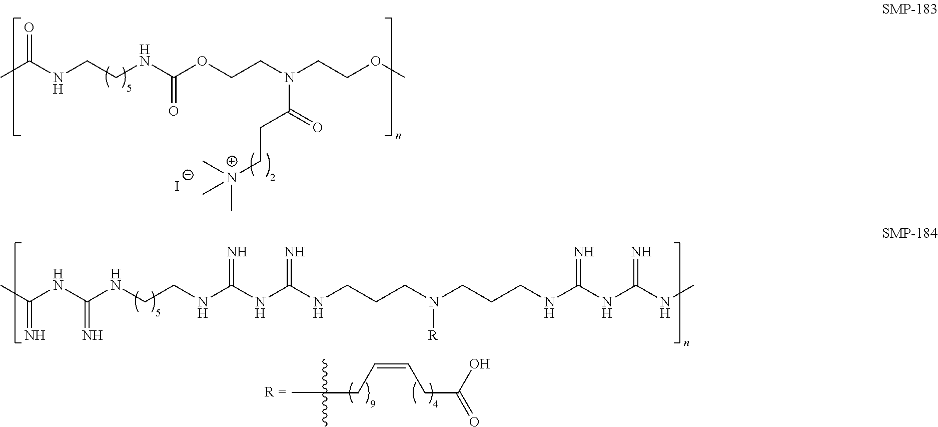

The present disclosure further provides a compound of Formula II:

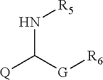

wherein ‘G’ is oxygen (—O—), sulphur (—S—), carbon (—C—), aryl, heteroaryl groups; Q is carboxylic acid, vinylacrylate, methylacrylate, halogen selected from fluoride, bromide, chloride or iodide, alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 and —(CH₂)p-CH═CH₂ with p=1-10, and substituted with carboxylic group, acrylic group, halogen selected from fluorine, chlorine, bromine or iodine, wherein ‘G’ is oxygen (—O—), sulphur (—S—), wherein R₅ and R₆ is independently hydrogen, alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 and —(CH₂)p-CH═CH₂ with p=1-10, and wherein each of R₅ and R₆ is optionally substituted with primary, secondary, tertiary or quaternary amino group, hydroxyl group, thiol group, carboxylic group, acrylic group, halogen selected from fluorine, chlorine, bromine or iodine, —COR₇, —COR₈ wherein R7 and R8 is alkyl, straight alkyl chain, branched alkyl chain, —(CH₂)n wherein n=1 to 30, alkenyl, alkynyl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10, —(CH₂)p-CH═CH₂ with p=1-10, aryl, heteroaryl and wherein each of the R₇ and R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide.

In an embodiment of the present disclosure, the compound of Formula II is

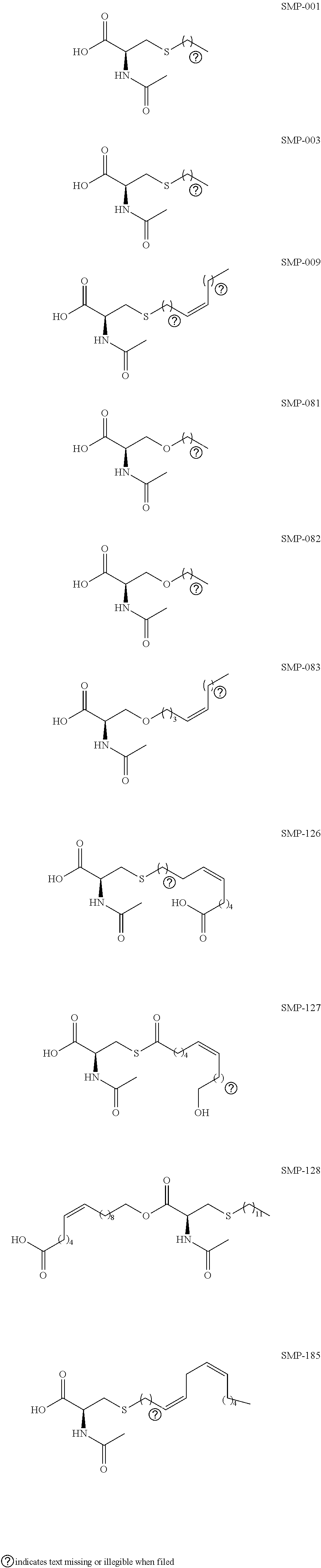

In another embodiment of the present disclosure, a process for preparing compound of Formula Ia as defined above is provided, wherein said process comprising step of reacting Formula II as defined above with polyamines, polyamine derivatives, or a combination thereof.

In yet another embodiment of the present disclosure, the compound of Formula I, Ib and Ic as defined above are obtained by self-polymerization of monomeric unit Formula Ia, or hetero-polymerization of Formula Ia with compound(s) selected from hexamethylenediamine, 1,6-bis(N³-cyano-N¹-guanidino)hexane, succinic anhydride, ethanol amine, PEG, acrylic or carboxylic derivative of ethanol amine or polyamines, bis(2-aminoethyl) hexane-1,6-diyldicarbamate, different monomeric units functionalized with guanidine, biguanidine, urethane, mixed guanidine-urethane, urea, ester, amide, carbonate, carbamate, or copolymerization or crosspolymerization with polymers or polymer derivatives selected from polyvinylpyrolidone (PVP), polyglycolic acid (PGA), polymethacrylate, polyacryl, polyacrylic acid (PAA), alginic acid, chitosan, PLGA, ethylene vinyl acetic acid, polyester, polyamide, polycarbamate, polycarbonate, PEG, PLA, PLA-PEG co-polymer, PHMB, polyguanidine, polybiguanidine, polyurethane, polybiguanidine-polyurethane, polyurea, polyester, polyamide or polycarbonate, or any combinations thereof.

In still another embodiment of the present disclosure, the polyamine described above is spermine, spermidine, norspermidine or putrescine, or any combinations thereof.

The present disclosure further relates to a composition comprising any of the compound as defined above, along with pharmaceutically acceptable excipient.

In an embodiment of the present disclosure, the composition comprises from about 0.1% to 20% (w/w) of the compound and about 80% to 99.9% (w/w) of the pharmaceutically acceptable excipient.

In another embodiment of the present disclosure, the composition comprises from about 0.1% to 5% (w/w) of the compound and about 95% to 99.9% (w/w) of the pharmaceutically acceptable excipient.

In yet another embodiment of the present disclosure, the excipient in the composition is selected from drug delivery carrier, emollient, moisturizer, emulsifier, stabilizer, surfactant, oil, lipid, wax, solubilizer, rheology modifier, thickening agent, gelling agent, preservative, antioxidant, film forming agent, pH modifier or other conventionally known pharmaceutically acceptable excipient, or any combination of excipients thereof.

In still another embodiment of the present disclosure, the composition is formulated into dosage forms selected from cream, gel, hydrogel, ointment, lotion, liposomal gel, micronized gel, powder, spray, solution, film, liquid bandage, patch, coating material on implant, coating material on a surface or matrix, wound dressing, or other suitable drug delivery vehicles, or any combination of dosage forms thereof.

In still another embodiment of the present disclosure, the composition treats a microbial infection or disease, and is administered to a subject in need thereof through modes selected from topical administration, local administration at wound infection or surgical site infection, intravenous administration, intramuscular administration, intraperitoneal administration, hepatoportal administration, intra articular administration or pancreatic duodenal artery administration, or any combination of modes thereof.

In still another embodiment of the present disclosure, the drug delivery carrier is biocompatible polymer, biodegradable polymer, bioabsorbable polymer or hydrogel forming polymer, or any combination of polymers thereof; and wherein the polymer is selected from polyvinylpyrolidine (PVP), polyglycolic acid (PGA), polyacrylic acid (PAA), alginic acid, chitosan, poly(lactic-co-glycolic acid) (PLGA), ethylene vinyl acetic acid, polyester, polyamide, polycarbamate, polycarbonate, polyethylene glycol (PEG), polylactic acid (PLA), PLA-PEG co-polymer, polyhexamethylene biguanide (PHMB), dextran, starch, polyguanidine, polybiguanidine, polyurethane, polybiguanidine-polyurethane, polyurea, polyester, polyamide or polycarbonate, or any combinations thereof.

The present disclosure also relates to a method of treating a microbial infection or disease comprising administering a compound or a composition as defined above, to a subject in need thereof.

In an embodiment of the present disclosure, the microbial infection described above is an bacterial infection, fungal infection, biofilm associated infection, or any combination thereof.

In another embodiment of the present disclosure, the microbial infection described above is a community acquired infection, health care-associated infection (HCAI) or a combination thereof; and wherein the community acquired infection is selected from superficial skin infection, topical wound infection, burn infection or diabetic foot infection, or any combinations thereof, and the health care-associated infection (HCAI) is selected from surgical site infections (SSIs), central line-associated bloodstream infections (CLABSI), catheter-associated urinary tract infections (CAUTI), ventilator-associated pneumonia (VAP), medical device associated infections or other health care-associated infection, or any combinations thereof.

In yet another embodiment of the present disclosure, the surgical site infection (S SI) described above is an implant associated infection caused by implant selected from orthopedic device, coronary stent, central venous and urinary catheters, heart valve, vascular graft, central nervous system implant, cochlear or dental implant, or any combinations thereof.

In still another embodiment of the present disclosure, the microbial infection described above is caused by microbe selected from Pseudomonas spp., Acinetobacter spp., Enterobacter spp., Klebsiella spp., Escherichia spp., Staphylococcus spp., Streptococcus spp., Enterococcus spp., Haemophilus spp., Propionibacterium spp. and Bacillus spp. Bacteroides spp., Fusobacterium spp., Clostridium spp., Candida spp. Malassezia spp. or Trichophyton spp., or any combinations thereof.

In still another embodiment of the present disclosure, the microbial infection described above is caused by microbe selected from P. aeruginosa, A. baumannii, E. aerogenes, K. pneumoniae, E. coli, S. epidermidis, S. aureus, E. faecium, S. pyogenes, H. influenzae, P. acnes, and Bacillus anthracis, B. fragilis, C. septicum, C. albicans, M. furfur or T. rubrum, or any combinations thereof; and drug resistant microbe strains of S. aureus, Pseudomonas spp., Acinetobacter spp., Enterobacter spp., Klebsiella spp., Escherichia spp., Staphylococcus spp., Propionibacterium spp., Bacillus spp., Streptococcus spp., Enterococcus spp., Haemophilus spp., Bacteroids, Fusobacterium, Clostridium, Candida spp., Malassezia spp. or Trichophyton spp., or any combinations of drug resistant microbes thereof; and wherein the drug resistant S. aureus is methicillin-resistant S. aureus (MRSA), methicillin resistant Staphylococcus epidermidis (MRSE), vancomycin resistant S. aureus (VRSA), vancomycin resistant S. epidermidis (VRSE) or vancomycin intermediate resistant S. aureus (VISA), or any combinations thereof.

In still another embodiment of the present disclosure, the compound described herein has a minimum inhibitory concentration ranging from about 1 microgram/ml to 500 microgram/ml, preferably 1 microgram/ml to 200 microgram/ml and/or a minimum biofilm disruption concentration (MBDC) ranging from about 0.01 milligram/ml to 20 milligram/ml, preferably about 0.01 milligram/ml to 10 milligram/ml.

In still another embodiment of the present disclosure, the method of treatment described above further comprises co-administering one or more additional anti-microbial agent to the subject.

In still another embodiment of the present disclosure, the compounds described herein possess wound healing activity.

The present disclosure further relates to a compound or a composition as defined above in the manufacture of a medicament.

In an embodiment of the present disclosure, use of a compound or a composition as defined above is provided for treating a microbial infection selected from bacterial infection, fungal infection, biofilm associated infection, or any combination thereof.

In another embodiment of the present disclosure, the compounds SMP-047, SMP-020, SMP-036, SMP-042, SMP-067, SMP-062, SMP-066, SMP-023, SMP-045, SMP-043 and SMP-026 possess potent antifungal activity, preferably against Candida species.

In yet another embodiment of the present disclosure, the compounds SMP-001, SMP-002, SMP-007, SMP-020, SMP-043, SMP-045, SMP-027, SMP-037, SMP-034, SMP-036, SMP-042, SMP-047, SMP-051, SMP-030 and SMP-126 possess biofilm disruption activity, preferably biofilm disruption activity against biofilm formed by gram positive, gram negative pathogen or a combination thereof.

The present disclosure provides SNAP (synthetic novel antimicrobial polymer) compounds/molecules. The compounds of the present disclosure have applications in managing microbial infections and associated diseases/complications including but not limiting to surgical site infections (SSIs).

SNAP technology of the present disclosure involves designing small molecules with potent antimicrobial activity that is either covalently linked to a polymeric backbone, or alternatively synthesis of a monomeric unit including said small molecule followed by polymerization to form synthetic novel antimicrobial polymer (SNAP). The bactericidal and anti-biofilm activities of SNAP molecules of the present disclosure have been evaluated against both antibiotic susceptible and resistant pathogens (bacteria and fungi). Polyamines like spermidine/norspermidine/spermine and N-Acetyl cysteine (NAC) derivatives were chosen as functional monomeric units which served as an important pharmacophoric moiety responsible for incorporating wide range of activity against broad range of clinically important pathogens. Mechanistically, SNAP molecules have electrostatic interaction with negatively charged bacterial and fungal cell walls resulting in membrane disruption, leakage of cellular components and cell death. Alternatively, the compounds of the present disclosure enter the cytosol and interact with chromosomal DNA resulting in inhibition of cellular replication and transcription processes and eventually cause cell death.

As used herein, “management” or “managing” refers to preventing a microbial infection, associated disease or disorder from occurring in a subject, decreasing the risk of death due to a microbial infection, associated disease or disorder, delaying the onset of a microbial infection, associated disease or disorder, inhibiting the progression of a microbial infection, associated disease or disorder, partial or complete treatment or cure of a microbial infection, associated disease or disorder and/or adverse effect attributable to the said disease or disorder, obtaining a desired pharmacologic and/or physiologic effect (the effect may be prophylactic in terms of completely or partially preventing a microbial infection, associated disease or disorder or condition or a symptom thereof and/or may be therapeutic in terms of a partial or complete cure for a microbial infection, associated disease or disorder and/or adverse effect attributable to the said microbial infection, associated disease or disorder), relieving a microbial infection, associated disease or disorder (i.e. causing regression of the microbial infection, associated disease or disorder). Thus, the present disclosure relates to aspects including but not limiting to prevention and/or treatment of a microbial infection and associated disease by administering therapeutically effective/efficacy dosage of the compounds or compositions disclosed herein.

As used herein, the terms such as Small anti-Microbial Polymer (SMP) molecules or SMP compounds, SNAP molecules or SNAP compounds are employed interchangeably within the instant disclosure and refer to the compounds or molecules of the present disclosure.

As used herein, the terms composition and formulation are used interchangeably in the present disclosure.

As mentioned above, the present disclosure provides compounds synthesized based on SNAP technology. The compounds are Formula I, Ia, Ib, Ic and II, respectively.

In an embodiment, a compound of the present disclosure is a compound having molecular scaffold of Formula I. The definition of substituents/groups in Formula I is as described above.

In some embodiments of the present disclosure, a pharmaceutically acceptable salt of a compound of Formula I is also provided. As used herein, the term “pharmaceutically-acceptable salts” refer to conventional nontoxic salts or quaternary ammonium salts of therapeutic agents, e.g., from non-toxic organic or inorganic acids. These salts can be prepared in situ in the administration vehicle or the dosage form manufacturing process, or by separately reacting a therapeutic agent in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), content of which is herein incorporated by reference in its entirety.

In an embodiment, ‘n’ in the compound of Formula I is 1.

In another embodiment, ‘n’ in the compound of Formula I is 2 to 1000.

In another embodiment, ‘n’ in the compound of Formula I is 2 to 500.

In yet another embodiment, ‘n’ in the compound of Formula I is 2 to 200.

In some embodiments of the present disclosure, all these polymeric scaffolds of Formula I can be present either alone or in combination resulting polyester or polyamide or polycarbamate or polycarbonate or alkyl linked hybrid or cross-link polymeric scaffolds with unique structural and antimicrobial properties. The examples and synthesis of the respective hybrid polymeric molecules based on Formula I is represented in schemes 22, 23, 24, 25 and 26 below.

In some embodiments, Formula I with polymeric scaffold (n=2-1000) are provided which contain different polymeric compounds with polybiguanidine (schemes 4-10) or polyurethane (schemes 13-16) or mixed polybiguanidine-polyurethane (scheme 11 and 12) or polyurea (schemes 17 and 18) or polyester (scheme 19) or polyamide (scheme 20) or polycarbonate (scheme 21) or polycarbamate or other functional linkages known to the person skilled in the art or combinations thereof.

In an embodiment, the compounds of Formula I comprise different L-amino acids such as arginine, ornithine, cysteine, histidine, glycine, serine, threonine, lysine, tyrosine, metabolic by-products of L-amino acids, or oligo-peptides such as TAT, Cholesterol-conjugated G3R6TAT (dodecapeptide, G3R6TAT), MP196 (hexapeptide, RWRWRW—NH₂), PAF-26 (hexapeptide , RKKWFW), Mastoparan(Polybia-MP1, tetradecapeptide, IDWKKLLDAAKQIL), D-IK8 (octapeptide, IRIKIRIK), L5K5W(undecapeptide, KKLLKWLKKLL-NH₂), Gramicidin-D (pentadecapeptide, VGALAVVVWLWLWLW), WR12 (dodecapeptide, RWWRWWRRWWRR) and Protegrins (PG-1, octadecapeptide, NH₂—RGGRLCYCRRRFCVCVGR—CONH₂)). In another embodiment, arginine, ornithine and their different metabolic by-products and small peptides (1 to 20 amino acid based) are involved in tissue repair including epithelization and collagen formation, and thus have important role towards wound healing process. Arginine is the sole precursor of nitric oxide, a signal molecule involved in immune responses, epithelization and formation of granulation tissue an essential aspect accompanying wound healing. In yet another embodiment, cysteine, histidine and glycine are known to reduce the activation of NF κB and IL-8 in THP-1 cells and provide anti-inflammatory effect. In other embodiments, oligopeptides such as TAT, D-IK8, MP196, L5K5W, cholesterol conjugated G3R6TAT and WR12 exhibit potent activity towards multi-drug resistant Gram positive and Gram-negative pathogens.

The present disclosure provides a polymer of Formula Ia. The definition of substituents/groups in Formula Ia is as described above.

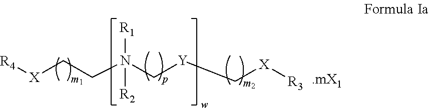

The present disclosure further provides a polymer of Formula Ib. The definition of substituents/groups in Formula Ib is as described above.

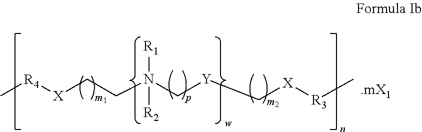

In some embodiments of Formula Ib with n=2-1000 and X═NH or O, self-polymerization of either monomeric unit represented as Formula Ia or other functional monomeric scaffolds result in different polymeric scaffolds with polybiguanidine or polyurethane or mixed polybiguanidine-polyurethane or polyurea or polyester or polyamide or polycarbonate or polycarbamate or other functional linkages known to the person skilled in the art or combinations thereof. In other embodiments, heteropolymerization between Formula Ia or other functional monomeric scaffolds and the known monomeric scaffolds like hexamethylenediamine or hexamethylenediisocyanate or succinic anhydride or ethanol amine or PEG or respective derivatives or others known to a person skilled in the art leads to polybiguanidine or polyurethane or polyamide or polyurea or mixed polyguanidium-polyurethane based compounds or other functional group known in the prior art, or any combinations thereof. In other embodiments, copolymerization or crosspolymerization between Formula Ia or other functional monomeric scaffolds and known polymer derivatives including polyvinylpyrolidone (PVP), polyglycolic acid (PGA), polymethacrylate, polyacryl, polyacrylic acid (PAA), alginic acid, chitosan, PLGA, ethylene vinyl acetic acid, polyester, polyamide, polycarbamate, polycarbonate, PEG, PLA, PLA-PEG co-polymer, PHMB, polyguanidine, polybiguanidine, polyurethane, polybiguanidine-polyurethane, polyurea, polyester, polyamide or polycarbonate, or combinations thereof leads to polybiguanidine or polyurethane or polyamide or polyurea or mixed polyguanidium-polyurethane based compounds or other functional groups known in the prior art, or any combinations thereof.

The present disclosure further provides a polymer of Formula Ic. The definition of substituents/groups in Formula Ic is as described above.

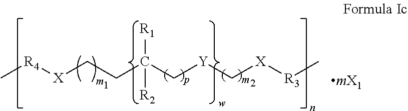

In an exemplary embodiment, the monomeric SMP molecules of Formula Ia are SMP-047, SMP-051, SMP-002, SMP-116, SMP-137, SMP-114, SMP-139.

In an exemplary embodiment, the polymeric SMP molecules of Formula Ib are SMP-020, SMP-042, SMP-007, SMP-010, SMP-060, SMP-067, SMP-110, SMP-108, SMP-140, SMP-146, SMP-080, SMP-077, SMP-078.

In another exemplary embodiment, the polymeric SMP molecules of Formula Ic are SMP-037, SMP-049.

The present disclosure further provides compound of Formula II. The definition/substituents of Formula II are as described above.

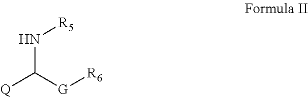

In yet another embodiment, few examples of SMP molecules represented by Formula II are follows

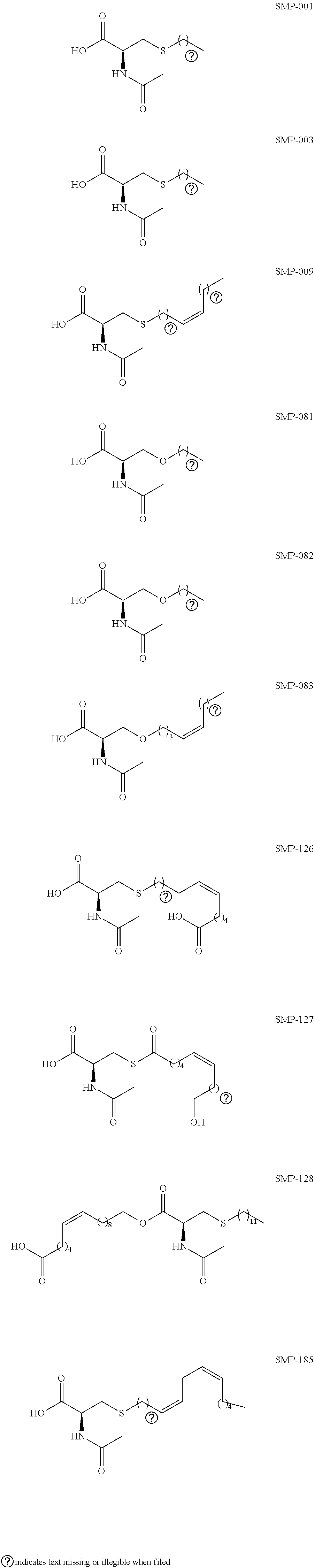

The present disclosure further provides compositions or formulations comprising the compounds described herein, along with pharmaceutically acceptable excipient(s). The compounds are provided in therapeutically effective amounts for management of microbial infections or associated disease/complications therein.

In an embodiment, the present disclosure provides topical anti-microbial compositions comprising antimicrobial compound(s) of Formula I, Ia, Ib, Ic, II as described herein, for the prevention and/or treatment of wound infections, surgical site and implant associated infection, wherein the antimicrobial agent(s) is present in solubilized or micronized or dispersed form in suitable topical dosage forms including but not limiting to cream, gel, ointment, lotion, liposomal gel, micronized gel, hydrogel, powders, sprays, solutions, liquid bandages, films, patches and/or other suitable drug delivery vehicles. Depending on the biodegradability, bio-compatibility, bio-absorbability and self-assembled hydrogel forming ability of the SMPs, they can be used as a film or coating material on implant or other foreign material (silicone or latex or titanium or others) surfaces for the prevention and/or treatment of bacterial and fungal infection. Further, SMPs prevent biofilm formation on foreign material surface thus lowering the chance of microbial infection lead to patient compliance. In some embodiments, SMPs can further be impregnated into polymeric implant or metal implant or bone cement or other polymeric carriers that would help to deliver SMPs at the site of action in a controlled, immediate or sustained manner.

The compounds present in different formulations including topical formulations range from 0.1 to 20% (w/w), preferably 0.1-10% (w/w) and more preferably 0.1-5% (w/w). The pharmaceutically acceptable excipient(s) present in the formulation ranges from 90-99.9% (w/w), preferably 80-99.9% (w/w), and more preferably 90-99.9% (w/w).

The pharmaceutically acceptable excipients in the present disclosure include suitable drug delivery carriers, emollients, moisturizers, emulsifiers, surfactants, oils, lipids, waxes, solubilizers, rheology modifiers, thickening agents, gelling agents, preservatives, antioxidants, film forming agents, pH modifiers and any combinations thereof.

In an embodiment, suitable drug delivery carriers include different polymers, lipids, oils and other known carriers, or any combinations thereof. In another embodiment, suitable surfactants, emulsifiers and stabilizers are used to solubilize and/or stabilize and/or enhance skin penetration of the active SMPs through stratum corneum. In another embodiment, the emollients and moisturizers are used to provide aesthetic feel as well as enhance skin penetration ability of the formulation. In yet another embodiment, the antioxidants, preservatives and pH modifiers are used to obtain stable SMP containing formulation including topical formulations.

In an exemplary embodiment, drug delivery carriers include polymers. The polymers are synthetic polymers, natural polymers or a combination thereof. In another embodiment, the polymers are biodegradable, biocompatible, bio-absorbable or any combination thereof. In a preferable embodiment of the present disclosure, the polymers include but are not limited to alginates, cellulose, carboxymethyl cellulose (CMC), hydroxyethyl cellulose (HEC), hydroxypropyl methyl cellulose (HPMC), cellulose ester derivatives, hyaluronic acids, hyaluronic acid derivatives, polyacrylic acid (PAA), polylactic acid (PLA) and derivatives, polycaprolactones, gelatin, sodium alginate, polyglycolic acid (PGA), poly(lactic-co-glycolic acid (PLGA), ethylene vinylacetate copolymer (EVA), dextran, triblock po-polymer of polyethylene oxide(PEO) and polypropylene oxide (PPO) like (PEO-PPO-PEO), polyvinyl alcohol (PVA), polyvinylpyrrolidone (PVP) and crosslink polymer of PVA-PVP and other suitable polymers. In another embodiment, carriers for hydrogel formulations are selected from the family of PVA with different molecular weights and different degrees of hydrolysis, PVP, ascorbic palmitate and respective ascorbic derivatives, polyethylene oxides, polyethylene glycols, polyglyceryl esters of fatty acids, acrylic acid co-polymers, sodium alginate, chondroitin sulfate, pectin, dextran, carboxymethyl cellulose, gelatin, gums and other cross-link polymers and co-polymeric compounds known to a person skilled in art. In a preferable embodiment, hydrogel carriers include but are not limited to carbopol, PLGA (poly(lactide-co-glycolic acid), PEG, PVA, PVP, polyacrylic acid, chitosan, alginate, dextran, sodium carboxymethyl cellulose, dextran and β-cyclodextrin, calcium-pectin, physically crosslink hydrogel like PVA-alginate, hyaluronic acid-methylcellulose, gelatin-agar, starch-carboxymethyl cellulose, and others, PLA (poly(1-lactic acid)), hyaluronic acid-PLA based co-polymer, poly(1-glutamic acid), PEG-PLA co-polymer, PEO-PPO-PEO based physically crosslink hydrogel or poly(N-isopropylacrylamide) (PNIPAM), or any combinations thereof.

In some embodiments, the formulations include liposome based topical compositions where hydrophobic SMP(s) are mixed with lipids at different ratios ranging from 1: 5 to 1:50. In an embodiment, the lipids in such formulations include are but not limited to saturated and unsaturated fatty acids of chain length C2-C24, hydrocarbons, fatty alcohols, glycerol derivatives of different fatty acids with C1-C36 alkanols, soy-lecithin, egg-lecithin, hydrogenated soy-lecithin, phospholipids, sphingolipids, glycolipids, cholesterol or cholesterol ester derivatives, phospholipids, ceramides with different degree of saturation and acyl chain length, or any combination of lipids thereof. In another embodiment, suitable hydrocarbons include, but are not limited to mineral oil, isohexadecane, squalane, hydrogenated polyisobutene, petrolatum, paraffin, microcrystalline wax; fatty alcohols include but are not limited to decanol, dodecanol, tetradecanol, hexadecanol, octadecanol or combinations thereof; fatty acids include but are not limited to C6-C24 alkanoic acids such as hexanoic acid, octanoic acid, decanoic acid, dodecanoic acid, tetradecanoic acid, hexadecanoic acid, octadecanoic acid, unsaturated fatty acids such as oleic acid and linoleic acid; glycerides include but are not limited to olive oil, castor oil, sesame oil, caprylic/capric acid triglyceride or glycerol mono, di and tri-esters with palmitic and/or stearic acid; esters of fatty acids include but are not limited to C1-C36 alkanols such as beeswax, carnauba wax, cetyl palmitate, lanolin, isopropyl myristate, isopropyl stearate, oleic acid decyl ester, ethyl oleate and C6-C12 alkanoic acid esters and other esters of fatty acids; solubilizers are selected from but are not limited to water, polyethylene glycol, isopropanol, propylene glycol, isopropyl myristate, diethylene glycol monoethyl ether, ethanol, oils, buffers and combinations thereof; oils include but are not limited to one or more of almond oil, apricot seed oil, borage oil, canola oil, coconut oil, corn oil, cotton seed oil, fish oil, jojoba bean oil, lard oil, linseed oil, boiled macadamia nut oil, mineral oil, olive oil, peanut oil, safflower oil, sesame oil, soybean oil, squalane, sunflower seed oil, tricaprylin (1,2,3 trioctanoyl glycerol), wheat germ oil and other oils known for antimicrobial applications, or any combinations thereof. In a preferable embodiment, fatty alcohols include but not limited to cetyl, myristyl, oleyl, cetearyl, stearyl, lauryl, isostearyl, behenyl, undecanol, palmitoleyl, heptadecyl, isostearyl, elaidyl, linoleyl, elaidolinoleyl, linolenyl, elaidolinolenyl, ricinoleyl, nonadecyl, arachidyl alcohol or any combinations thereof. In another preferable embodiment, hydrocarbons include but not limited to mineral oil, isohexadecane, squalane, hydrogenated polyisobutene, petrolatum, paraffin, microcrystalline wax, polyethylene or any combinations thereof. In yet another preferable embodiment, glycerides include but not limited to mono-, di-, and tri-glycerides, preferably di- and tri-glycerides, more preferably triglycerides. In most preferable embodiments of the compositions described herein, the glycerides are mono-, di-, and tri-esters of glycerol and long chain carboxylic acids, such as C10 to C22 carboxylic acids, variety of vegetable and animal fats and oils, such as castor oil, safflower oil, cottonseed oil, corn oil, olive oil, cod liver oil, almond oil, avocado oil, palm oil, sesame oil, lanolin, soybean oil, triolein, tristearin glyceryl dilaurate or any combinations thereof. In yet another preferable embodiment, esters of fatty acids include but not limited to isopropyl isostearate, hexyl laurate, isohexyl laurate, isohexyl palmitate, isopropyl palmitate, decyl oleate, isodecyl oleate, hexadecyl stearate, decyl stearate, dihexyldecyl adipate, lauryl lactate, myristyl lactate, cetyl lactate, oleyl stearate, oleyl oleate, oleyl myristate, lauryl acetate, cetyl propionate, and oleyl adipate. alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters or polyoxyethylene sorbitan fatty acid esters, or any combinations thereof. In yet another preferable embodiment, solubilizers include but not limited to surfactants, silicone oils, cationic silicones, silicone gums, high refractive silicones, and silicone resins, hydrocarbon oils, polyolefins, fatty acid esters as mentioned above, hydrocarbon oils like paraffin oil, mineral oil, isopropyl myristate, diethylene glycol monoethylether, PEG 400, PEG 4000, propylene glycol, 1,3-propane diol, ethanol, DMSO, isopropanol, propylene glycol caprylate, glycerol mono/di caprylate caprate, monoglycerides, diglycerides or fatty alcohols, or any combinations thereof. In yet another preferable embodiment, oils include but not limited to peanut oil, cottonseed oil, safflower oil, sesame oil, olive oil, corn oil, soybean oil, cod liver oil, almond oil, avocado oil, palm oil, sesame oil, lanolin, soybean oil, triolein, tristearin glyceryl dilaurate. silicone oils, hydrocarbon oils hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof, tea tree oil or jojoba oil, or any combinations thereof. The preferred quantity of oil used is in the range of about 5% w/w to about 95% w/w, and other important excipients like stabilizers, solubilizers, fatty alcohol, fatty acid esters are used in the range of about 0.1% w/w to about 30% w/w.

In an embodiment, emulsifying agents or surfactants include but not limited to anionic triethanolamine/potassium stearate, sodium lauryl stearate, sodium cetearyl sulfate, beeswax/borax, nonionic glycerol di-stearate, polyethyleneglycol-100-stearate, steareth-2, steareth-21 and cationic surfactants including but limited to distearyldimethylammonium chloride, benzalkonium chloride, steapyrium chloride, polyquaternium-37, acrylates/C10-30 alkyl acrylate, polyacrylamide, propylene glycol, dicaprylate/dicaprate and PPG-1 trideceth-6 and silicone based materials including but limited to alkyl modified dimethiconecopolyols, polyglyceryl esters and ethoxylated di-fatty esters. In another embodiment, emulsifiers or surfactants include but not limited to one or more of ionic polysorbate surfactants such as polysorbate20, polysorbate 40, polysorbate 60, polysorbate 80, ether based surfactants including but not limited to steareths, laureths, oleths, ceteths and other emulsifiers or surfactants known to the person skilled in the art, or any combinations thereof. The preferred quantity of the emulsifiers or surfactant in the is in the range of about 0.1% w/w to about 20% w/w and more preferably 0.1% to 10% of the total formulations.

In an embodiment, emollients used in the present formulations including topical formulations include but not limited to caprylic/capric triglycerides, castor oil, cetearyl alcohol, cetostearyl alcohol, cetyl alcohol, stearyl alcohol, cocoa butter, diisopropyl adipate, propylene glycol monocaprylate, glyceryl monooleate, glyceryl monostearate, glyceryl stearate, isopropyl myristate, isopropyl palmitate, lanolin, lanolin alcohol, lanolin esters, hydrogenated lanolin, liquid paraffins, linoleic acid, mineral oil, oleic acid, white petrolatum, polyethylene glycol, polyethylene glycols, fatty alcohols, ethers, polyoxypropylene 15-stearyl ether, propylene glycol stearate, squalane, stearic acid, urea and other emollients known to a person skilled in the art, or any combinations thereof.

In another embodiment, moisturizers used in the present formulations including topical formulations include but not limited to mineral oil, paraffin, squalene, vegetable fats such as cocoa butter, animal fats such as lanolin, fatty acids, lanolin acid, stearic acid, fatty alcohols such as lanolin alcohol and cetyl alcohol, polyhydric alcohols, wax esters, vegetable waxes, phospholipids, sterols, silicones and other moisturizers known to a person skilled in the art, or any combinations thereof.

In another embodiment, humectants include, but are not limited to propylene glycol, sorbitol, butylene glycol, butylene glycol, hexylene glycol, acetamide MEA (acetylethanolamine), honey, sodium PCA (sodium-2-pyrrolidone carboxylate), sorbitol, triacetin, and other humectants known to a person skilled in the art, or any combinations thereof.

In yet another embodiment, preservatives include but are not limited to one or more of benzalkonium chloride, cetrimonium bromide, benzethonium chloride, alkyltrimethyl ammonium bromide, methyl, ethyl, propyl, butyl parabens, benzyl alcohol, benzoic acid, sorbic acid, chloroacetamide, trichlorocarbon, thimerosal, imidurea, bronopol, chlorhexidine, 4-chlorcresol, chlorxylenol, dichlorophene, hexachlorophene, phenoxyethanol and other preservatives known to a person skilled in the art, or any combinations thereof. In still another embodiment, chelating agents include but are not limited to di or tri or tetra sodium EDTA, diethyleneamine pentaacetate and other chelating agents known to a person skilled in the art, or any combinations thereof.

In another embodiment, antioxidants include but are not limited to alpha tocopherol, ascorbic acid, ascorbyl palmitate, butylated hydroxy anisole, Butylated hydroxy toluene, citric acid, monohydrate, erythorbic acid, ethyl oleate, fumaric acid, malic acid, methionine, monothioglycerol, phosphoric acid, potassium metabisulfite, propionic acid, propyl gallate, sodium ascorbate, sodium bisulfate, sodium formaldehyde sulfoxylate, sodium metabisulfite, sodium sulfite, sodium thiosulfate, sulfur dioxide, tartaric acid, thymol, sodium metabisulfite, vitamin E, polyethylene glycol succinate and other antioxidants known to a person skilled in the art, or any combinations thereof.

In an embodiment, any pharmaceutically acceptable excipient(s) known to a person skilled in the art for antimicrobial applications can be employed in formulation the present compositions or formulations.

In an embodiment, pH of the present formulation ranges from about pH 2 to pH 8, preferably from about pH 3 to pH 8, and more preferably from about pH 5 to pH 7.5. pH modifying agents employed in the present disclosure include but not limited to one or more of organic or inorganic acids and bases including sodium hydroxide, potassium hydroxide, ammonium hydroxide, phosphate buffers, citric acid, acetic acid, fumaric acid, hydrochloric acid, malic acid, nitric acid, phosphoric acid, propionic acid, sulfuric acid, tartaric acid, triethyl amine, triethanolamine and other pH modifying agents know in the art to obtain desired pH, or any combination of pH modifying agents thereof.

In the present disclosure, different drug delivery vehicles, other excipient(s) and dosage forms including topical dosage forms are selected based on the nature (charged or neutral), solubility (aqueous or non-aqueous) and concentration of the active SMPs/compounds. In an embodiment, water soluble ointment, hydrogel, topical gel, wound dressings, polymeric patch and other dosage forms are possible formulations suitable for water soluble hydrophilic SMPs/compounds of the present disclosure. In another embodiment, oil/water (O/W)emulsion cream, O/W ointment or lipid based ointment, liposomal cream, liposomal gel, polymeric encapsulated topical gel, encapsulated hydrogel and other dosage forms are possible for hydrophobic SMPs/compounds of the present disclosure.

In an exemplary embodiment, the dosage form of the formulation of the present disclosure is hydrogel. Hydrogels are three-dimensional networks and are formed by highly hydrophilic polymers that can imbibe great amounts of aqueous fluids and hydrogel formation is facilitated by many external stimuli like pH, temperature (thermosensitive gel), freeze-thaw cycles, metal ions (mono/bi/tri-valent) and many others. Hydrogels behave like living tissues due to favorable mechanical, interfacial properties with flexibility and porosity that make them appropriate tool for biomedical and pharmaceutical applications. For example, PVA and PVP hydrogels are non-toxic and bio-adhesive in nature. PVA also shows high degree of swelling in water and forms rubbery and elastic material that can closely resemble to the natural tissues and can readily be accepted into the body. In preferred embodiments, polymeric antimicrobial hydrogels are suitable carriers for dosage form including wound dressings and topical gel formulations for maintaining moist environment that helps in healing, and additionally prevent bacterial infections in wound related surgical site and implant associated infection. Further, hydrogel materials provide a soft, stretchable and slippery exterior surface while they are also coated onto many medical devices like standard plastic or rubber devices, catheters, intravenous lines and other type of surgical tubings. The slippery exterior impacts lubricity thus significantly reduces pain and discomfort associated with the cathing/catheterization process and thereby leads to patient compliance. Additionally, lubricious surface imparted by hydrogel prevent bacterial adhesion and inhibit biofilm formation, thus significantly reducing the chance of bacterial infection and the rate of UTI (urinary tract infection).

In an exemplary embodiment of the present disclosure, preparation of hydrogel is provided. Different external stimuli were used to form hydrogel with various polymer matrices (Table 7). In an embodiment, calcium-pectin hydrogels were prepared by adding 25 mM calcium carbonate solution into about 2.4% pectin solution under stirring at about 1000 rpm at room temperature. The compound(s) of the present disclosure was then added to the prepared calcium-pectin hydrogel.

In an embodiment, starch-pectin hydrogel was made by dissolving starch and pectin at a particular molar ratio followed by incubation at about 110° C. for about 15 minutes. The compound(s) of the present disclosure was then added to the prepared hydrogel at particular concentration into the gel base followed by addition of calcium carbonate to form drug loaded cross-linked starch-pectin hydrogel. In another embodiment, alginate hydrogel is similarly made in the presence of particular concentration of Ca²⁺.

Topical wound gel or hydrogel film formation depends on the concentration of metal ion(s) and alginate solution. Dextran is natural polysaccharide. In an embodiment, dextran forms biodegradable hydrogel in presence of about 25 wt % potassium chloride solution at about 90° C. with continuous stirring until a homogeneous solution is formed which is followed by addition of the compound(s) of the present disclosure. The drug loaded dextran solution is allowed to cool to room temperature to form dextran based hydrogel. In another embodiment, dextran and β-cyclodextrin based hydrogel is obtained using sodium trimetaphosphate in presence of basic aqueous medium and the prepared hydrogel is used to encapsulate hydrophobic compound(s) of the present disclosure. In yet another embodiment, PLA-PEG-PLA triblock co-polymer is used to form hydrogel and hydrophobic compound(s) of the present disclosure is incorporated.

In an exemplary embodiment of the present disclosure, self-assembled hydrogel formation was examined with SMP polymers alone or using physical mixture of two or three SMP molecules or mixture of known polymer with SMP molecule of the present disclosure. In a preferred embodiment, SMP-105 and SMP-007 together, or SMP-071 and SMP-079 together demonstrate self-assembly. Such in situ self-assembled biodegradable or bio-absorbable hydrogel is used to load antibiotic to deliver the drug at the site of action for release modes including sustained release. In another exemplary embodiment of the present disclosure, known polymeric scaffolds are functionalized with effective monomeric unit (Formula Ia compound of the present disclosure) to form antimicrobial polymers that alone or in presence of other known polymeric scaffold are able to self-assemble and form hydrogel (Schemes 22, 23 and 24). In yet another exemplary embodiment of the present disclosure, known polymeric scaffold is reacted with diacrylate functionalized monomeric unit compound of the present disclosure that crosslinks different functional moieties of polymeric backbone and form chemically crosslink hydrogel (Scheme 25 and 26). In still another exemplary embodiment of the present disclosure, hydrogel was made with effective SMP molecules of the present disclosure in the presence of known hydrogel forming polymers (Table 7). In all the cases, hydrogel acts as a suitable carrier/matrix that could maintain sustained delivery of the active agent at the site of infection while maintaining no or minimal toxicity. SMP molecules loaded into different hydrogel matrices have various applications depending on the nature of polymer matrices used for hydrogel formation as discussed in Table 7. In an embodiment, PVA or PVA-acid or PVA-PVP co-polymer or alginate or pectin or starch-pectin based biocompatible and biodegradable hydrogel matrices are used for hydrophilic antimicrobial agents of the present disclosure including SMP-007, SMP-037 and/or other hydrophilic SMP molecules. Depending on the nature of hydrogel matrices, drug-loaded hydrogels are used either as a topical gel or hydrogel films or wound dressing materials at surgical site and wound related infection; or as coating material on implant and catheter surface. These hydrogel-based wound dressings and transparent-stretchable films helps to retain moisture, improve healing process and additionally prevents microbial infection and biofilm formation at the infection site. In another embodiment, hydrophobic antimicrobial agents including SMP-020 and/or other hydrophobic SMP molecules, along with L-ascorbyl palmitate or PLA-PEG-PLA co-polymer or PLGA or poloxamer or dextran-β-cyclodextrin-STMP (Sodium trimetaphosphate) or any other matrix or combinations thereof are used to form hydrogel. Said hydrogel is employed for topical application or thermosensitive injectable gel for implant associated infection or coating material for catheter or implant surfaces.

Amphiphile based scaffolds are fascinating for hydrogelation because they can be self-assembled alone or crosslinking between different active functional units via different non-covalent interactions including H-bonding, van der Waals forces, electrostatic or pi-pi interactions and able to cage large amount of water molecules and form hydrogel. To obtain antimicrobial amphiphilic scaffolds, acrylic functionalized PVA was reacted with mono or diacrylate functionalized polyamine derivatives either by UV irradiation or in the presence of AIBN to obtain either amphiphilic antimicrobial polymer or self-assembled 3D-gel network or hydrogel. On the other hand, the synthesized acrylic functionalized PVA (scheme 26 of Example 25 below) or N-vinyl pyrrolidone (schemes 22 and 23 of Examples 21 & 22 below) was reacted with polyamine scaffold to obtain self-assembled 3D-gel network or hydrogel. Alternatively, acid functionalized PVA scaffold was reacted with active polyamine derivatives to obtain polyamide scaffold with potent antimicrobial properties or having propensity to self-associate to form hydrogel (Schemes 24 and 25 of Example 23 & 24 below).

In another exemplary embodiment, hydrophilic antimicrobial SMP molecules/compound(s) of the present disclosure are used as a coating material on catheters or other implant materials. Hydrophilic catheters containing coated SMP molecules/compound(s) when submersed in water absorb and bind the water to the catheter surface to form smooth and slippery surface. Such surface lubrication or ultra-soft outer layer result in virtually friction free catheter insertion and removal which helps to minimize the risk of any bacterial infection.

In an embodiment of the present disclosure, monomeric and polymeric SMP molecules show effective in vitro antibacterial and antifungal activity along with biofilm inhibitory effect with limited toxicity profile against HaCaT cell line. Said SMP molecules are selected for different topical formulations using different drug delivery vehicles/carriers. For example, water soluble SMPs like SMP-047 (Formula Ia, monomeric analogue), polybiguanidine based SMP-007 (Formula Ib), polybiguanidine-polyurethane based mixed polymer SMP-037 (Formula Ic) are selected to prepare dosage forms—hydrogel or water-soluble ointment or wound gel or coating material for catheters and other implants. In another embodiment, sparingly water soluble and water insoluble polyurethane based SMP-020 and SMP-042 are formulated in the form of ointment or polymeric/liposome encapsulated gel or coating material for catheters and other implants. In an embodiment, the anti-microbial ointment contains excipient selected from a group comprising mineral oil, white soft paraffin, lanolin, lanolin alcohols, lanolin esters, white bees wax, yellow bees wax, microcrystalline wax, microcrystalline cellulose, cetostearyl alcohol, cetyl alcohol, stearyl alcohol, polyethylene glycols of different molecular weights, emulsifiers and other excipients for ointment preparation known to a person skilled in the art, or any combination of excipients thereof.

In an embodiment, different functional groups present in SMP compounds of the present disclosure have the ability to interact non-covalently with silver ion or silver nanoparticles. Thus, in some embodiments, co-administration of SNAP compounds with silver on dressings are provided which can prevent deactivation of silver ion or silver nano-particles in the presence of serum and simultaneously prevent leaching of silver from dressing surfaces.

In an embodiment, SMP compounds of the present disclosure are water soluble, stable and are easily impregnated into different delivery matrices including but not limiting to ointment, hydrogel, beads and dressing materials.

In an exemplary embodiment, the compounds of Formula I, Ia, Ib and Ic of the present disclosure mimics natural scaffolds and thus provide minimal toxicity within therapeutic doses. These compounds are well-tolerated by different human and murine cell lines. Specifically, these compounds do not interfere with the healing processes as tested in in vitro systems. This shows that the present compounds are compatible/safe for use in the treatment of wound infections.

In an embodiment of the present disclosure, bio-absorbable devices composed of polyester compounds described herein, primarily homopolymer and co-polymer of ethanol amine or PEG with succinic acid provide diverse application towards treatments ranging from ligament repair, wound closure including sutures, suture anchors, skin staples and adhesives, drug delivery carrier, wound dressing and other conditions known in the art.

In exemplary embodiment, compounds of Formula I and Formula II described herein have antibacterial, antifungal, anti-biofilm properties or any combinations thereof. Additionally, the compounds and respective formulations including topical formulations possess not only antimicrobial or anti-infective properties, but also have wound healing, anti-inflammatory or anti-oxidant properties, or any combinations thereof.

In exemplary embodiment, the compound of Formula I described herein not only act on drug resistant strains but can resist development of resistant strains because of their non-specific action on the pathogens. Hence, even after prolonged use of the compounds of the present disclosure, the frequency of mutation in the pathogens will be low. Thus, compounds of the present disclosure are attractive and very useful as anti-microbial agents in the present antibiotic resistance crisis.

The present disclosure further provides methods for management or treatment of microbial infections and/or associated disease. The microbial infections are preferably bacterial and/or fungal infections associated with SSIs. In an embodiment, the including microbial infection is selected from a group consisting of superficial skin infections, deep wound infections, burn infections, infections associated with diabetic foot ulcers, gangrenes, cellulitis, cuts and insect bites, infected wounds in military populations and combinations thereof. In another embodiment, the compounds and/or compositions are employed for the management or treatment of implant-associated orthopedic infections and/or medical device related infections. In an exemplary embodiment, the microbial infection is selected from a group consisting of orthopedic bone infections, implant associated infections arising due to the use of orthopedic prostheses, fracture fixation devices, coronary stents, central venous and urinary catheters, heart valves, vascular grafts, central nervous system implants, ophthalmic, otitic and dental implants, or any combination of infections thereof.

In an embodiment, the method of treatment uses compounds and/or compositions of the present disclosure against microbial infection caused by bacterial and/or fungal species. In another embodiment, the microbial infection is broad spectrum resistant microbial infection caused by resistant bacterial and/or fungal strains.

Different drug delivery dosage forms are adopted for the antimicrobial therapy employing the compounds or compositions described in the present disclosure. In an embodiment, for topical application, the compounds or compositions of the present disclosure is used in different forms but not limited to creams, ointments, gels, powders, sprays, impregnated dressings like adhesive bandages, transdermal patches, or any combinations thereof. In another embodiment, surface coating of implants including but not limited to catheters, stents and orthopedic implants with the compounds or compositions described in the present disclosure is carried out to combat infection by reducing microbial attachment and preventing biofilm formation. The surface coated implants prevent bacterial growth on these implants/devices.

The present disclosure also provides compounds or compositions described herein for use in the manufacture of a medicament. The present disclosure further provides a compound or a composition described herein for use in treating a microbial infection selected from anti-bacterial infection, anti-fungal infection, biofilm associated infection, or any combination thereof. In an embodiment, the microbial infection is an infection as described in the above-mentioned embodiments.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

Additional embodiments and features of the present disclosure will be apparent to one of ordinary skill in art based upon description provided herein. The embodiments herein and the various features and advantageous details thereof are explained with reference to the non-limiting embodiments in the description. Descriptions of well-known/conventional methods and techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1

Synthesis of Compounds SMP-002, SMP-046 and SMP-047

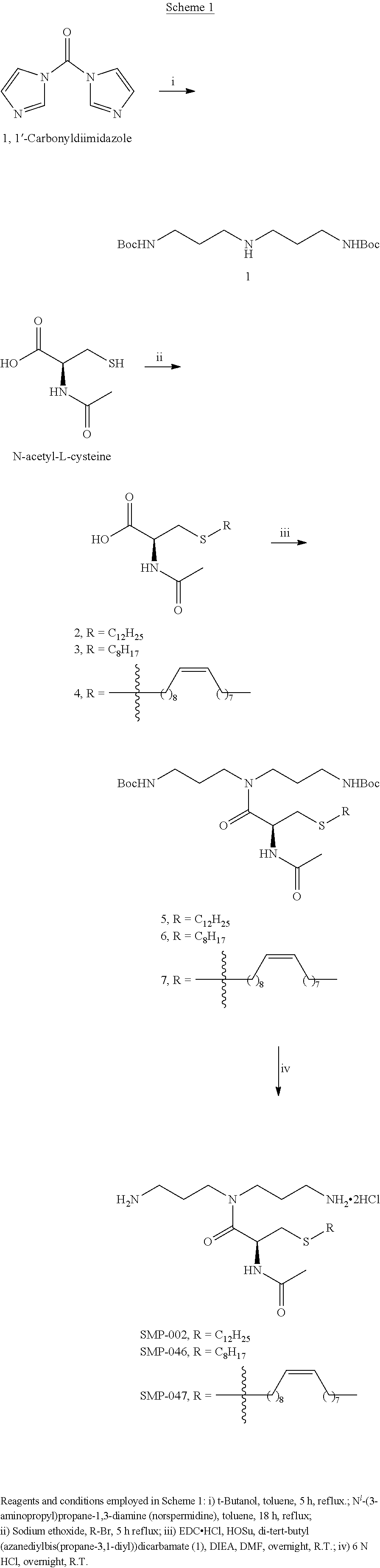

di-tert-Butyl (azanediylbis(propane-3,1-diyl))dicarbamate (1): 1,1′-Carbonyldiimidazole (CDI) (13.60 g, 84 mmol) was suspended in a mixture of toluene (100 ml) and t-butanol (11.91 g, 15 ml, 161 mmol) under nitrogen atmosphere and was heated to 60° C. for 5 h. A solution of norspermidine (5.77 g, 6.2 ml 44 mmol) in toluene (60 ml) was added dropwise. The reaction mixture was refluxed at 120° C. for 18 h, then cool and concentrated under vacuum. The resulting liquid was extracted in dichloromethane, washed with distilled water and finally dried over anhydrous Na₂SO₄, filtered and concentrated in vacuo to give di-tert-butyl (azanediylbis(propane-3,1-diyl))dicarbamate as a white powder (12 g, 86%). ¹H NMR (CDCl₃): δ 5.22 (brs, 2H, —NHCO), 3.21-3.10 (m, 4H, —CH₂NHCO), 2.63-2.60 (m, 4H, —CH₂NH), 1.65-1.59 (m,4H, —CH₂), 1.40 (s 18H, C(CH₃)₃).

N-Acetyl-S-dodecyl-L-cysteine (2): Freshly cut sodium metal (180 mg, 7.8 mmol) was dissolved in anhydrous ethanol (15 mL) under nitrogen atmosphere. To this solution N-acetyl-L-cysteine (500 mg, 3.1 mmol) was added followed by 1-bromododecane (0.89 mL, 3.72 mmol) and the reaction mixture was heated at reflux for 5 h. Upon cooling, the reaction was quenched with small amount of water, the solvent was removed under reduced pressure followed by extraction with ethyl acetate. The solution was washed with 1 M HCl, brine, dried over anhydrous sodium sulphate and finally the solvent removed under reduced pressure to obtain N-acetyl-S-dodecyl-L-cysteine (2) as a white solid (810 mg, 80%). ¹H NMR (CDCl₃): δ 4.78 (q, 1H, J_{AB}=6 Hz, —CHNH), 3.50-3.45 (m, 1H, —CH₂S), 3.41 (t, 1H, J_{AB}=6 Hz, —CH₂S), 3.03 (t, 2H, J_{AB}=4.5 Hz, —CH₂S). 2.55-2.52. (m, 2H, —CH₂), 2.1(s, 3H, —COCH₃), 1.58-1.52 (m, 2H, —CH₂), 1.25 (s, 16H, —CH₂), 0.87 (t, 3H, J_{AB}=6.5 Hz, —CH₃). ESI-MS (m/z): 331.99 (M+H).

N-Acetyl-S-octyl-L-cysteine (3): Compound 3 was synthesized from N-acetyl-L-cysteine and 1-bromooctane by following the similar synthetic procedure as mentioned for synthesizing compound 2. (63%). ¹H NMR (CDCl₃): δ 6.65 (brs, 1H, NHCO), 4.70-4.65 (m, 1H, CH), 3.08-2.93 (m, 2H, —CH₂S), 2.53 (t, 2H, J_{AB}=7 Hz, —CH₂S), 2.1(s, 3H, —COCH₃), 1.58-1.52 (m, 2H, —CH₂), 1.38-1.32 (m, 2H, —CH₂), 1.30-1.1.18 (m, 8H, —CH₂), 0.87 (t, 3H, J_{AB}=6.5 Hz, —CH₃).

(Z)-N-Acetyl-S-(nonadec-9-en-1-yl)-L-cysteine (4): Compound 4 was synthesized from N-acetyl-L-cysteine and (Z)-1-bromooctadec-9-ene by following the similar synthetic procedure as mentioned for synthesizing compound 2. (80%). ¹H NMR (CDCl₃): δ 6.60 (brs, 1H, NHCO), 5.35-5.32 (m, 2H, —CH═CH—), 4.78-4.68 (m, 1H, —CHNHCO), 3.05-2.98 (m, 2H, —CH₂S), 2.53 (t, 2H, J_{AB}=7 Hz, —CH₂S), 2.01 (s, 3H, —COCH₃), 2.01-1.98 (m, 2H, —CH₂)1.58-1.52 (m, 2H, —CH₂), 1.32-1.244 (m, 24H, —CH₂), 0.87 (t, 3H, J_{AB}=6.5 Hz, —CH₃); ESI-MS (m/z): 414.25 (M+H).

di-tert-Butyl (((N-acetyl-S-dodecyl-L-cysteinyl)azanediyl)bis(propane-3,1-diyl))dicarbamate (5): To a stirred solution of 1-ethyl-3-(3-dimethylaminopropyl)-carbodiimide hydrochloride (EDC.HCl, 576 mg, 3 mmol) in DMF at 0° C., N-Acetyl-S-dodecyl-L-cysteine (2), (662 mg, 2.0 mmol) was added, followed by addition of N-hydroxysuccinimide (HOSu, 345 mg, 3 mmol). After 1 h di-tert-butyl (azanediylbis(propane-3,1-diyl))dicarbamate (1), (598 mg, 1.8 mmol) and N,N-diisopropylethylamine (DIEA, 0.53 mL, 3 mmol) were added and the reaction mixture was allowed to stir at room temperature for overnight. After completion, the reaction mixture was extracted with ethyl acetate, evaporated, the organic solvent to obtain crude mass which was purified by flash column chromatography over silica gel using 3% methanol-dichloromethane as eluent to obtain di-tert-butyl (((N-acetyl-S-dodecyl-L-cysteinyl)azanediyl)bis(propane-3,1-diyl))dicarbamate as (5) an off white solid (522 mg, 45%). ¹H NMR (CDCl₃): δ 6.39 (brs, 1H, —NHCO), 5.26 (brs, 2H, —NHCO), 5.08 (q, 1H, J_{AB}=8 Hz, —CHNH), 3.72-3.70 (m, 1H, —CH₂S), 3.63-3.57 (m, 1H, —CH₂S), 3.33-3.14 (m, 4H, —CH₂N), 3.13-3.07 (m, 2H, —CH₂S), 2.88-2.84 (m, 4H, —CH₂N), 2.57-2.54 (m, 2H, —CH₂), 2.03 (s, 3H, —COCH₃), 1.61-1.58 (m, 6H, —CH₂). 1.51-1.49 (m, 2H, —CH₂), 1.46 (s, 18H, —C(CH₃)₃), 1.37-1.28 (s, 14H, —CH₂), 0.87 (t, 3H, J_{AB}=6.5 Hz, —CH₃).

di-tert-Butyl (((N-acetyl-S-octyl-L-cysteinyl)azanediyl)bis(propane-3,1-diyl))dicarbamate (6): Compound 6 was synthesized from compound 3 and compound 1 by following the similar synthetic procedure as mentioned for synthesizing compound 5. (53%). ¹H NMR (CDCl₃): δ 5.35-5.26 (brs, 1H, NHCO), 5.24-5.17 (m, 1H, —CHNH), 3.56-3.52 (m, 2H, —CH₂S), 3.29-3.22 (m, 4H, —CH₂NHCO), 3.16-3.09 (m, 4H, —CH₂NCO), 2.93-2.87 (m, 2H, —CH₂S), 2.85-2.80 (m, 2H, —CH₂), 1.75-1.66 (m, 7H, —CH₂, —CH₃), 1.62-1.56 (m, 2H, —CH₂), 1.47 (s, 18H, —C(CH₃)₃), 1.40-1.32 (m, 2H, —CH₂), 1.34-1.26 (m, 8H, —CH₂), 0.94-0.87 (m, 3H, —CH₃).

di-tert-Butyl ((((Z)-N-acetyl-S-(nonadec-9-en-1-yl)-L-cysteinyl)azanediyl)bis(propane-3,1-diyl))(Z)-dicarbamate (7): Compound 7 was synthesized from compound 4 and compound 1 by following the similar synthetic procedure as mentioned for synthesizing compound 5. (42%). ¹H NMR (CDCl₃): δ 6.38 (brs, 1H, —NHCO), 5.34-5.25 (m, 2H, —CH═CH—), 5.22 (brs, 2H, —NHCO), 5.09-5.02 (m, 1H, —CHNHCO), 3.65-3.53 (m, 2H, —CH₂S), 2.89-2.81 (m, 2H, —CH₂S), 3.26-3.15 (m, 4H, —CH₂NCO), 3.10-2.92 (m, 4H, —CH₂N), 2.59-2.52 (m, 2H, —CH₂), 2.00 (s, 3H, —COCH₃), 1.75-1.62 (m, 4H, —CH₂), 1.59-1.52 (m, 2H, —CH₂), 1.43 (s, 18H, —C(CH₃)₃), 1.26-1.18 (m, 24H, —CH₂), 0.91-0.83(m, 3H, —CH₃).

(S)-2-Acetamido-N,N-bis(3-aminopropyl)-3-(dodecylthio)propanamide di-hydrochloride (SMP-002): A suspension of compound 5 (645 mg, 1 mmol) in 6N HCl (10 mL) was stirred for overnight, solvent was evaporated and crude mass was dissolved in methanol-dichloromethane followed by addition of diethyl ether, the process was repeated for 2-3 times and finally obtain pure (S)-2-acetamido-N,N-bis(3-aminopropyl)-3-(dodecylthio)propanamide di-hydrochloride (SMP-002) as brown yellow sticky mass (403 mg, 78%). ¹H NMR (DMSO-d₆): 8.84 (brs, 1H, —NH) 8.19 (brs, 4H, —NH), 8.09 (brs, 4H, —NH), 4.76 (q, 1H, J_{AB}=7.5 Hz, —CHNH), 3.32-3.22 (m, 4H, —CH₂NH₂), 3.02-2.99 (m, 1H, —CH₂S), 2.95-2.94 (m, 1H, —CH₂S), 2.78-2.73 (m, 2H, —CH₂S), 1.85 (s, 3H, —COCH₃), 1.84-1.79 (m, 4H, —CH₂NCO), 1.51-1.47 (m, 2H, —CH₂). 1.28-1.18 (s, 18H, —CH₂), 0.86 (t, 3H, J_{AB}=6.5 Hz, —CH₃). ESI-MS (m/z): 445.29 (M+H).

di-tert-Butyl (((N-acetyl-S-octyl-L-cysteinyl)azanediyl)bis(propane-3,1-diyl))dicarbamate di-hydrochloride (SMP-046): Compound SMP-046 was synthesized from compound 6 by following the similar synthetic procedure as mentioned for synthesizing compound (SMP-002) (79%). ¹H NMR (D₂O): 4.16-3.98 (m, 1H, —CH), 3.56-3.52 (m, 2H, —CH₂S), 3.29-3.22 (m, 8H, —CH₂N), 2.76-2.74 (m, 2H, —CH₂), 1.83-1.75 (m, 7H, —CH₂, —CH₃), 1.62-1.56 (m, 2H, —CH₂), 1.41-1.15 (m, 8H, —CH₂), 0.78-0.73 (m, 3H, —CH₃). ESI-MS (m/z): 391.26 (M+H).

(S,Z)-2-Acetamido-N,N-bis(3-aminopropyl)-3-(nonadec-9-en-1-ylthio)propanamide di-hydrochloride (SMP-047): Compound SMP-047 was synthesized from compound 7 by following the similar synthetic procedure as mentioned for synthesizing compound (SMP-002) (27%). ¹H NMR (D₂O): δ 5.34-5.25 (m, 2H, —CH═CH—), 5.10-5.04 (m, 1H, —CHNCO), 3.65-3.53 (m, 2H, —CH₂S), 2.89-2.81 (m, 2H, —CH₂S), 3.26-3.15 (m, 4H, —CH₂NH₂), 3.10-2.96 (m, 4H, —CH₂N), 2.59-2.52 (m, 2H, —CH₂), 2.00 (s, 3H, —COCH₃), 1.51-1.45 (m, 4H, —CH₂) 1.26-1.18 (m, 26H, —CH₂), 0.91-0.83 (m, 3H, —CH₃); ESI-MS (m/z): 526.43 (M+H).

### Example 2

Synthesis of Compound SMP-022

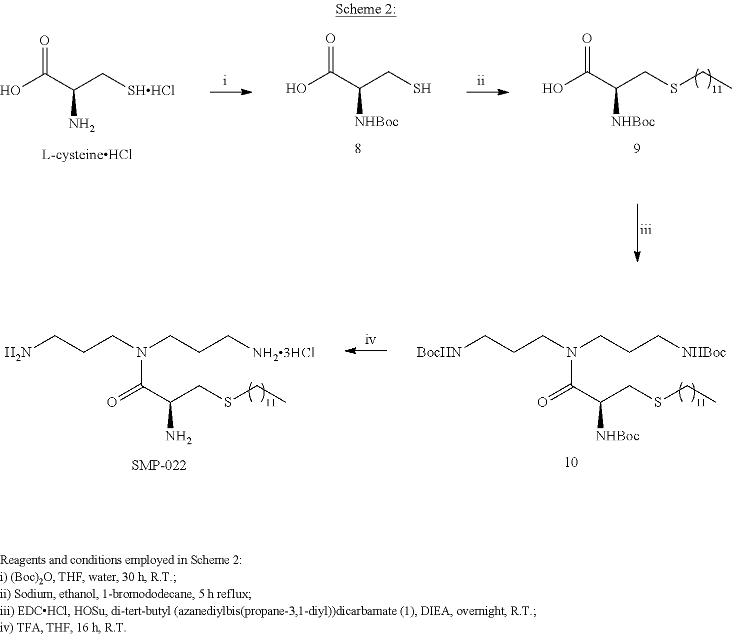

(tert-Butoxycarbonyl)-L-cysteine (8): A mixture of L-cysteine hydrochloride (1.23 g, 8.25 mmol), (Boc)₂O (1.801 g, 8.25 mmol) and NaHCO₃ (2.5 g, 29.8 mmol) in THF (7 mL) and water (18 mL) was stirred under argon at room temperature for 30 hours under nitrogen atmosphere. After completion of the reaction pH was adjusted to 3 and extracted with ethyl acetate and evaporated in vacuo to give an oil of (tert-butoxycarbonyl)-L-cysteine (1.54 g, 84%). ¹H NMR (CDCl₃) δ 9.05 (brs, 1H, —COOH), 5.51 (brs, 1H, —NHCO), 4.64-4.61 (m, 1H, —CHNHCO), 3.07-3.02 (m, 1, —CH₂S), 2.99-2.93 (m, 1H, —CH₂S), 1.44 (s, 9H, C—(CH₃)₃).

N-(tert-Butoxycarbonyl)-S-dodecyl-L-cysteine (9): Freshly cut sodium metal (180 mg, 7.8 mmol) was dissolved in anhydrous ethanol (15 mL) under nitrogen atmosphere. To this solution compound 8 (686 mg, 3.1 mmol) was added followed by 1-bromododecane (0.89 mL, 3.72 mmol) and the reaction mixture was heated at reflux for 5 h. Upon cooling, the reaction was quenched with small amount of water, the solvent was removed under reduced pressure followed by extraction with ethyl acetate. The solution was washed with 1 M HCl, brine, dried over anhydrous sodium sulphate and finally the solvent removed under reduced pressure to obtain N-(tert-butoxycarbonyl)-S-dodecyl-L-cysteine (9) as a white solid (965 mg, 80%). ¹H NMR (CDCl₃): δ 5.40 (brs, 1H, —NHCO) 4.51 (q, 1H, J_{AB}=6 Hz, —CH), 3.04-2.98 (m, 2H, —CH₂S), 2.55 (t, 2H, J_{AB}=7.5 Hz, —CH₂S), 1.60-1.54 (m, 2H, —CH₂), 1.46 (s, 9H, —C(CH₃)₃), 1.38-1.32 (m, 2H, —CH₂), 1.29-1.23 (m, 16H, —CH₂), 0.88 (t, 3H, J_{AB}=6.5 Hz, —CH₃).

di-tert-Butyl (((N-(tert-butoxycarbonyl)-S-dodecyl-L-cysteinyl)azanediyl)bis(propane-3,1-diyl))dicarbamate (10): To a stirred solution of 1-ethyl-3-(3-dimethylaminopropyl)-carbodiimide hydrochloride (EDC.HCl, 576 mg, 3 mmol) in DMF at 0° C., compound 9 (778 mg, 2.0 mmol) was added, followed by addition of N-hydroxysuccinimide (HOSu, 345 mg, 3 mmol). After 1 h di-tert-butyl (azanediylbis(propane-3,1-diyl))dicarbamate (1) (598 mg, 1.8 mmol) and N,N-diisopropylethylamine (DIEA, 0. 53 mL, 3 mmol) were added and reaction mixture was allowed to stir at room temperature for overnight. After completion, the reaction mixture was extracted with ethyl acetate. After evaporation of the organic solvent the crude mass was purified by flash column chromatography over silica gel using 3% methanol-dichloromethane as eluent to obtain di-tert-Butyl (((N-(tert-butoxycarbonyl)-S-dodecyl-L-cysteinyl)azanediyl)bis(propane-3,1-diyl))dicarbamate (10) as an off white solid (816 mg, 58%). ¹H NMR (CDCl₃): δ 5.75 (brs, 1H, —NHCO), 4.26 (q, 1H, J_{AB}=8 Hz, —CHNHCO), 3.74-3.71 (m, 1H, —NH), 3.69-3.68 (m, 1H, —NH), 3.49-3.45 (m, 2H, —CH₂S), 3.33-3.12 (m, 4H, —CH₂NCO), 3.14-3.07 (m, 2H, —CH₂S), 2.96-2.91 (m, 4H, —CH₂NCO), 2.54-2.51 (m, 2H, —CH₂), 1.70-1.61 (m, 2H, —CH₂), 1.53-1.49 (m, 2H, —CH₂), 1.42 (s, 18H, —C(CH₃)₃), 1.33-1.18 (m, 27H, —CH₂, —C(CH₃)₃), 0.86 (t, 3H, J_{AB}=6.5 Hz, —CH₃).

(S)-2-Amino-N,N-bis(3-aminopropyl)-3-(dodecylthio)propenamide tris-tetrafluoroacetate (SMP-022): To an ice cold solution of compound 10 (300 mg, 0.43 mmol) in THF (10 ml), trifluoroacetic acid (10 ml) was added and the reaction mixture was allowed to stir at room temperature for 16 h. On completion, solvent was evaporated and remaining crude mass was dissolved in methanol and precipitated by addition of diethyl ether. This procedure was repeated 3-4 times and finally the crude mass was dried under vacuum to give SMP-022 as brown solid (330 mg, 45%).

### Example 3

Synthesis of compound SMP-051

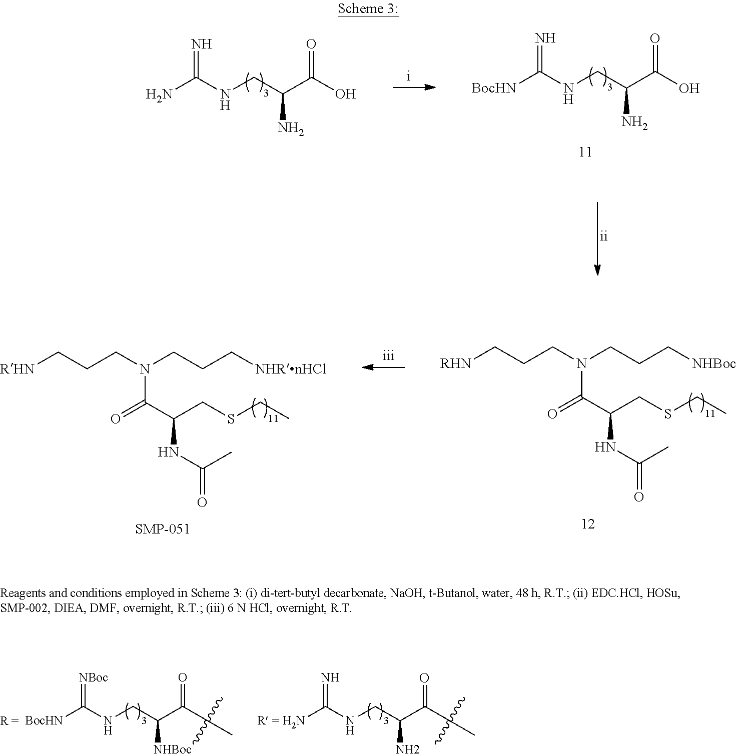

N²,N^{ω},N^{ω′}-tris(tert-butoxycarbonyl)-L-arginine (11): L-arginine (8.7 g, 50 mmol) was added into a solution of tert-butanol (150 mL) and water (150 mL) in a 500 mL round-bottom flask. The mixture was cooled to 0° C. in an ice bath and sodium hydroxide (7.0 g, 175 mmol) was added. The solution was stirred for 5 min at 0° C. and to it was added (Boc)₂O (43.7 g, 200 mmol) in portions. The reaction mixture was stirred for 48 h at room temperature. Reaction mixture was concentrated under reduced pressure and ethyl acetate was added and pH of the medium was adjusted to 3 by addition solid citric acid. The extract was dried with anhydrous sodium sulfate and evaporated in a vacuum to give compound 11 as white solid. (16.2 g, 64%) ¹H NMR (DMSO-d₆) δ 11.51 (brs, 1H, COOH), 9.41 (brs, 1H, NH), 8.42 (brs, 1H, NH), 5.77 (d, 1H, J_{AB}=6.5 Hz, NHCH), 4.34 (q, 1H, J_{AB}=7.0 Hz, CH), 3.92-3.82 (m, 2H, CH₂N), 1.84-1.82 (m, 2H, CH₂), 1.76-1.65 (m, 2H, CH₂), 1.52 (s, 9H, C(CH₃)₃), 1.50 (s, 9H, C(CH₃)₃), 1.45 (s, 9H, C(CH₃)₃).

Hexa-tert-butyl carbonate (S)-2-acetamido-N-(4-((S)-2-amino-3 guanidinopropanamido)butyl)-N-(3-((S)-2-amino-3-guanidinopropanamido)propyl)-3-(dodecylthio)propanamide (12): Compound 12 was synthesized from compound 11 and compound SMP-002 by following the similar synthetic procedure as mentioned for synthesizing compound 5. (52%). ¹H NMR (CDCl₃): δ 5.33-5.28 (m, 2H, —CHNHCO), 4.44-4.36 (m, 1H, —CHNHCO), 3.97-3.45 (m, 8H, —CH₂NCO), 2.99-2.96 (m, 4H, —CH₂NCH), 2.94-2.88 (m, 4H, CH₂S), 2.1 (s, 3H, —COCH₃), 1.94-1.69 (m, 10H, —CH₂), 1.54 (s, 18H, —C(CH₃)₃), 1.52 (s, 18H, —C(CH₃)₃), 1.50-1.49 (m, 4H, —CH₂), 1.47 (s, 18H, —C(CH₃)₃), 1.30-1.27 (m, 18H, —CH₂), 0.93-0.90 (m, 3H, —CH₃).

(S)-2-acetamido-N-(44(S)-2-amino-3-guanidinopropanamido)butyl)-N-(34(S)-2-amino-3-guanidinopropanamido)propyl)-3-(dodecylthio)propanamide hydrochloride salt (SMP-051): Compound SMP-051 was synthesized was synthesized from compound 12 and following the similar synthetic procedure as mentioned for synthesizing compound (SMP-002) (86%). ¹H NMR (D₂O): δ 5.13-5.02 (m, 2H, —CHNHCO), 3.97-3.45 (m, 8H, —CH₂NCO), 2.99-2.96 (m, 4H, —CH₂NCH), 2.94-2.88 (m, 4H, CH₂S), 2.1 (s, 3H, —COCH₃), 1.98-1.72 (m, 4H, —CH₂), 1.73-1.67 (m, 6H, CH₂) 1.50-1.49 (m, 4H, —CH₂), 1.30-1.27 (m, 18H, —CH₂), 0.93-0.90 (m, 3H, —CH₃). ESI-MS (m/z): 745.38 (M+K).

### Example 4

Synthesis of Compounds SMP-007 and SMP-010

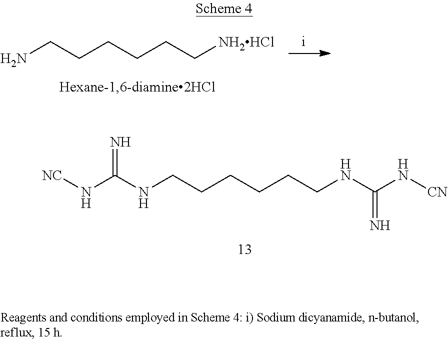

1,6-Bis(N³-cyano-N¹-guanidino)hexane (13): A solution of 1,6-hexamethylenediamine dihydrochloride (3.78 g, 20.0 mmol) and sodium dicyanamide (3.56 g, 40.0 mmol) in n-butanol (28 mL) was heated to reflux for 15 h. After cooling to room temperature, the solid was filtered off and washed with butanol and cold water. Recrystallization from water afforded pure 1,6-bis(N³-cyano-N¹-guanidino)hexane (4 g, 80%). ¹H NMR (DMSO-d₆): δ 8.06 (brs, 2H, —NH), 6.91 (brs, 4H, —NH), 3.00 (t, 4H, J_{AB}=7.5 Hz, —CH₂), 1.69-1.65 (m, 4H, —CH₂), 1.42-1.41 (m, 4H, —CH₂).

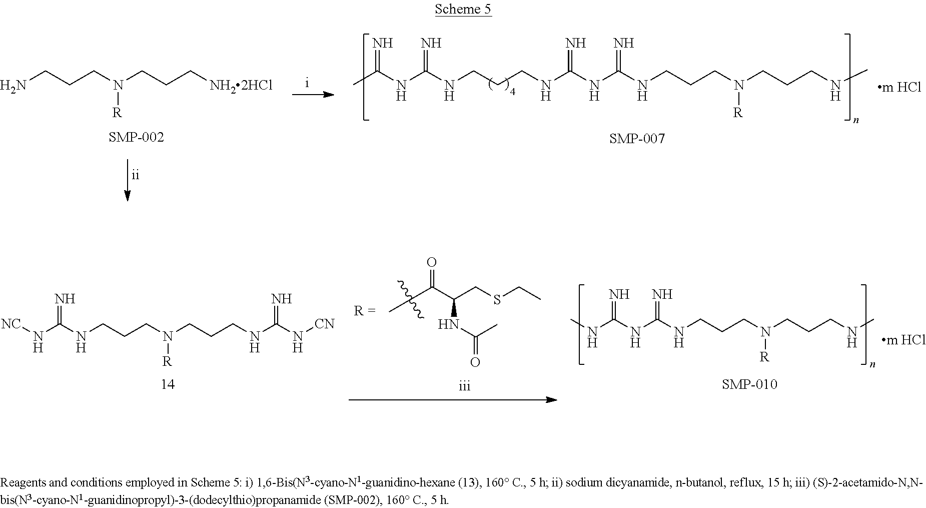

Synthesis of Polymer SMP-007: A mixture of (S)-2-acetamido-N,N-bis(3-aminopropyl)-3-(dodecylthio)propanamide di-hydrochloride (SMP-002) (100 mg, 0.19 mmol) and compound 13 (55 mg, 0.22 mmol) was heated slowly to 160° C. After reaching the temperature the mixture melt was kept at that particular temperature for another 5 h. After which the reaction mixture was cooled, water was added to it and the mixture was filtered through a 0.22-micron syringe filter to remove suspended particles. Filtrate was evaporated and dried under vacuum for several hours to obtain desired polymer SMP-007 as brown colour mass (55 mg). ¹H NMR (D₂O): δ 3.17-3.14 (m, 6H, —CH₂N, —CH₂S), 3.1-2.98 (m, 6H, —CH₂N, —CH₂S), 2.69-2.68 (m, 4H, —CH₂N), 2.57-2.54 (m, 2H, —CH₂), 1.98 (s, 3H, —COCH₃), 1.69-1.49 (m, 8H, —CH₂), 1.4-1.28 (m, 8H, —CH₂), 1.27-1.15 (m, 16H, —CH₂), 0.89-0.78 (m, 3H, —CH₃). Mₙ=5000, M_{w}=3050 PDI=1.48.

(S)-2-Acetamido-N,N-bis(N³-cyano-N¹-guanidinopropyl)-3-(dodecylthio)propanamide (14): Compound 14 was synthesized was synthesized from SMP-002 and following the similar synthetic procedure as mentioned for synthesizing compound (13).

Synthesis of Polymer SNIP-010: A mixture of SNIP-002 (50 mg, 0.10 mmol) and compound 14 (64 mg, 0.11 mmol) was heated slowly to 160° C. After reaching the temperature the mixture melt was kept at that particular temperature for another 5 h. After which the reaction mixture was cooled, water was added to it and the mixture was filtered through a 0.22 micron syringe filter to remove suspended particles. Filtrate was evaporated and dried under vacuum for several hours to obtain desired polymer SMP-010 as brown colour mass (40 mg). ¹H NMR (D₂O): δ 3.43-3.33 (m, 8H, —CH₂N), 3.13-3.07 (m, 2H, —CH₂S), 2.77-2.69 (m, 2H, —CH₂S), 2.57-2.54 (m, 2H, —CH₂), 2.07 (s, 3H, —COCH₃), 1.51-1.49 (m, 4H, —CH₂), 1.4-1.28 (m, 2H, —CH₂), 1.28-1.16 (m, 16H, —CH₂), 0.87 (t, 3H, J_{AB}=6.5 Hz, —CH₃). Mₙ (predicted): 3600.

### Example 5

Synthesis of Compounds SMP-043, SMP-045, SMP-017 and SMP-060

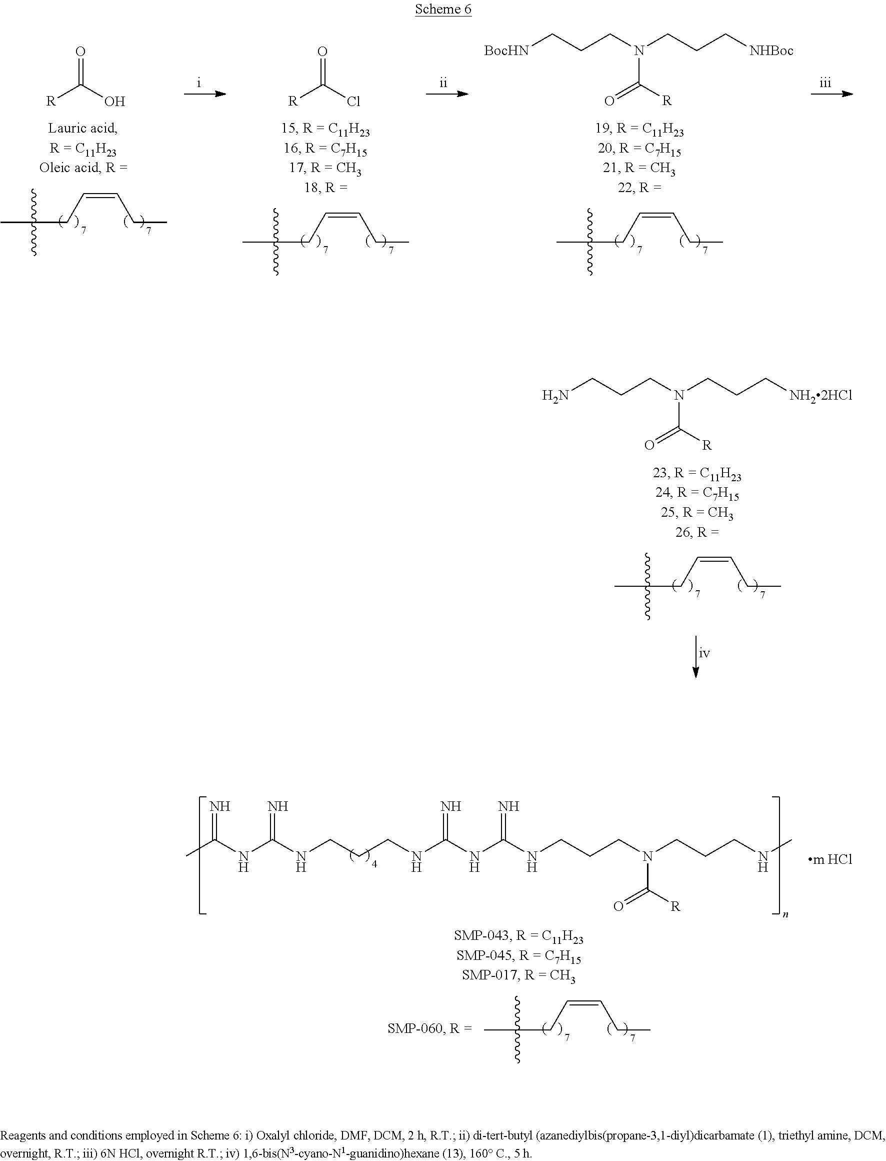

Lauroyl Chloride (15): Lauric acid (5 g, 25 mmol) was dissolved in dry DCM (10 mL) with a catalytic amount of dry DMF, and oxalyl chloride (2.56 mL, 30 mmol) was added slowly at 0° C. After complete addition reaction mixture was allowed to stir at room temperature for 3 h. Excess oxalyl chloride was removed under reduced pressure in rotary evaporator. The residue left upon vacuum drying afforded the desired lauroyl chloride (5.2 g, 95% yield).

Oleoyl chloride (18): Compound 18 was synthesized from oleic acid and following the similar synthetic procedure as mentioned for synthesizing compound (15).

di-tert-Butyl ((dodecanoylazanediyl)bis(propane-3,1-diyl))dicarbamate (19): To a solution of di-tert-butyl (azanediylbis(propane-3,1-diyl))dicarbamate (1) (332 mg, 1 mmol) and triethyl amine (202.4 mg, 0.28 mL, 2 mmol) in dichloromethane (20 mL), lauroyl chloride (290 mg, 1.3 mmol) in dichloromethane was added slowly at 0° C. After complete addition, the reaction mixture was allowed to stir at room temperature for overnight. After which reaction mixture was washed with water and organic layer was concentrated under vacuum to obtain crude with was purified by flash column chromatography over silica gel using 3% methanol-dichloromethane as eluent to obtain di-tert-butyl ((dodecanoylazanediyl)bis(propane-3,1-diyl))dicarbamate as semisolid mass (410 mg, 80%) ¹H NMR (CDCl₃): δ 3.30-3.17 (m, 4H, —CH₂N), 3.14-3.04 (m, 4H, —CH₂N), 2.86-2.78 (m, 2H, —CH₂CO), 1.80-1.70 (m, 2H, —CH₂), 1.70-1.58 (m, 4H, —CH₂), 1.42 (s, 18H, —C(CH₃)₃), 1.35-1.37 (m, 16H, —CH₂), 0.86 (t, 3H, J_{AB}=6.5 Hz, —CH₃).

di-tert-Butyl ((octanoylazanediyl)bis(propane-3,1-diyl))dicarbamate (20): Compound 20 was synthesized from octanoyl chloride and compound 1 and following the similar synthetic procedure as mentioned for synthesizing compound 19 (52%). di-tert-butyl ((acetylazanediyl)bis(propane-3,1-diyl))dicarbamate (21): Compound 21 was synthesized from acetyl chloride and compound 1 and following the similar synthetic procedure as mentioned for synthesizing compound 19 (72%). ¹H NMR (CDCl₃): 3.42-3.38 (m, 4H, —CH₂N), 3.29-3.24 (m, 4H, —CH₂N), 2.10 (s, 3H, —COCH₃), 1.69-1.63 (m, 4H, —CH₂), 1.43 (s, 18H, —C(CH₃)₃).

di-tert-Butyl ((nonadec-9-enoylazanediyl)bis(propane-3,1-diyl))(Z)-dicarbamate (22): Compound 22 was synthesized from oleoyl chloride and compound 1 and following the similar synthetic procedure as mentioned for synthesizing compound 19 (69%). ¹H NMR (CDCl₃): 5.38-5.26 (m, 2H, —CH═CH—), 3.42-3.21 (m, 4H, —CH₂N), 3.17-2.99 (m, 4H, —CH₂N), 2.32-2.25 (m, 2H, —CH₂CO), 2.07-1.93 (m, 2H, CH₂), 1.80-1.72 (m, 2H, CH₂), 1.67-1.57 (m, 4H, —CH₂), 1.43 (s, 18H, —C(CH₃)₃), 1.33-1.22 (m, 20H, CH₂), 0.90-0.84 (m, 3H, —CH₃).

N,N-Bis(3-aminopropyl)dodecanamide di-hydrochloride (23): A solution of di-tert-butyl ((dodecanoylazanediyl)bis(propane-3,1-diyl))dicarbamate (19) (300 mg, 0.58 mmol) in 6N HCl (6 mL) and tetrahydrofuran (6 mL) was stirred for overnight, after which solvent was evaporated and crude mass was dissolved in methanol-dichloromethane followed by addition of diethyl ether, the process was repeated for 2-3 times and finally obtain pure N,N-bis(3-aminopropyl)dodecanamide di-hydrochloride, as brown yellow sticky mass (403 mg, 78%). ¹H NMR (DMSO-d₆): δ 3.30-3.17 (m, 4H, —CH₂N), 3.14-3.04 (m, 4H, —CH₂N), 2.86-2.78 (m, 2H, —CH₂CO), 1.80-1.70 (m, 2H, —CH₂), 1.70-1.58 (m, 4H, —CH₂), 1.35-1.37 (m, 16H, —CH₂), 0.86 (t, 3H, J_{AB}=6.5 Hz, —CH₃). ESI-MS (m/z): 314.35 (M+H).

N,N-Bis(3-aminopropyl)octanamide di-hydrochloride (24): Compound 24 was synthesized from compound 20 and following similar synthetic procedure as mentioned for synthesizing compound (23). (78%). ¹H NMR (D₂O): δ 3.50-3.44 (m, 4H, —CH₂N), 3.20-3.15 (m, 4H, —CH₂N), 2.42-2.39 (m, 2H, —CH₂CO), 2.02-1.88 (m, 4H, —CH₂), 1.59-1.53 (m, 2H, —CH₂), 1.32-2.1 (m, 8H, —CH₂), 0.82-0.81 (m, 3H, —CH₃).

N,N-Bis(3-aminopropyl)acetamide di-hydrochloride (25): Compound 25 was synthesized from compound 21 and following similar synthetic procedure as mentioned for synthesizing compound (23). (68%).

(Z)-N,N-bis(3-aminopropyl)nonadec-9-enamide di-hydrochloride (26): Compound 26 synthesized from compound 22 and following similar synthetic procedure as mentioned for synthesizing compound (23). (52%) ¹H NMR (D₂O): 5.34-5.18 (m, 2H, —CH═CH—), 3.46-3.33 (m, 4H, —CH₂N), 3.05-2.90 (m, 4H, —CH₂N), 2.39-2.30 (m, 2H, —CH₂CO), 2.01-1.88 (m, 6H, CH₂), 1.56-1.47 (m, 4H, —CH₂), 1.24-1.16 (m, 20H, CH₂), 0.86-0.76 (m, 3H, —CH₃).

Synthesis of Polymer SMP-043: Polymer SMP-043 was synthesized by from compound 23 and compound 13 and following similar synthetic procedure as mentioned for synthesizing polymer SMP-007. ¹H NMR (D₂O): δ 3.54-3.43 (m, 4H, —CH₂N), 3.24-3.16 (m, 4H, —CH₂N), 3.15-3.09 (m, 4H, —CH₂N), 2.17-2.07 (m, 4H, —CH₂), 1.98-1.91 (m, 2H, —CH₂), 1.64-1.54 (m, 4H, —CH₂), 1.45-1.36 (m, 4H, —CH₂), 1.31-1.28 (m, 18H, —CH₂), 0.90-0.84 (m, 3H, —CH₃). Mₙ (predicted): 3900.

Synthesis of Polymer SMP-045: Polymer SMP-045 was synthesized from compound 24 and compound 13 and following similar synthetic procedure as mentioned for synthesizing polymer SMP-007. ¹HNMR (D₂O): δ 3.24-3.20 (m, 4H, —CH₂N), 3.20-3.04 (m, 8H, —CH₂N), 2.30-2.23 (m, 2H, —CH₂CO), 2.15-2.07 (m, 2H, —CH₂), 1.94-1.88 (m, 2H, —CH₂), 1.62-1.55 (m, 4H, —CH₂), 1.46-1.36 (m, 2H, —CH₂), 1.32-1.26 (m, 12H, —CH₂), 0.88-0.84 (m, 3H, —CH₃). Mₙ (predicted): 3500.

Synthesis of Polymer SMP-017: Synthesized from compound 25 and compound 13 and following similar synthetic procedure as mentioned for synthesizing polymer SMP-007. ¹H NMR (D₂O): δ 3.32-3.19 (m, 4H, —CH₂N), 3.05-2.94 (m, 8H, —CH₂N), 1.84 (s, 3H, —CH₃CO), 1.75-1.61 (m, 4H, —CH₂), 1.43-1.33 (m, 4H, —CH₂), 1.22-1.31 (m, 4H, —CH₂). Mₙ (predicted): 2900.

Synthesis of Polymer SMP-060: Synthesized from compound 26 and compound 13 and following similar synthetic procedure as mentioned for synthesizing polymer SMP-007. ¹H NMR (D₂O): 5.36-5.19 (m, 2H, —CH═CH—), 3.49-3.36 (m, 4H, —CH₂NCO), 3.26-2.94 (m, 8H, —CH₂N), 2.24-2.13 (m, 2H, —CH₂CO), 1.96-1.85 (m, 8H, CH₂), 1.63-1.44 (m, 6H, —CH₂), 1.33-1.17 (m, 24H, CH₂), 0.87-0.79 (m, 3H, —CH₃). Mₙ (predicted): 5500.

### Example 6

### Synthesis of Compound SMP-052

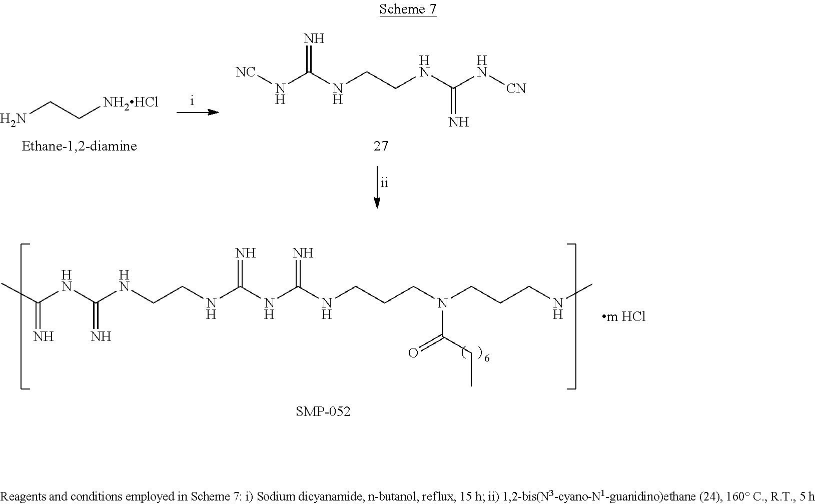

1,2-bis(N³-cyano-N¹-guanidino)ethane (27): Compound 27 was synthesized from ethane 1,2 diamine hydrochloride and compound 24 and following the procedure as mentioned for synthesizing compound 13 (72%). ¹H NMR (DMSO-d₆): δ 7.21-7.08 (brs, 2H, NH), δ 6.97-6.77 (brs, 4H, NH), 3.15-3.05 (m, 4H, CH₂N).

Synthesis of SMP-052: Polymer SMP-052 was synthesized by following the procedure of SMP-007. ¹H NMR (D₂O): δ 3.26-3.17 (m, 4H, —CH₂N), 3.12-3.03 (m, 8H, —CH₂N), 1.96-1.88 (m, 2H, —CH₂CO), 1.63-1.54 (m, 4H, —CH₂), 1.44-1.34 (m, 2H, —CH₂), 1.28-1.22 (m, 8H, —CH₂), 0.90-0.83 (m, 3H, —CH₃). Mₙ (predicted): 3200

### Example 7

Synthesis of Compound SMP-026

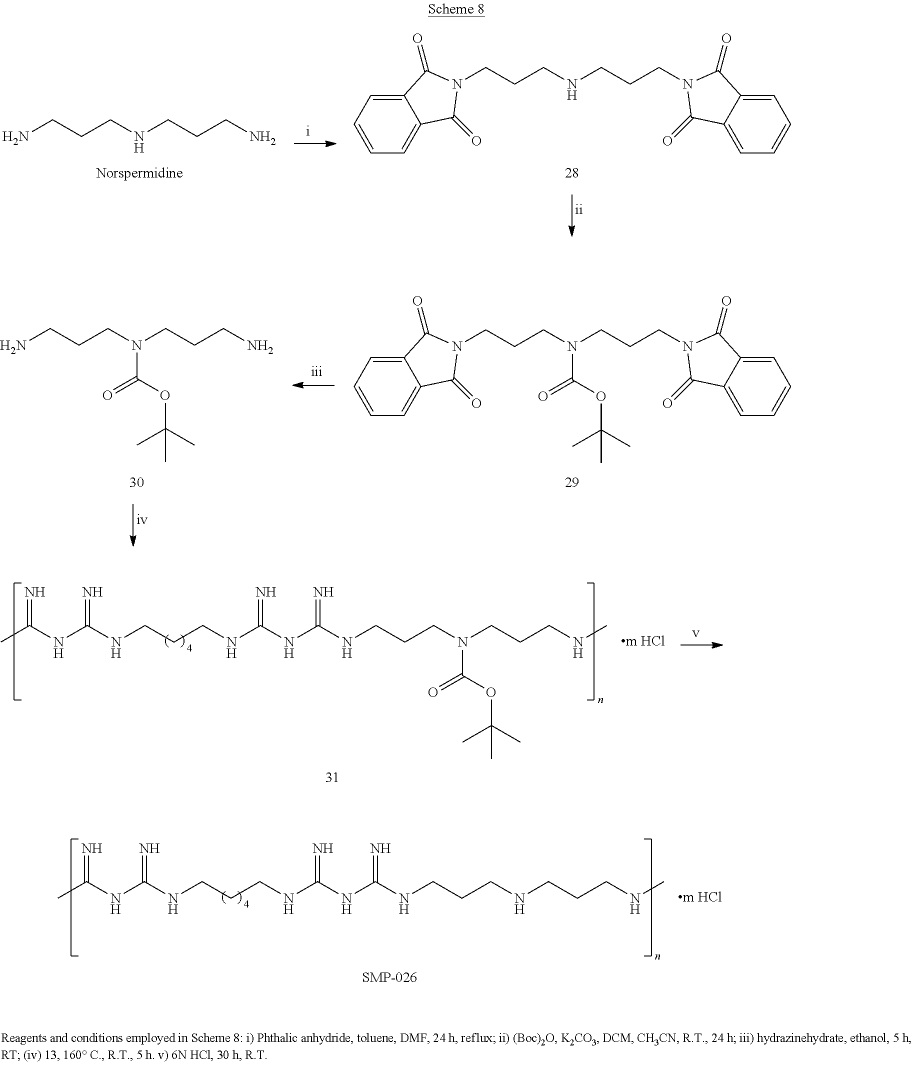

2,2′-(Azanediylbis(propane-3,1-diyl))bis(isoindoline-1,3-dione) (28): Phthalic anhydride (10.0 g, 67.5 mmol) was added to a solution of norspermidine (4.07 g, 4.37 mL, 31.0 mmol) in toluene/DMF (100 mL/10 mL). The reaction mixture was stirred under reflux condition for 1 day. The solvent was evaporated off and 300 mL of ethanol was added to the residue. After stirring for 5 h, the precipitate was filtered, collected and dried to give 2,2′-(azanediylbis(propane-3,1-diyl))bis(isoindoline-1,3-dione) (10 g, 85%). ¹H NMR (CDCl₃): δ=7.84-7.76 (m, 4H, ArH), 7.74-7.64 (m, 4H, ArH), 3.78-3.68 (m, 4H, —CH₂N), 2.69-2.54 (m, 4H, —CH₂N), 1.89-1.76 (m, 4H, —CH₂).

tert-Butyl bis(3-(1,3-dioxoisoindolin-2-yl)propyl)carbamate (29): Di-tert-butyldicarbonate (0.70 mL, 3.07 mmol) was added to a solution of 2,2′-(azanediylbis(propane-3,1-diyl))bis(isoindoline-1,3-dione), (28) (1.00 g, 2.56 mmol) with K₂CO₃ (0.50 g, 3.63 mmol) in dichloromethane:acetonitrile (100 mL:100 mL). The reaction mixture was stirred for 1 day at room temperature. The solvent was evaporated and the residue was dissolved in 200 mL of dichloromethane. The organic phase was washed with water, dried with sodium sulphate and concentrated to give tert-butyl bis(3-(1,3-dioxoisoindolin-2-yl)propyl)carbamate (1 g, 80%) as a white power which was directly used in next step without further purification.

tert-Butyl bis(3-aminopropyl)carbamate (30): tert-butyl bis(3-(1,3-dioxoisoindolin-2-yl)propyl)carbamate (29) (1.00 g, 2.04 mmol) and hydrazine monohydrate (1.00 mL, 20.6 mmol) in ethanol (10 mL) was stirred for 5 h at room temperature. After the reaction, the precipitate was removed by filtration. The filtrate was evaporated and extracted with dichloromethane. The combined organic layers were evaporated to give light yellow oily tert-butyl bis(3-aminopropyl)carbamate (236 mg, 50%). This compound was used for the next reaction without further purification. ¹H NMR (CDCl₃): δ 3.1-3.18 (m, 4H, —CH₂), 2.69 (t, 4H, J_{AB}=6.5 Hz, —CH₂), 1.69-1.63 (m, 4H, —CH₂), 1.45 (s, 9H, —C(CH₃)₃).

Synthesis of Polymer 31: A mixture of tert-butyl bis(3-aminopropyl)carbamate (230 mg, 1 mmol) and 1,6-bis(N³-cyano-N¹-guanidino)hexane (13) (300 mg, 1.2 mmol) was heated slowly to 160° C. in presence of 1 drop of conc. HCl. After reaching the temperature the mixture melt was kept at that particular temperature for another 5 h. After which reaction mixture was cooled and water was added to it and the mixture was filtered through a 0.22 μm syringe filter. Filtrate was evaporated and dried under vacuum for several hours to obtain desired polymer 31 as brown colour mass (55 mg). ¹H NMR (DMSO-d₆): δ 7.12 (brs, 1H, —NH), 6.70 (brs, 1H, —NH), 3.46-3.29 (m, 8H, —CH₂N(CNH)), 3.03-2.99 (m, 4H, —CH₂NCO), 1.61-1.49 (m, 4H, —CH₂), 1.40-1.36 (m, 13H; —CH₂, C(CH₃)₃), 1.28-1.02 (m, 4H, —CH₂).

Synthesis of Polymer SMP-26: Compound 31 (90 mg) was suspended in 6 N hydrochloric acid (5 mL) at 0° C. and reaction mixture was allowed to warm slowly at R.T. and stirring was continued for another at stirred at R.T for 30 h. After Solvent was evaporated under reduced pressure and crude was washed several times with dichloromethane to obtain desired polymer SMP-026 which was dried under vacuum for several hours (55 mg). ¹H NMR (D₂O): δ 3.32-3.25 (m, 4H, —CH₂N), 3.21-3.15 (m, 4H, —CH₂N(CNH)), 3.11-3.09 (m, 4H, —CH₂N(CNH)), 2.13-2.06 (m, 4H, —CH₂), 1.68-1.61 (m, 4H, —CH₂), 1.41-1.34 (m, 4H, —CH₂). Mₙ=6600 M_{w}=9300 PDI=1.4.

### Example 8

Synthesis of Compound SMP-027

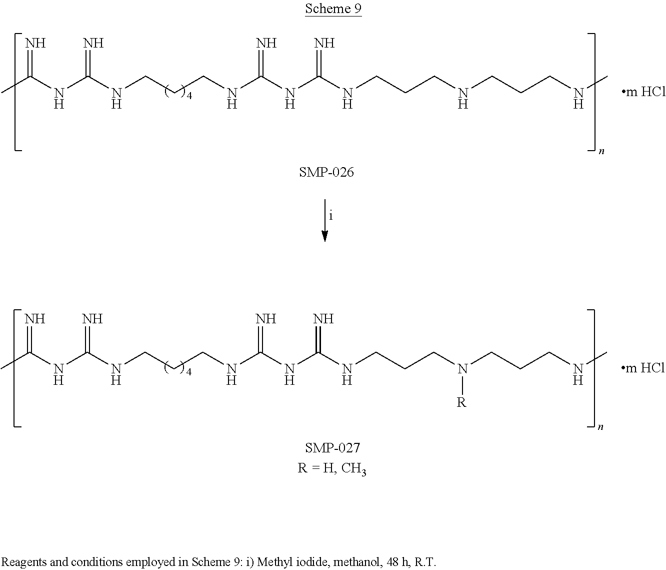

Synthesis of Polymer SMP-27: To a solution of SMP-026 (50 mg) in methanol methyl iodide (2 mL,) was added reaction mixture was stirred at room temperature for 2 days. After that solvent was evaporated under reduced pressure and crude was washed several times with dichloromethane to obtain desired polymer SMP-027 as brown colour solid which was dried under vacuum for several (60 mg). ¹H NMR (D₂O): δ 3.32 (s, 3H, —CH₃), 3.28-3.26 (m, 4H, —CH₂), 3.20-3.17 (m, 4H, —CH₂), 3.12-3.09 (m, 4H, —CH₂), 2.11-2.07 (m, 4H, —CH₂), 1.70-1.59 (m, 4H, —CH₂), 1.42-1.34 (m, 4H, —CH₂). Mₙ (predicted)=6700

### Example 9

Synthesis of Compound SMP-057

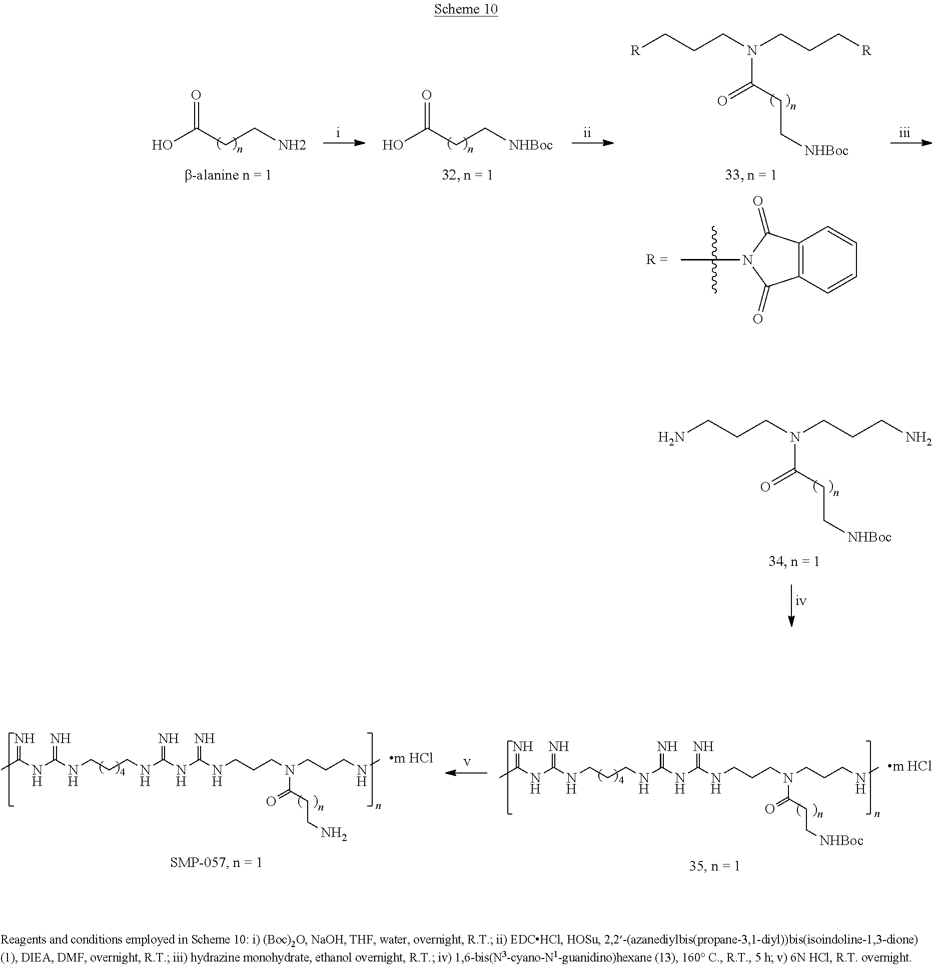

3-((tert-Butoxycarbonyl)amino)propanoic acid (32): To a solution of β-Alanine (2.0 g, 22.4 mmol) in 1:2 1M NaOH:THF (30 mL), (Boc)₂O (5.88 g, 26.88 mmol) was added at 0° C. The solution was stirred overnight at room temperature, and then concentrated under vacuo. The aqueous layer was washed with ethyl acetate, acidified to pH 2.0 with 4M HCl, extracted with ethyl acetate, washed with brine, and dried over sodium sulfate and concentrated in-vacuo to obtain 1 as colorless crystals. (3.96 g, 95%). ¹H NMR (CDCl₃): δ 5.1 (brs, 1H, —NH), 3.50-3.31 (m, 2H, —CH₂N), 2.61-2.54 (m, 2H, —CH₂), 1.47 (s, —C(CH₃)₃).

tert-Butyl (3-(bis(3-(1,3-dioxoisoindolin-2-yl)propyl)amino)-3-oxopropyl)carbamate (33): Synthesized from compound 32 and compound 1 and following the procedure as mentioned for synthesizing compound 5. ¹H NMR (CDCl₃): δ 8.03 (brs, 1H, —NH), 7.86-7.83(m, 2H, —ArH), 7.76-7.73 (m, 2H, —ArH), 3.72-3.70 (m, 2H, —CH₂N), 3.43-3.37 (m, 6H, —CH₂, —CH₂N), 2.50-2.48 (m, 2H, —CH₂CO), 1.78-1.76 (m, 4H, —CH₂), 1.46 (s, —C(CH₃)₃).

tert-Butyl (3-(bis(3-aminopropyl)amino)-3-oxopropyl)carbamate (34): Synthesized from compound 33 and following the procedure as mentioned for synthesizing compound 30. ¹H NMR (DMSO-d₆): δ 3.85-2.78 (m, 4H, —CH₂N), 3.13-3.00 (m, 4H, —CH₂N), 3.43-3.37 (m, 4H, CH₂, —CH₂N), 2.45-2.42 (m, 2H, —CH₂N), 2.22-2.1 (m, 2H, —CH₂N), 1.59-1.51 (m, 4H, —CH₂), 1.37 (s, —C(CH₃)₃).

Synthesis of Polymer 35: Polymer 35 was synthesis from compound 34 and compound 13 and following the procedure as mentioned for synthesizing compound 31. ¹H NMR (DMSO-d₆): δ 3.56-3.42 (m, 4H, —CH₂N), 3.40-3.29 (m, 4H, CH₂N), 3.28-3.19 (m, 4H, —CH₂N), 3.18-3.07 (m, 2H, —CH₂N), 2.44-2.34 (m, 4H, —CH₂), 2.02-1.93 (m, 2H, —CH₂CO), 1.91-1.76 (m, 4H, —CH₂), 1.45 (s, 9H, —C(CH₃)₃), 1.37-1.29 (m, 4H, —CH₂).

Synthesis of Polymer SMP-057: Polymer SMP-037 was synthesis from compound 35 and following the procedure as mentioned for synthesizing polymer SMP-26. ¹H NMR (D₂O): δ 3.38-3.30 (m, 8H, —CH₂N), 3.17-3.3.09 (m, 4H, —CH₂N), 2.79-2.72 (m, 2H, —CH₂NH₂), 2.44-2.34 (m, 4H, —CH₂), 2.32-2.29 (m, 2H, —CH₂CO), 1.99-1.91 (m, 4H, —CH₂), 1.51-1.40 (m, 4H, —CH₂). Mₙ (predicted)=6700.

### Example 10

### Synthesis of Compound SMP-037

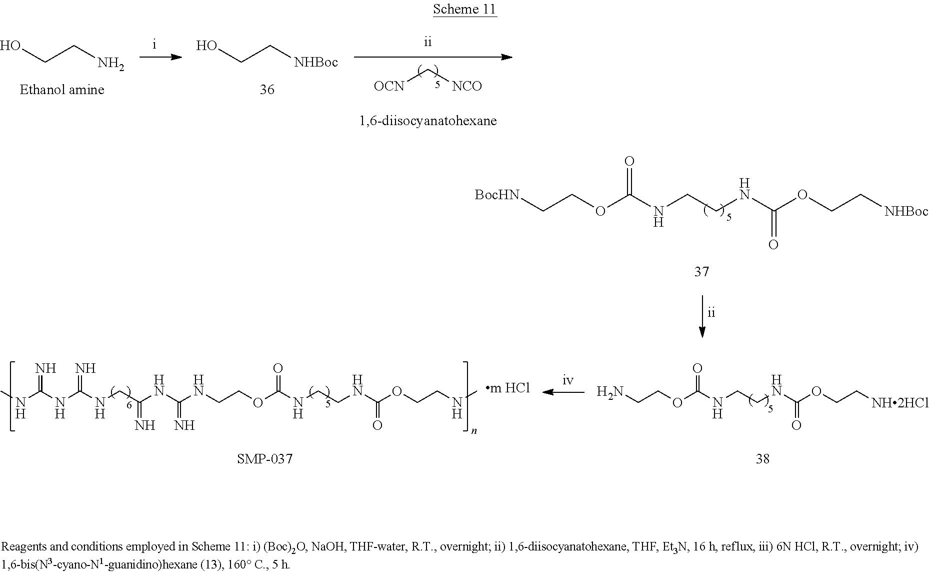

tert-butyl (2-hydroxyethyl)carbamate (36): To a solution of ethanolamine (0.7 mL, 12 mmol) in THF (15 mL) and NaOH (5M, 3 mL) in an ice bath was added di-tert-butyldicarbonate (Boc)₂O (3.12 g, 14 mmol), and the mixture was stirred at room temperature for overnight. The solution was concentrated under reduced pressure, and the resulting crude was extracted with ethyl acetate, and 10% citric acid, saturated, NaHCO₃(aq), and brine. Finally dried over sodium sulfate and filtered. The solvent was evaporated under reduced pressure to give compound 36 as a yellow liquid which used in next step without further purification (1.5 g, 80%).

bis(2-((tert-butoxycarbonyl)amino)ethyl) hexane-1,6-diyldicarbamate (37): A solution of tert-butyl (2-hydroxyethyl)carbamate (36) (1.61 g, 10 mmol), 1,6-diisocyanatohexane (0.8 mL, 4 mmol) and triethylamine (4.1 mL, 30 mmol) was heated to refluxed for 16 h. After completion of the reaction solvent was evaporated under reduced pressure to obtain crude which was further portioned between ethyl acetate and water. Desired compound 37 was obtained after evaporation of the organic layer. ¹H NMR (CDCl₃): δ 7.25-7.23 (m, 1H, —NH), 7.12-7.13 (m, 1H, —NH), 4.18-4.05 (m, 4H, —CH₂O), 3.42-3.32 (m, 4H, —CH₂N), 3.20-3.01 (m, 4H, —CH₂N), 1.42-1.34 (m, 4H, —CH₂), 1.43 (s, 18H, —C(CH₃)₃), 1.35-1.31 (m, 4H, —CH₂).

bis(2-aminoethyl) hexane-1,6-diyldicarbamate dihydrochloride (38): Compound 38 was synthesized from compound 37 and 1,6-diisocyanatohexane and following the procedure as mentioned for synthesizing compound SMP-002.

Synthesis of polymer SMP-037: Polymer SMP-037 was synthesized from compound 38 and compound 13 and following the procedure as mentioned for synthesizing compound SMP-007. ¹H NMR (DMSO-d₆): 6.66 (brs, 6H, NH), 7.15 (brs, 2H, NH), 4.13 (t, J_{AB}=5.5 Hz, 4H, —CH₂O), 3.00-2.94 (m, 12H, —CH₂N), 1.44-1.36 (m, 8H, —CH₂), 1.28-1.22 (m, 8H, —CH₂). Mₙ (predicted)=3900

### Example 11

### Synthesis of Compound SMP-049

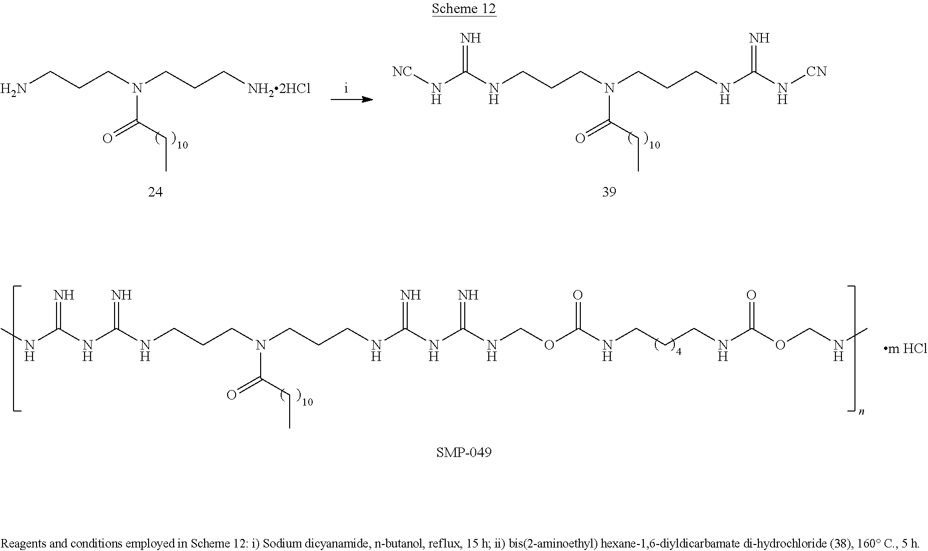

N,N-bis(3-(3-cyanoguanidino)propyl)dodecanamide (39): Compound 39 was synthesized from compound 24 and following the procedure as mentioned for synthesizing compound (13). ¹H NMR (DMSO-d₆): δ 3.20-3.02 (m, 4H, —CH₂N), 2.98-2.93 (m, 4H, —CH₂N), 2.09-2.01 (m, 2H, —CH₂CO), 1.70-1.52 (m, 4H, —CH₂), 1.52-1.42 (m, 2H, —CH₂), 1.30-1.20 (m, 16H, —CH₂), 0.89-0.86 (m, 3H, —CH₃). (50 mg, 45%).

Synthesis of polymer SMP-049: Polymer SMP-049 was synthesized from compound 39 and compound 38 and following the procedure as mentioned for synthesizing polymer SMP-007. ¹H NMR (DMSO-d₆): δ 8.40-8.10 (brs, 12H, NH), 4.03-3.98 (m, 2H, —CH₂O), 3.91-3.86 (m, 2H, —CH₂O), 3.24-3.18 (m, 4H, —CH₂N), 2.99-2.93 (m, 8H, —CH₂N), 2.77-2.69 (m, 6H, —CH₂N, —CH₂CO), 1.62-1.53 (m, 6H, —CH₂), 1.36-1.30 (m, 6H, —CH₂), 1.27-1.17 (m, 18H, —CH₂), 0.90-0.82 (m, 3H, —CH₃). Mₙ (predicted)=4800.

### Example 12

Synthesis of Compounds SMP-018, SMP-019, SMP-020, SMP-033, SMP-035, SMP-034 and SMP-036

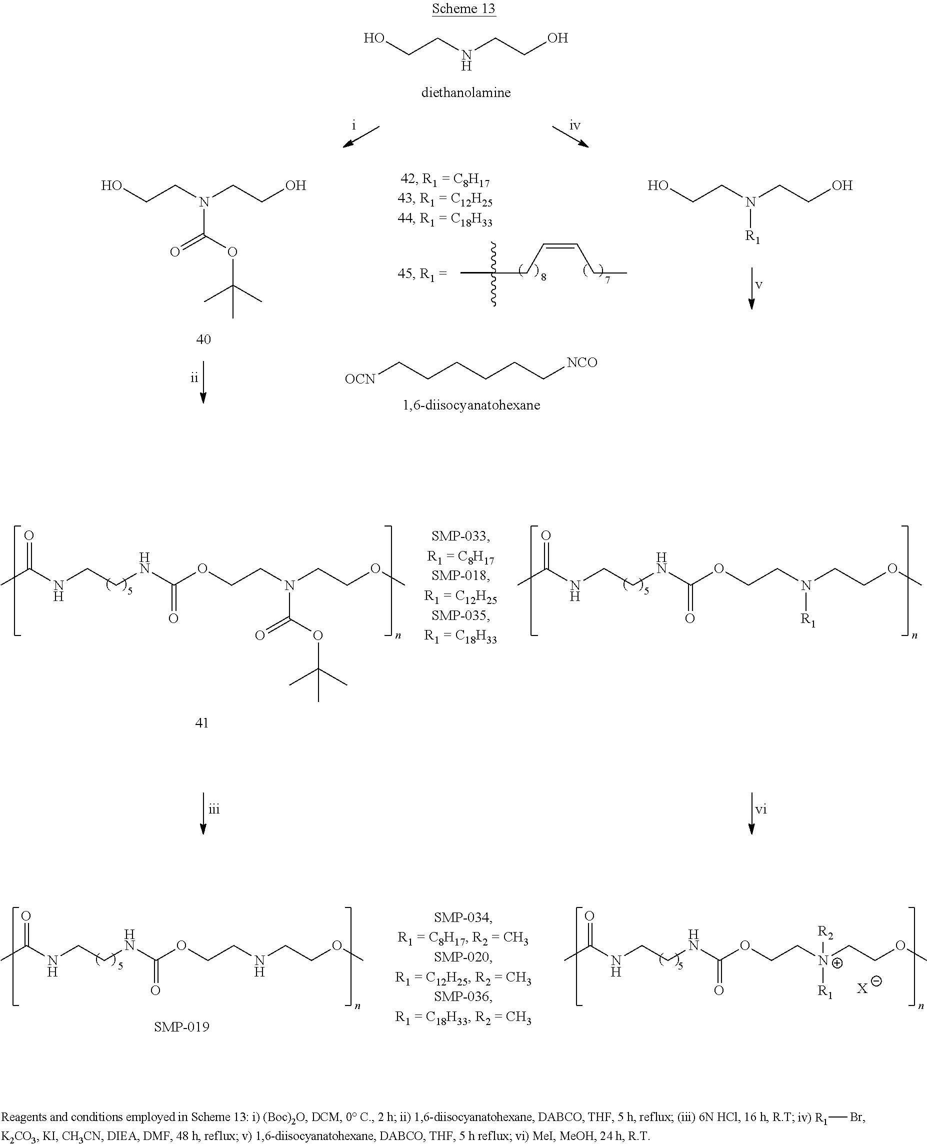

tert-Butyl-bis(2-hydroxyethyl)carbamate (40): To a stirred solution of diethanolamine (2.50 g, 23.80 mmol) in dichloromethane (30 mL), (Boc)₂O (7.26 g, 33.29 mmol) was added at 0° C. and allowed to stir for another 2 h at same temperature. After completion, the reaction mixture was washed with water and the product was extracted in dichloromethane. The organic layer was then washed with brine, dried over sodium sulfate and concentrated under vacuum to obtain the crude compound 40 as colorless liquid (3.5 g, 71%) which was further utilized for subsequent steps without purification. ¹H NMR (CDCl₃): δ 3.81-3.79 (m, 4H, —CH₂O), 3.46-3.41 (m, 4H, —CH₂N),1.47 (s, 9H, —C(CH₃)₃).

Synthesis of Polymer 41: To a mixture of compound 40 (0.5 g, 2.44 mmol) and 1,6-diisocyanatohexane (0.41 g, 2.44 mmol) were reacted in THF (1 mL), a solution of DABCO (11 mg in 3.25 mL THF) was added and the reaction mixture was stirred for 5 h at 60° C. under nitrogen atmosphere. Then the reaction mixture was allowed to cool to room temperature and the polymer was precipitated in presence of excess diethyl ether. Crude mass was then centrifuged, washed with diethyl ether and dried under vacuum to isolate the polymer as white sticky mass. (320 mg). ¹H NMR (DMSO-d₆): δ 4.09-0.01 (m, 4H, —CH₂O), 2.95-2.94 (m, 8H, —CH₂N), 1.42-1.37 (m, 4H, —CH₂), 1.39 (s, 9H, —C(CH₃)₃), 1.23-1.21 (m, 4H, —CH₂).

Synthesis of Polymer SMP-019: Compound 1 (100 mg) was suspended in 6 N hydrochloric acid (10 mL) at 0° C. and reaction mixture was allowed to stir at R.T. and stirring was continued for another 16 h to form a transparent solution. At the end, the solvent was then evaporated under reduced pressure and crude was washed several times with dichloromethane, dried to obtained to obtain desired polymer SMP-019 (55 mg). ¹H NMR (DMSO-d₆): δ 4.27-4.22 (m, 4H, —CH₂O), 3.29-3.12 (m, 4H, —CH₂N), 3.00-2.93 (m, 4H, —CH₂N), 1.45-1.35 (m, 4H, —CH₂), 1.28-1.24 (m, 4H, —CH₂). Mₙ (predicted): 7,000.

2,2′-(Octylazanediyl)bis(ethan-1-ol) (42): 1-bromooctane (3.0 g, 1.5 mmol), diethanolamine (2.4 g, 2.3 mmol), anhydrous potassium carbonate (4.28 g, 3.1 mmol) were taken in acetonitrile (40 mL) and the contents were refluxed for 12 h nitrogen atmosphere. After cooling reaction mixture was evaporated and extracted with dichloromethane and dried over anhy. Na₂SO₄ removed under reduced pressure to get the crude product which was further purified by column chromatography using dichloromethane and methanol as eluent to obtain pure 2,2′-(octylazanediyl)bis(ethan-1-ol) as colorless oil (590 mg, 91%). ¹H NMR (CDCl₃): δ 4.22 (brs, 2H, OHCH₂), 3.86 (t, 4H, J_{AB}=5 Hz, —CH₂OH), 3.10 (t, 4H, J_{AB}=4.5 Hz, —CH₂N), 2.94 (t, 2H, J_{AB}=8 Hz, —CH₂N), 1.69-1.60 (m, 2H, —CH₂), 1.30-1.25 (m, 10H, —CH₂), 0.87 (t, 3H, J_{AB}=7 Hz, —CH₃).

2,2′-(Dodecylazanediyl)bis(ethan-1-ol) (43): Compound 43 was synthesized by using diethanolamine and 1-bromododecane, and following the procedure of compound 42. ¹H NMR (CDCl₃): δ 3.61 (t, 4H, J_{AB}=5 Hz, —CH₂OH), 2.65 (t, 4H, J_{AB}=5.5 Hz, —CH₂N), 2.51 (t, 2H, J_{AB}=7.5 Hz, —CH₂N), 1.49-1.41 (m, 2H, —CH₂), 1.29-1.20 (m, 18H, —CH₂), 0.87 (t, 3H, J_{AB}=6.5 Hz, —CH₃).

(Z)-2,2′-(Octadec-9-en-1-ylazanediyl)bis(ethan-1-ol) (45): Compound 45 was synthesized by using diethanolamine and (Z)-1-Bromooctadec-9-ene, and following the procedure of compound 42. ¹H NMR (CDCl₃): δ 5.37-5.30 (m, 2H, —CH═CH—), 3.64-3.61 (m, 4H, —CH₂OH), 2.69-2.66 (m, 4H, —CH₂N), 2.55-2.50 (m, 2H, —CH₂N), 2.19 (brs, 2H, —OH), 2.04-1.98 (m, 2H, —CH₂),1.49-1.44 (m, 2H, —CH₂), 1.33-1.22(m, 24H, —CH₂), 0.90-0.86 (m, 3H, —CH₃).

Synthesis of Polymer SMP-018: To a mixture of compound 43 (0.5 g, 1.80 mmol), 1,6-diisocyanatohexane (0.24 g, 1.40 mmol) in THF (1.3 mL), a solution of DABCO (6.3 mg in 2 mL THF) was added and the reaction mixture was stirred for 5 h at 60° C. under nitrogen atmosphere. Reaction mixture was cooled to room temperature and polymer was precipitated in presence of excess diethyl ether. It was then centrifuged, washed with diethyl ether and dried under vacuum to isolate the polymer as white sticky mass. (344 mg). ¹H NMR (DMSO-d₆): δ 3.97-3.92 (m, 4H, —CH₂O), 2.98-2.90 (m, 4H, —CH₂N), 2.67-2.58 (m, 4H, —CH₂N), 2.47-2.41 (m, 2H, —CH₂N), 1.38-1.33 (m, 6H, —CH₂), 1.30-1.20 (m, 22H, —CH₂), 0.85 (t, J_{AB}=6.5 Hz, 3H, —CH₃). Mₙ (predicted): 12,400.

Synthesis of Polymer SMP-020: To a solution of SMP-018 (100 mg) in methanol (1 mL), methyl iodide (2 mL,) was added to the reaction mixture was stirred at room temperature for 24 h at room temperature. After completion, the solvent was evaporated under reduced pressure and crude was washed several times with dichloromethane to obtain desired polymer SMP-020 as yellow colour sticky solid which was dried under vacuum(70 mg). ¹H NMR (DMSO-d₆): δ 4.41-4.30 (m, 4H, —CH₂O), 3.61-3.57 (m, 4H, —CH₂N⁺), 3.51-3.47 (m, 2H, —CH₂N⁺), 3.10 (s, 3H, —CH₃), 3.0-2.93 (m, 4H, —CH₂NH), 1.38-1.35 (m, 6H, —CH₂), 1.28-1.20 (m, 22H, —CH₂), 0.85 (t, J_{AB}=6.8 Hz, 3H, —CH₃). Mₙ (predicted): 12,800.

### Example 13

Synthesis of Compound SMP-024

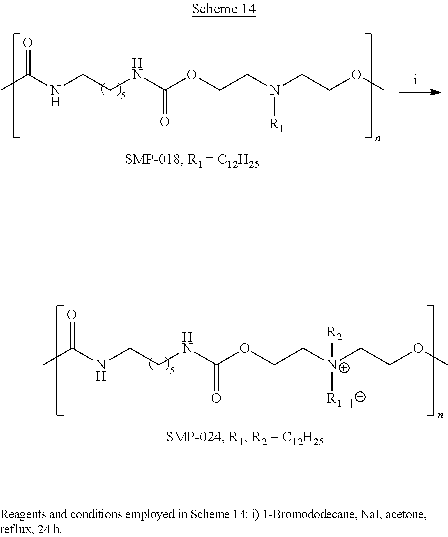

Synthesis of Polymer SMP-024: To a solution of SMP-018 (124 mg, 9.67 μmol) in acetone (4 mL), NaI (10% wt/wt of SMP-018) and 1-Bromododecane (2 mL, 0.29 mmol) was added and reaction mixture was refluxed for 24 h. After completion of the reaction, the solvent was decanted off and solid so obtained was washed several times with methanol to remove excess amount of 1-Bromododecane and other soluble salts to obtain the desired polymer SMP-024 as yellow colour sticky solid which was dried under vacuum(70 mg). ¹H NMR (DMSO-d₆): δ 5.8 (brs, 2H, NH), 4.20-3.94 (m, 4H, —CH₂O), 3.64-3.40 (m, 4H, —CH₂N⁺), 3.23-3.05 (m, 4H, —CH₂N⁺ merged with DMSO-d₆-H₂O residual peak); 2.95-2.94 (m, 4H, —CH₂NH), 1.41-1.3 (m, 8H, —CH₂), 1.30-1.15 (m, 40H, —CH₂), 0.86 (t, J_{AB}=10 Hz, 6H, —CH₃). Mₙ (predicted): 17,000.

### Example 14

Synthesis of Compound SMP-042

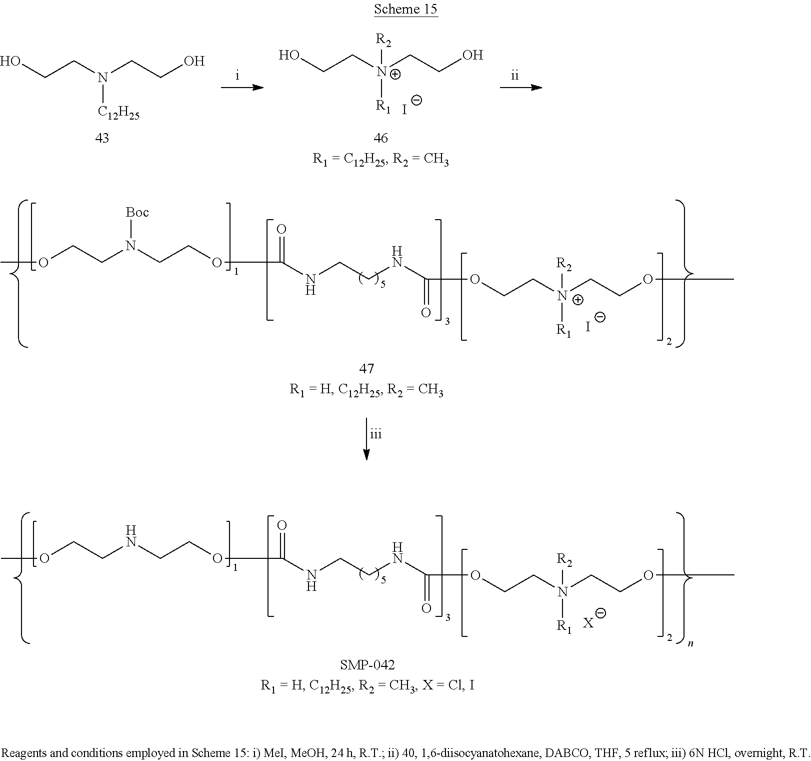

N,N-bis(2-hydroxyethyl)-N-methyldodecan-1-aminium (46): Compound 46 was synthesized by using compound 43 and methyl iodide and following the procedure of SMP-020. (158 mg, 75%). ¹H NMR (CDCl₃): δ4.18-4.12 (m, 4H—CH₂O), 4.10 (brs, 2H, —OH), 3.81-3.73 (m, 4H, —CH₂N⁺), 3.58-3.51 (m, 2H, —CH₂N), 3.34 (s, 3H, —CH₃), 1.81-1.72 (m, 2H, —CH₂), 1.36-1.33 (m, 2H, —CH₂), 1.31-1.21 (m, 16H, —CH₂), 0.91-0.84 (m, 3H, —CH₃).

Synthesis of Polymer 47: A mixture of compound 40 (0.25 g, 1.22 mmol), compound 46 (0.1 g, 0.35 mmol), 1,6-diisocyanatohexane (0.3 g, 1.8 mmol) and dried THF (˜1 mL) was taken in a round bottom flask under continuous flow of argon. To this, a solution of DABCO (8.2 mg in 2.4 mL THF) was added and the reaction mixture was stirred for 5 h at 60° C. The reaction mixture was allowed to cool to room temperature and the polymer was precipitated in presence of excess diethyl ether. It was then centrifuged, washed with diethyl ether and dried under vacuum to isolate the polymer as white sticky mass (275 mg). ¹H NMR (DMSO-d₆): δ 4.41-4.46 (m, 4H, —CH₂O), 4.05-3.95 (m, 8H, —CH₂O), 3.65-3.55 (m, 4H, CH₂N⁺), 3.50-3.44 (m, 8H, —CH₂NCO), 2.93 (s, 3H, —CH₃), 3.00-2.84 (m, 14H, —CH₂N⁺, —CH₂NCO), 1.4-1.33 (m, 32H, —CH₂, —C(CH₃)₃), 1.30-1.20 (m, 30H, —CH₂), 0.87-0.70 (m, 3H, —CH₃).

Synthesis of Polymer SMP-042: Synthesis of polymer SMP-042 was done by using compound 47 and following the procedure of polymer SMP-019 and the polymer was obtained as yellow sticky solid (220 mg). ¹H NMR (DMSO-d₆): δ 4.35-4.20 (m, 8H, —CH₂O), 3.95-3.75 (m, 4H, —CH₂O), 3.58-3.47 (m, 8H, CH₂N⁺), 3.40-3.28 (m, 4H, —CH₂NCO), 3.15 (s, 3H, —CH₃), 3.05-2.92 (m, 14H, —CH₂N⁺, —CH₂NCO), 1.42-1.33 (m, 14H, —CH₂), 1.25-1.18 (m, 30H, —CH₂), 0.83-0.72 (m, 3H, —CH₃).

Mₙ (predicted): 15,200.

### Example 15

Synthesis of Compound SMP-062

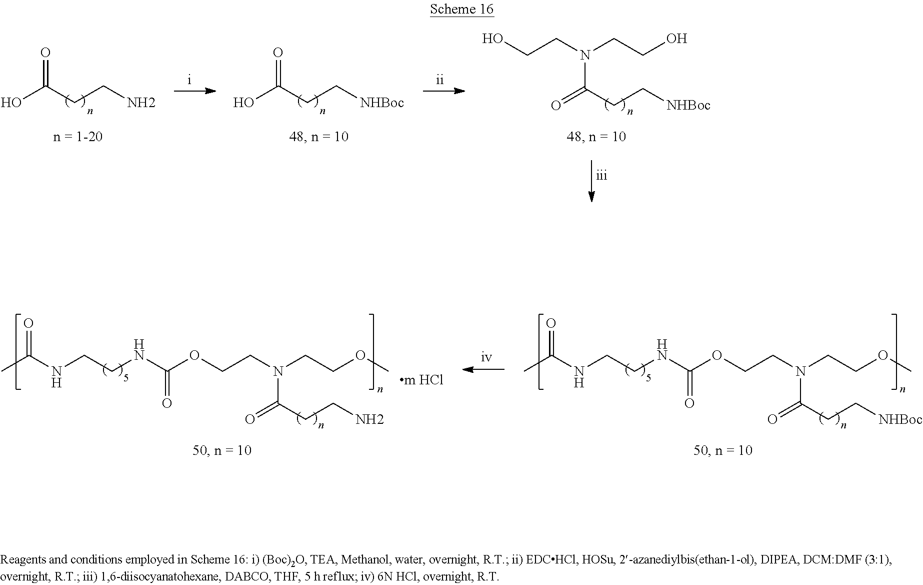

Synthesis of 11-((tert-butoxycarbonyl)amino)undecanoic acid (48): 12-aminododeconic acid (1.1 g, 5.0 mmol), triethylamine (0.8 mL, 5.7 mmol), and Boc₂O (1.053 g, 4.8 mmol) were combined in MeOH (15 mL) and refluxed at 60° C. overnight. After completion, the reaction mixture was concentrated under reduced pressure. The resultant residue was re-dissolved in ethyl acetate, washed with 0.25 M HCl, dried over MgSO₄, filtered, and concentrated. The desired colorless, crystalline solid was obtained by recrystallization with hexanes (1.250 g, 80%). ¹H NMR (CDCl₃): δ 4.54 (bs, 1H, NHCO), 3.18-3.08 (m, 2H, —CH₂N), 2.40-2.35 (m, 2H, —CH₂CO), 1.70-1.62 (m, 2H, —CH₂), 1.47(s, 9H, —C(CH₃)₃), 1.40-1.25 (m, 16H, —CH₂).

Synthesis of tert-butyl (11-(bis(2-hydroxyethyl)amino)-11-oxoundecyl)carbamate (49): To a solution of compound 48 (1 g, 3.2 mmol) in DCM:DMF (3:1, 20 mL), added HOSu (0.55 g, 4.8 mmol) and EDC.HCl (0.9 mg, 4.8 mmol) at 0° C. and the reaction mixture was stirred overnight at room temperature and was concentrated under reduced pressure. The resultant residue was extracted with ethyl acetate, dried over anhy. Na₂SO₄ and concentrated to obtain the HOSu ester as pale-yellow solid (1 g, 77%). Then to a solution of diethanolamine (0.306 g, 3 mmol) in DCM:DMF (3:1, 20 mL), added TEA (0.84 mL, 6.1 mmol) at 0° C. It was then followed by the addition of HOSu ester (1 g, 3.2 mmol) and the reaction mixture was stirred for another 16 h at room temperature. After completion, the reaction mixture was concentrated under reduced pressure. The resultant residue was re-dissolved in ethyl acetate, dried over anhy. Na₂SO₄, and concentrated to obtain crude mass which was further purified by flash column chromatography over silica gel using 3% methanol-dichloromethane as eluent to obtain compound 49 as white solid (0.8 g, 63%). ¹H NMR (CDCl₃): δ 4.53 (bs, 1H, NH), 3.89-3.78 (m, 4H, —CH₂O), 3.58-3.50 (m, 4H, —CH₂N), 3.14-3.04 (m, 2H, —CH₂N), 2.42-2.35 (m, 2H, —CH₂CO), 1.67-1.59 (m, 2H, —CH₂), 1.43 (s, 9H, —C(CH₃)₃), 1.35-1.23 (m, 16H, —CH₂).

Synthesis of Polymer 50: Synthesis of polymer 50 was done by using compound 49 and 1,6-diisocyanatohexane, and following the procedure of polymer 41 and the polymer was obtained as white solid. (450 mg). ¹H NMR (DMSO-d₆): δ 4.05-3.97 (m, 4H, —CH₂O), 3.59-3.39 (m, 4H, —CH₂N), 3.00-2.8 (m, 4H, —CH₂N), 2.75-2.73 (m, 2H, —CH₂N), 2.30-2.20 (m, 2H, —CH₂CO), 1.48-1.40 (m, 2H, —CH₂), 1.36 (s, 9H, —C(CH₃)₃), 1.30-1.15 (m, 24H, —CH₂). Mₙ (predicted): 16,400.

Synthesis of Polymer SMP-062: Synthesis of polymer SMP-062 was done by using compound 50 and following the procedure of polymer SMP-019 and the polymer was obtained as white solid. (367 mg). ¹H NMR (DMSO-d₆): δ 4.25-4.20 (m, 4H, —CH₂O), 3.54-3.47 (m, 4H, —CH₂N), 3.00-2.83 (m, 6H, —CH₂N), 2.30-2.23 (m, 2H, —CH₂CO), 1.32-1.14 (m, 26H, —CH₂). Mₙ (predicted): 14,500.

### Example 16

Synthesis of Compound SMP-041

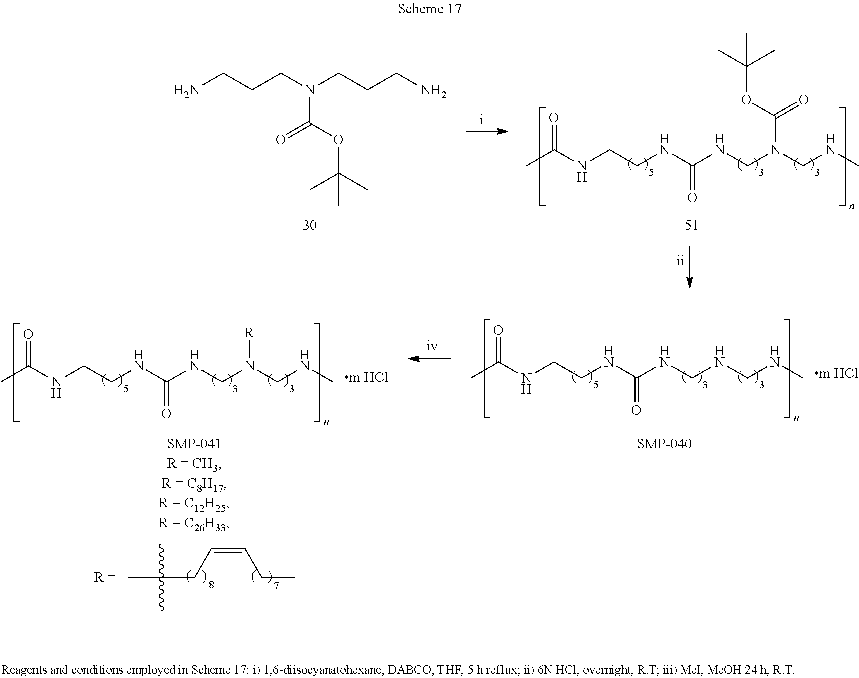

Synthesis of polymer 51: A mixture of compound 30 (0.5 g, 2.2 mmol), 1,6-diisocyanatohexane (0.36 g, 2.2 mmol) and dried THF (1.14 mL) was taken in a round bottom flask under continuous flow of argon. To this, a solution of DABCO (9.6 mg in 3.4 mL THF) was added and the reaction mixture was stirred for 5 h at 60° C. Then the reaction mixture was cool to room temperature and the polymer was precipitated in presence of diethyl ether. The resultant mass was centrifuged, washed with diethyl ether and dried under vacuum to isolate the polymer as white sticky mass (350 mg). ¹H NMR (DMSO-d₆): δ5.76 (brs, 2H, —NH), 3.13-3.10 (m, 4H, —CH₂NHCO), 2.99-2.91 (m, 8H, —CH₂NHCO, CH₂NCO), 1.57-1.49 (m, 4H, —CH₂), 1.39 (s, 9H, —C(CH₃)₃), 1.38-1.30 (m, 4H, —CH₂), 1.26-1.19 (m, 4H, —CH₂).

Synthesis of polymer SMP-040: Synthesis of polymer SMP-040 was done by using polymer 51 and following the procedure of SMP-019 and the polymer was obtained as pale-yellow sticky solid (380 mg). ¹H NMR (D₂O): δ3.22-3.18 (m, 2H, —CH₂N), 3.09-3.00 (m, H, —CH₂N), 1.88-1.80 (m, 4H, —CH₂), 1.49-1.40 (m, 4H, —CH₂), 1.32-1.26 (m, 4H, —CH₂). Mₙ (predicted): 8,900

Synthesis of polymer SMP-041: Synthesis of polymer SMP-041 was done by using SMP-040 and methyl iodide, and following the procedure of SMP-020 and the polymer was obtained as reddish brown sticky solid (220 mg). ¹H NMR (DMSO-d₆): δ 3.14-3.02 (m, 7H, —CH₂N, —CH₃), 2.98-2.92 (m, 4H, —CH₂N), 2.90-2.81 (m, 4H, —CH₂N), 1.72-1.63 (m, 4H, —CH₂), 1.2-1.21 (m, 4H, —CH₂), 1.20-1.16 (m, 4H, —CH₂). Mₙ (predicted): 9,300.

### Example 17

Synthesis of Compound SMP-064

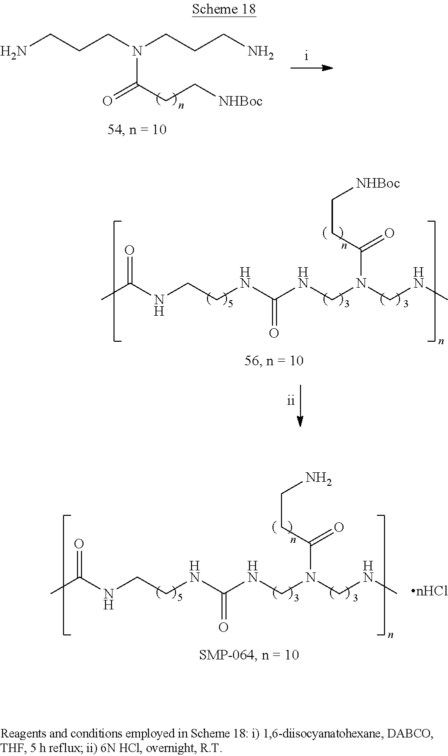

### Example 18

Synthesis of Compounds SMP-030, SNIP-053 and SNIP-029

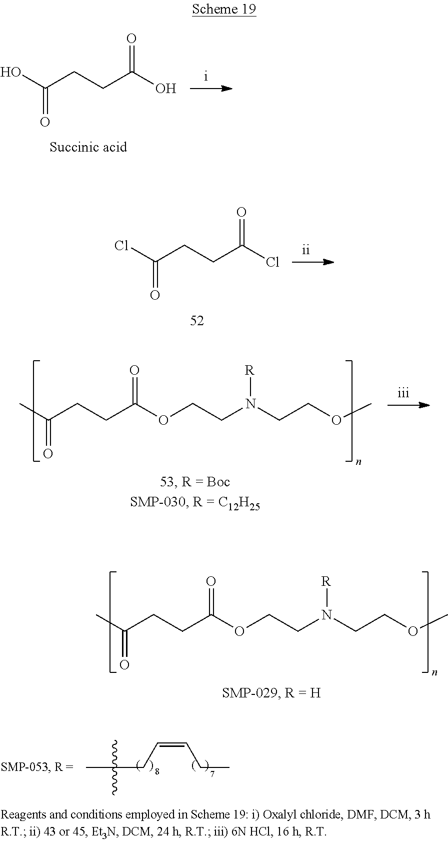

Succinyl dichloride (52): Succinic acid (2.95 g, 25 mmol) was dissolved in dry DCM (10 mL) with a catalytic amount of dry DMF, and oxalyl chloride (2.56 mL, 30 mmol) was added slowly at 0° C. and reaction mixture was allowed to stir at room temperature for another 3 h. Excess oxalyl chloride was removed under reduced pressure in rotary evaporator. The residue left upon vacuum drying afforded the desired succinyl dichloride (3.4 g, 95% yield).

Synthesis of Polymer SMP-030: To a solution compound 43 (820 mg, 3 mmol) and triethyl amine (0.5 mL) in dichloromethane (50 ml) at 0° C., a solution of succinyl dichloride (558 mg, 3.6 mmol) in dichloromethane was added slowly and the reaction mixture was stirred at room temperature for 24 h. After that reaction mixture was washed with water and sodium bicarbonate solution. On evaporation of the solvent polymer SMP-030 was obtained as brown mass, (200 mg). ¹H NMR (CDCl₃): δ 4.71-4.48(m, 4H, —CH₂OH), 4.09-3.92 (m, 4H, —CH₂N), 3.24-3.21 (m, 2H, —CH₂N), 1.88-1.78 (m, 4H, —CH₂), 1.35-1.32 (m, 4H, —CH₂CO), 1.26-1.24 (m, 18H, —CH₂), 0.87 (t, 3H, J_{AB}=6.5 Hz, —CH₃).

Synthesis of Polymer SMP-053: Synthesis of polymer SMP-053 was done by using compound 45 and compound 53, and following the procedure of SMP-019. ¹H NMR (CDCl₃): δ 5.36-5.30 (m, 2H, —CH═CH—), 4.56-4.50 (m, 4H, —CH₂OH), 3.55-3.46 (m, 4H, —CH₂N), 3.38-3.32 (m, 2H, —CH₂N), 2.68-2.59 (m, 4H, —CH₂CO), 2.03-2.00 (m, 2H, —CH₂), 1.81-1.75 (m, 2H, —CH₂), 1.33-1.24 (m, 24H, —CH₂), 0.89-0.86 (m, 3H, —CH₃).

### Example 19

Synthesis of Compound SMP-066

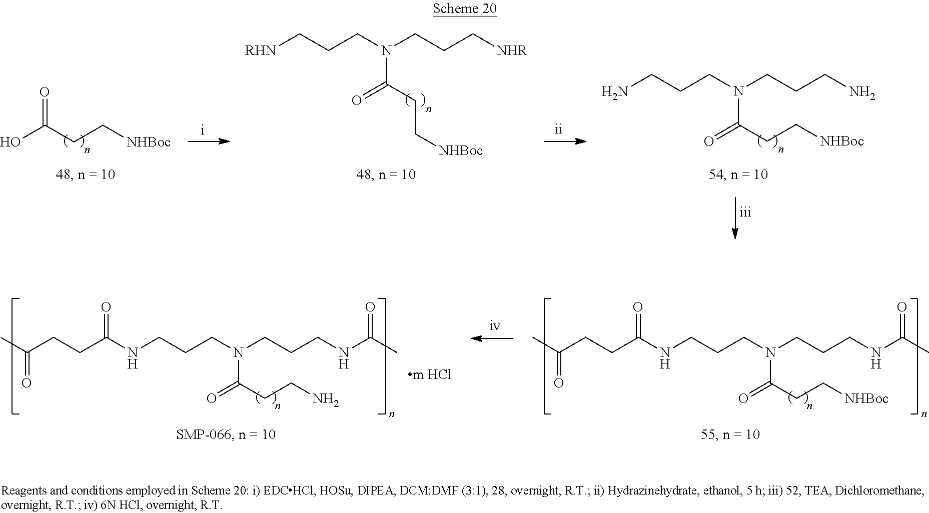

### Example 20

Synthesis of Compound SMP-070

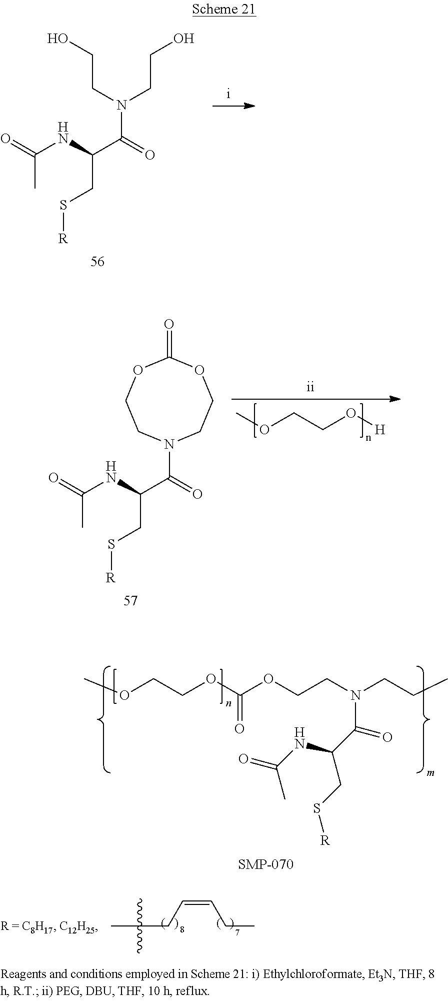

### Example 21

Synthesis of Compounds SMP-071 to SMP-075

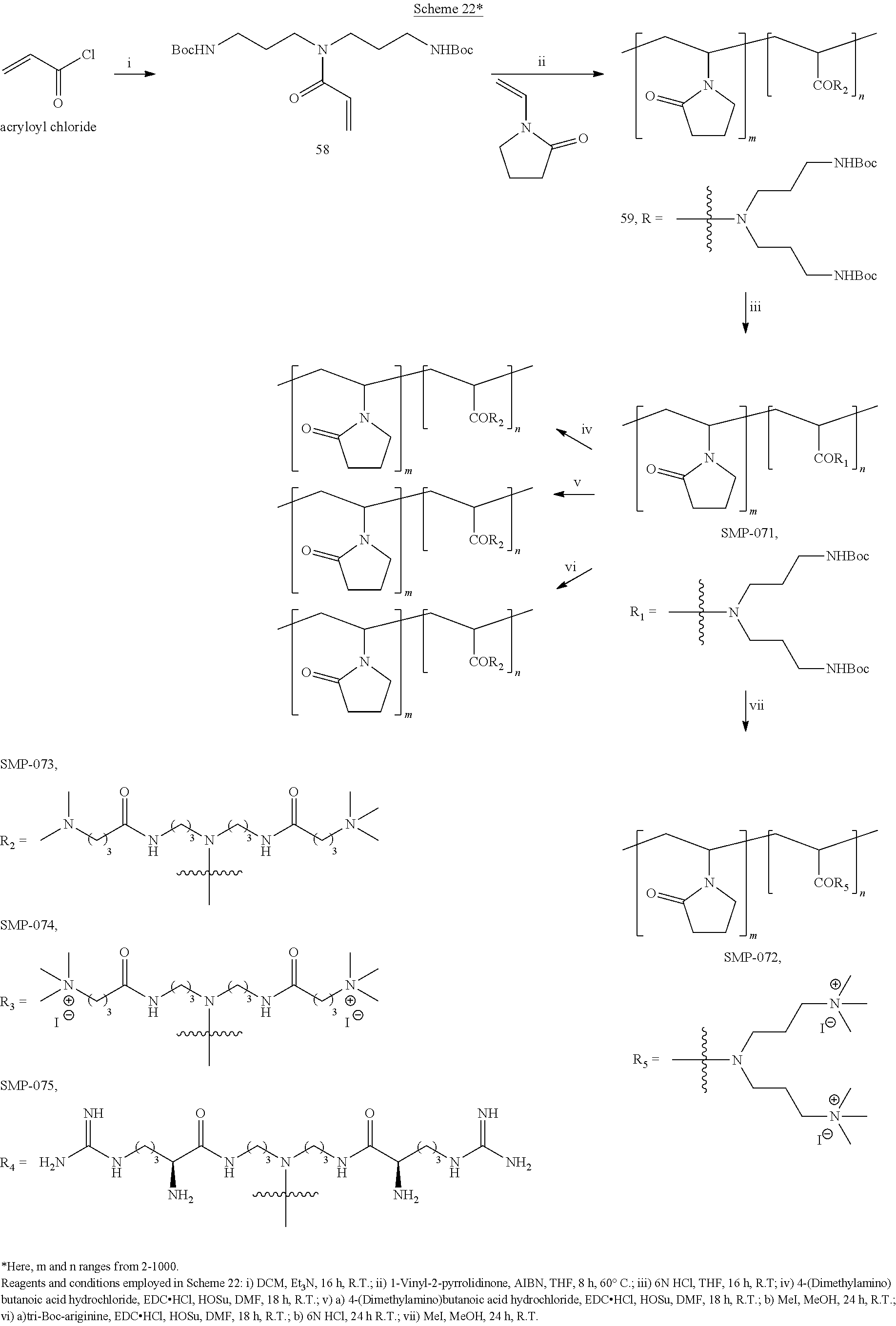

### Example 22

Synthesis of Compound SMP-082*

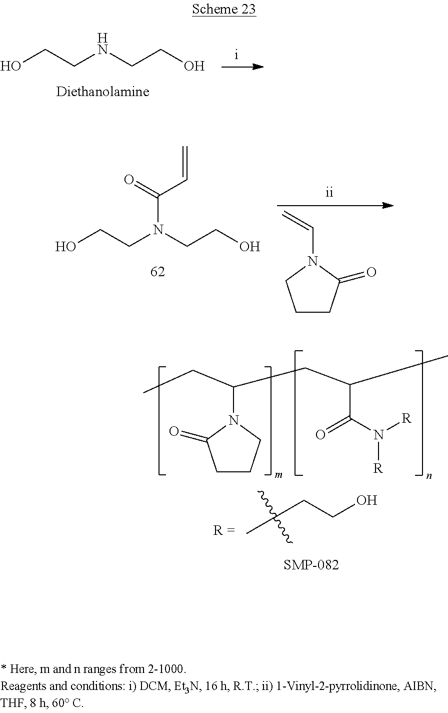

### Example 23

Synthesis of compounds SMP-083 and SMP-084

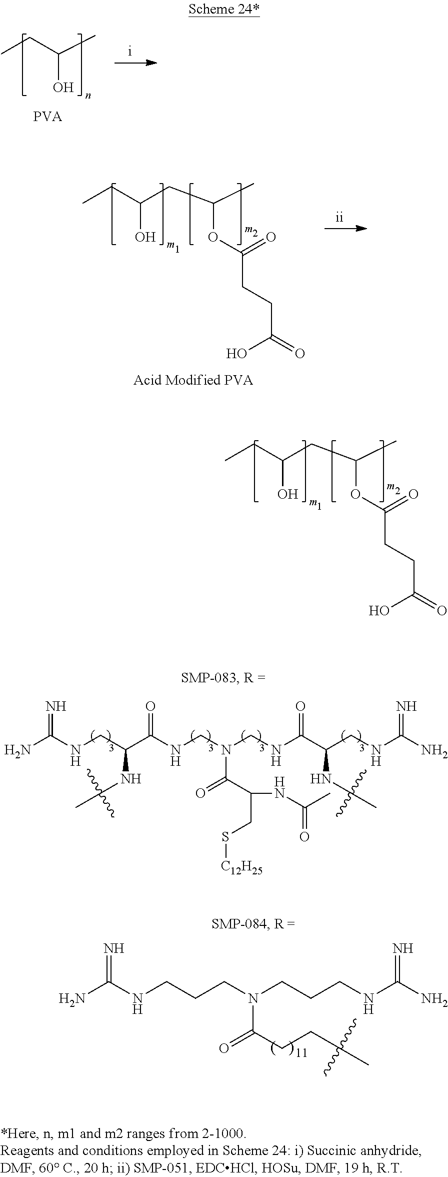

### Example 24

Synthesis of Compounds SMP-076, SMP-077 and SMP-078

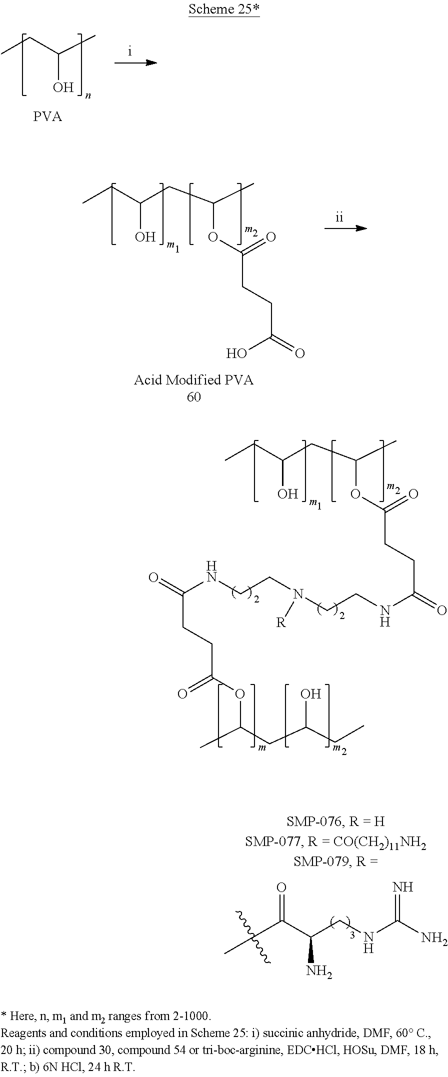

Acid modified PVA in Schemes 24 and 25 is prepared by either of the following protocols:

Synthesis of Acid Modified PVA (28%) (60): PVA (M.wt. 9-10 kDa, 80% hydrolyzed form) (10 g.) was dissolved in DMF (150 mL) at 120° C., cool to 60° C. and succinic anhydride (4.00 g, 40 mmol, 28% w.r.t. hydroxy group) was added into the solution and stirred at 60° C. for 20 h, followed by cooling to ambient temperature. Excess ethyl acetate was added into the reaction mixture that lead to precipitation which was collected by filtration. The resulting crude was dissolved in methanol and further precipitated by addition of excess diethyl ether. The precipitate was separated and extensively washed with diethyl ether and dried under reduced pressure to obtain acid modified PVA (28%) as off-white material (6.2 g).

Synthesis of Acid Modified PVA (14%) (60): PVA (10 g.) was dissolved in DMF (150 mL) at 120° C., cool to 60° C. and succinic anhydride (2.00 g, 20 mmol, 14% w.r.t. hydroxy group) was added into the solution and stirred at 60° C. for 20 h, followed by cooling to ambient temperature. Excess ethyl acetate was added into the reaction mixture that lead to precipitation which was collected by removing ethyl acetate from the mixture. The resulting crude was dissolved in methanol followed by precipitation by addition of excess diethyl ether. The precipitate was separated by filtration, extensively washed with diethyl ether and dried under reduced pressure to give acid modified PVA (14%) as off-white material (4.2 g).

### Example 25

Synthesis of Compounds SMP-079, SMP-080 and SMP-081

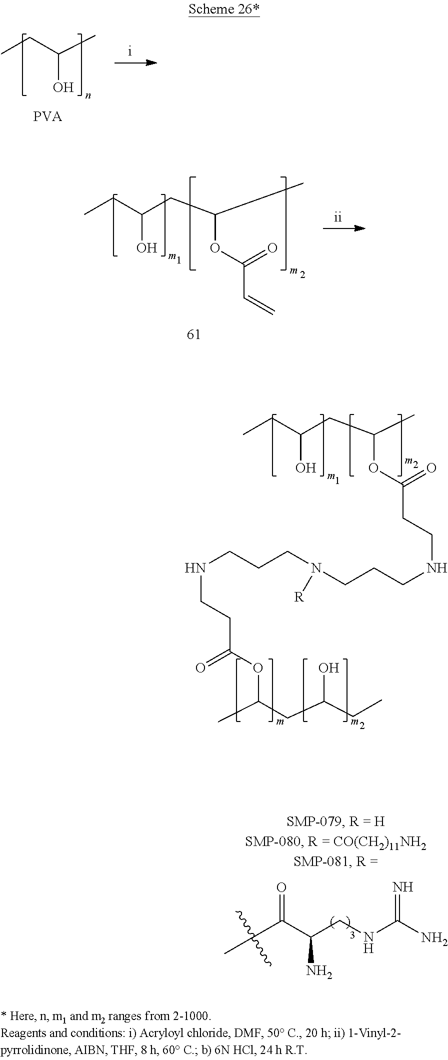

### Example 26

Topical Antimicrobial Gel Compositions

The following antimicrobial gels comprising the compounds of the present disclosure were formulated for applications including wound or surgical site infection.

TABLE 1
(A) SMP-007 (0.5 and 1%) containing topical antimicrobial gel for wound or
surgical site infection.

| | | | Composition (% w/w) | | | | |
|---|---|---|---|---|---|---|---|
| | | | General | | | | |
| S. | | | composition | | | | |
| No. | Ingredients | Role | (%) | F1 | F2 | F3 | F4 |
| 1. | SMP-007 (hydrophilic) | Active | 0.1-5 | 1 | 1 | 0.5 | 1 |
| 2. | Hydroxyethyl cellulose | Gelling agent | 0.1-3 | | 2 | | |
| | (HEC) | | | | | | |
| 3. | Propylene glycol | Emollient | 1-40 | 6 | 3 | | 3 |
| 4. | Glycerine | Humectant | 1-100 | | 1 | 3 | |
| 5. | Di-sodium edentate | Chelating | 0.1-1 | | | 0.5 | |
| | | agent | | | | | |
| 6. | Carbopol 980 | Gelling agent | 0.1-3 | 1 | | 1 | |
| 7 | Sodium alginate | Gelling agent | 0.1-5 | | | | 3 |
| 8. | Sodium benzoate | Preservative | 0.1-3 | 1 | | 1 | 1 |
| 9. | Water | Solubilizer | q.s. | q.s. | q.s. | q.s. | q.s. |
| 10. | Triethanolamine | pH modifier | q.s. | q.s. | q.s. | q.s. | q.s. |

Method of Preparation (F1):
1. SMP-007 was dissolved in water and propylene glycol was added into it.
2. In the main mixing vessel, aqueous solution of carbopol 980 was allowed to hydrate at 150-250 rpm at room temperature.
3. The prepared water-soluble drug solution was added into main mixing vessel and stirred at 150-400 rpm for 30 min to 1 hour to obtain homogenous mixture at room temperature.
4. Sodium benzoate is dissolved in water and added to the main mixing vessel.
5. Finally, 10% Triethanolamine was added to obtain final gel formulation by maintaining pH at 5.5-6.5. The resultant solution was stirred for another 30-40 min to obtain homogenous formulation.
   Similar experimental procedure was followed using other ingredients as mentioned in Table 1 for preparing F2-F4 formulations with SMP-007.

TABLE 2
(B) SMP-020 (0.5 and 1%) containing topical antimicrobial gel for wound or
surgical site infection

| | | | Composition (% w/w) | | | |
|---|---|---|---|---|---|---|
| | | | General | | | |
| S. | | | composition | | | |
| No. | Ingredients | Role | (%) | F5 | F6 | F7 |
| 1 | SMP-020 (hydrophobic) | Active | 0.1-5 | 0.5 | 0.5 | 1 |
| 2 | Allantoin | Anti-inflammatory | 0.1-2 | 0.2 | | 0.2 |
| | | or healing agent | | | | |
| 3 | Collagen | Healing agent | 0.1-2 | | 0.1 | |
| 4 | Carbopol 980 | Gelling agent | 0.1-3 | 1 | 1 | 1 |
| 5 | Glycerin | Dispersing agent | 0.5-50 | 5 | | 5 |
| 6 | Polyethylene glycol 400 | Dispersing agent | 1-20 | | 5 | |
| 7 | Sodium hyaluronate | Moisturizing agent | 0.1-2 | 0.4 | 0.4 | 0.4 |
| | (EDTA) | and emollient | | | | |
| 8 | Phenoxyethanol | Preservative | 0.1-3 | 0.7 | 0.7 | 0.7 |
| 9 | Di-sodium edentate | Chelating agent | 0.1-2 | 0.1 | 0.1 | 0.1 |
| 10 | Alpha tocopherolacetate | Antioxidant | 0.1-2 | | 0.1 | |
| 11 | Sodium hydroxide | pH modifier | q.s. | q.s. | q.s. | q.s. |
| | solution (6N) | | | | | |
| 12 | Purified water | Gel base | q.s. | q.s. | q.s. | q.s. |

Method of Preparation (F5)
1) In a main mixing vessel, EDTA and allantoin were dissolved in water. Then carbopol 980 and hyaluronate sodium were added and allowed to swell at 150-250 rpm for one hour at room temperature (RT).
2) In a separate vessel, SMP-020 was allowed to disperse homogenously in the presence of glycerin with continuous mixing at 200-300 rpm for 10 min at RT.
3) The contents of the above mixture containing SMP-020 were added into the main mixing vessel with stirring at 150-250 rpm for 2 hours at RT.
4) Phenoxyethanol solution was added to the main mixing vessel and mixed for further 20 minutes maintaining stirring speed at 150-250 rpm to obtain homogenous gel formulation.
5) Finally, 6N sodium hydroxide solution was added to obtain final gel formulation at pH 5.5-6.5. The resultant solution was stirred for another 30-40 min to obtain homogenous formulation.

Similar experimental procedure was followed using other ingredients as mentioned in Table 2 for preparing F6 and F7 formulations with SMP-020.

TABLE 3
(C) SMP-020 or SMP-037 (0.5 and 1%) containing topical liposomal
gel for wound or surgical site infection

| | | | Composition (% w/w) | | |
|---|---|---|---|---|---|
| | | | General | | |
| S. | | | composition | | |
| No. | Ingredients | Role | (%) | F8 | F9 |
| 1. | SMP-020 | Active | 0.1-5 | 1 | 1 |
| | (hydrophobic) or | | | | |
| | SMP-037 | | | | |
| | (hydrophilic) | | | | |
| 2. | Lecithin | Phospholipid | 0.1-10 | 1 | 1 |
| 3. | Cholesterol | lipid | 0.1-10 | 0.3 | 0.5 |
| 4. | Disodium edentate | Chelating agent | 0.1-0.5 | | 0.1 |
| 5. | Carbomer 974 P | Gelling agent | 0.1-3 | 1.25 | 1.25 |
| 6. | Benzyl alcohol | Preservative | 0.5-3 | 1 | 1 |
| 7. | Butylated hydroxy | Anti-oxidant | 0.1-1 | 0.1 | 0.1 |
| | toluene (BHT) | | | | |
| 8. | Triethanol amine | pH modifier | q.s. | q.s. | q.s. |
| 9. | pH 7.4 phosphate | Gel base | q.s. | q.s. | q.s. |
| | buffer | | | | |

Method of Preparation (F8):
1. Liposomes were prepared by solvent injection method. Here SMP-020, lecithin and cholesterol at particular w/w ratio were dissolved in minimum volume of ethanol and dichloromethane mixture.
2. The resultant solution was injected into pH 7.4 phosphate buffer solution at 45-65° C. with stirring at 300-500 rpm for 30 min-1.5 h. The resultant mixture was allowed to stir till the organic solvent evaporates and remaining solvent was evaporated by reduced pressure using rotary evaporator.
3. This results formation of liposomes at particular size which was characterized by TEM.
4. In the main mixing vessel, aqueous solution of carbomer 974 P was allowed to hydrate at 200-300 rpm at room temperature.
5. The drug entrapped liposomal solution was added into main mixing vessel and stirred at 150-400 rpm for 30 min to 1 hour to obtain homogenous mixture at room temperature.
6. Benzyl alcohol and butylated hydroxy toluene were added at end while stirring the solution at RT.
7. Finally, 10% Triethanolamine was added to obtain final gel formulation at pH 5.5-6.5. The resultant solution was stirred for another 30-40 min to obtain homogenous formulation.

Similar experimental procedure was followed using other ingredients as mentioned in Table 3 for preparing F9 formulation with SMP-020 or SMP-037.

### Example 27

Topical Antimicrobial Ointment Compositions

The following antimicrobial ointment comprising the compounds of the present disclosure were formulated for applications including wound or surgical site infection.

TABLE 4
(A) SMP-020 or SMP-042 (0.5 and 1%) containing topical ointment for wound or
surgical site infection

| | | | General | | | | |
|---|---|---|---|---|---|---|---|
| S. | | | composition | Composition (% w/w) | | | |
| No. | Ingredients | Role | (%) | F10 | F11 | F12 | F13 |
| 1. | SMP-042 or SMP-020 | Active | 0.1-10 | 1 | 1 | 1 | 0.5 |
| | (hydrophobic) | | | | | | |
| 2. | Polyethylene glycol 400 | Ointment base | 1-80 | 70 | | | |
| 3. | Polyethylene glycol | Ointment base | 1-50 | 25 | | | |
| | 4000 | | | | | | |
| 4. | Light mineral oil | Emollient/ | 1-50 | | 10 | 20 | 10 |
| | | Ointment base | | | | | |
| 5. | White soft paraffin | Ointment base | 1-100 | | 73 | 65 | 55 |
| 6. | Microcrystalline wax | Ointment base | 1-30 | | | | 30 |
| 7. | White wax | Ointment base | 1-10 | | | 5 | |
| 8 | Lanolin | Ointment base | 1-100 | | 10 | 5 | 5 |
| | Capric/caprylic | Emollient | 1-80 | 3 | 5 | 5 | |
| | triglyceride | | | | | | |
| 9 | Benzyl alcohol | Preservative | 0.5-3 | 1 | 1 | | |
| 10 | Triethanol amine | pH modifier | q.s | q.s. | q.s. | | |
| | (optional) | | | | | | |

Method of Preparation (F10):
1. In a glass lined melting vessel, 67 gm of polyethylene glycol 400 and 22 gm of polyethylene glycol 4000 were added and melted at 60-70° C.
2. The above mixture was transferred to homogenizer and cooled to 50-55° C.
3. In another vessel SMP-020 was dispersed in 3 gm of polyethylene glycol 400 and 3 gm of polyethylene glycol 4000 at 50-55° C. and slowly added to the homogenizer.
4. Homogenization was carried out at 50-55° C. for 30 min-1.5 h to obtain homogenous ointment formulation and allowed to cool to RT.
5. Benzyl alcohol was added at end while the solution reached at RT.
6. Finally, 10% Triethanolamine was added to obtain final ointment formulation at pH 5.5-6.5. The resultant solution was stirred for another 10-30 min to obtain homogenous formulation.

Similar experimental procedure was followed using other ingredients as mentioned in Table 4 for preparing F11-F13 formulations with SMP-020 or SMP-042.

TABLE 5
(B) SMP-047 (0.5 and 1%) containing topical ointment for wound or surgical site
infection

| | | | General | Composition (% w/w) | | |
|---|---|---|---|---|---|---|
| S. | | | composition | F14 | F15 | F16 |
| No. | Ingredients | Role | (%) | | | |
| 1. | SMP-047 (hydrophilic) | Active | 0.1-5 | 1 | 1 | 0.5 |
| 2. | Cetostearyl alcohol | Lipid | 1-10 | 0.4 | | |
| 3. | Cetomacrogol 1000 | Emulsifier | 1-10 | 0.1 | | 2 |
| 4. | Mineral oil | Emollient | 1-40 | 2.5 | | |
| 5. | White soft paraffin | Ointment base | 1-100 | 94 | 25 | |
| 6. | Stearyl alcohol | Emollient | 1-10 | | 25 | 2 |
| 7. | Sodium lauryl sulfate | Emulsifier | 1-10 | | 1 | |
| 8. | Polyethylene glycol | Ointment base | 1-50 | | | 32 |
| | 3350 | | | | | |
| 9. | Polyethylene glycol | Ointment base | 1-80 | | | 40 |
| | 400 | | | | | |
| 10. | Propylene glycol | Solubilizer/ | 1-50 | | 12 | 25 |
| | | moisturizing agent | | | | |
| 11. | Benzyl alcohol | Preservative | 0.5-3 | | 1 | |
| 12. | Purified water | Solubilizer/ointment | q.s. | 2 | q.s. | 2 |
| | | base | | | | |
| 13 | Triethanol amine | pH modifier | q.s. | q.s. | q.s. | |
| | (optional) | | | | | |

Manufacturing Procedure (F14):
1. Cetostearyl alcohol, cetomacrogol 1000 and mineral oil were melted in a vessel at 65-70° C.
2. SMP-047 was dissolved in purified water and heated at 50-60° C. The water phase was added into the lipid phase as mentioned in step 1 followed by homogenization at 50-60° C. to obtain homogenous phase A solution,
3. In another vessel white soft paraffin was taken and melted at 70° C. and the molten mass was transferred through filter to a mixer vessel and cooled to 50-60° C. (phase B)
4. Phase A described in step 2 was added into melted soft paraffin base (phase B) at 50-60° C. by maintaining stirring or homogenization for 10-15 min at 50° C.
6. The ointment was cooled slowly to room temperature followed by addition of preservative.
7. Finally, 10% Triethanolamine was added to obtain final ointment formulation at pH 5.5-6.5. The resultant solution was stirred for another 10-30 min to obtain homogenous formulation.

Similar experimental procedure was followed using other ingredients as mentioned in Table 5 for preparing F15 and F16 formulations with SMP-047.

### Example 28

Topical Antimicrobial Cream Compositions

The following antimicrobial cream comprising the compounds of the present disclosure were formulated for applications including wound or surgical site infection.

TABLE 6
(A) SMP-020 (0.5 and 1%) containing topical cream for wound or surgical site
infection

| | | | General | | | |
|---|---|---|---|---|---|---|
| S. | | | composition | Composition (% w/w) | | |
| No. | Ingredients | Role | (%) | F17 | F18 | F19 |
| 1. | SMP-020 | Active | 0.5-5 | 1 | 0.5 | 1 |
| | (hydrophobic) | | | | | |
| 2. | Capric/capryic | Emollient | 1-20 | 5 | | 5 |
| | triglyceride | | | | | |
| 3. | Isopropyl myristate | Emollient | 1-20 | | 5 | |
| 4. | Cetostearyl alcohol | Consistency agent | 1-10 | 5 | 3 | |
| 5. | Cetyl alcohol | Consistency agent | 1-10 | | | 3 |
| 6. | Stearyl alcohol | Consistency agent | 1-10 | | | 3 |
| 7. | Cetyl palmitate | Lubricant | 1-10 | | | 3 |
| 8. | Dimethicone | Skin conditioning | 0.1-5 | 1 | 1 | 1 |
| | | agent | | | | |
| 9. | Steareth -2 | Emulsifier | 1-5 | 3 | 3 | 3 |
| 10. | Steareth -21 | Emulsifier | 1-5 | 3 | 3 | 3 |
| 11. | Glycerol | Dispersing agent | 1-5 | 2 | 2 | 2 |
| 12. | Oat meal | Moisturizing | 0.5-10 | 2 | 2 | 2 |
| | | agent | | | | |
| 13. | Carbopol 980 | Gelling agent | 0.1-3 | 0.2 | 0.2 | 0.2 |
| 14. | Butylated hydroxy | Anti-oxidant | 0.1-0.5 | 0.1 | 0.1 | 0.1 |
| | toluene | | | | | |
| 15. | Benzyl alcohol | Preservative | 0.5-3 | 1 | 1 | 1 |
| 16. | Purified water | Base | q.s. | q.s. | q.s. | q.s. |
| 17. | Triethanolamine | pH modifier | q.s. | q.s. | q.s. | q.s. |

Method of Preparation (F17):
1. In the main mixing vessel carbopol 980 solution was added and allowed to hydrate at 200-250 rpm.
2. In another vessel oat meal was dispersed into glycerol and allowed to stir at 150-200 rpm for 10-15 min to obtain a homogenous dispersion. The above solution was added slowly into carbopol solution by maintaining stirring speed at 150-250 rpm. The resultant solution was heated at 55-60° C. (Phase A).
3. In another vessel, capric/caprylic triglyceride, dimethicone, cetostearyl alcohol, steareth-2 and steareth-21 were added and SMP-020 was dispersed into the lipid phase. The final mixture was heated at 60-65° C. while maintaining stirring at 150-200 rpm (phase B)
4. Phase B was added slowly into phase A by maintaining stirring speed at 250-450 rpm at 55-60° C. to obtain homogenous mixture.
5. The resultant solution was allowed to reach at 40° C., followed by addition of both preservative (benzyl alcohol) and antioxidant (Butylated hydroxy toluene).
6. Finally 10% Triethanolamine was added to obtain final pH 5.5-6.5. The resultant solution was cool to RT and allowed to stir for another 30 min to 1 h at 250-450 rpm to finally obtain homogenous cream formulation.

Similar experimental procedure was followed using other ingredients as mentioned in Table 6 for preparing F18 and F19 formulations with SMP-020.

### Example 29

Topical Antimicrobial Hydrogel or Wound Dressing Based Compositions

The following antimicrobial preparations comprising the compounds of the present disclosure were formulated for applications including wound or surgical site infection.

TABLE 7
(A) SMP-007, SMP-037 and SMP-020 (0.5 and 1%) containing topical hydrogel or
wound dressing or hydrogel film or stretchable hydrogel for wound or surgical site infection.

| | | | General | | Composition | | | | | |
|---|---|---|---|---|---|---|---|---|---|---|
| S. | | | composition | | (% w/w) | | | | | |
| No. | Ingredient | Role | (%) | F20 | F21 | F22 | F23 | F24 | F25 | F26 |
| 1 | SMP-007 (hydrophilic) | Active | 0.1-5 | 2 | | 1 | 1 | | | |
| 2 | SMP-037 (hydrophilic) | Active | | | 1 | | | 0.5 | | |
| 3 | SMP-020 | Active | | | | | | | 0.5 | 0.5 |
| | (Hydrophobic) | | | | | | | | | |
| 4 | Sodium alginate | Gelling agent | | | | | 10 | | | |
| 5 | Norspermidine | Crosslinking | 1-30 | 6 | 8.5 | | | | | |
| | | agent | | | | | | | | |
| 6 | spermidine | Crosslinking | 1-30 | | | 6 | | | | |
| | | agent | | | | | | | | |
| 7 | PVA (9-10 KDa) | Gelling agent | 1-60 | 17.5 | | 17.5 | | | | |
| | (17.5%) | | | | | | | | | |
| 8 | PVA-acid (28%) | Gelling agent | 1-60 | | 17.5 | | | | | |
| 9 | PLA (Poly-L-lactic | Gelling agent | 1-50 | | | | | | 20 | |
| | acid)-PEG-PLA co- | | | | | | | | | |
| | polymer | | | | | | | | | |
| 10 | Hyaluronic acid (HA) | Gelling agent | | | | | 0.2 | | 1 | 1 |
| 11 | Starch-pectin | Gelling agent | 1-30 | | | | | 3 | | |
| 12 | Dextran, β- | Gelling agent | 1-30 | | | | | | | 10 |
| | cyclodextrin, Sodium | | | | | | | | | |
| | trimetaphosphate | | | | | | | | | |
| | (STMP) | | | | | | | | | |
| 13 | Collagen | Healing | 10-30 | | | | | | 0.2 | 0.2 |
| | | agent | | | | | | | | |
| 14 | Metal ion | Crosslinking | | | | | q.s. | q.s. | | |
| | | agent | | | | | | | | |
| 15 | Organic solvent | Solubilizer | | | q.s. | | | | q.s. | q.s. |
| | (DMSO or MeOH or | | | | | | | | | |
| | EtOH) | | | | | | | | | |
| 16 | Water | Solubilizer | 0.1-2 | q.s. | q.s. | q.s. | q.s. | q.s. | q.s. | q.s. |

Method of Preparation (F20):
1. Water soluble SMP molecule (SMP-007) was dissolved in water to obtain (17.5% w/v). Sparingly water soluble SMP molecule (SMP-037) were dissolved in DMSO to obtain stock concentration (17.5% w/v).
2. A solution of 17.5% w/v PVA/PVA-acid (28%) were prepared by desolving 1.75 g PVA/PVA-acid (28%) in 10 ml of water by heating the solution at 90° C.
3. Different amount of SMP molecules from a stock solution (17.5% w/v) was added sequentially to a fixed ratio of spermidine/norspermidine to PVA/PVA-acid solution that are known to form hydrogel. The resultant mixture was sonicated for five minutes and were passed through 3-6 times consecutive freeze/thaw cycle to obtain SMP loaded hydrogel after 3-4 days.

Different external stimuli were used to form hydrogel with various polymer matrices that facilitate loading of either hydrophobic or hydrophilic SMP molecules to obtain final hydrogel formulation as described in F21-F26 in Table 7.

Similar to schemes 22-24 of the present disclosure, different reactive monomeric scaffolds like vinyl substituted or methacrylate or acrylate substituted spermidine, norspermidine, spermine, diethanol amine and many other derivatized molecules or functionalized SMP monomeric molecules react with acrylate or other reactive functionalized polymeric backbones like dextran, albumin, (hydroxyethyl)starch, polyaspartamide, poly(2-hydroxyethyl methacrylic acid) pHEMA, PVA, polyacrylic acid, PEG, PEG-PLA, poly (D, L-lactic acid), polyglycolic acid, PLGA, hyaluronic acid and others to form cross-link gel/hydrogel via radical polymerization in the presence of suitable radical generators. Radicals were generated in-situ in reaction medium by using AIBN (2,2′-Azobisisobutyronitrile) as a radical initiator or after exposure of UV light alone or in presence of suitable photoinitiator like (2,2-dimethoxy-2-phenyl acetophenone) or photoinitiator composed of peroxysulfate and N,N,N′,N′-Tetramethylene diamide (TEMED). Effective SMP molecules SMP-047, SMP-020, SMP-37, SMP-007, SMP-010, SMP-042, SMP-034, SMP-036 and others were loaded into different polymeric matrices or polymeric derivatives like PVA (polyvinyl alcohol), PVA acid, poly-L-lactic acid (PLA), PEG-PLA co-polymer, hyaluronic acid, starch-pectin, sodium alginate, dextran, dextran in combination with β-cyclodextrin, chitosan, alone or in combination thereof to form bio-degradable or bio-compatible gel or hydrogel at room temperature and pH 7. These antimicrobial polymeric systems were formulated into different topical dosage forms for the treatment of various kind of wounds, implants and other surgical site related infections caused by both Gram positive and Gram negative pathogens.

The SMP monomeric and polymeric molecules of the present disclosure were finally formulated into different topical dosage forms to deliver the active(s) at the site of action at effective concentration. Depending on the presence of hydrolysable and non-hydrolysable groups, the polymers were recognized as bio-degradable or bio-compatible or partially biodegradable form. For example, polyester, polyamide, polycarbonate, polylactide, polyglycolide, chitosan, polyhydroxobutyrate, chitosan, hyaluronic acid based linkages are hydrolysable and recognized as biodegradable in nature. On the other hand, polyurethane, polyurea, polymethacrylate based scaffolds are non-biodegradable, but some of them were found to be bio-compatible. Such bio-compatible antimicrobial polymers are used in different orthopedic applications.

### Example 30

Antimicrobial Coating Compositions

The following antimicrobial preparations comprising the compounds of the present disclosure were formulated for applications including implant coatings.

TABLE 8
(A) Coating composition of SMP-042 (0.1-0.1%) on
latex catheter surface

| | | | General | | | |
|---|---|---|---|---|---|---|
| Sr | | | composition | Composition (%) | | |
| No | Ingredient | Role | (%) | F27 | F28 | F29 |
| 1 | SMP-042 | Active | 0.1-3 | 0.5 | | 0.5 |
| | (Hydrophobic) | | | | | |
| 2 | SMP-007 | Active | 0.1-3 | | 0.5 | |
| | (hydrophilic) | | | | | |
| 3 | PVA (M. Wt. 9-10 | Solubilizer, | 1-30 | | 20 | |
| | KDa or 13-23 | coating | | | | |
| | KDa) | material | | | | |
| 4 | Spermidine | Solubilizer, | 1-20 | | 6 | |
| 5 | Propylene glycol | Solubilizer | 1-50 | 2 | | 2 |
| 6 | PEG-12- | Emulsifier or | 1-10 | 3 | | 2 |
| | Dimethicone | Solubilizer | | | | |
| 7 | Dimethicone | Solubilizer | 1-10 | 1 | | |
| | copolyols | | | | | |
| 8 | Transcutol | Emulsifier | 1-20 | | | 2 |
| 9 | Silicone based | Coating agent | 1-100 | q.s. | | q.s. |
| | medical fluid | | | | | |

Method of Preparation (F27):
1. SMP-042 was dispersed in dimethicone, PEG-12-dimethicone and propylene glycol.
2. Above solution (mention in step 1) was mixed with silicone based medical fluid to form transparent to opaque solution.
3. The latex catheter was dipped into final mixture (mention in step 2) for 10 min-30 min at room temperature.
4. SMP-042 containing silicone coated latex catheters were cured at 25° C. and 60% RH for 24 h and used for further characterization, drug release studies and bio-activity studies.

Similar experimental procedure was followed using other ingredients or solubilizers as mentioned in Table 8 for preparing F29 formulations with SMP-020. In case of hydrophilic polymer SMP-007, PVA-spermidine mixture was used to solubilize SMP-007 followed by dipping the latex catheter into the final mixture for 10 min-30 min at room temperature. Finally, SMP-007 containing silicone coated latex catheters were cured at 25° C. and 60% RH for 24 h and used for further characterization, drug release studies and bio-activity studies.

TABLE 9
(B) Chemical cross-linking of SMPs (0.1-0.1%) on latex catheter surface

| | | | General | Composition | | |
|---|---|---|---|---|---|---|
| Sr | | | composition | (% w/w) | | |
| No | Ingredient | Role | (%) | F30 | F31 | F32 |
| | PVA-acrylate (9-10 | Coating | 3-60 | 10 | 30 | 20 |
| | KDa) (Compound 61, | agent | | | | |
| | scheme 26) | | | | | |
| | Acrylate derivative of | Active | 0.1-30 | 10 | 5 | 20 |
| | SMPs (Compound 62 | | | | | |
| | or derivative of 58 | | | | | |
| | from scheme 23 and | | | | | |
| | 22) | | | | | |
| | Silicone based medical | Coating | 1-100 | q.s. | q.s. | |
| | fluid (Optional) | agent | | | | |

Method of Preparation (F30):
1. The latex catheter was dipped into 20% aqueous solution of PVA-acrylate for 10 min-30 min at room temperature
2. Next PVA-acrylate coated catheter was immersed into aqueous solution of acrylate derivatized molecules, (compound 62) followed by UV irradiation for 5-10 min.
3. Finally PVA coated catheters were cured at 25° C. and 60% RH for 24 h and used for further characterization, drug release studies and bio-activity studies.

As an alternative, of before doing step 3, the PVA coated catheter was finally dipped into medical fluid for 10 min-30 min at room temperature to obtain slippery surface. Finally, silicone-PVA coated catheters were cured at 25° C. and 60% RH for 24 h and used for further characterization, drug release studies and bio-activity studies.

### Example 31

Determination of Minimum Inhibitory Concentration (MIC)

The MIC of compounds of the present disclosure were determined as follows

Materials: Brain heart infusion broth, Bacterial and fungal cultures, 96 wells plate, Autoclave, Incubator. The microbial strains used in the validation study/testing were procured from MTCC (Chandigarh, India)—S. aureus (MTCC-6908), E. coli (MTCC-1687); NCIM (Pune, India)—E. aerogenes (NCIM-5139); ATCC (USA): S. aureus (ATCC-43300), S. epidermidis (ATCC-35984), P. aeruginosa (ATCC-27853), A. baumanii (ATCC-19606), K. pneumoniae (ATCC-13883), E. coli (ATCC-BAA196); CCARM (Korea)—Candida albicans (CCARM-14009), P. aeruginosa (CCARM-2161) and A. baumanii (CCARM-12001).

Method: MIC of present molecules were determined by micro broth dilution method as per the Clinical and Laboratory Standards Institute (CLSI) guidelines. Microbial strains were cultured in appropriate media [Brain Heart Infusion Agar (BHIA) for bacteria and Sabouraud dextrose broth (SDB) for Candida] at 37° C. for 24 hours. For MIC test, sterile BHI/SD broth (100 μl) was added into all 96 wells and 100 μl of broth containing drug was added to first well (1A to 1H) and serial (double) dilution was carried out for up to 10 wells (column 1 to column 10 of 96 well plate). For inoculum, microbial culture turbidity was adjusted to 0.1 optical density (OD) at 600 nm in UV—Visual spectrophotometer (approximately 1.5×10⁸ cells/ml) and further diluted (100 times with sterile media). Diluted culture suspension (100 μl) was added to each well except sterility control wells (column 12 of 96 well plate). Column 11 of 96 well plate was used as growth control and vehicle control. The plates were incubated at 37° C. for 24 h. The MIC of the test compounds were determined by observing the lowest concentration of test compound that prevented the visual bacterial growth. The MIC test results and conclusions/inference are provided below in Tables 10-13.

TABLE 10
MIC values of the compounds described in Formula Ia

| | MIC (μg/ml) | | | | |
|---|---|---|---|---|---|
| | S. aureus | | S. epidermidis | | C. albicans |
| | MTCC | ATCC | ATCC | | CCARM |
| Compounds | 6908 | 43300 | 35984 | S12-1 | 14009 |
| SMP-002 | 25 | 100-50 | 8-13 | 25 | 100 |
| SMP-047 | 6.25 | 6.25 | 6.25 | 50 | 50 |
| SMP-015 | 50 | 200 | 100 | 100 | 100 |
| SMP-023 | 25 | 100 | 50-100 | 50 | 25 |
| SMP-051 | 6.25 | 6.25 | 1.6 | ND | 200 |
| SMP-063 | 12.5 | 100 | 50 | 25 | ND |

Results: SMP-047 with unsaturated C-18 alkyl chain was found to have potent antibacterial activity against MRSA as well as potent towards resistant E. coli (Table 10). Modification of SMP-002 and SMP-047 by arginine residues at terminal amino group resulted SMP-051 and SMP-065 respectively. Antibacterial activity of SMP-051 was found to be improved by 10-20-fold against Gram positive pathogen and 100-150-fold against Gram negative pathogen in comparison to SMP-002. Both SMP-051 and SMP-047 are found to be small antiseptic molecules with broad spectrum antibacterial activity (Table 10).

Inference: All the SMP molecules tested showed good antimicrobial activity. The above results also suggest that the presence of specific hydrophobic groups like C-12 or unsaturated C-18 long chain like lauryl, oleyl, linoleic group and net positive charge together influence the antimicrobial activity of a given molecule as seen by antimicrobial activity of different SMP molecules. In conclusion, overall spatial orientation and geometry of different functional moieties like the presence of S-alkylated-NAC pharmacophoric moiety plays an important role towards antibacterial activity of a given molecule.

TABLE 11
MIC values of the compounds described in Formula Ib

| | MIC (μg/ml) | | | | |
|---|---|---|---|---|---|
| | S. aureus | | S. epidermidis | | C. albicans |
| | MTCC | ATCC | ATCC | | CCARM |
| Compounds | 6908 | 43300 | 35984 | S12-1 | 14009 |
| SMP-010 | 3.2 | 6.25 | TBD | TBD | >200 |
| SMP-007 | 3.2 | 6.25 | 6.25-8 | 12.5 | >200 |
| SMP-043 | 6.25 | 12.5 | 6.3 | 3.1 | 100 |
| SMP-045 | 6.25 | 25 | 12.5 | 25 | 50 |
| SMP-052 | 50 | 50 | 50 | ND | 200 |
| SMP-060 | 3.2 | 12.5 | 6.25 | 6.25 | 200 |
| SMP-026 | 3.2 | 100 | 50 | 25 | 100 |

Results: SMP-002 monomer was further investigated as a potent building block for obtaining numerous homo and hetero polymers. For example, SMP-002 was utilized, either to carry out self-polymerization to obtain SMP-010 or polymerization with hexamethylene biguanidinedicyano moiety to obtain heteropolymer SMP-007 (Table 11). Both SMP-007 and SMP-010 showed good antibacterial activity. To ascertain significant activity contributing from long C-12 alkyl chain containing NAC pharmacophore in case of SMP-007, other polybiguanidium linked norspermidine based heteropolymers like SMP-043, SMP-045 and SMP-060 with C12, C8 and mono-unsaturated C18 alkyl functionalized polymers were designed and synthesized respectively. All these polybiguanidine based polymers like SMP-043, SMP-045 and SMP-060 were found to show similar activity against MRSA. Both SMP-043 and SMP-045 possess similar antifungal activity against Candida species. Although being similar in many aspects, SMP-043 is potent antibacterial agent than SMP-045 because of possessing 8-fold enhanced activity against resistant S. epidermidis strain and 4-fold enhanced activity against E. coli strain (Table 11).

Inference: All the SMP molecules tested showed good antimicrobial activity. In particular, SMP-043 and SMP-007 were shown as the most effective polybiguanidine based antimicrobial polymers and different topical formulations were made with different drug delivery carriers.

TABLE 12
MIC values of the compounds described in Formula I, Ib, Ic

| | MIC (μg/ml) | | | | |
|---|---|---|---|---|---|
| | S. aureus | | S. epidermidis | | C. albicans |
| | MTCC | ATCC | ATCC | | CCARM |
| Compounds | 6908 | 43300 | 35984 | S12-1 | 14009 |
| SMP-021 | 50 | 100 | 50 | 50 | 200 |
| SMP-062 | 6.25 | 50 | 25 | 25 | 100 |
| SMP-020 | 3.2 | 3.2 | 3.1 | 3.1 | 6.25 |
| SMP-034 | 12.5 | 12.5 | 13 | 13 | >200 |
| SMP-036 | 3.2 | 3.2 | 1.6 | 3.1 | 6.25 |
| SMP-024 | 50 | 50 | 25 | 25 | 100 |
| SMP-042 | 3.2 | 6.25 | 3.1 | 3.1 | 25 |
| SMP-064 | 50 | >200 | 100 | 100 | 200 |
| SMP-067 | 12.5 | 25 | 12.5 | 12.5 | 25 |
| SMP-037 | 1.6 | 6.25 | 6.3 | 6.3 | 200 |
| SMP-049 | 25 | 50 | 25 | ND | 200 |
| SMP-030 | 50 | 100 | 25 | 25 | >200 |

Results: Another set of polyurethane based polymers were designed and synthesized to explore antimicrobial activity. Diethanolamine or norspermidine derivatives were chosen as monomeric units and subsequently polymerized with hexamethylene diisocyanate to obtain various polyurethane derivatives (Table 12). SMP-018 was reacted with excess methyl iodide to obtain C-12 alkyl chain and methyl containing quarternized SMP-020 molecule. Quaternization provides an exceptional antibacterial and antifungal activity specially against resistant pathogens. SMP-033 and SMP-035 molecules were quaternized by reacting with methyl iodide to obtain SMP-034 and SMP-036 respectively. Both SMP-036 with C-16 alkyl and methyl quarternized polymeric scaffold and SMP-034 with C-8 alkyl and methyl quarternized molecules were found to be specific against Gram positive pathogens. SMP-042 was a tri-block co-polymer with methyl and C-12 alkyl quaternized unit like SMP-020 and found to have similar antimicrobial and anti-biofilm activity like SMP-020. Other than quarternization, long chain (C12) alkyl amine functionalized polyurethane derivatives were made like SMP-061 and SMP-062. Interestingly, SMP-062 was found to have similar antimicrobial properties like SMP-020 against Gram positive pathogen. SMP-067 was found to show potent activity against resistant gram positive pathogens. Another set of polybiguanidine and polyurethane based mixed polymers were designed and synthesized by choosing norspermidine derivatives, hexamethylene diisocyanate, ethanol amine and hexamethylene or dimethylene cyanopolyguanidium derivatives as monomeric units (Table 12). Subsequent polymerization between different monomeric scaffolds yield various mixed polymers like SMP-037, SMP-049, SMP-059 and many others. Among all the polymers SMP-037 was found to have potent antibacterial activity against wide spectrum of bacterial and fungal species and specially against MRSA and resistant S. epidermidis. Another set of polyester and polyamide based polymeric scaffolds were designed and synthesized by reacting diethanol amine and norsperimidine derivatives with succinic anhydride at different molar ratios (Table 12, SMP-30 and SMP-049). C-12 alkyl functionalized polyester analogue showed good antibacterial activity.

Inference: All the SMP molecules tested showed good antimicrobial activity. The above results indicate that the importance of charge and hydrophobicity as well as right balance of both characteristics in a polymeric scaffold is necessary to provide active antimicrobial polymer(s). In that case, methyl and C-12 alkyl quarternized polyurethane scaffold resulted in most potent antibacterial, antifungal and anti-biofilm activity among all the SMP molecules synthesized so far with all different linkages. Further, monounsaturated C-18 alkyl chain had selective interaction with Gram positive bacterial membrane. Among all, polyurethane, polyurea and mixed polyurethane-polybiguanidine based scaffolds SMP-020, SMP-042, SMP-037 and SMP-062 were selected as the active APIs for topical formulation by using different drug delivery carriers. The above results further support the significance of charge and hydrophobicity balance in polymeric scaffolds for obtaining optimum antimicrobial activity.

TABLE 13
MIC values of compounds in Gram-negative pathogens

| | MIC (μg/ml) | | | | | | | | |
|---|---|---|---|---|---|---|---|---|---|
| | P. | P. | A. | A. | E. | | K. | | |
| | aeruginosa | aeruginosa | baumannii | baumannii | aerogenes | E. | pneumoniae | E. coli | E. coli |
| | [ATCC | [CCARM | [ATCC | [CCARM | [NCIM | aerogenes | [ATCC | [MTCC | ATCC |
| Compounds | 27853] | 2161] | 19606] | 12001] | 5139] | [M2-1] | 1388] | 1687] | BAA196 |
| SMP-037 | 4.0 | 256.0 | 128.0 | 32.0 | 8.0 | 64.0 | 4.0 | 50 | >200 |
| SMP-020 | 4.0 | 32.0 | 16.0 | 64.0 | 8.0 | 64.0 | 2.0 | 25 | 25 |
| SMP-007 | 64.0 | 64.0 | 16.0 | 32.0 | 16.0 | 32.0 | 4.0 | 25 | >200 |
| SMP-047 | >256.0 | ND | >256.0 | ND | >256.0 | ND | >256.0 | 0.8 | 100 |
| SMP-051 | 16.0 | ND | 128.0 | ND | 8.0 | ND | 8.0 | 1.6 | 100 |
| SMP-043 | 128.0 | ND | 128.0 | ND | 128.0 | ND | 128.0 | 25 | >200 |
| SMP-060 | 128.0 | ND | 128.0 | ND | 128.0 | ND | 128.0 | 12.5 | >200 |
| SMP-062 | >256.0 | ND | 256.0 | ND | >256.0 | ND | 256.0 | 12.5 | >200 |
| SMP-042 | 64.0 | ND | 32.0 | ND | 64.0 | ND | 8.0 | 12.5 | >200 |

Results: The activity of some of the compounds of the present disclosure were studied against Gram negative pathogens such as P. aeruginosa, A. baumannii, E. aerogenes, K. pneumoniae and others (Table 14). SMP-020, a polyurethane based polymer was found to be the most effective molecule against both susceptible and resistant gram negative pathogens and behaves like commercial antiseptic agent PHMB. Polymers like SMP-037, SMP-007 and monomeric analogue, SMP-051 were also found to be very efficient against Gram negative pathogens and can be recognized as broad spectrum antiseptics.

Inference: SMP-007 was found to have good antimicrobial activity around 4-20 μg/ml against other Gram-negative pathogens like P. aeruginosa, A. baumannii, E. aerogenes, and K. pneumoniae. SMP-020 was found to have potent antibacterial activity even against other Gram-negative pathogens like P. aeruginosa, A. baumannii, E. aerogenes, K. pneumoniae and others.

The above results suggest that monounsaturated C-18 alkyl chain have selective interaction with Gram positive bacterial membrane whereas strong cationic charge and proper balance of charge and hydrophobicity results in strong interaction with Gram-negative bacterial membrane. The above structure function activity results suggest that hydrophobicity, net charge and orientation of active functional groups around the backbone of the monomeric or polymeric molecules play significant role towards determining their interaction with different bacterial membranes. The above results also show that some molecules are specific towards interacting with Gram positive bacterial membrane and very few are found to be potent against Gram negative pathogens. This fact can be easily justified due to the presence of different membrane morphology for Gram positive and negative pathogens. The above bio-activity results for all different SMP molecules conclude that the presence of either hard charge or specific pharmacophoric moieties or combinations thereof along with proper 3D-geometries/orientation of active functional groups are essential for disrupting Gram negative membrane whereas either specific hydrophobic unit like C12 or C18 (mono unsaturated)alkyl chain or soft charge or combinations thereof along with specific orientation of the molecule are necessary factors for controlling interaction with Gram positive membranes.

### Example 31

### S. Epidermidis Biofilm Disruption Assay

The compounds of the present disclosure were tested for their biofilm disruption activity against S. epidermidis as follows

Materials: Brain heart infusion broth, S. epidermidis ATCC 35984, 96 well plates, Autoclave, Incubator, 0.05% MTT (3-(4,5-Dimethylthiazol-2-yl)-2,5-Diphenyltetrazolium Bromide) reagent prepared in water, Multiwell Plate Reader.

Method: S. epidermidis ATCC 35984 was grown in Brain Heart Infusion Agar (BHIA) at 37° C. for 24 h. The loop full of bacterial culture was suspended in sterile water and adjusted the turbidity to 0.1 OD at 600 nm in UV—Visual spectrophotometer (approximately 1.5×10⁸) and further diluted (100 times with sterile BHI broth), 100 μL of culture suspension was added into 96 well plate and plates were incubated at 37° C. for 48 h for biofilm formation. The biofilm was washed twice with sterile water to remove the planktonic cells. Then biofilm was treated with 100 μL of BHI broth suspended with various concentrations of compounds of the present disclosure, and plates were further incubated at 37° C. for 24 h. The minimum biofilm disruption concentration (MBDC) was determined by staining the biofilm with MTT reagent incubated plates at 37° C. for 2 h. The precipitate was dissolved in 100 μl of dimethyl sulfoxide (DMSO) and the absorbance was measured at 600 nm in multi well plate reader. The minimum biofilm disruption concentration was calculated as subtracting the MTT OD value of drug treated with 48 h growth control. The activity results are provided in Table 14.

TABLE 14
Biofilm disruption ability of the compounds in
S. epidermidis ATCC 35984

| | Concentration (mg/ml) to |
|---|---|
| Compounds | disrupt 50% biofilm |
| NAC | >4 |
| SMP-001 | 0.02-0.125 |
| SMP-003 | >2 |
| SMP-009 | 2 |
| SMP-002 | 0.01 |
| SMP-047 | >2 |
| SMP-015 | 1 |
| SMP-022 | 2 |
| SMP-023 | 2 |
| SMP-051 | >2 |
| SMP-010 | 2 |
| SMP-007 | 0.5 |
| SMP-043 | 0.25 |
| SMP-045 | 0.5 |
| SMP-060 | >2 |
| SMP-026 | >2 |
| SMP-027 | 0.5 |
| SMP-021 | >2 |
| SMP-062 | >2 |
| SMP-020 | 0.125 |
| SMP-034 | 0.5 |
| SMP-036 | 0.125 |
| SMP-024 | >2 |
| SMP-040 | >2 |
| SMP-041 | >2 |
| SMP-042 | 0.125 |
| SMP-037 | 2 |
| SMP-049 | 2 |
| SMP-030 | 0.25 |

Results: NAC (N-acetyl cysteine) group was functionalized with C12 long chain results (SMP-001) and was found to destroy biofilm effectively developed by S. epiermidis (ATCC 35984). Interestingly, SMP-002 (Formula Ia) was found to maintain anti-biofilm activity like SMP-001. SMP-020, SMP-042, SMP-034, and SMP-036 (Formula Ib) was found to destroy bio-film formed by S. epidermidis at low drug (0.125 mg/ml) concentration despite of their differences in hydrophobicity. Some of the specific molecules like SMP-007, SMP-043, SMP-045 belonging to polybiguanidine functional group were also found to possess potent activity towards S. epidermidis biofilm. SMP-037 belonging to mixed polybiguanidine and polyurethane scaffold was also found to have good anti-biofilm activity.

Inference: The structure-activity relationship concluded that quaternized charge and long-C12 alkyl chain appended to NAC has significant influence towards destroying biofilm formed by S. epidermidis.

### Example 32

E. Coli Biofilm Disruption Assay

The compounds of the present disclosure were tested for their biofilm disruption activity against E. coli as follows

Materials: Brain heart infusion broth+1% glucose, Escherichia coli ATCC BAA196, 96 wells plate, Autoclave, Incubator, MTT, Multiwell Plate Reader.

Method: E. coli ATCC BAA196 was grown in Brain Heart Infusion Agar (BHIA) at 37° C. for 24 h. The loop full of bacterial culture was suspended in sterile water and the turbidity to 0.1 OD at 600 nm in UV—Visual spectrophotometer (approximately 1.5×10⁸) and further diluted (100 times with sterile BHI broth+1% glucose), 100 μL of culture suspension was added into 96 well plate and plates were incubated at 37° C. for 24 h for biofilm formation. The biofilm was washed twice with sterile water to remove the planktonic cells. Then biofilm was treated with 100 μL of BHI broth+1% glucose suspended with various concentrations of compounds of the present disclosure, and plates were further incubated at 37° C. for 24 h. The minimum biofilm disruption concentration (MBDC) was determined by staining the biofilm with MTT reagent incubated plates at 37° C. for 2 h, and the precipitate was dissolved in 100 μl of dimethyl sulfoxide (DMSO) and the absorbance was measured at 600 nm in multi well plate reader. The minimum biofilm disruption concentration was calculated by subtracting the MTT OD value of drug treated with 24 h growth control. The activity results are provided in Table 15.

TABLE 15
Biofilm disruption ability of the compounds in E. coli ATCC BAA196

| | Minimum biofilm disruption |
|---|---|
| | concentration |
| Compounds | (mg/ml) |
| SMP-001 | 0.5-2.0 |
| SMP-002 | 2-4 |
| SMP-020 | 0.5-2.0 |
| SMP-037 | >2 |
| SMP-043 | 2 |
| SMP-051 | 0.25-0.5 |
| PHMB | 2-4 |

Results: All the compounds tested demonstrated disruption of E. coli biofilm among which SMP-051 was the best followed by SMP-001 and SMP-020.

Inference: The results suggest that arginine based and quarternized based polymers have better ability to disrupt Gram-negative biofilms.

### Example 33

Cytotoxicity Assay

Cytotoxicity of the compounds of the present disclosure was tested in HaCat cells.

Materials: RPMI 1640 medium, 96 well plate, Fetal bovine serum, CO₂ Incubator, inverted Microscope, Water bath, HaCat cell line.

Method: MTT assay was performed as described by Alley et al, 1988 with some modifications. Different dilutions of compounds of the present disclosure in DMSO were used for the MTT assay so that the final concentration of DMSO in the assay was <5%. Cell suspensions prepared in RPMI 1640 medium contains 5% fetal bovine serum (of volume 200 μl were seeded in a 96-well plate (20,000 cells per well), without the test agent and was allowed to grow for about 12 hours. The appropriate volumes of working stock solutions of test compounds were then added to the wells to achieve final concentrations of 25, 50, 100, 200 and 400 μg/ml in triplicates. The reference inhibitor camptothecin was added to achieve a final concentration of 50 μM. The cells were incubated for 48 h at 37° C. in a 5% CO₂ atmosphere. After the incubation period, the plates were removed from the incubator, spent media was removed followed by addition of MTT reagent to a final concentration of 0.5 mg/ml. After 3 h incubation, MTT was removed and 100 μl of DMSO was added. Absorbance was measured in an ELISA reader at 570 nm. The assay controls were (a) medium control (medium without cells), (b) negative control (medium with cells but without the experimental compound), (iii) positive control (medium with cells and with 50 μM camptothecin). The experiment was done in triplicates and the data plotted as viability of cells in percentage. The results are provided in Table 16.

TABLE 16
In vitro cytotoxicity of SMPs against different cell lines

| | | IC₅₀ in HaCat cell line |
|---|---|---|
| Sl. No. | Compounds | (μg/ml) |
| 1 | SMP-007 | ~138.0 |
| 2 | SMP-051 | ~25.0 |
| 3 | SMP-037 | ~100-200 |
| 4 | SMP-036 | 55.0 |
| 5 | SMP-043 | 36.0 |
| 6 | SMP-047 | 200-400 |
| 7 | SMP-020 | ~400.0 |
| 8 | SMP-060 | >400.0 |
| 9 | SMP-062 | >400.0 |
| 10 | SMP-042 | >400.0 |
| 11 | SMP-071 | >400.0 |
| 12 | Chlorohexidine | ~200 |
| 13 | PHMB | ~100-200 |

Results: The toxicity assay of some of the effective broad spectrum compounds of the present disclosure were done against HaCaT cell lines (Table 16). Among monomeric analogues (Formula Ia), SMP-047 was found to have better safety profile in comparison to commercial PHMB and chlorohexidine molecules. Among polymeric molecules (Formula I, Formula Ib, Formula Ic) most of the polyurethane based molecules like SMP-020, SMP-042 and SMP-062 were found to have good safety profile and comparable or better than PHMB. Further, some of the polybiguanidine and mixed polyurethane-polybiguanidine scaffolds like SMP-007, SMP-060 and SMP-037 were found to have good safety profile with respect to reference standard PHMB.

Inference: The above data suggests that the SMP compounds are well-tolerated by human cell lines at much higher concentrations than their MICS and hence will have a good therapeutic window for use as anti-microbial agents.

### Example 34

Scratch Assay to Test Wound Healing Properties

Scratch assay was carried out to test wound healing properties of the compounds of present disclosure

Materials: NIH-3T3 cell line, Dulbecco's modified eagle medium (DMEM), 6 well plates, fetal bovine serum, CO₂ incubator, inverted microscope, sterile micro-tips.

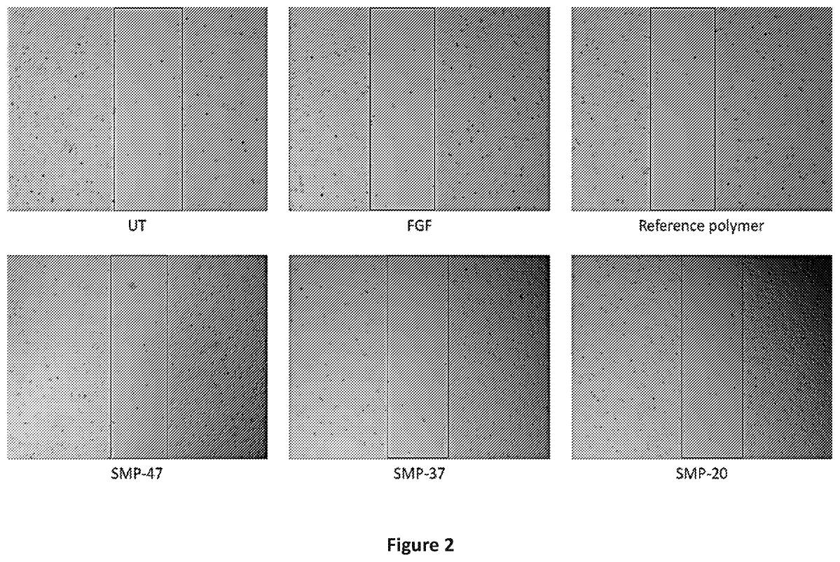

Method: NIH-3T3 cells were seeded in 6-well tissue culture plates in Dulbecco's modified eagle medium (DMEM) supplemented with 10% FBS. The cells were incubated at 37° C. (5% CO₂) for 24 h to reach 70-80% confluency in a monolayer. At this stage, a sterile micropipette tip was used to introduce a scratch in the culture monolayer along the center of the well (end to end in a straight line) by holding the long axis of the tip perpendicular to the bottom of the well. The resulting width of the scratch was thus equivalent to the outer diameter of the pointed end of the tip. After introduction of the scratch, the cells were gently washed with medium two times to remove detached cells and then wells were replenished with fresh medium containing test agents at desired concentrations. The cells were then incubated at 37° C. (5% CO₂) for 6 h prior to image acquisition using uniform microscope settings (objective magnification 10×) for all the samples. Photomicrographs of the cultures (all wells) were taken at 6 h post treatment to qualitatively assess the state of the scratch (appearance of cells within the scratch, integrity of the boundary of the scratch and general health of the cells on either side of the scratch) in each well. The filling up of the gap created due to the scratch in each well was compared across samples and interpretations about any effect of test agents on healing were drawn based on comparisons with the untreated controls. The results are provided in FIG. 2.

Results: All the SMP compounds tested herein were well-tolerated by the NTH-3 T3 cells and did not show any negative effect on the process of scratch healing as compared to the untreated (UT) and fibroblast growth factor supplemented (FGF) samples. As observed in the demarcated scratch area for each sample (vertical rectangular box), some cells migrated into the scratch area for every sample within 6 hours post introduction of the scratch. There were no visible differences between the samples.

Inference: Based on the in vitro scratch test assay, it is observed that none of the compounds demonstrate properties that may impede wound healing.

Further, some of the molecules such as monomeric analogues, SMP-047, polybiguanidine analogue SMP-007, polyurethane analogue SMP-020 and mixed polyurethane and polybiguanidine analogue SMP-037 were selected for preparing topical compositions based on the in vitro MIC values, toxicity values against HaCaT cell lines and bio-film inhibitory effect against both gram positive and gram negative pathogens as described above. The compositions were formulated into different topical dosage forms such as hydrogel, hydrogel film, wound dressing, gel, ointment, spray, solution or in the form of coat on catheter or implant surfaces to deliver the active at the site of infection in a sustained manner.

The above examples and results show the efficiency of the presently developed compounds in antimicrobial applications. Significance of the compounds of the present disclosure developed using SNAP technology and some of the advantages are as follows

All the antiseptics widely prescribed in present scenario including silver were developed prior to 1960s. There are not many recent clinical studies/reports that discusses on the development of new, broad spectrum antiseptics and their use in developing improved medical devices. The technology of present disclosure involves cost effective, synthetically feasible (short steps) process to obtain the final compounds with improved yield. The compounds provide broad spectrum antimicrobial effect against pathogenic bacteria, fungi, including antibiotic resistant strains. The multiple positive charges and repetition of biofilm inhibitory segment in polymeric scaffold improves both antimicrobial and biofilm inhibitory action thus improving the bio-efficacy in comparison to other known antiseptics/compounds especially against resistant strains. In addition, this technology has low propensity in developing resistant strains unlike antibiotics or other compounds. Further, the present compounds remain stable in the presence of blood or serum proteins because they are devoid of any reactive functionalities. This would take care of preventing deactivation process of antiseptics in in vivo condition which is one of the recent challenges with iodophor and silver based antiseptic formulations of prior art.

High antimicrobial potency of present compounds enable therapeutic efficacy at low dosage within short durations which also minimize toxicity side-effects—a phenomenon common in the currently used antiseptics or compounds. The unique chemistry of SNAP technology also makes it compatible with different delivery matrices like hydrogels, beads and pads, or wound dressing materials. Additionally, SNAP technology increases the antimicrobial action of silver nanoparticle or silver wound dressings when co-administered through dressings or hydrogels at particular concentrations to accomplish sustained release of both the actives and overall improvement in antimicrobial and biofilm inhibitory action for an extended duration. Thus, the compounds of the present disclosure developed using SNAP technology offers not only a safe and improved effectiveness against resistant strains associated with SSIs, but also deliver a unique, non-leaching, sustained drug release approach to maintain optimal drug concentration at the infection site to reduce patient morbidity and health care costs. Thus, the present SNAP technology aims to address, in part, the unmet need to develop a safe, effective, broad spectrum antimicrobial agent in the antiseptic space that would provide excellent antimicrobial property along with potent biofilm inhibitory actions against both gram positive and gram negative pathogens for use in the prevention and cure of SSIs through various modes of application), which in the long run promises to reduce the overall financial burden of therapy.

1. A compound of Formula I: 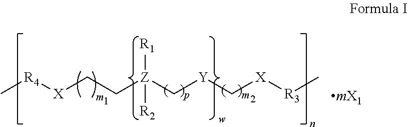
wherein, n=1 to 1000;
p=1 to10;
w=1 to10;
m₁=0 to10;
m₂=0 to 10;
X is —O— or —NH—;
m=1-1000;
X₁ is bromide, chloride, iodide, sulfate, bisulfate, phosphate, nitrate, trifluoroacetate, acetate, propionate, glycolate, succinate, valerate, oleate, palmitate, stearate, laurate, benzoate, lactate, phosphate, tosylate, citrate, maleate, fumarate, succinate, tartrate, ascorbate, napthylate, hydroxymaleate, mesylate, glucoheptonate, lactobionate, laurylsulphonate, phenylacetate, glutamate, benzoate, salicylate, sulfanilate, 2-acetoxybenzoate, fumarate, toluenesulfonate, methanesulfonate, ethane disulfonate, oxalate, isothionate, quaternary ammonium salt, or any other pharmaceutically acceptable salt, or any combinations thereof;
Z is carbon or nitrogen;
Y is —CH₂— or —NH— or functionalized amine;
R₁ and R₂ is independently 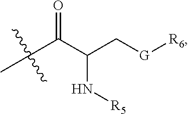
hydrogen, alkyl, straight alkyl chain, branched alkyl chain, —(CH₂)ₙ wherein n=1 to 30, alkenyl, alkynyl, aryl, heteroaryl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 and —(CH₂)ₚ—CH═CH₂ with p=1-10, polymer or polymer derivatives are —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), PEG, polylactic acid (PLA), PEG-PLA co-polymer, alginic acid, chitosan, PLGA, ethylene vinyl acetic acid, acrylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, carboxylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, polyester, polyamide, polycarbamate, polycarbonate, alkyl linked hybrid scaffold, cross-link polymeric scaffold, polybiguanidine, polyurethane, mixed polybiguanidine-polyurethane, polyurea, polyester, polyamide, polycarbonate, polycarbamate, polymethacrylate, polyvinyl, or any combinations of R₁ and R₂ thereof;
and wherein each of the R₁ and R₂ is optionally substituted with primary, secondary, tertiary, quaternary amino group, hydroxyl group, thiol group, acrylic group, halogen selected from fluorine, chlorine, bromine or iodine, —COR₈ wherein R₈ is alkyl, alkenyl, monoenes, polyenes, terminally substituted alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, or C-terminal amino acids with D or L configuration, oligo-peptide, or any combinations thereof, wherein R₁ is optionally present;
and wherein in 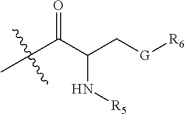
‘G’ is oxygen (—O—) or sulphur (—S—), R₅ and R₆ is independently selected from hydrogen, alkyl, straight alkyl chain, branched alkyl chain, —(CH₂)ₙ wherein n=1 to 30, alkenyl, alkynyl, aryl, heteroaryl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 or —(CH₂)ₚ—CH═CH₂ with p=1-10, and wherein each of R₅ and R₆ is optionally substituted with primary, secondary, tertiary or quaternary amino group, hydroxyl group, thiol group, carboxylic group, acrylic group or halogen selected from fluorine, chlorine, bromine or iodine, or any combinations thereof;
and wherein in —COR₇ and —COR₈, R₇ and R₈ is independently selected from alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl or heteroaryl, and wherein each of the R₇ and R₈ is optionally substituted with primary, secondary, tertiary or quaternary amino group, hydroxyl group, thiol group, carboxylic group, acrylic group, halogen selected from fluorine, chlorine, bromine or iodine; C-terminal amino acids with D or L configuration, or oligo-peptide, or any combinations thereof;
and
R₃ and R₄ is independently hydrogen, alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl or heteroaryl group, wherein each of the R₃ and R₄ is optionally substituted with primary, secondary, tertiary or quaternary amino group, hydroxyl group, thiol group, carboxylic group, acrylic group, halogen selected from fluorine, chlorine, bromine or iodine, —COR₈ with R₈ selected from alkyl, alkenyl, monoenes, polyenes, terminally substituted alkyl, straight alkyl chain, branched alkyl chain, —(CH₂)n with n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, oligopeptide, polymer or polymer derivatives selected from —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PLGA, ethylene vinyl acetic acid, polymethacrylate, polyvinyl, acrylic derivatives of —CH₂—(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA or ethylene vinyl acetic acid, carboxylic derivatives of —CH₂-(CH₂)ₙ—COO-PVA, —CO—CH₂—(CH₂)ₙ-PVA with n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA, ethylene vinyl acetic acid, polyester, polyamide, polycarbamate, polycarbonate, alkyl linked hybrid scaffold, cross-link polymeric scaffold, polybiguanidine, polyurethane, mixed polybiguanidine-polyurethane, polyurea, polyester, polyamide, polycarbonate or polycarbamate or any combinations thereof; —C(NH)—NH—C(NH)—, —C(NH)—NH—C(NH)—NH—(CH₂)ₙ—NH—C(NH)—NH—C(NH)— with n=1-20, —C(NH)—NH—C(NH)—NH-M-NH—C(NH)—NH—C(NH)— wherein M is 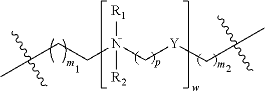
wherein R₁, R₂, Y, p, w, m₁ and m₂ is as defined above and R₁ is optional, —C(NH)—NH—C(NH)—NH—CH₂—(CH₂)ₘ—O(CO)NH—(CH₂)ₙ—NH(CO)—OCH₂—(CH₂)ₙ—NH—C(NH)—NH—C(NH)— with m=1-20, n=1-20, —CO—NH—(CH₂)ₙ—NH(CO)— with n=1-20, —CO—(CH₂)ₙ—(CO)— with n=1-20, —CO—NH—(CH₂)ₙ—NH(CO)— with n=1-20, [—CO—NH—(CH₂)ₙ—NH(CO)—(OCH₂—CH₂)ₘ—O(CO)—NH—] with m=1-20, n=1-20, —CO—(CH₂)ₙ—(CO)— with n=1-20, —COO—, —OCO—(CH₂CH₂—O)ₙ—CO— with n=1-20, —C(NH)—NH—C(NH)—NH—(CH₂)ₘ—O(CO)NH—(CH₂)ₙ—NH(CO)—O—(CH₂)ₙ—NH—C(NH)—NH—C(NH)— with m=1-20, n=1-20.

2.-40. (canceled)

41. A compound of Formula I of claim 1, wherein the compound is

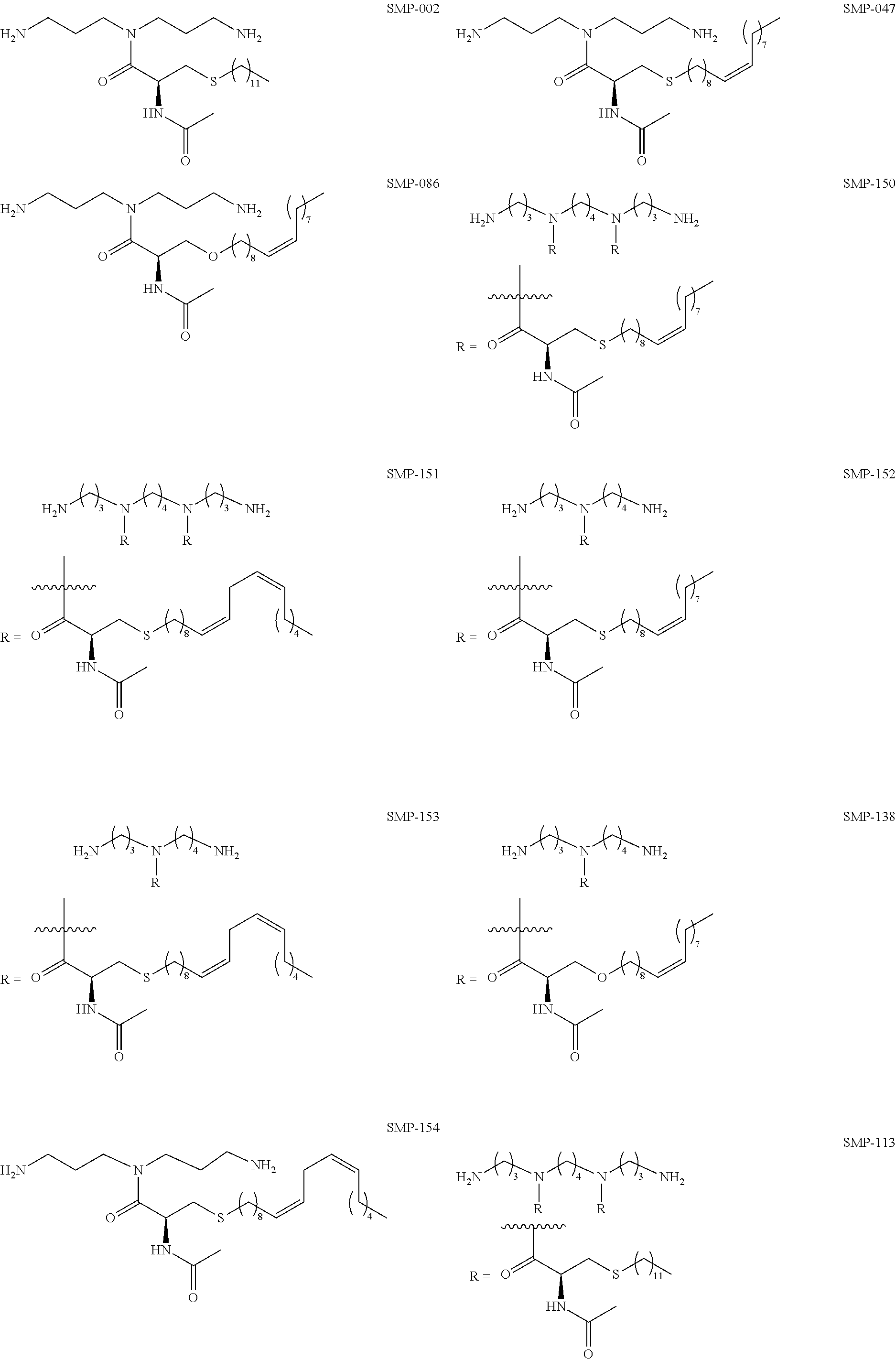

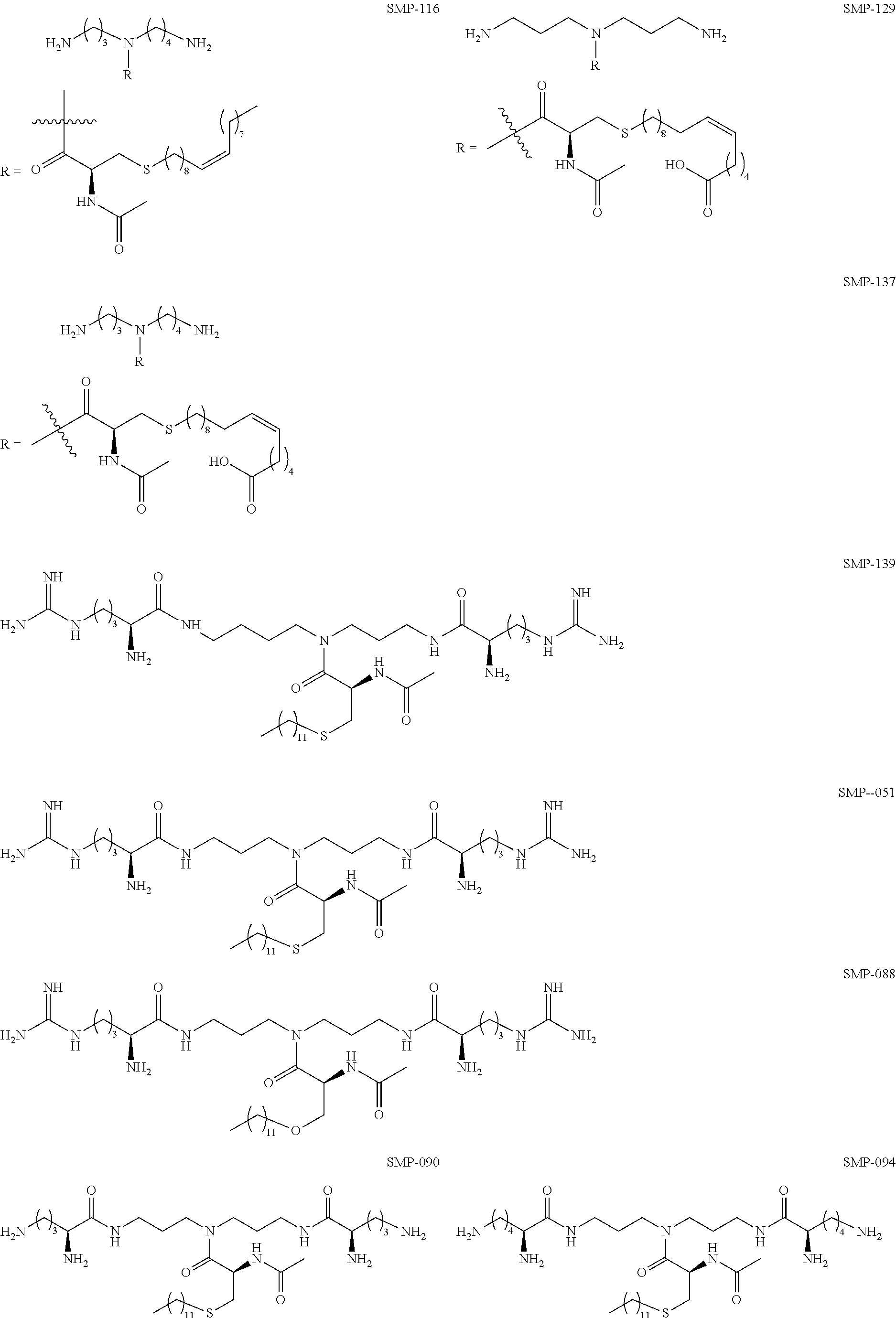

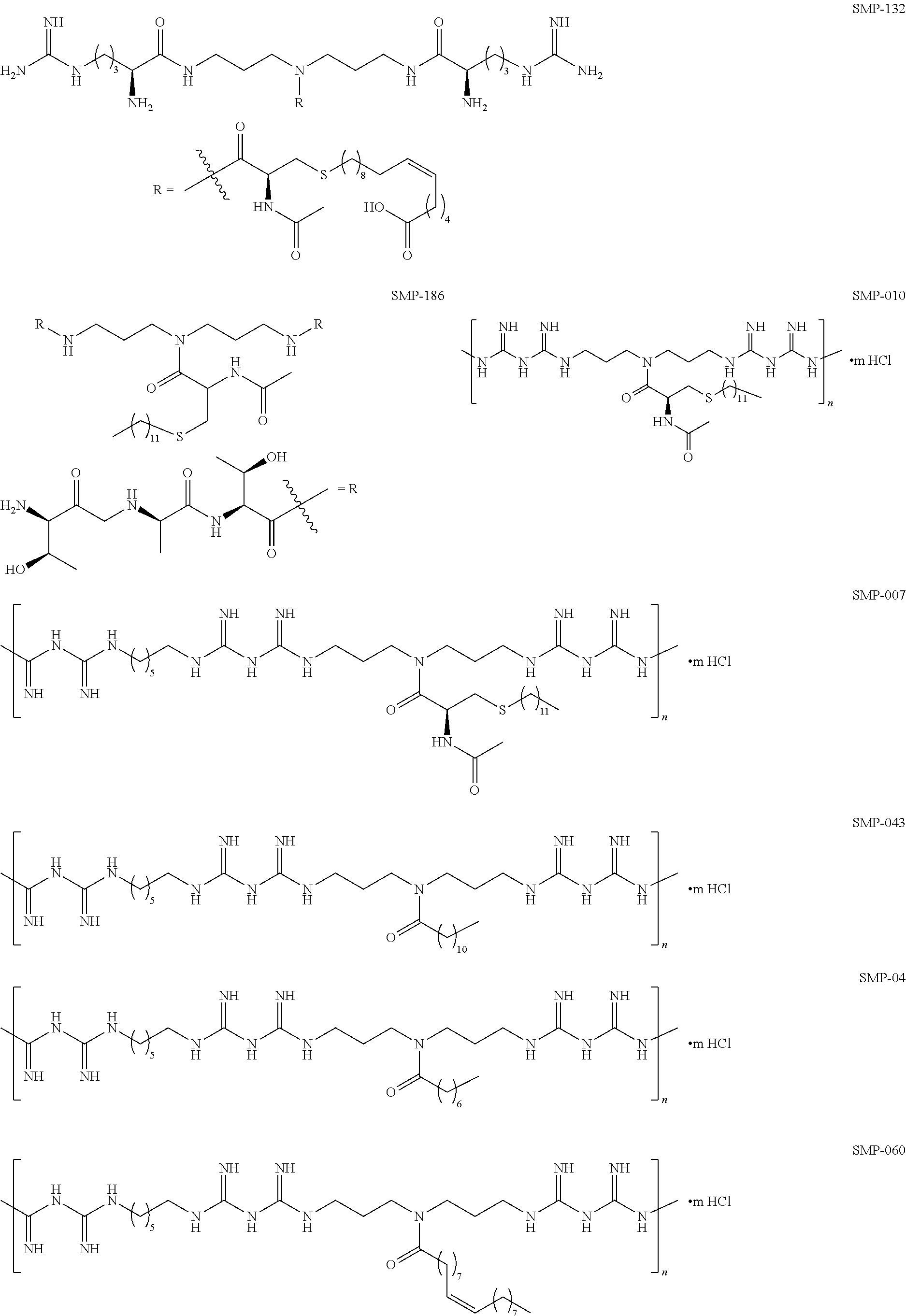

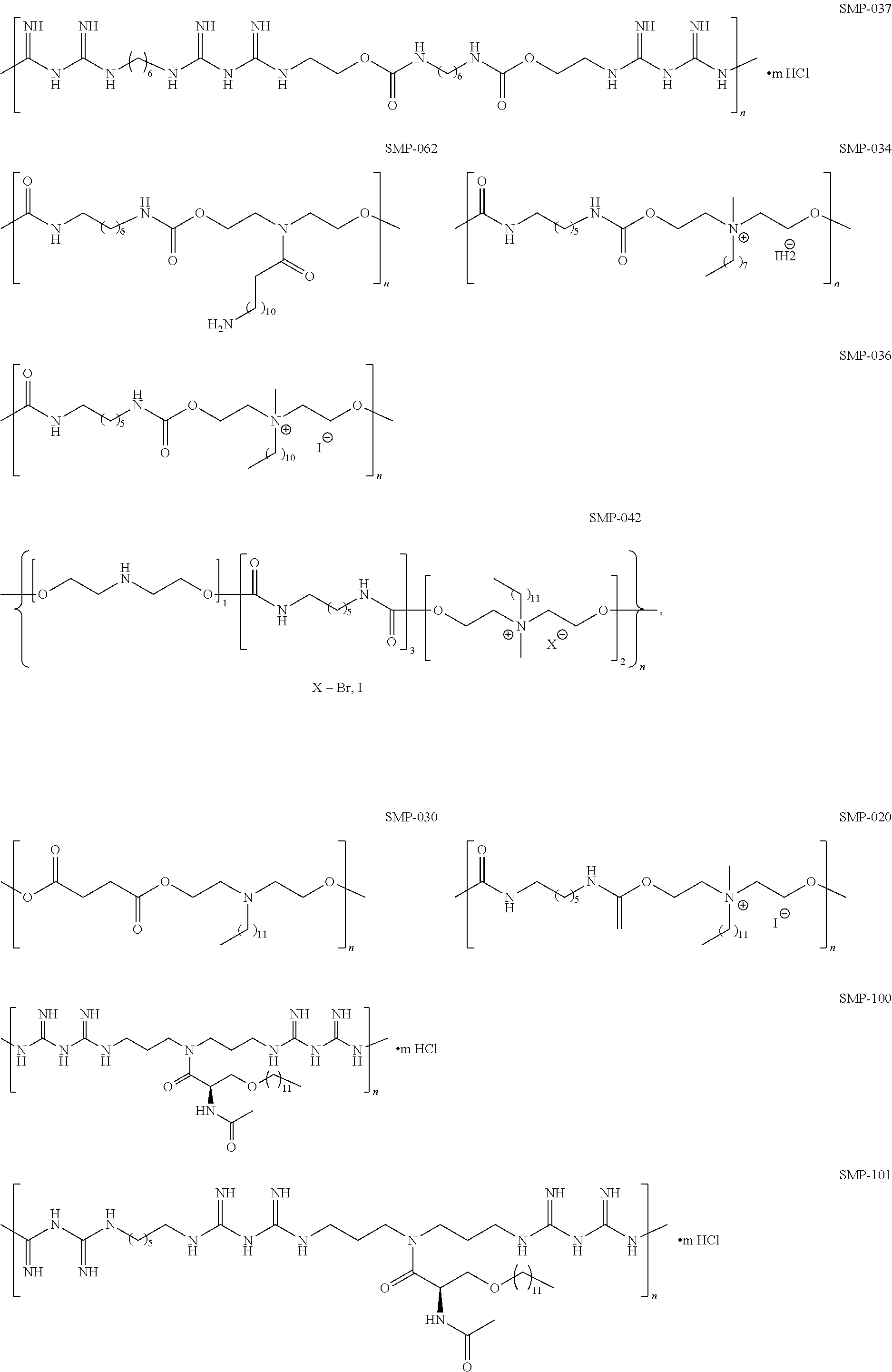

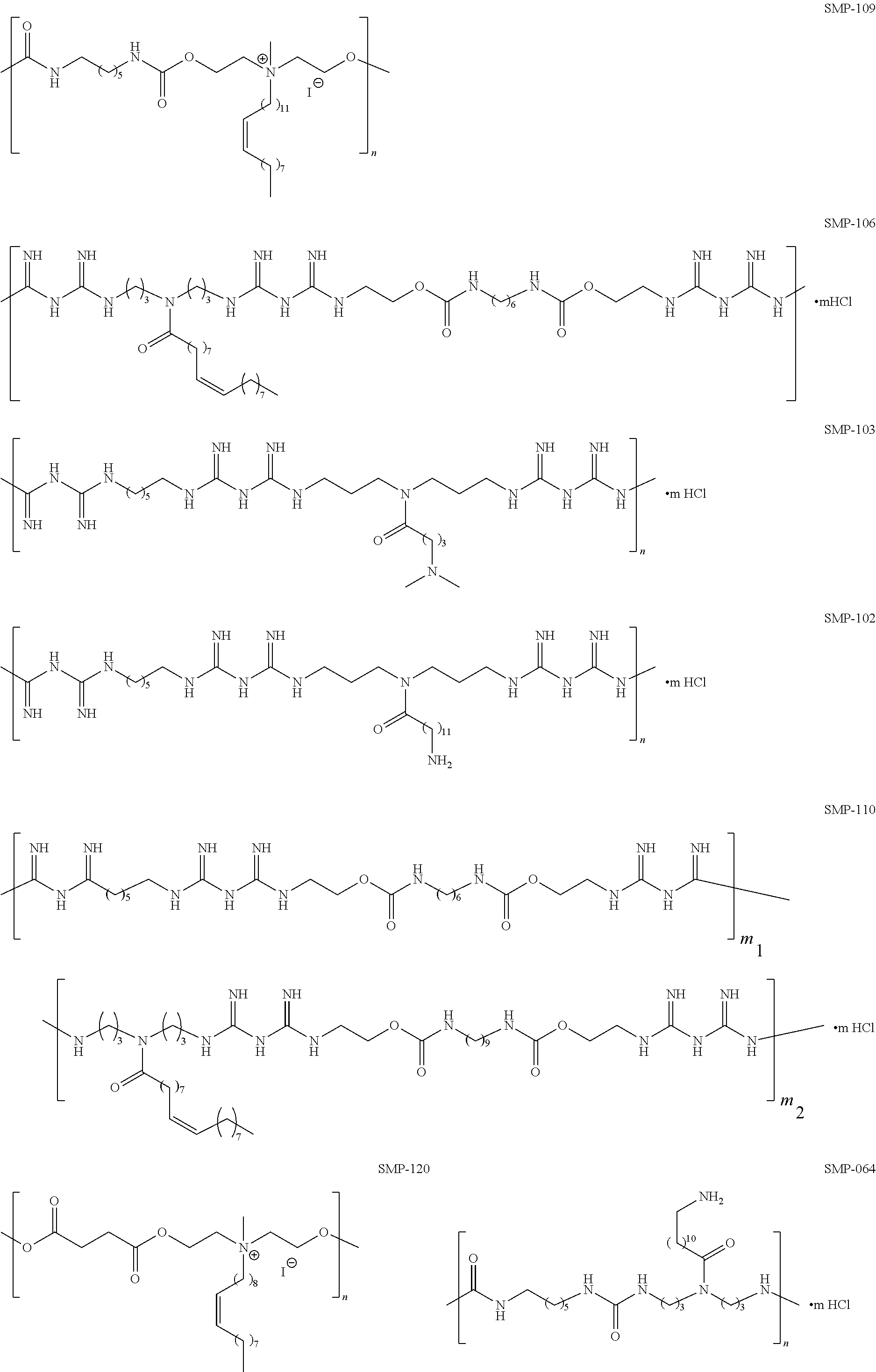

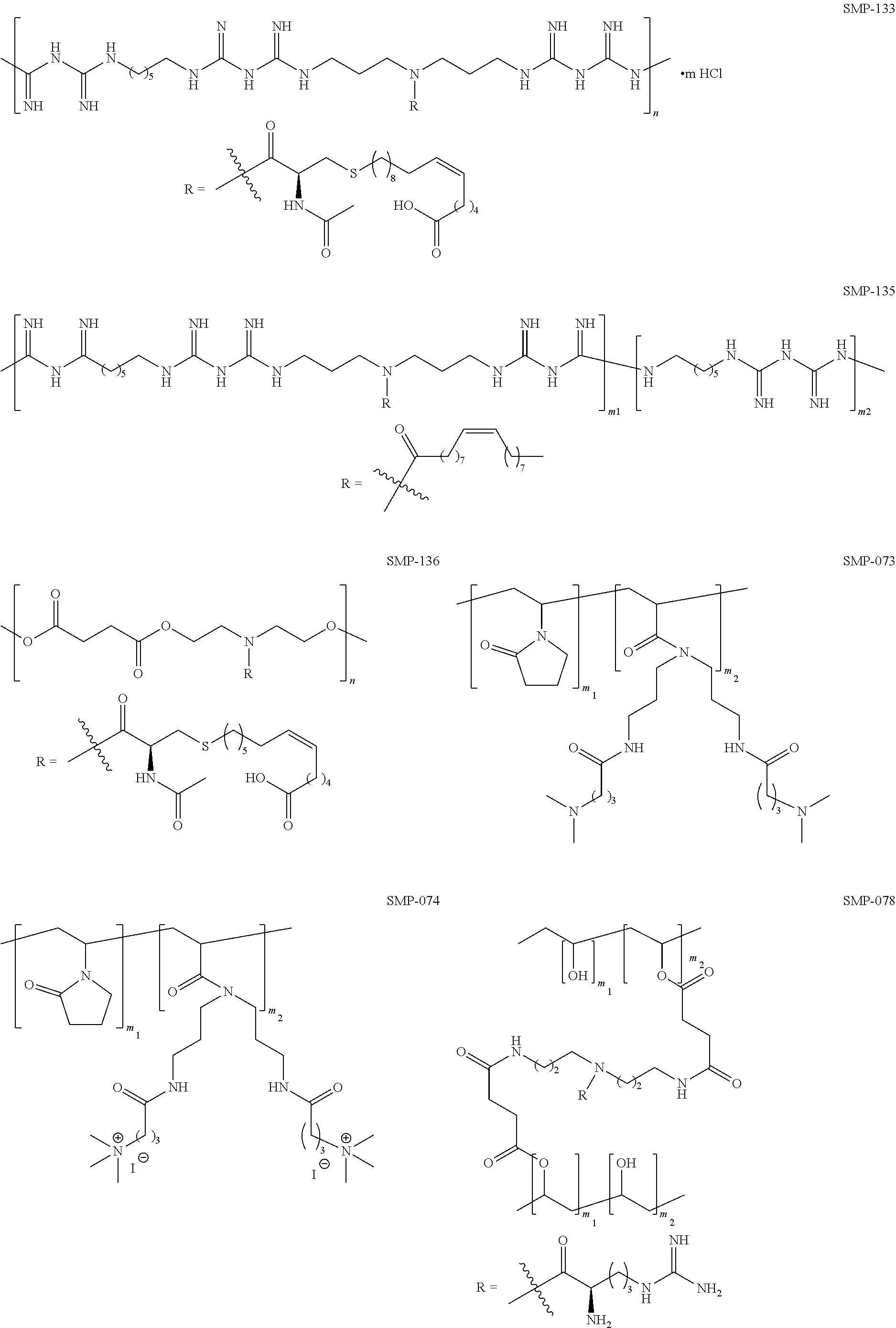

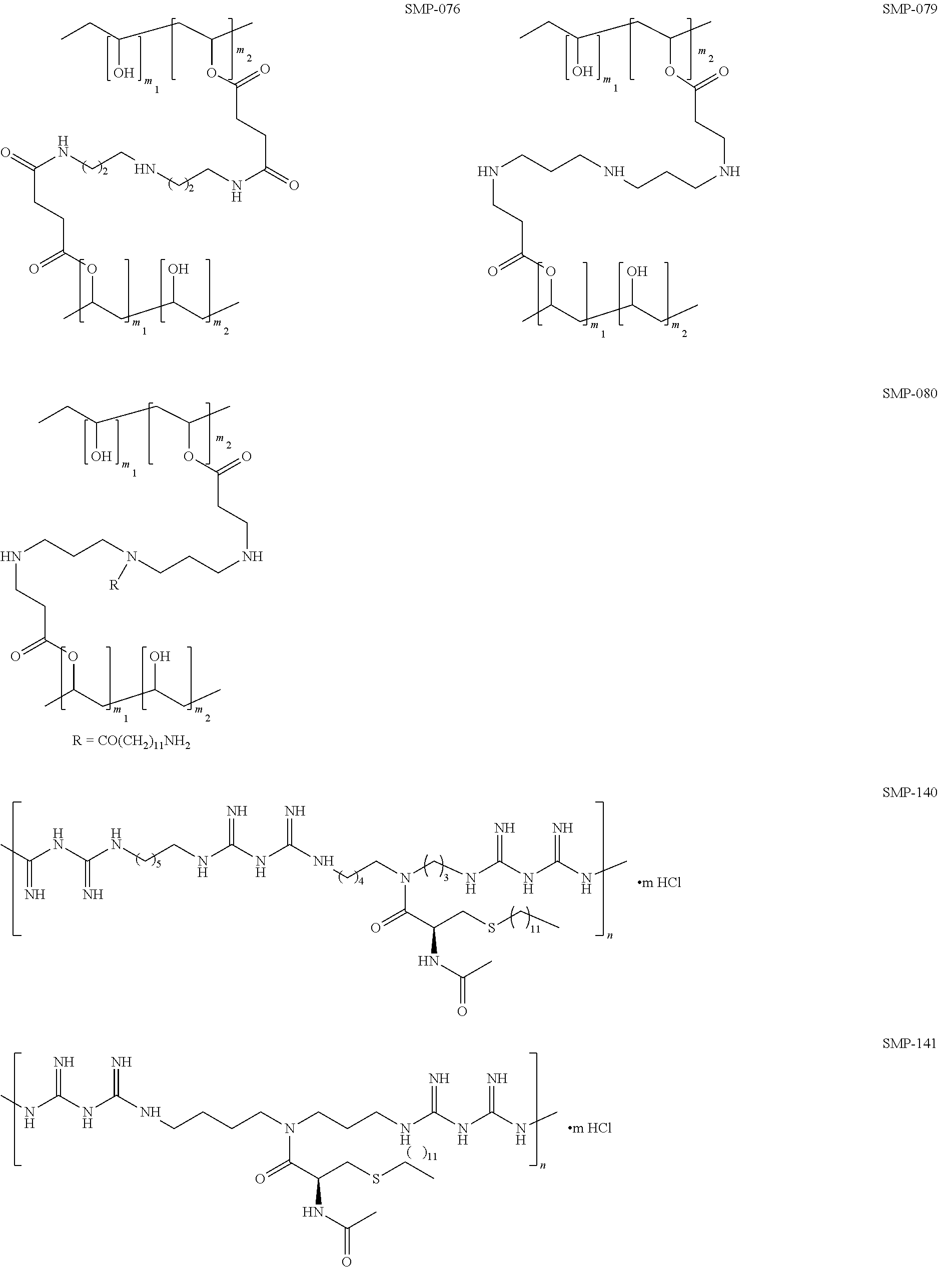

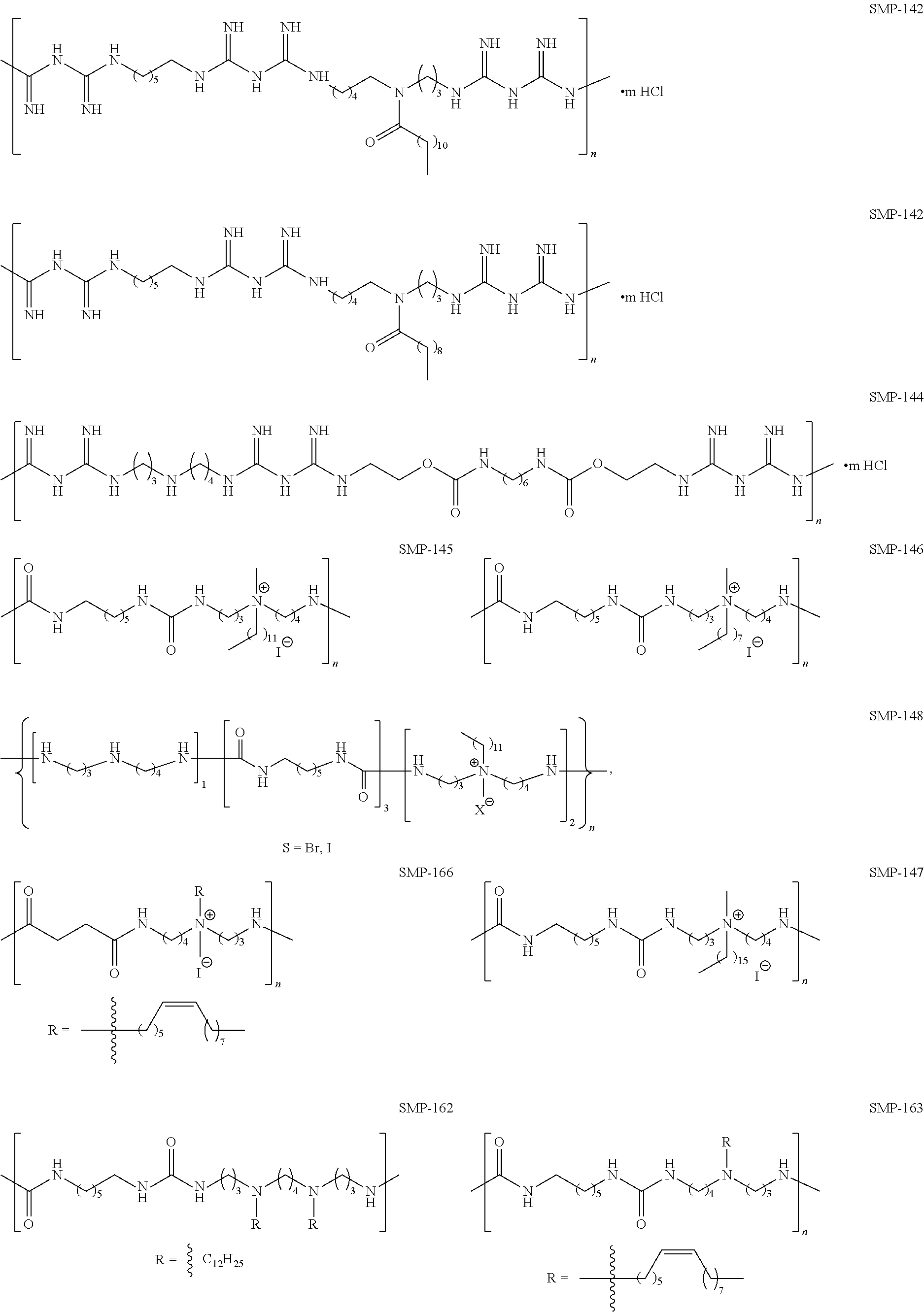

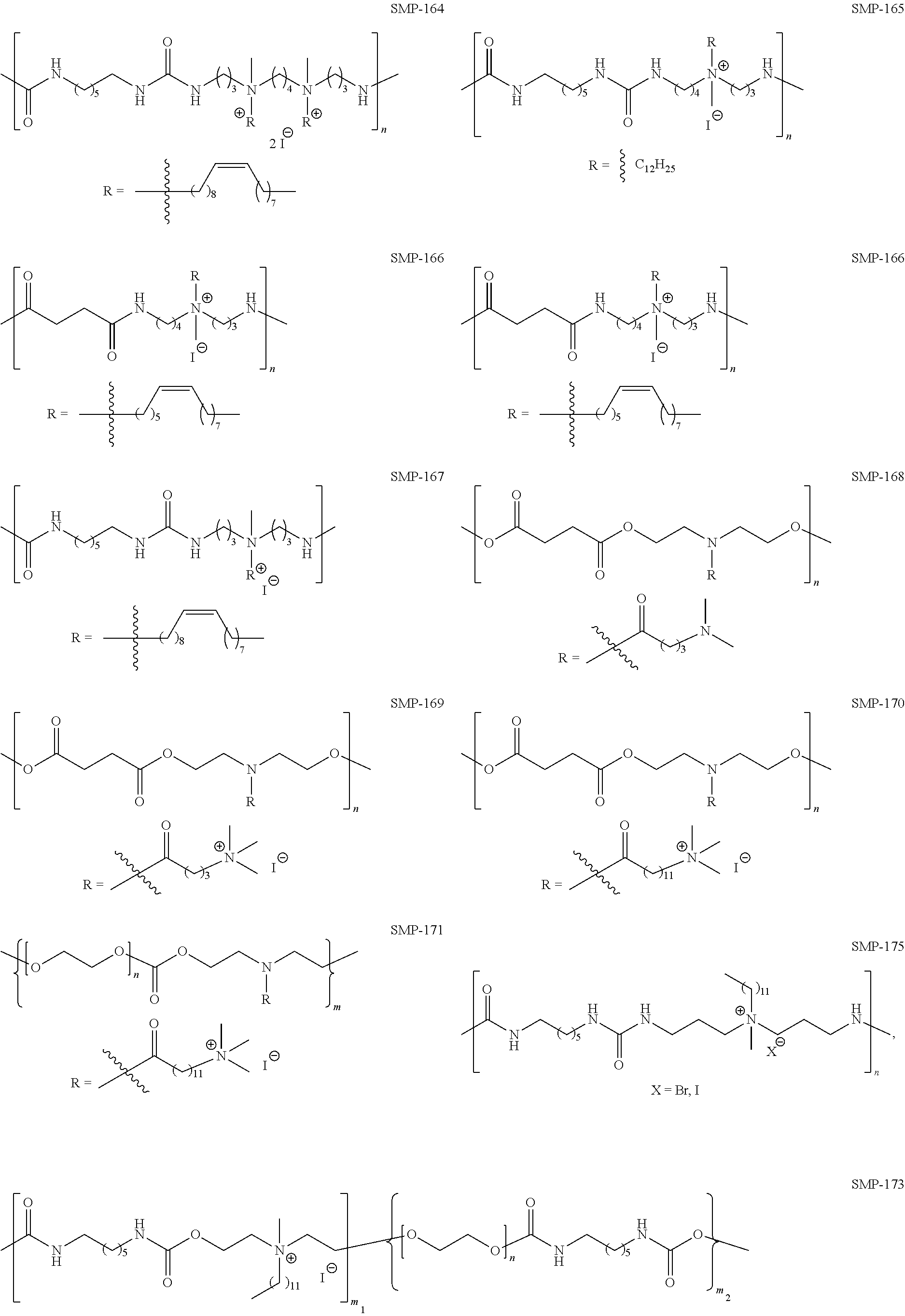

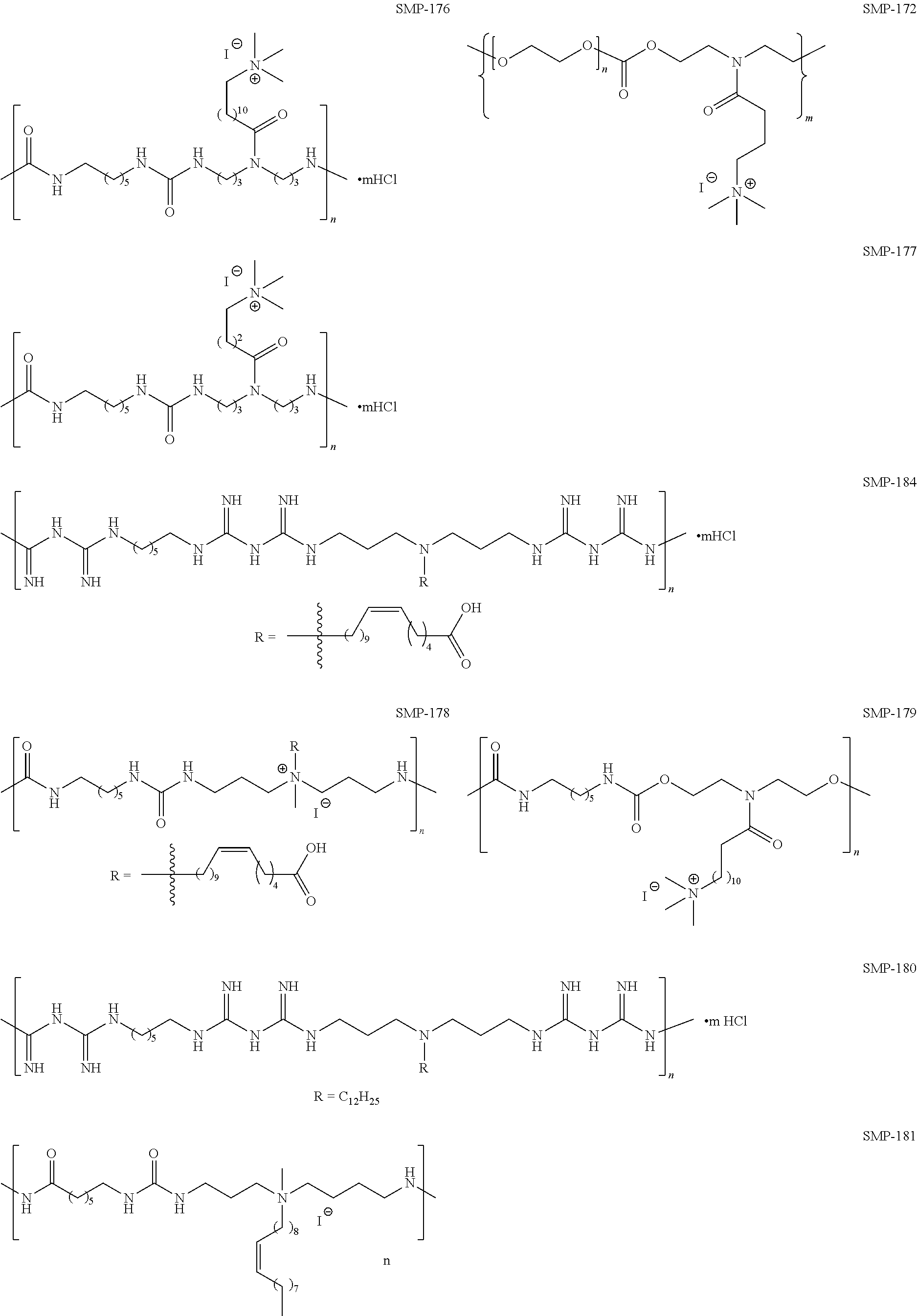

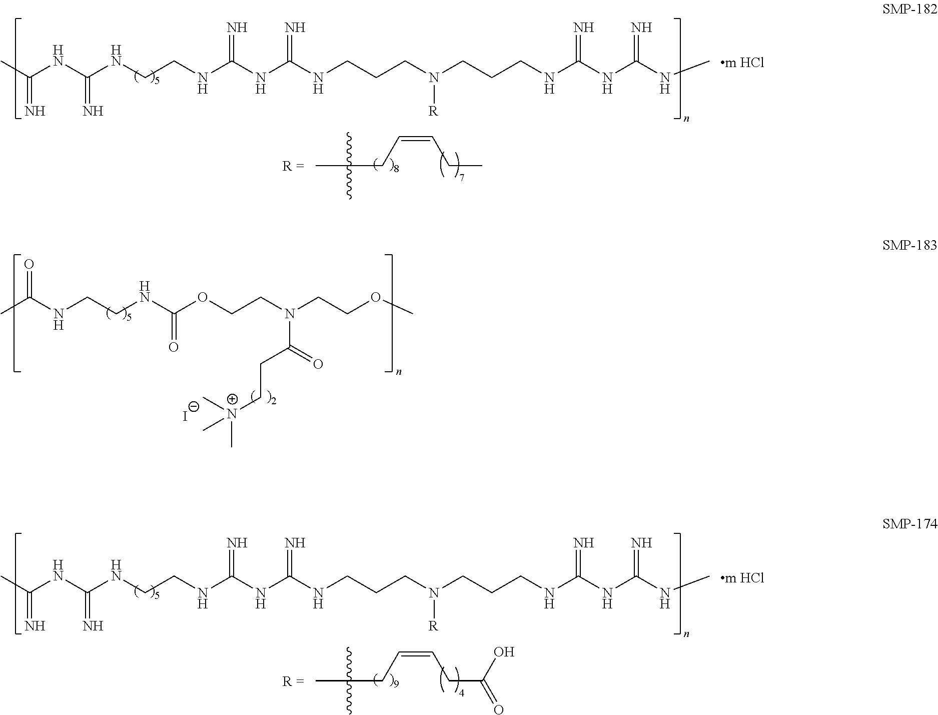

42. The compound of claim 1, wherein the compound is of Formula Ia 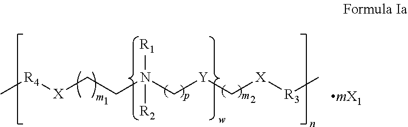
wherein,
p=1 to10;
w=1 to10;
m₁=0to10;
m₂=0 to10;
R₁ is absent;
R₂ is independently hydrogen, 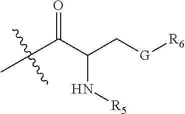
wherein ‘G’ is oxygen (—O—) or sulphur (—S—), wherein R₅ and R₆ is independently hydrogen, alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 or —(CH₂)p-CH═CH₂ with p=1-10, and wherein each of R₅ and R₆ is optionally substituted with primary, secondary, tertiary or quaternary amino group, hydroxyl group, thiol group, carboxylic group, acrylic group, halogen selected from fluorine, chlorine, bromine or iodine, —COR₇ wherein R₇ is alkyl, straight alkyl chain, branched alkyl chain, —(CH₂)n wherein n=1 to 30, alkenyl, alkynyl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10, —(CH₂)p-CH═CH₂ with p=1-10, aryl, heteroaryl and wherein each of the R₇ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide;
R₃ and R₄ is independently hydrogen, alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl, or heteroaryl group, and wherein each of the R₃ and R₄ is optionally substituted with primary, secondary, tertiary, quaternary amino group, hydroxyl group, thiol group, carboxylic group, acrylic group, halogen selected from fluorine, chlorine, bromine or iodine, —COR₈ wherein R₈ is alkyl, alkenyl, monoenes, polyenes, terminally substituted alkyl, straight alkyl chain, branched alkyl chain, —(CH₂)ₙ wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligopeptide;
Y is —CH₂—, —NH— and a functionalized amine, wherein the functionalized amine is —N(R₂), wherein R₂ is 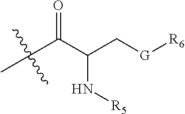
with ‘G’ being oxygen (—O—) or sulphur (—S—);
X=—NH— or —O—;
m=1-1000; and
X₁, R₅ and R₆ is as defined in claim 1.

43. The compound of claim 1, wherein:
n=2 to 1000;
Z═NH or C;
Y═NH or CH₂;
X═NH or O;
p=1 to10;
w=1 to10;
m₁=0 to 10;
m₂=0 to 10;
X₁ as defined in claim 1;
R₃ and R₄ is independently hydrogen, alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, aryl or heteroaryl group and wherein each of the R₃ and R₄ is optionally substituted with primary, secondary, tertiary or quaternary amino group, hydroxyl group, thiol group, carboxylic group, acrylic group, halogen selected from fluorine, chlorine, bromine or iodine, —COR₈ wherein R₈ is alkyl, alkenyl, monoenes, polyenes, terminally substituted alkyl, straight alkyl chain, branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide;
R₁ is absent;
R₂ is a polymer or polymer derivative is —CH₂—(CH₂)ₙ—COO-PVA, —CO—CH₂—(CH₂)ₙ-PVA wherein n=1-20; polyvinylpyrolidone (PVP), polyglycolic acid (PGA), polyacrylic acid (PAA), polymethacrylate, polyvinyl, alginic acid, chitosan, PEG, polylactic acid (PLA), PEG-PLA co-polymer, PLGA, ethylene vinyl acetic acid, acrylic derivatives of —CH₂—(CH₂)ₙ—COO-PVA, —CO—CH₂—(CH₂)ₙ-PVA wherein n=1-20, polyvinylpyrolidone (PVP), polyglycolic acid (PGA), polyacrylic acid (PAA), alginic acid, chitosan, PEG, polylactic acid (PLA), PEG-PLA co-polymer, PLGA, ethylene vinyl acetic acid, carboxylic derivatives of —CH₂—(CH₂)ₙ—COO-PVA, —CO—CH₂—(CH₂)ₙ-PVA wherein n=1-20, polyvinylpyrolidone (PVP), polyglycolic acid (PGA), polyacrylic acid (PAA), alginic acid, chitosan, PLGA, PEG, polylactic acid (PLA), PEG-PLA co-polymer, ethylene vinyl acetic acid, polyester, polyamide, polycarbamate, polycarbonate, alkyl linked hybrid, cross-link polymeric scaffolds, polybiguanidine, polyurethane, mixed polybiguanidine-polyurethane, polyurea, polyester, polyamide, polycarbonate or polycarbamate, or any combinations thereof.

44. The compound of claim 43, wherein n=2 to 500, R₁, R₂, R₃, R₄, X, Y, Z, m₁, p, m₂, m, X₁ are as defined in claim 43.

45. The compound of claim 1 comprising conjugate polymers of compound of Formula I, wherein compound of Formula I with ‘n’=2 to 1000 conjugates with polymer or polymer derivative selected from polyvinylpyrolidone (PVP), polyglycolic acid (PGA), polyacrylic acid (PAA), polymethacrylate, polyvinyl, alginic acid, chitosan, PLGA, ethylene vinyl acetic acid, polyester, polyamide, polycarbamate, polycarbonate, PEG, PLA, PLA-PEG co-polymer, PHMB, polyguanidine, polybiguanidine, polyurethane, polybiguanidine-polyurethane, polyurea, polyester, polyamide or polycarbonate, or any combinations thereof.

46. The compound of claim 1, wherein the compound is a compound of Formula Ib 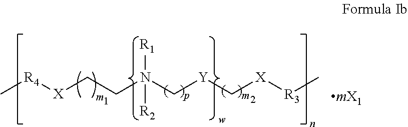
wherein, n=2 to 1000;
p=1 to 10;
w=1 to 10;
m₁=0 to 10;
m₂=0 to10;
R₃ and R₄ is independently —C(NH)—NH—C(NH)—; —C(NH)—NH—C(NH)—NH—(CH₂)ₙ—NH—C(NH)—NH—C(NH)— wherein n=1-20; —C(NH)—NH—C(NH)—NH-M-NH—C(NH)—NH—C(NH)— wherein M is 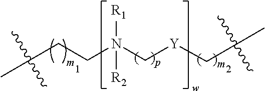
wherein R₁, R₂, Y, p, w, m₁ and m₂ as defined above, wherein R₁ is optional;
—C(NH)—NH—C(NH)—NH—CH₂—(CH₂)ₘ—O(CO)NH—(CH₂)ₙ—NH(CO)—OCH₂—(CH₂)ₙ—NH—C(NH)—NH—C(NH)—, wherein m=1-20, n=1-20; —CO—NH—(CH₂)ₙ—NH(CO)— wherein n=1-20; —CO—(CH₂)ₙ—(CO)—, wherein n=1-20; —CO—NH—(CH₂)ₙ—NH(CO)— wherein n=1-20; [—CO—NH—(CH₂)ₙ—NH(CO)—(OCH₂—CH₂)ₘ—O(CO)—NH—] wherein m=1-20, n=1-20; —CO—(CH₂)ₙ—(CO)— wherein n=1-20; —COO—; —OCO—(CH₂CH₂—O)ₙ—CO—, wherein n=1-20;
X is oxygen or —NH—;
m=1-1000;
X₁ is as defined in claim 1;
Y is —NH—;
wherein R₁ and R₂ is independently alkyl, alkenyl, alkynyl, straight alkyl chain, branched alkyl chain, —(CH₂)ₙ wherein n=1 to 30, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 and —(CH₂)ₚ—CH═CH₂, with p=1-10, and wherein each of the R₁ and R₂ is optionally substituted with primary, secondary, tertiary or quaternary amino group, hydroxyl group, thiol group, acrylic group, halogen selected from fluorine, chlorine, bromine or iodine, —COR₈ wherein R₈ is alkyl, alkenyl (mono or polyenes) or terminally substituted alkyl, straight or branched alkyl chain, —(CH₂)n wherein n=1 to 30, alkenyl, alkynyl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10, —(CH₂)ₚ—CH═CH₂ with p=1-10, and wherein each of the R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligopeptide, 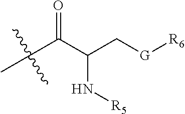
wherein ‘G’ is oxygen (—O—) or sulphur (—S—), wherein R₅ and R₆ is independently hydrogen or alkyl, straight alkyl chain, branched alkyl chain, —(CH₂)n wherein n=1 to 30, alkenyl, alkynyl, aryl, heteroaryl, —(CH₂)ₚ—CH═CH—CH₂—(CH₂)ₚ—CH₃ with p=1-10, —(CH₂)ₚ—CH═CH—(CH₂)ₚ—CH═CH—(CH₂)ₚ—CH₃ with p=1-10 or —(CH₂)p-CH═CH₂ with p=1-10, and wherein each of R₅ and R₆ is optionally substituted with primary, secondary, tertiary or quaternary amino group, hydroxyl group, thiol group, carboxylic group, acrylic group, halogen selected from fluorine, chlorine, bromine or iodine, —COR₇, —COR₈ wherein R₇ and R₈ is alkyl, straight alkyl chain, branched alkyl chain, —(CH₂)n with n=1 to 30, alkenyl, alkynyl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10, —(CH₂)p-CH═CH₂ with p=1-10, aryl, heteroaryl and wherein each of the R₇ and R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide; wherein the C-terminal amino acids with D or L configuration is lysine, arginine, ornithine, proline, histidine, serine, threonine, tyrosine, tryptophan, phenyl alanine, cysteine, cystine, isoleucine, leucine, glycine, asparagine, glutamine, aspartic acid or glutamic acid, or any combinations thereof;
wherein the C-terminal oligopeptide consists of 2-30 amino acids with D or L configuration wherein C-terminal oligopeptide is TAT (Threonine-Alanine-Threonine), Cholesterol-conjugated G3R6TAT (dodecapeptide), MP196 (hexapeptide, RWRWRW—NH₂), PAF-26 (hexapeptide, RKKWFW), Mastoparan (Polybia-MP1, tetradecapeptide, IDWKKLLDAAKQIL), D-IK8 (octapeptide, IRIKIRIK), L5K5W (undecapeptide, KKLLKWLKKLL-NH₂), Gramicidin-D (pentadecapeptide, VGALAVVVWLWLWLW), WR12 (dodecapeptide, RWWRWWRRWWRR), Protegrins (PG-1, octadecapeptide or NH₂—RGGRLCYCRRRFCVCVGR—CONH₂)), or any combinations thereof.

47. The compound of claim 46, wherein the Formula Ib is a compound wherein X═NH; m₁, R₁, R₂, p, Y, m₂, m, X₁ is bromide, chloride, iodide, sulfate, bisulfate, phosphate, nitrate, trifluoroacetate, acetate, propionate, glycolate, succinate, valerate, oleate, palmitate, stearate, laurate, benzoate, lactate, phosphate, tosylate, citrate, maleate, fumarate, succinate, tartate, ascorbate, naphthylate, hydroxymaleate, mesylate, glucoheptonate, lactobionate, laurylsulphonate, phenylacetate, glutamate, benzoate, salicylate, sulfanilate, 2-acetoxybenzoate, fumarate, toluenesulfonate, methanesulfonate, ethane disulfonate, oxalate, isothionate, quaternary ammonium salt, or any other combinations thereof; and R₃ and R₄ are combinations selected from:
R₃═R₄═—C(NH)—NH—C(NH)—;
R₃═—C(NH)—NH—C(NH)— and R₄═—C(NH)—NH—C(NH)—NH—(CH₂)ₙ—NH—C(NH)—NH—C(NH)—, wherein n=1-20;
R₃═R₄═—C(NH)—NH—C(NH)—NH—(CH₂)ₙ—NH—C(NH)—NH—C(NH)—, wherein n=1-20;
R₃═—C(NH)—NH—C(NH)— and R₄ is —C(NH)—NH—C(NH)—NH-M-NH—C(NH)—NH—C(NH)—, wherein M is 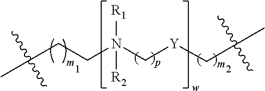
wherein R₁, R₂, Y, p, w, m₁ and m₂ are same as defined in Formula Ia of claim 2;
R₃═—C(NH)—NH—C(NH)— and R₄═—C(NH)—NH—C(NH)—NH—CH₂—(CH₂)ₘ—O(CO)NH—(CH₂)ₙ—NH(CO)—OCH₂—(CH₂)ₙ—NH—C(NH)—NH—C(NH)—, wherein m=1-20, n=1-20;
R₃═R₄═—CO—NH—(CH₂)ₙ—NH(CO)— wherein n=1-20; or
R₃═R₄═—CO—(CH₂)ₙ—(CO)—, wherein n=1-20.

48. The compound of claim 42 of Formula Ia wherein X═O; m₁, R₁, R₂, p, Y, m₂, m, X₁ is bromide, chloride, iodide, sulfate, bisulfate, phosphate, nitrate, trifluoroacetate, acetate, propionate, glycolate, succinate, valerate, oleate, palmitate, stearate, laurate, benzoate, lactate, phosphate, tosylate, citrate, maleate, fumarate, succinate, tartate, ascorbate, naphthylate, hydroxymaleate, mesylate, glucoheptonate, lactobionate, laurylsulphonate, phenylacetate, glutamate, benzoate, salicylate, sulfanilate, 2-acetoxybenzoate, fumarate, toluenesulfonate, methanesulfonate, ethane disulfonate, oxalate, isothionate, quaternary ammonium salt, or any other combinations thereof; and R₃ and R₄ are combinations selected from:
R₃═R₄═—CO—NH—(CH₂)ₙ—NH(CO)— wherein n=1-20;
R₃═—CO—NH—(CH₂)ₙ—NH(CO)— wherein n=1-20 and R₄═[—CO—NH—(CH₂)ₙ—NH(CO)—(OCH₂—CH₂)ₘ—O(CO)—NH—] wherein n=1-20, m=1-20;
R₃═R₄═—CO—(CH₂)ₙ—(CO)—, wherein n=1-20;
R₃═R₄═—COO—; or
R₃═—COO— and R₄═—OCO—(CH₂CH₂—O)ₙ—CO—, wherein n=1-20.

49. The compound of claim 1, wherein said compound is a compound of Formula Ic 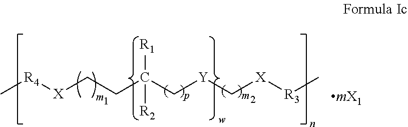
wherein n=2-1000,
Y═C,
X═NH or O,
p=1 to 10,
w=1 to 10,
m₁=0 to 10,
m₂=0 to 10, and
R₁ R₂, R₃, R₄ are as defined in claim 1.

50. A compound of Formula I of claim 1, wherein the compound is selected from

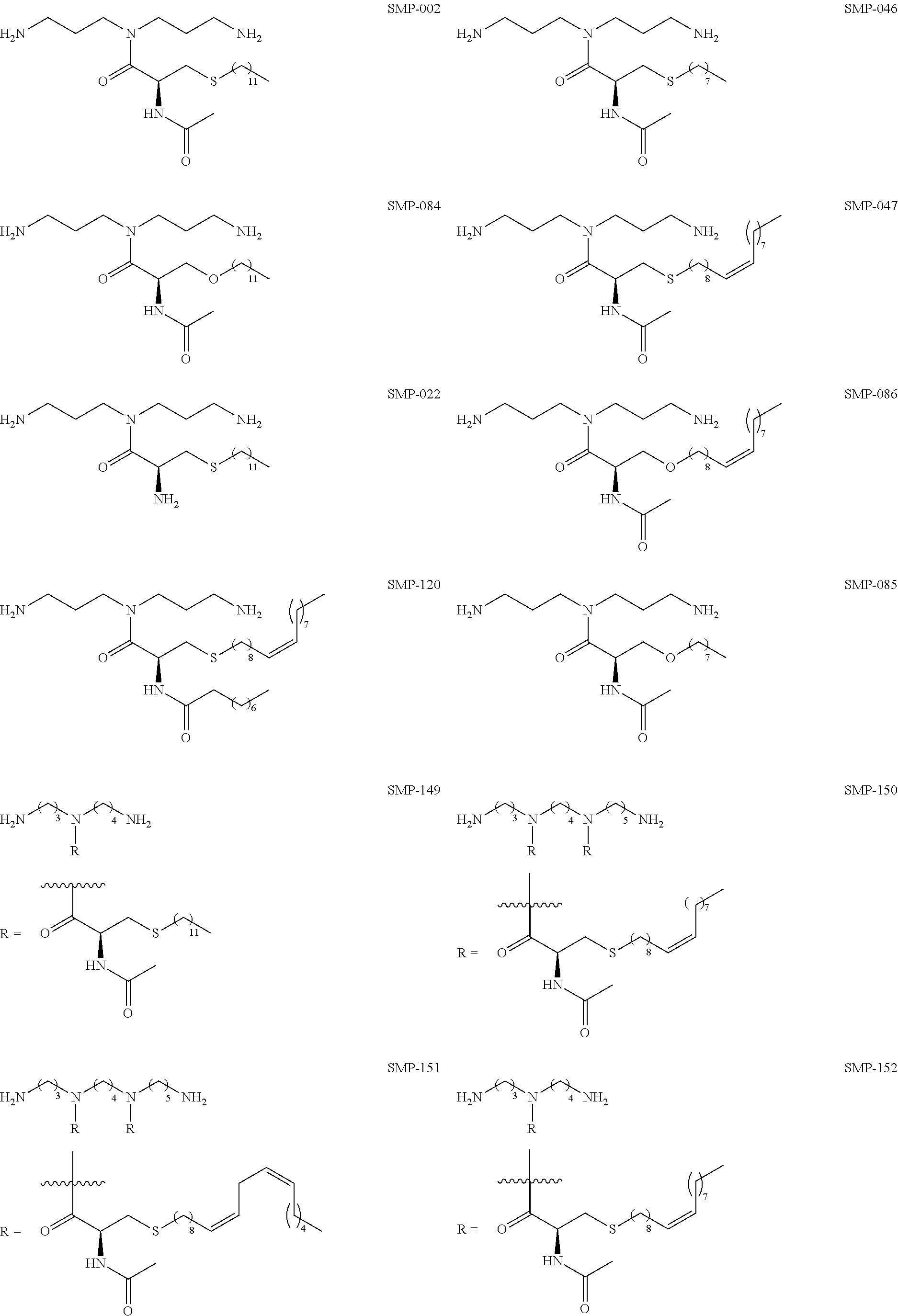

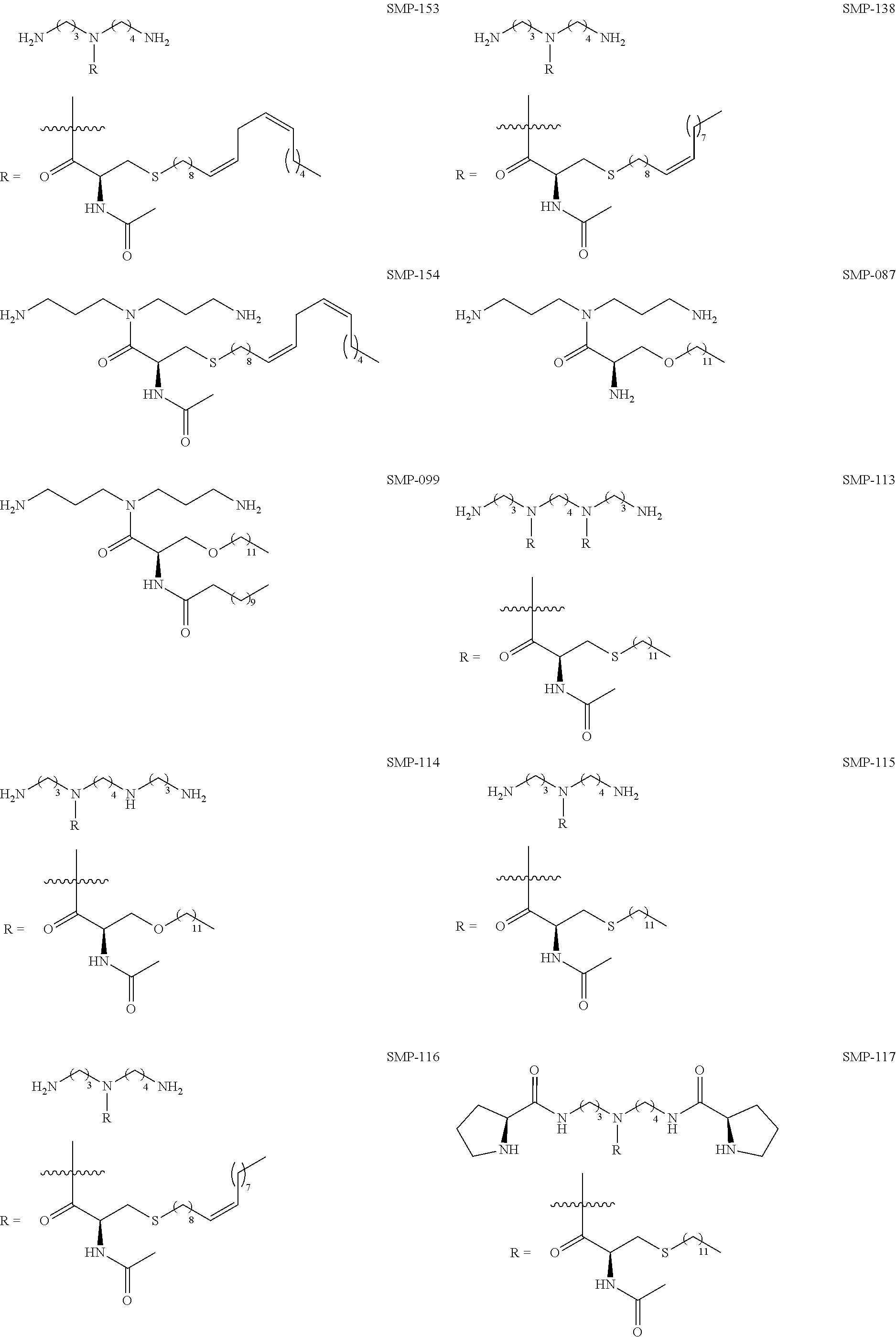

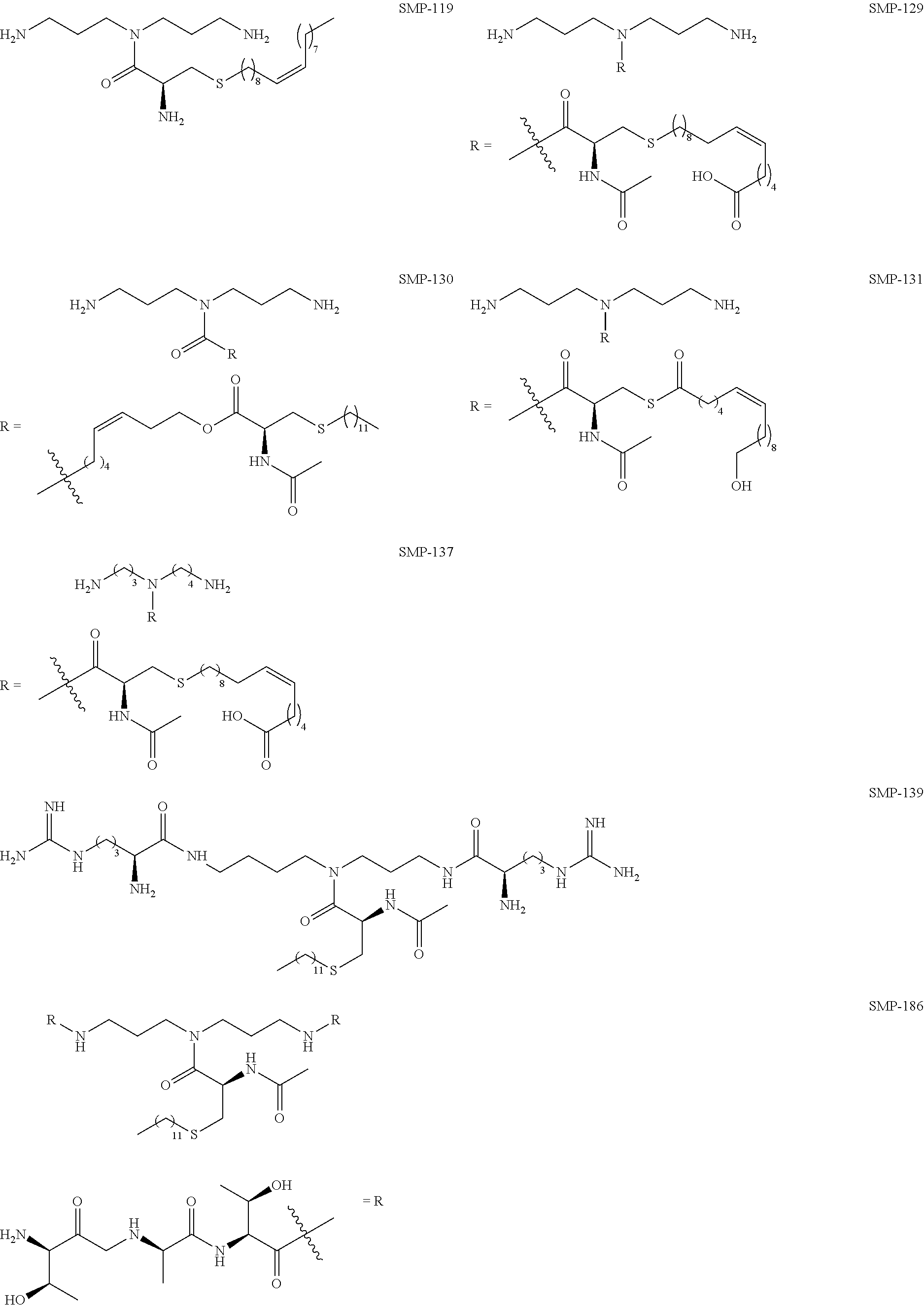

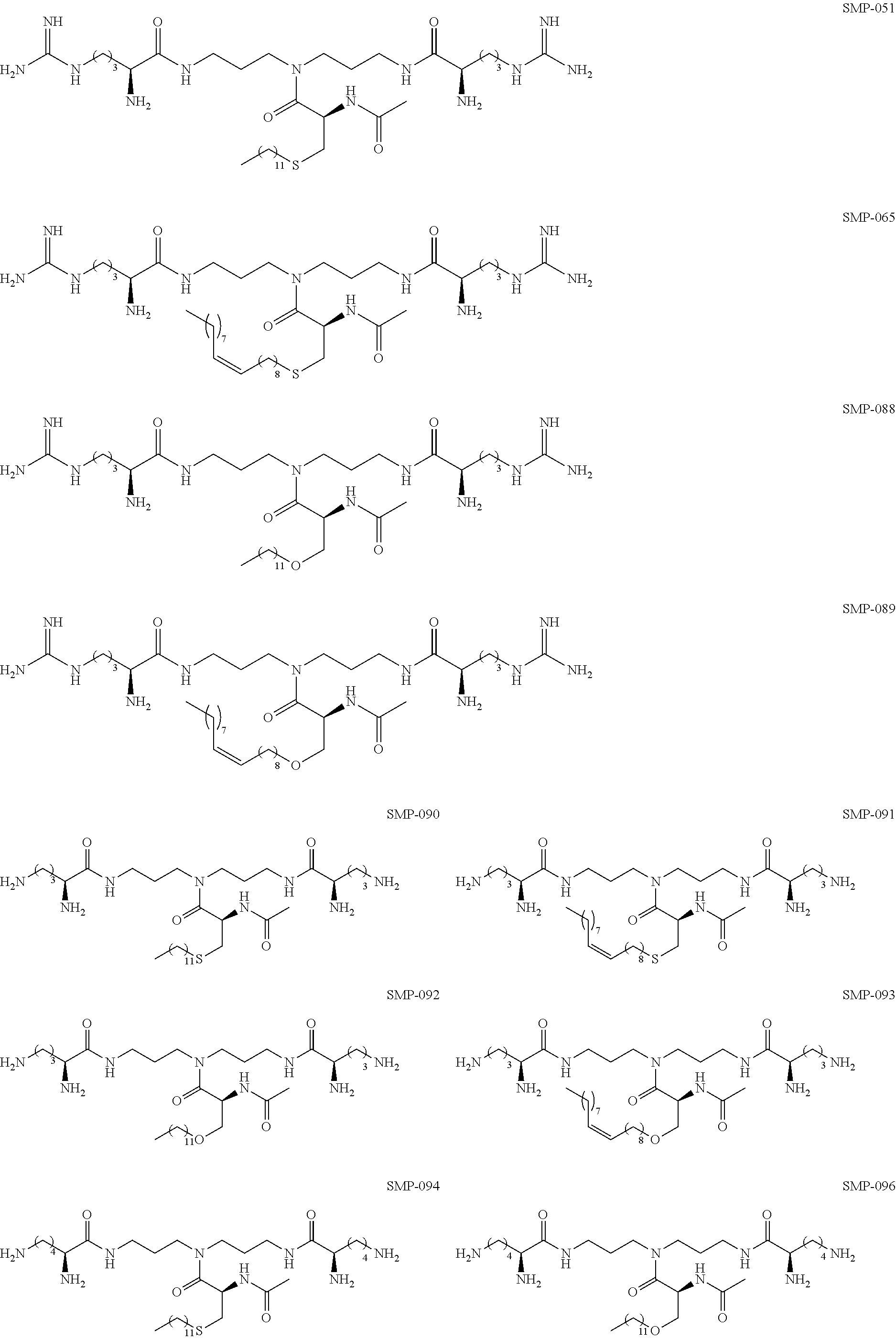

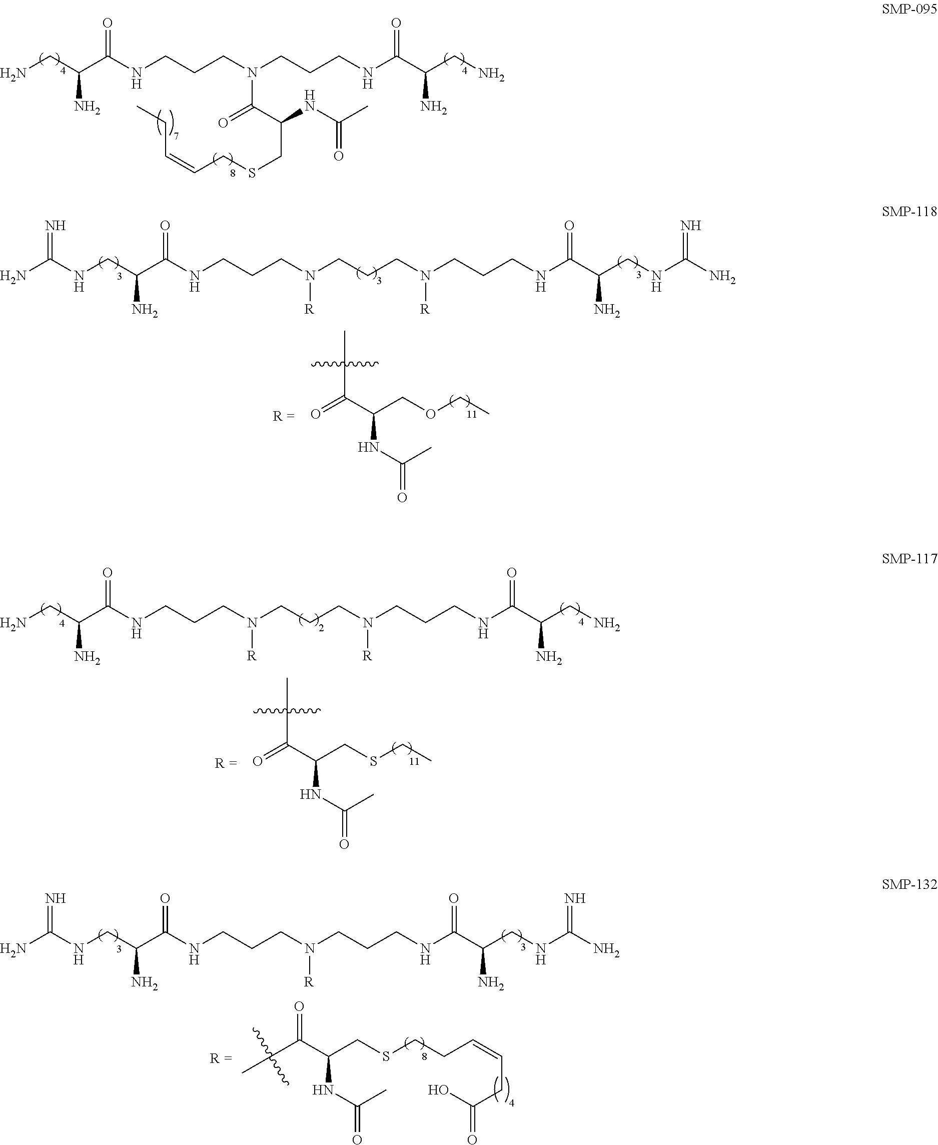

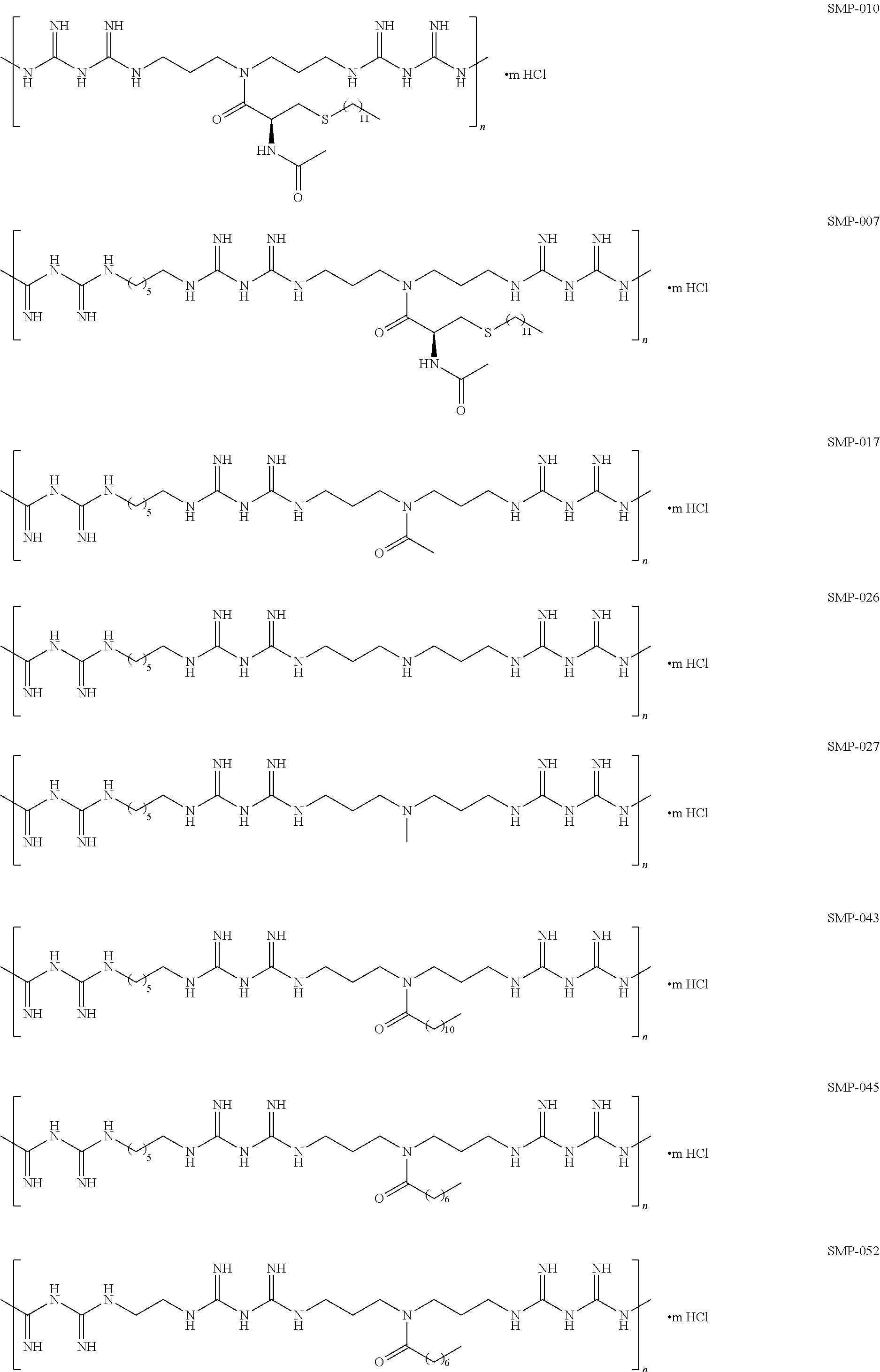

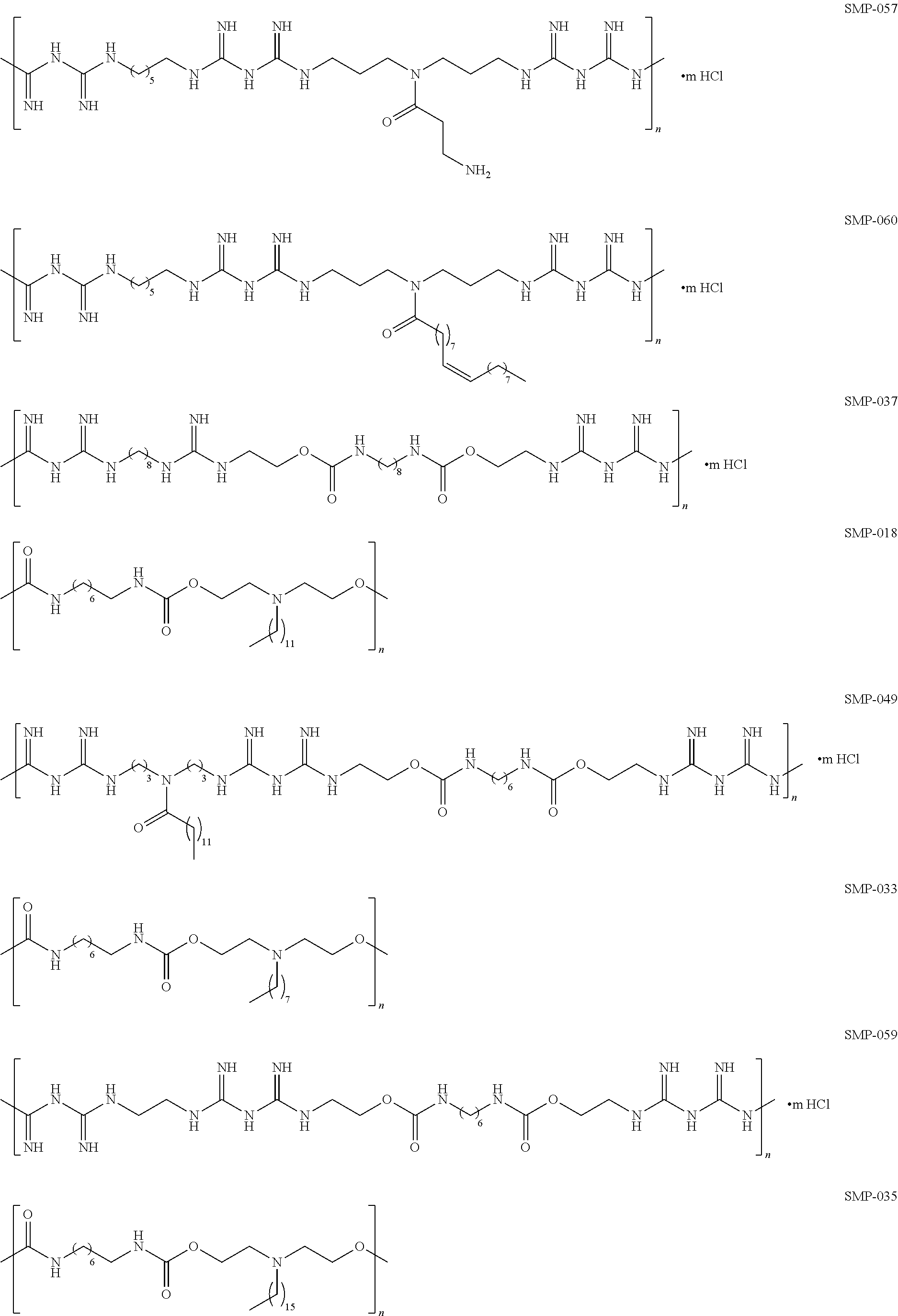

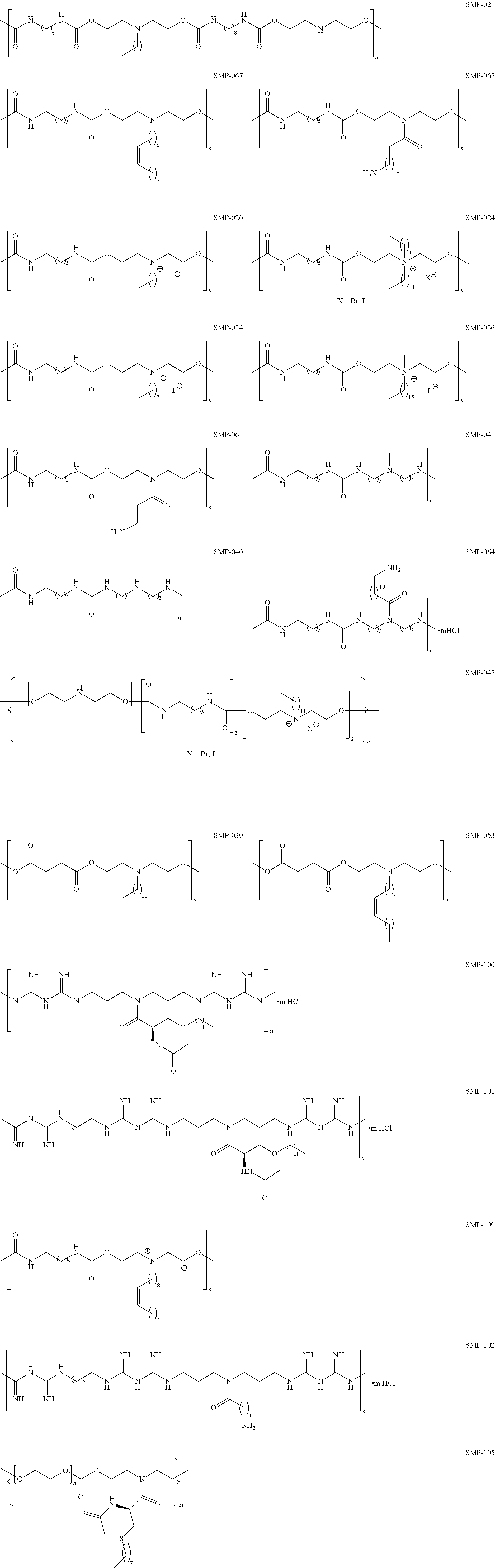

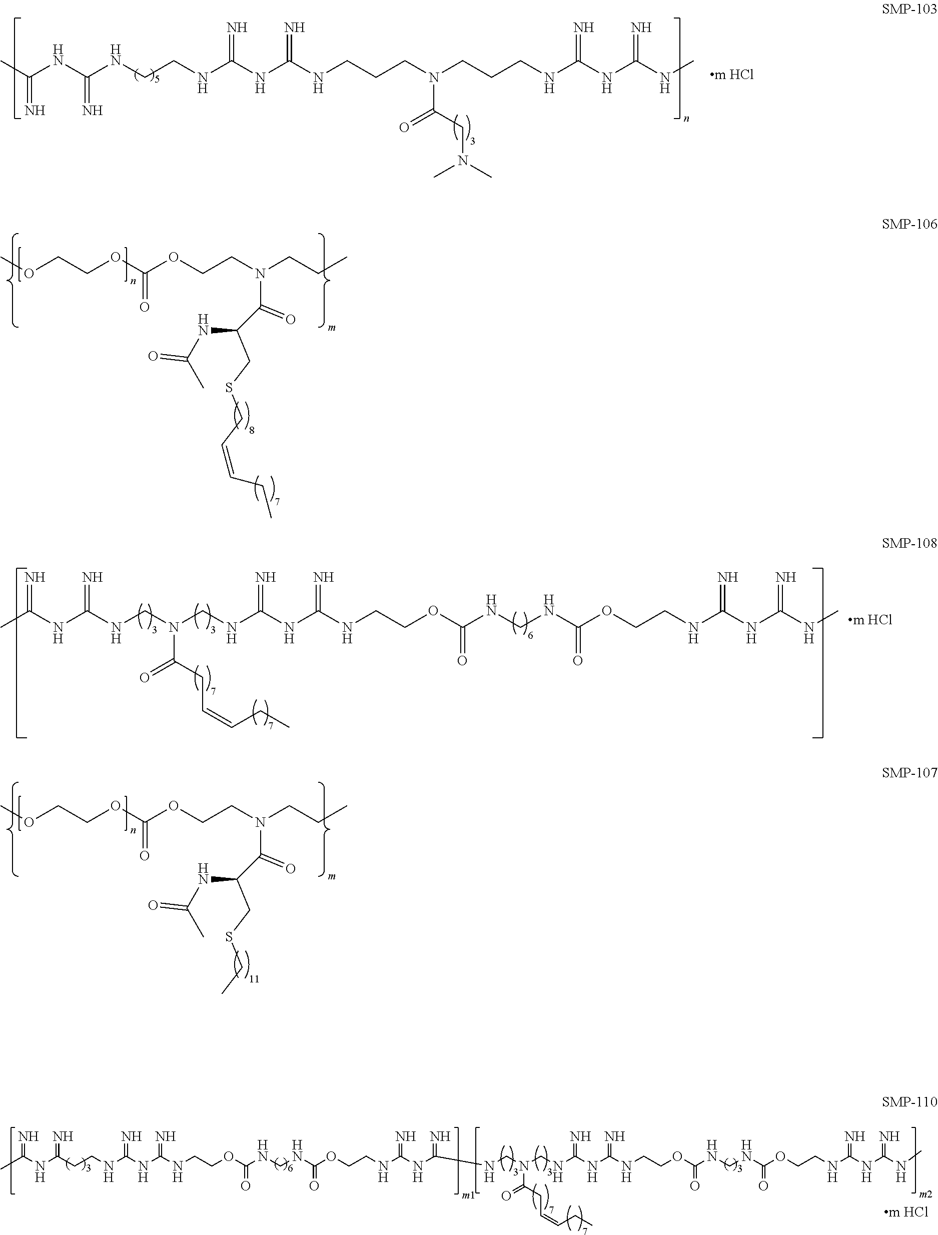

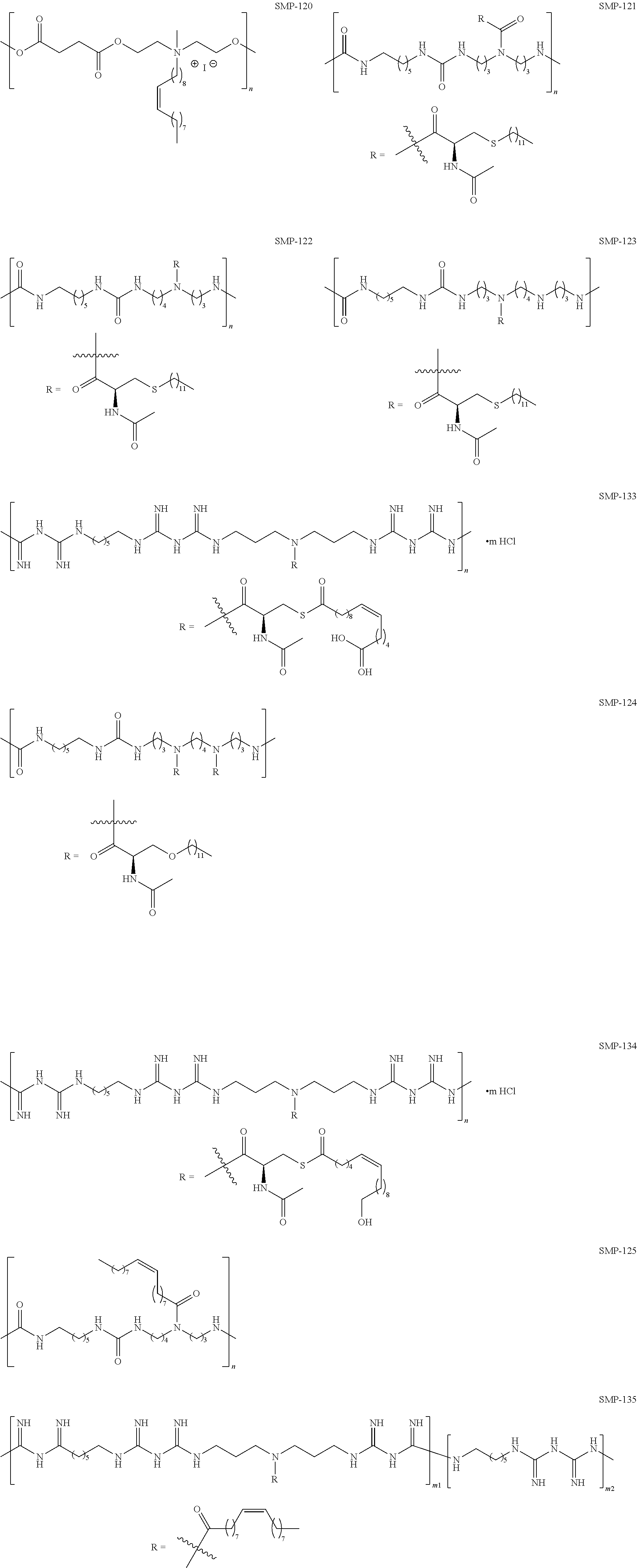

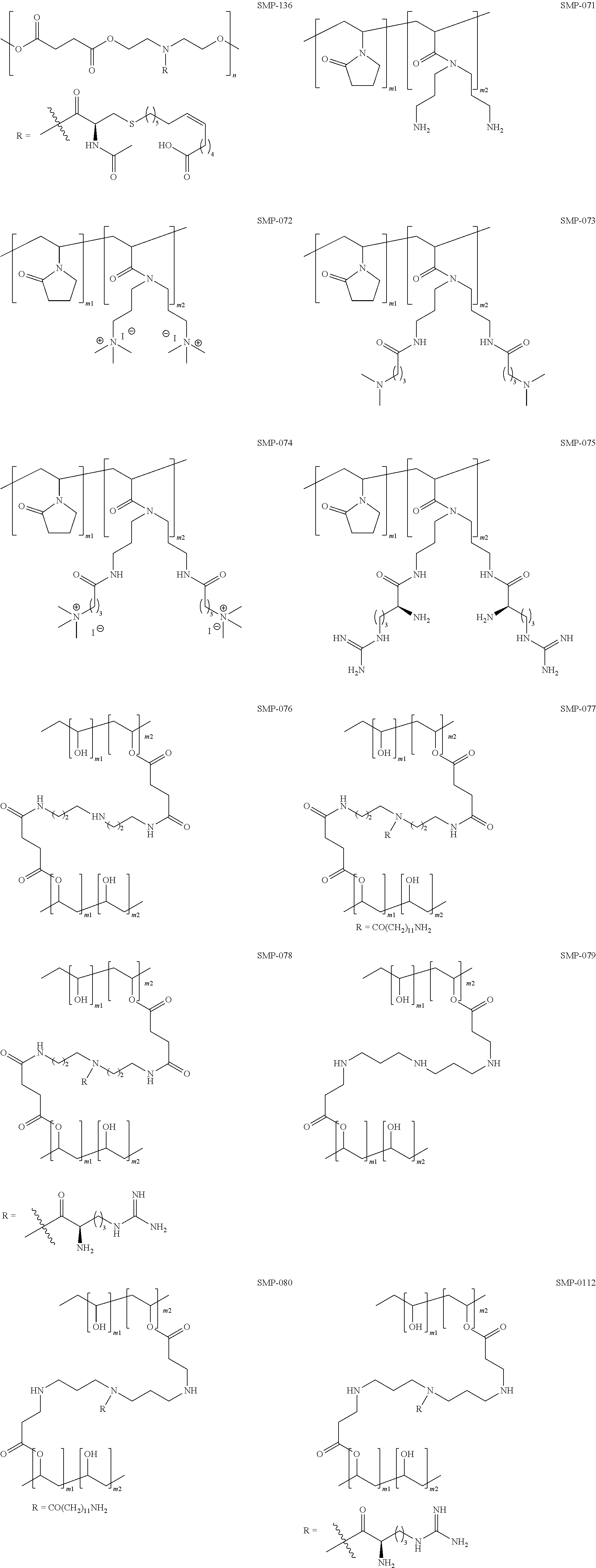

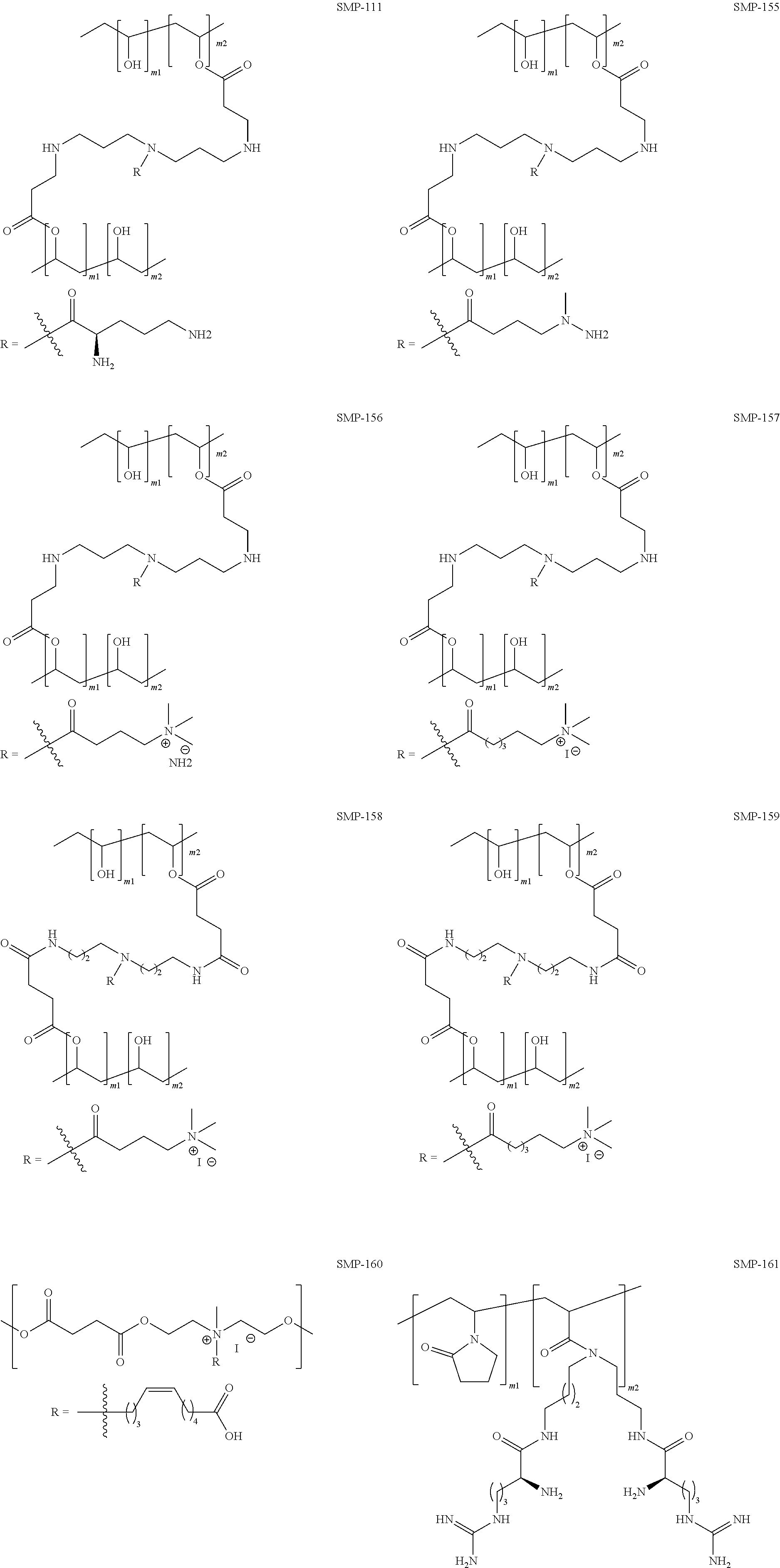

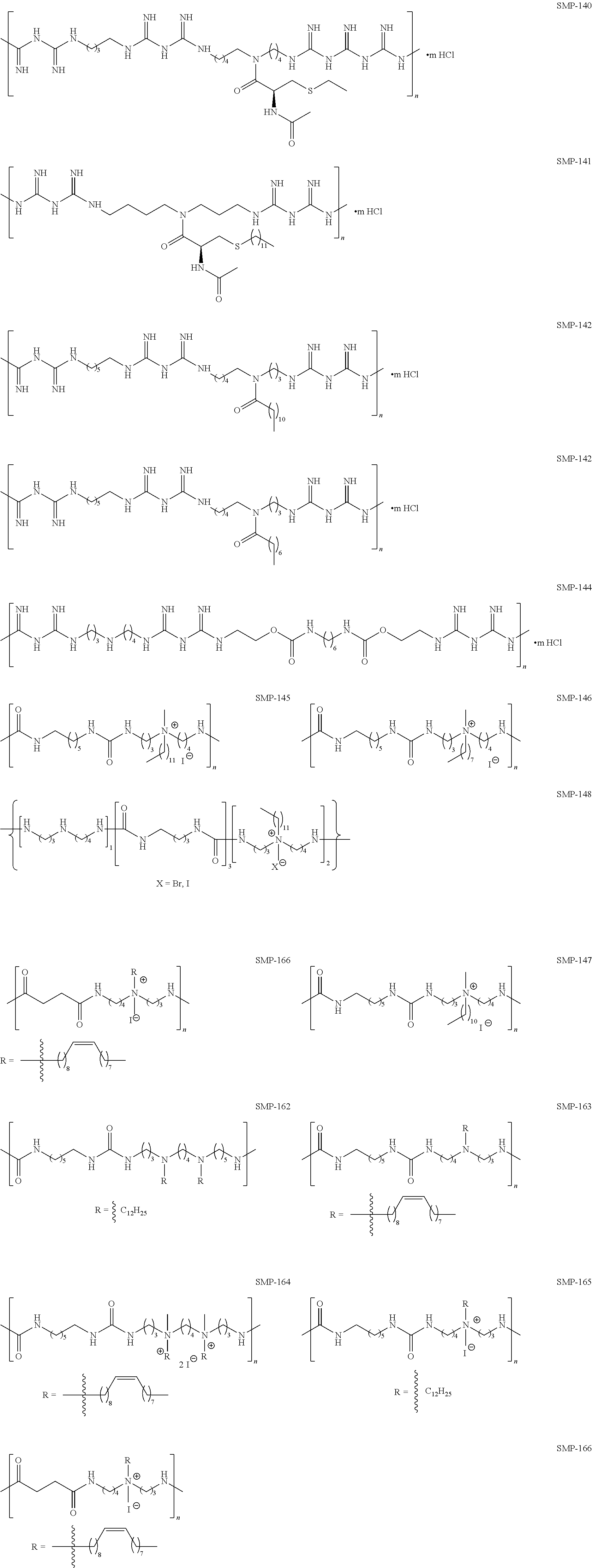

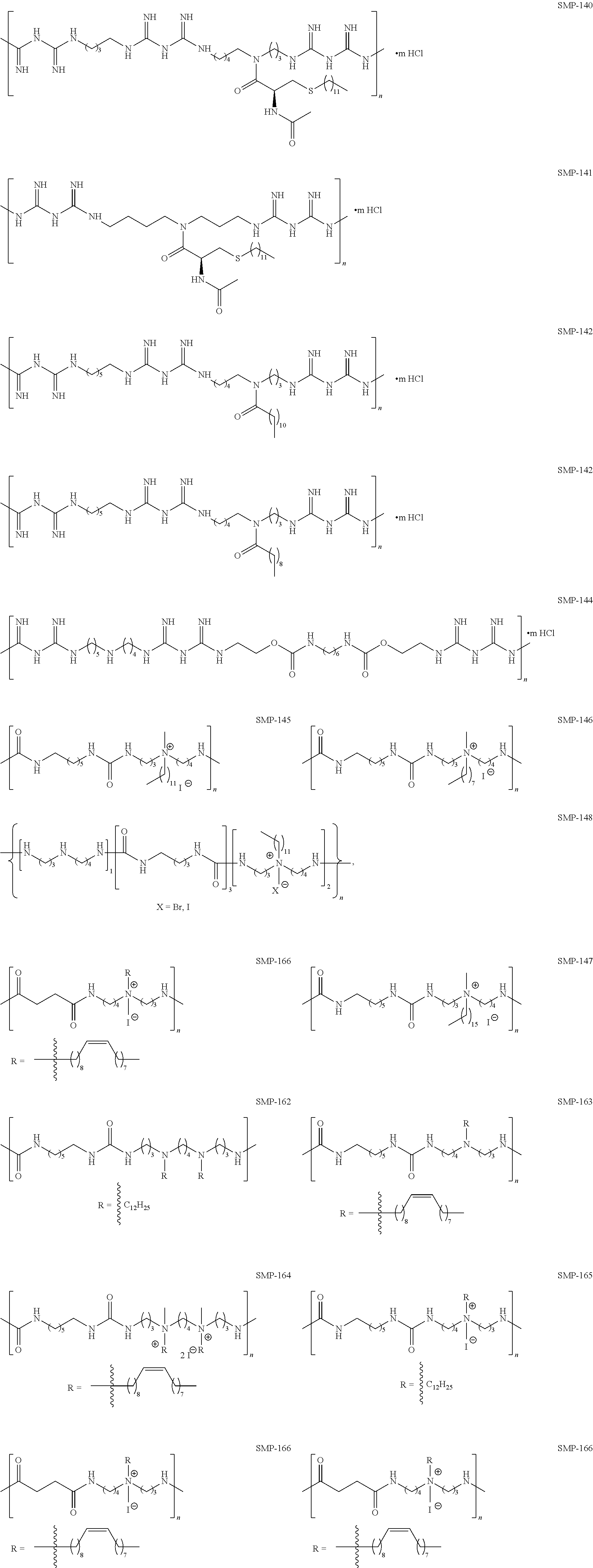

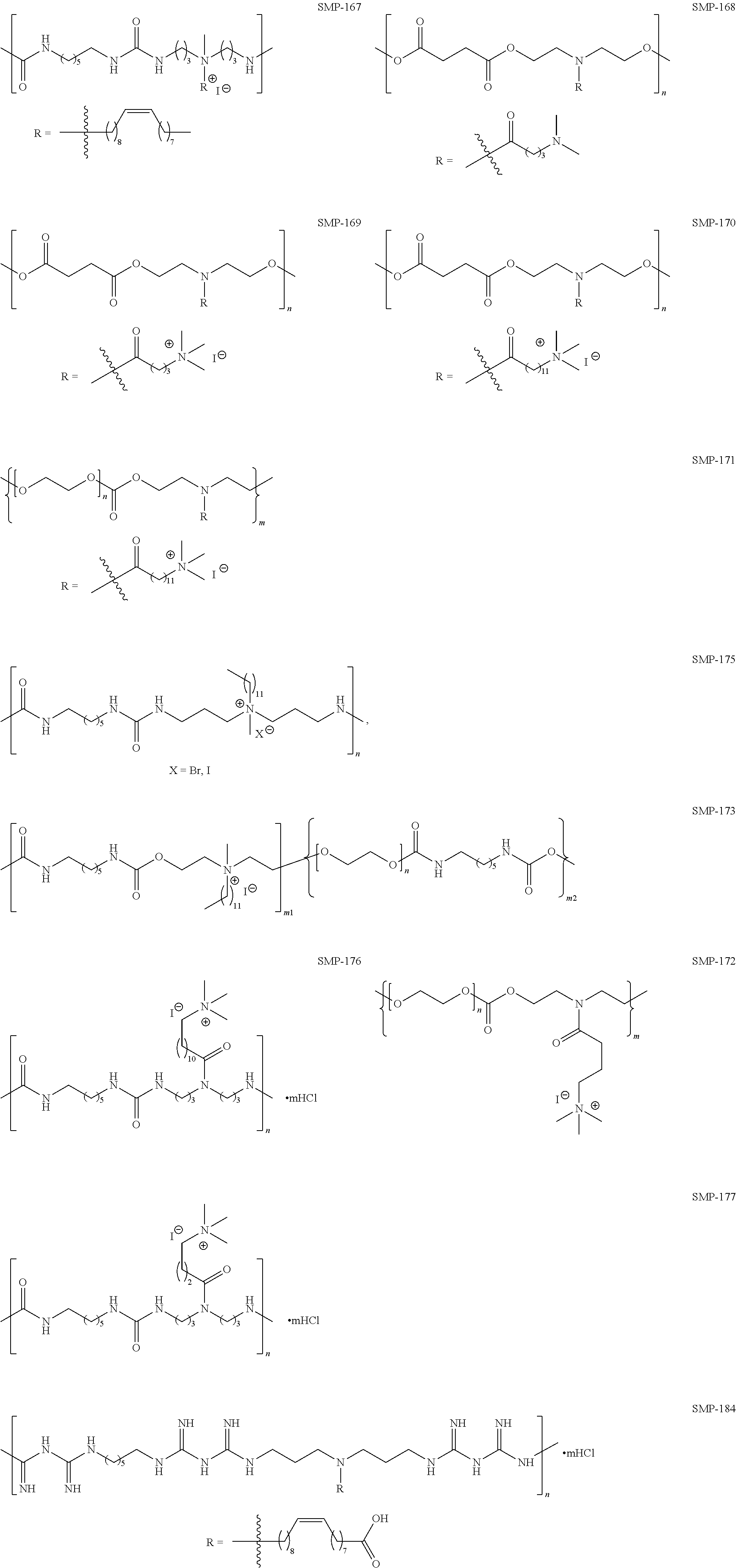

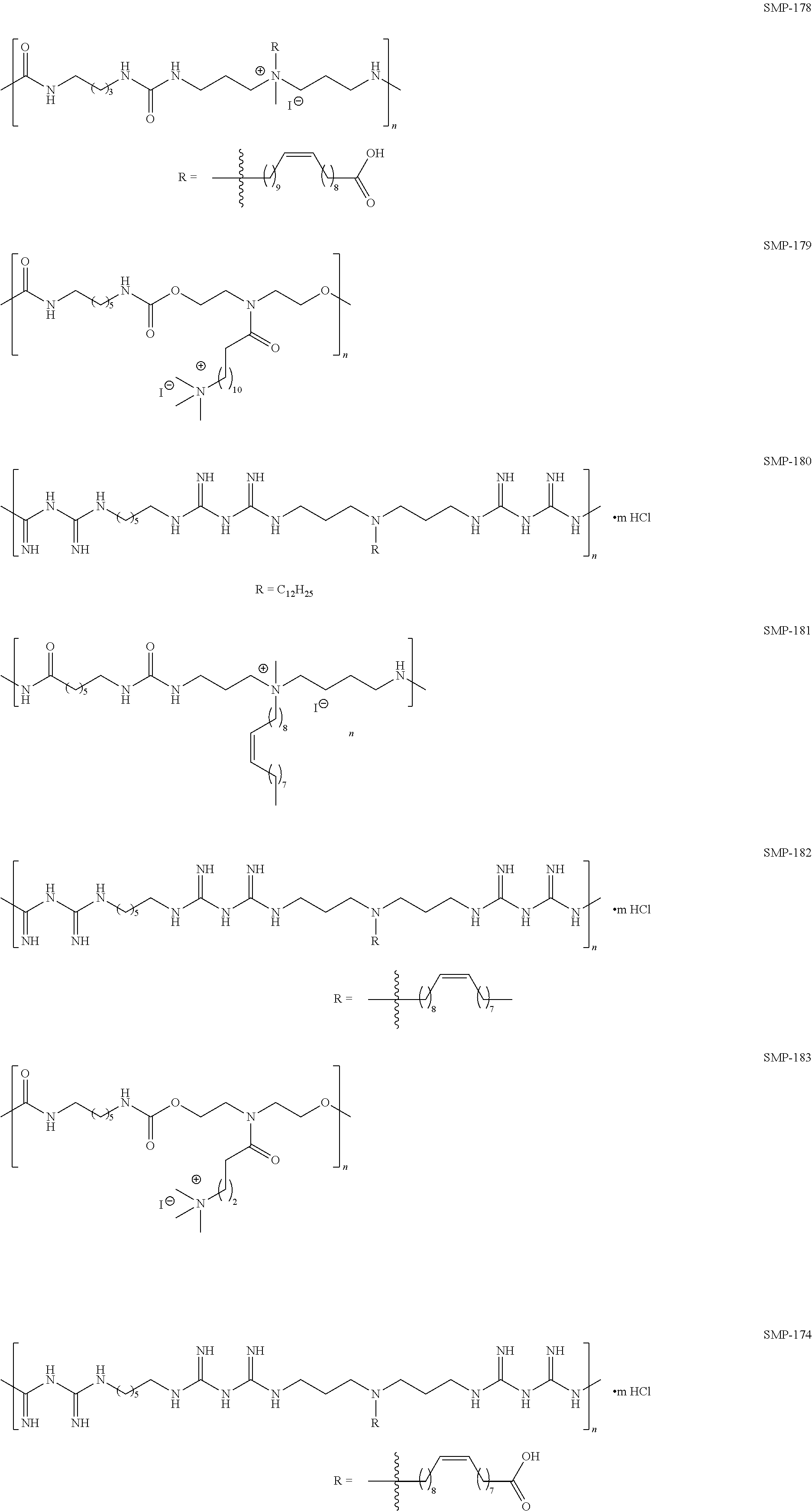

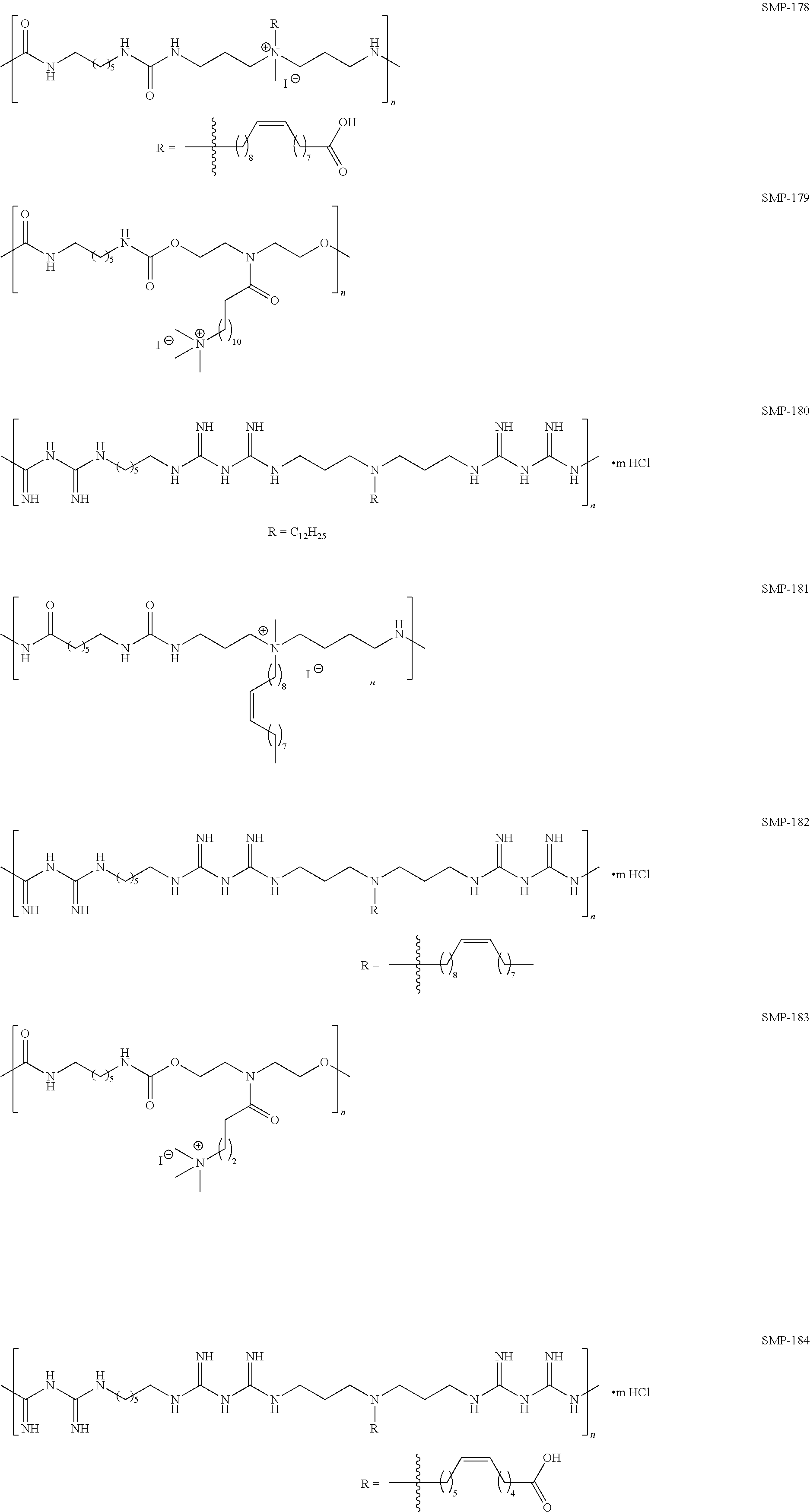

51. A compound of Formula II: 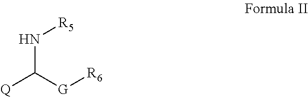
wherein ‘G’ is oxygen (—O—), sulphur (—S—), carbon (—C—), aryl, heteroaryl groups;
Q is carboxylic acid, vinylacrylate, methylacrylate, halogen selected from fluoride, bromide, chloride or iodide, alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 and —(CH₂)p-CH═CH₂ with p=1-10, and substituted with carboxylic group, acrylic group, halogen selected from fluorine, chlorine, bromine or iodine, wherein ‘G’ is oxygen (—O—), sulphur (—S—), wherein R₅ and R₆ is independently hydrogen, alkyl, straight alkyl chain, branched alkyl chain, —(CH₂)n wherein n=1 to 30, alkenyl, alkynyl, aryl, heteroaryl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10 and —(CH₂)p-CH═CH₂ with p=1-10, and wherein each of R₅ and R₆ is optionally substituted with primary, secondary, tertiary or quaternary amino group, hydroxyl group, thiol group, carboxylic group, acrylic group, halogen selected from fluorine, chlorine, bromine or iodine, —COR₇, —COR₈ wherein R₇ and R₈ is alkyl, straight alkyl chain, branched alkyl chain, —(CH₂)n wherein n=1 to 30, alkenyl, alkynyl, —(CH₂)p-CH═CH—CH₂—(CH₂)p-CH₃ with p=1-10, —(CH₂)p-CH═CH—(CH₂)p-CH═CH—(CH₂)p-CH₃ with p=1-10, —(CH₂)p-CH═CH₂ with p=1-10, aryl, heteroaryl and wherein each of the R₇ and R₈ is optionally substituted with primary, secondary, tertiary or quaternary amino group, hydroxyl group, thiol group, carboxylic group, acrylic group, halogen selected from fluorine, chlorine, bromine or iodine, C-terminal amino acids with D or L configuration, or oligo-peptide.

52. The compound of claim 51, wherein the compound of Formula II is

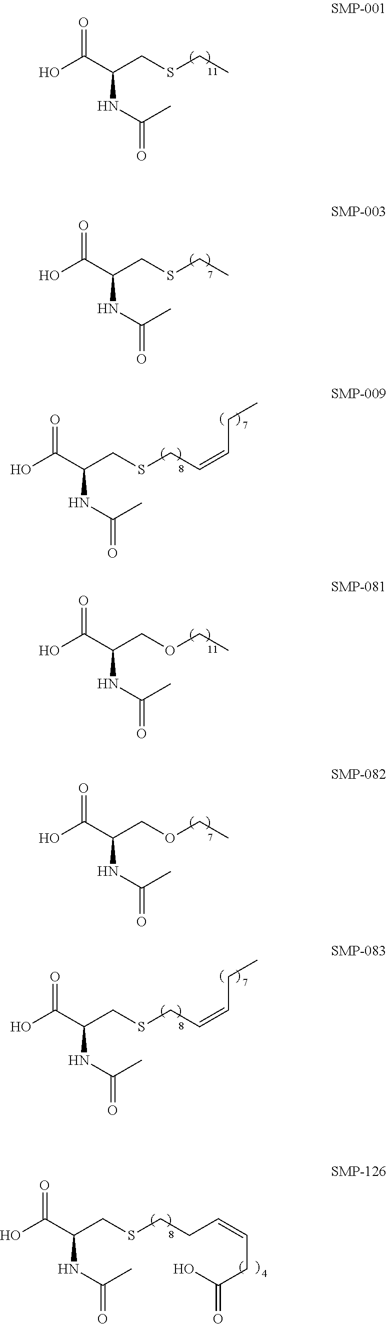

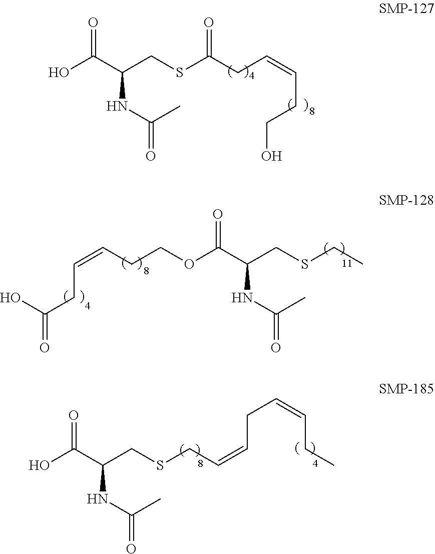

53. A process for preparing compound of Formula Ia as defined in claim 42 comprising step of reacting Formula II with polyamines, polyamine derivatives, or a combination thereof.

54. The compound of claim 1, wherein the compound of Formula I, Ib and Ic are obtained by self-polymerization of monomeric unit Formula Ia, or hetero-polymerization of Formula Ia with compound(s) selected from hexamethylenediamine, 1,6-bis(N³-cyano-N¹-guanidino)hexane, succinic anhydride, ethanol amine, PEG, acrylic or carboxylic derivative of ethanol amine or polyamines, bis(2-aminoethyl) hexane-1,6-diyldicarbamate, different monomeric units functionalized with guanidine, biguanidine, urethane, mixed guanidine-urethane, urea, ester, amide, carbonate, carbamate, or copolymerization or crosspolymerization with polymers or polymer derivatives selected from polyvinylpyrolidone (PVP), polyglycolic acid (PGA), polymethacrylate, polyacryl, polyacrylic acid (PAA), alginic acid, chitosan, PLGA, ethylene vinyl acetic acid, polyester, polyamide, polycarbamate, polycarbonate, PEG, PLA, PLA-PEG co-polymer, PHMB, polyguanidine, polybiguanidine, polyurethane, polybiguanidine-polyurethane, polyurea, polyester, polyamide or polycarbonate, or any combinations thereof, wherein the polyamine is spermine, spermidine, norspermidine or putrescine, or any combinations thereof.

55. A composition comprising a compound of formula I as defined in claim 1 along with pharmaceutically acceptable excipient.

56. The composition of claim 55 comprising from about 0.1% to 20% (w/w) of the compound and about 80% to 99.9% (w/w) of the pharmaceutically acceptable excipient, preferably the composition comprises from about 0.1% to 5% (w/w) of the compound and about 95% to 99.9% (w/w) of the pharmaceutically acceptable excipient; wherein the excipient is selected from drug delivery carrier, emollient, moisturizer, emulsifier, stabilizer, surfactant, oil, lipid, wax, solubilizer, rheology modifier, thickening agent, gelling agent, preservative, antioxidant, film forming agent, pH modifier or other conventionally known pharmaceutically acceptable excipient, or any combination of excipients thereof; wherein the composition is formulated into dosage forms selected from cream, gel, hydrogel, ointment, lotion, liposomal gel, micronized gel, powder, spray, solution, film, liquid bandage, patch, coating material on implant, coating material on a surface or matrix, wound dressing, or other suitable drug delivery vehicles, or any combination of dosage forms thereof.

57. The composition of claim 55, wherein the composition treats a microbial infection or disease, and is administered to a subject in need thereof through modes selected from topical administration, local administration at wound infection or surgical site infection, intravenous administration, intramuscular administration, intraperitoneal administration, hepatoportal administration, intra articular administration or pancreatic duodenal artery administration, or any combination of modes thereof.

58. The composition of claim 55, wherein the drug delivery carrier is biocompatible polymer, biodegradable polymer, bioabsorbable polymer or hydrogel forming polymer, or any combination of polymers thereof; and wherein the polymer is selected from polyvinylpyrolidine (PVP), polyglycolic acid (PGA), polyacrylic acid (PAA), alginic acid, chitosan, poly(lactic-co-glycolic acid) (PLGA), ethylene vinyl acetic acid, polyester, polyamide, polycarbamate, polycarbonate, polyethylene glycol (PEG), polylactic acid (PLA), PLA-PEG co-polymer, polyhexamethylene biguanide (PHMB), dextran, starch, polyguanidine, polybiguanidine, polyurethane, polybiguanidine-polyurethane, polyurea, polyester, polyamide or polycarbonate, or any combinations thereof.

59. A method of treating a microbial infection or disease comprising administering a compound of claim 1 or a composition comprising said compound, to a subject in need thereof.

60. The method of claim 59, wherein the microbial infection is a bacterial infection, fungal infection, biofilm associated infection, a community acquired infection, health care-associated infection (HCAI) or a combination thereof; and wherein the community acquired infection is selected from superficial skin infection, topical wound infection, burn infection or diabetic foot infection, or any combinations thereof, and the health care-associated infection (HCAI) is selected from surgical site infections (SSIs), central line-associated bloodstream infections (CLABSI), catheter-associated urinary tract infections (CAUTI), ventilator-associated pneumonia (VAP), medical device associated infections or other health care-associated infection, or any combinations thereof; wherein the surgical site infection (SSI) is an implant associated infection caused by implant selected from orthopedic device, coronary stent, central venous and urinary catheters, heart valve, vascular graft, central nervous system implant, cochlear or dental implant, or any combinations thereof; wherein the microbial infection is caused by microbe selected from Pseudomonas spp., Acinetobacter spp., Enterobacter spp., Klebsiella spp., Escherichia spp., Staphylococcus spp., Streptococcus spp., Enterococcus spp., Haemophilus spp., Propionibacterium spp. and Bacillus spp. Bacteroides spp., Fusobacterium spp., Clostridium spp., Candida spp. Malassezia spp. or Trichophyton spp., P. aeruginosa, A. baumannii, E. aerogenes, K. pneumoniae, E. coli, S. epidermidis, S. aureus, E. faecium, S. pyogenes, H. influenzae, P. acnes, and Bacillus anthracis, B. fragilis, C. septicum, C. albicans, M. furfur or T. rubrum, or any combinations thereof; and drug resistant microbe strains of S. aureus, Pseudomonas spp., Acinetobacter spp., Enterobacter spp., Klebsiella spp., Escherichia spp., Staphylococcus spp., Propionibacterium spp., Bacillus spp., Streptococcus spp., Enterococcus spp., Haemophilus spp., Bacteroids, Fusobacterium, Clostridium, Candida spp., Malassezia spp. or Trichophyton spp., or any combinations of drug resistant microbes thereof; and wherein the drug resistant S. aureus is methicillin-resistant S. aureus (MRSA), methicillin resistant Staphylococcus epidermidis (MRSE), vancomycin resistant S. aureus (VRSA), vancomycin resistant S. epidermidis (VRSE) or vancomycin intermediate resistant S. aureus (VISA), or any combinations thereof; and wherein the compound has a minimum inhibitory concentration ranging from about 1 microgram/ml to 500 microgram/ml, preferably 1 microgram/ml to 200 microgram/ml and/or a minimum biofilm disruption concentration (MBDC) ranging from about 0.01 milligram/ml to 20 milligram/ml, preferably about 0.01 milligram/ml to 10 milligram/ml.

61. The method of claim 60, further comprising co-administering one or more additional anti-microbial agent to the subject.

62. The compound of claim 1 or composition comprising said compound, wherein the compound has wound healing activity.

63. The compound of claim 1, composition comprising said compound or method of said compound , wherein the compounds SMP-047, SMP-020, SMP-036, SMP-042, SMP-067, SMP-062, SMP-066, SMP-023, SMP-045, SMP-043 and SMP-026 possess potent antifungal activity, preferably against Candida species wherein the compounds SMP-001, SMP-002, SMP-007, SMP-020, SMP-043, SMP-045, SMP-027, SMP-037, SMP-034, SMP-036, SMP-042, SMP-047, SMP-051, SMP-030 and SMP-126 possess biofilm disruption activity, preferably biofilm disruption activity against biofilm formed by gram positive, gram negative pathogen or a combination thereof.
